(19) United States
(12) Patent Application Publication
(10) Pub. No.: US 2026/0216243 A1
(43) Pub. Date: Jul. 30, 2026

(54) ANTIGEN-BINDING MOLECULES

(71) Applicant: TWAIN THERAPEUTICS PTE. LTD., Singapore (SG)

(72) Inventors: John Edward Connolly, Connolly (SG); Anna-Marie Fairhurst, Singapore (SG); Xiaohua Zhou, Singapore (SG)

(73) Assignee: TWAIN THERAPEUTICS PTE. LTD., Singapore (SG)

(21) Appl. No.: 19/145,764

(22) PCT Filed: Jan. 5, 2024
(86) PCT No.: PCT/EP2024/050242

§ 371 Date: Jul. 3, 2025

Related U.S. Application Data
(60) Provisional application No. 63/437,470, filed on Jan. 6, 2023.

(51) Int. Cl. A61K 35/17; A61K 40/31; A61K 40/42; C07K 14/705; C07K 16/28

(52) U.S. Cl. CPC A61K 35/17; A61K 40/31; A61K 40/4217; C07K 14/7051; C07K 16/2866; A61K 2239/13; C07K 2317/22; C07K 2317/31; C07K 2317/565; C07K 2317/567; C07K 2317/569; C07K 2317/75; C07K 2317/76

(57) ABSTRACT

Antigen binding molecules comprising a γc-binding moiety, and a moiety that binds to IL-9Rα, are disclosed herein. Also disclosed are compositions comprising such antigen binding molecules, and uses and methods using the same.

This application claims priority from U.S. 63/437,470 filed 6 Jan. 2023, the contents and elements of which are herein incorporated by reference for all purposes.

### TECHNICAL FIELD

The present disclosure relates to the fields of molecular biology and methods of medical treatment and prophylaxis. In particular, the present disclosure relates to antigen-binding molecules that bind to polypeptides of γc-containing cytokine receptors.

### BACKGROUND

Interleukins play a central role in maintaining T cell homeostasis and mediating proper immune responses. Specifically, interleukins and associated cytokines serve as the means of communication for innate and adaptive immune cells as well as non-immune cells and tissues. Thus, interleukins have a critical role in cancer development, progression and control (Briukhovetska D. et al. Nat Rev Cancer 21, 481-499 (2021)).

The use of interleukins in therapy has shown much promise but has been associated with drawbacks and disappointing results.

The administration of IL-9 has been shown to be effective in the reduction of tumor growth in mice (Purwar et al., Nat Med. 2012; 18(8): 1248-1253). However, downsides of cytokine therapy are known.

IL-2 was the first interleukin to be approved for cancer treatment, although its use entails major safety concerns. The high dose of IL-2 that is required for effective treatment of certain diseases is highly toxic. Major adverse effects of such therapy include vascular leak syndrome (VLS), which results in accumulation of the intravascular fluid in organs such as lung and liver with subsequent pulmonary edema and liver damage. There is no treatment for VLS except withdrawing therapy.

Additionally, monotherapy with IL-15 and several engineered variants was ineffective (Waldman et al. (2020) Front Immunol. May 19; 11:868).

The common cytokine receptor gamma chain (common gamma chain, γc, or CD132) is a cytokine receptor polypeptide that is common to the cytokine receptor complexes of at least six different interleukin receptors (i.e. receptors of IL-2, IL-4, IL-7, IL-9, IL-15, and IL-21).

Cells expressing γc can form functional receptors for cytokine proteins and can transmit signals from one cell to another and direct programs of cellular differentiation. Heterodimerization of γc and other polypeptide(s) is necessary and sufficient for effective signal transduction through the interaction of their cytoplasmic domains and subsequent kinase activation of multiple signaling pathways. For example, heterodimerization of IL-9Rα and γc is necessary for effective IL-9 signal transduction.

Antigen-binding molecules that bind to γc and IL-2RP are disclosed e.g. in WO 2017/021540 A1.

### SUMMARY

In one aspect, the present disclosure provides an antigen-binding molecule, optionally isolated, comprising: (i) a γc-binding moiety, and (ii) a moiety that binds to IL-9Rα.

In some embodiments, the antigen-binding molecule is an agonist of a γc-containing cytokine receptor.

In some embodiments, the antigen-binding molecule is an antagonist of a γc-containing cytokine receptor.

In some embodiments, the antigen-binding molecule is an agonist of a γc:IL-9Rα receptor.

In some embodiments, the antigen-binding molecule is an antagonist of a γc:IL-9Rα receptor.

In some embodiments, the antigen-binding molecule increases signalling mediated by a γc:IL-9Rα receptor.

In some embodiments, the antigen-binding molecule decreases signalling mediated by a γc:IL-9Rα receptor.

In some embodiments, the IL-9Rα-binding moiety comprises a single-domain antibody sequence the IL-9Rα-binding moiety comprises a single-domain antibody sequence incorporating the following CDRs:
(2SAT363) CDR1 having the amino acid sequence of SEQ ID NO:378
   CDR2 having the amino acid sequence of SEQ ID NO:379
   CDR3 having the amino acid sequence of SEQ ID NO:380; or
(2SAT57) CDR1 having the amino acid sequence of SEQ ID NO:386
   CDR2 having the amino acid sequence of SEQ ID NO:387
   CDR3 having the amino acid sequence of SEQ ID NO:388.

In some embodiments, the IL-9Rα-binding moiety comprises, or consists of, an amino acid sequence having at least 70% sequence identity to the amino acid sequence of SEQ ID NO:385 or 393.

In some embodiments, the IL-9Rα-binding moiety comprises a single-domain antibody sequence incorporating the following FRs:
(2SAT363) FR1 having the amino acid sequence of SEQ ID NO:381
   FR2 having the amino acid sequence of SEQ ID NO:382
   FR3 having the amino acid sequence of SEQ ID NO:383
   FR4 having the amino acid sequence of SEQ ID NO:384; or
(2SAT57) FR1 having the amino acid sequence of SEQ ID NO:389
   FR2 having the amino acid sequence of SEQ ID NO:390
   FR3 having the amino acid sequence of SEQ ID NO:391
   FR4 having the amino acid sequence of SEQ ID NO:392.

In some embodiments, the γc-binding moiety comprises a single-domain antibody sequence incorporating the following CDRs:
CDR1 having the amino acid sequence of SEQ ID NO:370
CDR2 having the amino acid sequence of SEQ ID NO:371
CDR3 having the amino acid sequence of SEQ ID NO:372.

In some embodiments, the γc-binding moiety comprises, or consists of, an amino acid sequence having at least 70% sequence identity to the amino acid sequence of SEQ ID NO:377.

In some embodiments, the γc-binding moiety comprises a single-domain antibody sequence incorporating the following FRs:
FR1 having the amino acid sequence of SEQ ID NO:373
FR2 having the amino acid sequence of SEQ ID NO:374
FR3 having the amino acid sequence of SEQ ID NO:375
FR4 having the amino acid sequence of SEQ ID NO:376.

In a second aspect, the disclosure provides an antigen-binding molecule, optionally isolated, which binds to IL-9Rα, wherein the antigen-binding molecule comprises a single-domain antibody sequence incorporating the following CDRs:
(2SAT363) CDR1 having the amino acid sequence of SEQ ID NO:378
   CDR2 having the amino acid sequence of SEQ ID NO:379
   CDR3 having the amino acid sequence of SEQ ID NO:380; or
(2SAT57) CDR1 having the amino acid sequence of SEQ ID NO:386
   CDR2 having the amino acid sequence of SEQ ID NO:387
   CDR3 having the amino acid sequence of SEQ ID NO:388.

In some embodiments, the antigen-binding molecule which binds to IL-9Rα comprises, or consists of, an amino acid sequence having at least 70% sequence identity to the amino acid sequence of SEQ ID NO:385 (2SAT363) or 393 (2SAT57).

In some embodiments, the antigen-binding molecule comprises a single-domain antibody sequence having at least 70%, e.g. one of ≥80%, ≥85%, ≥90%, ≥91%, ≥92%, ≥93%, ≥94%, ≥95%, ≥96%, ≥97%, 298%, ≥99% or 100% amino acid sequence identity to an amino acid sequence of SEQ ID NO: 385 (2SAT363) or 393 (2SAT57).

In some embodiments, the antigen-binding molecule which binds to IL-9Rα comprises a single-domain antibody sequence incorporating the following FRs:
(2SAT363) FR1 having the amino acid sequence of SEQ ID NO:381
   FR2 having the amino acid sequence of SEQ ID NO:382
   FR3 having the amino acid sequence of SEQ ID NO:383
   FR4 having the amino acid sequence of SEQ ID NO:384; or
(2SAT57) FR1 having the amino acid sequence of SEQ ID NO:389
   FR2 having the amino acid sequence of SEQ ID NO:390
   FR3 having the amino acid sequence of SEQ ID NO:391
   FR4 having the amino acid sequence of SEQ ID NO:392.

In some embodiments, the antigen-binding molecule is a multispecific antigen-binding molecule, and further comprises an antigen-binding moiety that binds to an antigen other than IL-9Rα. In some embodiments, the antigen other than IL-9Rα is γc.

In a third aspect, the present disclosure provides an antigen-binding molecule, optionally isolated, which binds to γc, wherein the antigen-binding molecule comprises a single-domain antibody sequence incorporating the following CDRs:
CDR1 having the amino acid sequence of SEQ ID NO:370
CDR2 having the amino acid sequence of SEQ ID NO:371
CDR3 having the amino acid sequence of SEQ ID NO:372.

In some embodiments, the antigen-binding molecule which binds to γc comprises, or consists of, an amino acid sequence having at least 70% sequence identity to the amino acid sequence of SEQ ID NO:377. In some embodiments, the antigen-binding molecule comprises a single-domain antibody sequence having at least 70%, e.g. one of ≥80%, ≥85%, ≥90%, ≥91%, ≥92%, ≥93%, ≥94%, ≥95%, ≥96%, 297%, ≥98%, ≥99% or 100% amino acid sequence identity to an amino acid sequence of SEQ ID NO:377.

In some embodiments, the antigen-binding molecule which binds to γc comprises a single-domain antibody sequence incorporating the following FRs:
FR1 having the amino acid sequence of SEQ ID NO:373
FR2 having the amino acid sequence of SEQ ID NO:374
FR3 having the amino acid sequence of SEQ ID NO:375
FR4 having the amino acid sequence of SEQ ID NO:376.

In some embodiments, the antigen-binding molecule which binds to γc is a multispecific antigen-binding molecule, and wherein the antigen-binding molecule further comprises an antigen-binding moiety that binds to an antigen other than γc. In some embodiments, the antigen other than γc is IL-9Rα.

In some embodiments, the antigen-binding molecule which binds to IL-9Rα or the antigen-binding molecules which binds to γc is a bispecific antigen-binding molecule.

In some embodiments, the antigen-binding molecule further comprises: (iii) an antigen-binding moiety that binds to a target antigen other than a γc-containing cytokine receptor polypeptide.

In some embodiments, the target antigen other than a γc-containing cytokine receptor polypeptide is a disease-associated antigen or an antigen expressed by an immune cell.

The present disclosure also provides a chimeric antigen receptor (CAR), comprising an antigen-binding molecule according to the present disclosure.

The present disclosure also provides a nucleic acid, or a plurality of nucleic acids, optionally isolated, encoding an antigen-binding molecule according to the present disclosure or a CAR according to the present disclosure.

The present disclosure also provides an expression vector, or a plurality of expression vectors, comprising a nucleic acid or a plurality of nucleic acids according to the present disclosure.

The present disclosure also provides a cell comprising an antigen-binding molecule according to the present disclosure, a CAR according to the present disclosure, a nucleic acid or a plurality of nucleic acids according to the present disclosure, or an expression vector or a plurality of expression vectors according to the present disclosure.

The present disclosure also provides a method comprising culturing a cell according to the present disclosure under conditions suitable for expression of an antigen-binding molecule or CAR by the cell.

The present disclosure also provides a composition comprising an antigen-binding molecule according to the present disclosure, a CAR according to the present disclosure, a nucleic acid or a plurality of nucleic acids according to the present disclosure, an expression vector or a plurality of expression vectors according to the present disclosure, or a cell according to the present disclosure, and a pharmaceutically acceptable carrier, diluent, excipient or adjuvant.

The present disclosure also provides an antigen-binding molecule according to the present disclosure, a CAR according to the present disclosure, a nucleic acid or a plurality of nucleic acids according to the present disclosure, an expression vector or a plurality of expression vectors according to the present disclosure, a cell according to the present disclosure, or a composition according to the present disclosure, for use in a method of treatment or prophylaxis.

The present disclosure also provides a use of an antigen-binding molecule according to the present disclosure, a CAR according to the present disclosure, a nucleic acid or a plurality of nucleic acids according to the present disclosure, an expression vector or a plurality of expression vectors according to the present disclosure, a cell according to the present disclosure, or a composition according to the present disclosure, in the manufacture of a medicament for use in a method of treatment or prophylaxis.

The present disclosure also provides a method of treatment or prophylaxis, comprising administering to a subject in need thereof a therapeutically or prophylactically effective amount of an antigen-binding molecule according to the present disclosure, a CAR according to the present disclosure, a nucleic acid or a plurality of nucleic acids according to the present disclosure, an expression vector or a plurality of expression vectors according to the present disclosure, a cell according to the present disclosure, or a composition according to the present disclosure.

In some embodiments, the method of treatment or prophylaxis is a method of treating or preventing a disease/condition characterised by T cell dysfunction, a cancer, an infectious disease, or an autoimmune disease.

In some embodiments, the cancer is selected from the group consisting of: colon cancer, colon carcinoma, colorectal cancer, nasopharyngeal carcinoma, cervical carcinoma, oropharyngeal carcinoma, gastric carcinoma, hepatocellular carcinoma, head and neck cancer, head and neck squamous cell carcinoma (HNSCC), oral cancer, laryngeal cancer, prostate cancer, lung cancer, small cell lung cancer, non-small cell lung cancer, bladder cancer, urothelial carcinoma, melanoma, advanced melanoma, renal cell carcinoma, ovarian cancer or mesothelioma.

In some embodiments, the method of treatment or prophylaxis is a method of treating or preventing a disease/condition in which a Th2 immune response is pathologically-implicated.

The present disclosure also provides an in vitro complex, optionally isolated, comprising an antigen-binding molecule according to the present disclosure, or a CAR according to the present disclosure, bound to γc and a polypeptide of a γc-containing cytokine receptor other than γc.

The present disclosure also provides a method for generating or expanding a population of cells expressing a γc-containing cytokine receptor, comprising contacting a cell expressing a γc-containing cytokine receptor in vitro, in vivo or ex vivo with an antigen-binding molecule according to the present disclosure.

The present disclosure also provides a method for increasing the proliferation, survival and/or effector activity of a cell expressing a γc-containing cytokine receptor, comprising contacting a cell expressing a γc-containing cytokine receptor in vitro, in vivo or ex vivo with an antigen-binding molecule according to the present disclosure.

The present disclosure also provides a method for reducing the number/proportion of (e.g. depleting or increasing the depletion of) cells expressing a γc-containing cytokine receptor, comprising contacting a cell expressing a γc-containing cytokine receptor in vitro, in vivo or ex vivo with an antigen-binding molecule according to the present disclosure, or a CAR according to the present disclosure.

The present disclosure also provides a method for decreasing the proliferation, survival and/or effector activity of a cell expressing a γc-containing cytokine receptor, comprising contacting a cell expressing a γc-containing cytokine receptor in vitro, in vivo or ex vivo with an antigen-binding molecule according to the present disclosure.

In some embodiments, the cell is an effector immune cell.

In some embodiments, the cell is a T cell or a NK cell.

In some embodiments, the γc-containing cytokine receptor is a γc:IL-9Rα receptor.

The present disclosure also provides a method of promoting heteromultimerization of γc and IL-9Rα, comprising contacting γc and a polypeptide of a γc-containing cytokine receptor complex in vitro, in vivo or ex vivo with an antigen-binding molecule according to the present disclosure, or a CAR according to the present disclosure.

The present disclosure also provides a method of inhibiting heteromultimerization of γc and IL-9Rα, comprising contacting γc and a polypeptide of a γc-containing cytokine receptor complex in vitro, in vivo or ex vivo with an antigen-binding molecule according to the present disclosure, or a CAR according to the present disclosure.

In some embodiments, the γc-binding moiety comprises:
A VH region comprising the heavy chain CDRs, and a VL region comprising the light chain CDRs, of a
   clone selected from: P1A3, P1A3_B3, P1A3_E8, P1A3_E9, P2B9, P1A3_B4, P1A3_FW2, P1A10, P1B6, P1C10, P1D7, P1E8, P2B2, P2B7, P2D11, P2F10, P2H4, P2D3, P1G4, P1B12, P1C7, P1A3_A, P1A3_Q, P1A3_AQ, P1A3_ANQ, P1A10_AQ, and P1A10_ANQ, as shown in Table A1 herein.

In some embodiments, the γc-binding moiety comprises:
a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:62; and
a VL region incorporating the following CDRs:
   LC-CDR1 having the amino acid sequence of SEQ ID NO:44
   LC-CDR2 having the amino acid sequence of SEQ ID NO:88
   LC-CDR3 having the amino acid sequence of SEQ ID NO:46.

The present disclosure includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1. Dose-dependent STAT5 phosphorylation by agonist bispecific γc- and IL-2RP-binding antibodies. STAT5 phosphorylation was assessed in different leukocyte subsets. The percentage of phosphorylated STAT5 (pSTAT5) was measured following stimulation with different concentrations of bispecific γc- and CD122-binding antibodies. Efficient stimulation was demonstrated for CD4+, CD8+, and NK cells, but no stimulation was demonstrated for the ISO control.

FIG. 2. Agonist bispecific γc- and IL-2RP-binding antibodies enhance tumor killing by EBV specific T cells. EBV-BCL tumor-engrafted mice were treated with EBV specific T cells and Treg cells in the presence of IL-2, and agonist bispecific γc- and CD122-binding antibodies (Adk-1 or Adk-2). Treatment comprising the use of agonist bispecific γc- and CD122-binding antibodies lead to significant reductions in absolute tumor cell count.

FIG. 3. Agonist bispecific γc- and IL-2RP-binding antibodies stimulate non-human primate T-cell proliferation in vivo. Expression of the proliferation marker Ki67 was used as a pharmacodynamic marker of immune stimulation in T cells. CD8 T cell proliferation occurred as early as 24 hours, persisting up to 120 hours post-dose.

FIG. 4. Schematic overview of cell signalling assays. Assays involved the stimulation of (HEK) 293 Cytokine Reporter Cells with bispecific antibodies (or control antibodies) and the assessment of pSTAT5 levels using the QUANTI-Blue assay. The different steps performed in (A) agonist assays, and (B) antagonist assays, are highlighted.

FIG. 5. Bispecific γc- and IL-9Rα-binding antibodies modulate γc:IL-9Rα receptor mediated signalling. (A) Agonist bispecific γc- and IL-9Rα-binding antibodies increase STAT5 phosphorylation, (B) Antagonist bispecific γc- and IL-9Rα-binding antibodies inhibit STAT5 phosphorylation.

### DETAILED DESCRIPTION OF THE INVENTION

The present disclosure encompasses the nucleotide and amino acid sequences of antigen-binding molecules with specificity for the common γ chain (γc; CD132) and a IL-9Rα (CD129). In some embodiments, the antigen-binding molecule comprises a γc-binding moiety and a moiety that binds to IL-9Rα (CD129).

In one aspect, this disclosure describes the design of cytokine receptor agonists in which receptor activation is achieved through heterodimerization of the receptor components by multispecific antigen binding molecules (e.g. bispecific antibodies or bi-functional proteins) possessing anti-γc specificity and specificity for IL-9Rα.

In another aspect, this disclosure describes the design of cytokine receptor antagonists in which receptor activation is reduced through the inhibition of heterodimerization of the receptor components by multispecific antigen binding molecules (e.g. bispecific antibodies or bi-functional proteins) possessing anti-γc specificity and specificity for IL-9Rα.

The antigen binding molecules of the present disclosure are associated with beneficial properties which overcome deficiencies and problems associated with the therapeutic administration of cytokines or engineered cytokines (e.g. PEGylated cytokines and antibody-coupled-cytokines).

The administration of cytokines and engineered cytokines may be associated with negative characteristics such as: short half-life, the requirement of toxic dosing levels, non-specific binding, and high levels of immunogenicity. These drawbacks can lead to problems in patients, such as: reduced efficacy, the development of anti-drug antibodies, activation of non-optimal signalling pathways, adverse events and the development of serious side-effects such as vascular leak syndrome (VLS).

Antigen binding molecules which bind γc-containing cytokine receptor complexes provide technical advances over cytokine-based therapies. For example, the antigen binding molecules of the present disclosure may be considered as drug-like molecules, and are more stable, have a longer half-life, increased durability of response, and can be tuned for individual patients and specific diseases.

The use of the antigen binding molecules of the present disclosure is associated with effective cell signalling. The affinity of binding and the level of signalling induction can be tuned to achieve optimum downstream effects, depending on the target cytokine receptor, disease to be treated, and the status of individual patients.

### Common γ Chain (γc)

Human common gamma (γ) chain (γc; also known as CD132, IL-2RG and CIDX) is the protein identified by UniProt P31785-1. The structure and function of γc is reviewed e.g. in Waickman et al., Cell Mol Life Sci. (2016) 73(2): 253-269 and Leonard et al., Immunity (2019) 50(4):832-850, both of which are hereby incorporated by reference in their entirety.

The canonical isoform of human γc (isoform 1) has the amino acid sequence shown in SEQ ID NO:437. The N-terminal 22 amino acids of SEQ ID NO:437 constitute a signal peptide (SEQ ID NO:503), and so the mature form (i.e. after processing to remove the signal peptide) of human γc has the amino acid sequence shown in SEQ ID NO:438. Amino acids 23 to 262 of SEQ ID NO:437 constitute the extracellular domain of γc (SEQ ID NO:439), positions 263 to 283 form a single-pass transmembrane domain (SEQ ID NO:518), and positions 284 to 369 form the cytoplasmic domain (SEQ ID NO:519). The extracellular domain comprises a fibronectin type III (FNIII) domain (shown in SEQ ID NO:547) comprising a WSXWS motif shown in SEQ ID NO:548. WSXWS motifs are conserved among type 1 cytokine receptor polypeptides, and the WSXWS motif of γc is thought to be important for conformational changes of the receptor.

All receptors of the γc receptor family comprise γc as a constituent polypeptide. Janus kinas 3 (JAK3) associates with γc, and upon activation of a γc-containing cytokine receptor, JAK3 becomes phosphorylated and activated. Phosphorylated JAK3 then phosphorylates and activates downstream signalling proteins such as STAT5, and also triggers signalling through the MAPK/ERK and PI3K/Akt signal transduction pathways. Signalling through γc family receptors promotes immune cell activation, proliferation and survival.

In this specification ‘common γ chain’, ‘common gamma chain’, ‘γc’, or ‘CD132’ refers to common γ chain from any species, and includes isoforms, fragments, variants or homologues of γc from any species. In some embodiments γc is γc from a mammal (e.g. a therian, placental, epitherian, preptotheria, archontan, primate (rhesus, cynomolgous, non-human primate or human)). In some embodiments, the γc is human γc.

As used herein, isoforms, fragments, variants or homologues of a given reference protein (e.g. γc) may be characterised as having at least 70% sequence identity, preferably one of ≥80%, ≥85%, ≥90%, ≥91%, 292%, ≥93%, ≥94%, ≥95%, ≥96%, ≥97%, ≥98%, 199% or 100% amino acid sequence identity to the amino acid sequence of the reference protein.

A ‘fragment’ generally refers to a fraction of the reference protein. A ‘variant’ generally refers to a protein having an amino acid sequence comprising one or more amino acid substitutions, insertions, deletions or other modifications relative to the amino acid sequence of the reference protein, but retaining a considerable degree of sequence identity (e.g. at least 60%) to the amino acid sequence of the reference protein. An ‘isoform’ generally refers to a variant of the reference protein expressed by the same species as the species of the reference protein. A ‘homologue’ generally refers to a variant of the reference protein produced by a different species as compared to the species of the reference protein. Homologues include orthologues. For example, homologues of human γc include e.g. mouse γc (UniProt P34902).

Isoforms, fragments, variants or homologues of a given reference protein may optionally be characterised as having at least 70%, preferably one of ≥80%, ≥85%, ≥90%, ≥91%, ≥92%, ≥93%, ≥94%, ≥95%, ≥96%, 297%, ≥98%, ≥99% or 100% amino acid sequence identity to the amino acid sequence of an immature or mature (i.e. after processing to remove signal peptide) form of a specified isoform of the relevant protein from a given species, e.g. human.

Isoforms, fragments, variants or homologues of γc according to the present disclosure may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature γc isoform from a given species, e.g. human.

Isoforms, fragments, variants or homologues may optionally be functional isoforms, fragments, variants or homologues, e.g. having a functional property/activity of the reference γc (e.g. human γc isoform 1), as determined by analysis by a suitable assay for the functional property/activity. For example, an isoform, fragment, variant or homologue of γc may display one or more of: association with one or more of IL-2Rβ, IL-2Rα, IL-15Rα, IL-4Rα, IL-9Rα, IL-21Rα, or IL-7Rα, or binding to one or more of IL-2, IL-15, IL-4, IL-9, IL-21 or IL-7.

A fragment of γc may have a minimum length of one of 10, 20, 30, 40, 50, 100, 150, 200, 250, 300 or 350 amino acids, and may have a maximum length of one of 20, 30, 40, 50, 100, 150, 200, 250, 300 or 350 amino acids.

In some embodiments, the γc has at least 70%, preferably one of 80%, 85%, 90%, 91%, 92%, 93%, 94%, 95%, 96%, 97%, 98%, 99% or 100% amino acid sequence identity to SEQ ID NO:437 or 438.

In some embodiments, a fragment of γc comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:439.

### Signalling Through Cytokine Receptors Comprising γc

There are a number of cytokines that signal through cytokine receptors comprising γc (also referred to herein as γc-containing receptor complexes), e.g. IL-2, IL-4, IL-7, IL-9, IL-15, and IL-21. Such cytokines are considered to belong to the γc family of cytokines. The biology of the γc family of cytokines is reviewed e.g. in Lin and Leonard, Cold Spring Harb Perspect Biol (2018) 10(9):a028449, Leonard et al., Immunity (2019) 50(4):832-850 and Pulliam et al., Immunol Lett. (2016) 169: 61-72, both of which are hereby incorporated by reference in their entirety.

Interleukin-9 (IL-9) is a cytokine which stimulates cell proliferation and prevents apoptosis. IL-9 is a pleiotropic cytokine and was primarily studied in the context of T helper 2 (TH2)-associated immuno-pathological conditions such as asthma and parasitic infections. There was a paradigm shift in the biology of IL-9 after the recent discovery of TH9 cells, a new subtype of TH cells which secrete IL-9 in copious amounts. This has resulted in renewed interest in this cytokine, which was neglected since discovery because it was considered it to be just another TH2 cytokine. Recent studies have shown that it has multiple cellular sources and is critically involved in the immune pathogenesis of inflammatory diseases and in guarding immune tolerance (Chakraborty et al. Int J Mol Sci. (2019) 20(9): 2113). IL-9 functions through the interleukin-9 receptor complex, which comprises IL-9 receptor alpha (IL-9Rα) and γc. When IL-9 binds to the IL-9 receptor complex, it activates different signal transducer and activator (STAT) proteins namely STAT1, STAT3 and STAT5 and thus connects this cytokine to various biological processes. IL-9 is a pleiotropic cytokine that has both direct and indirect effects on hematopoietic progenitor cells, lymphocytes, mast cells, as well as airway smooth muscle cells and epithelial cells (Lee at al. Pathology & Oncology Research (2020) 26:2017-2022). IL-9 also activates insulin receptor substrates (IRS) 1 and 2. Following JAK mediated phosphorylation, IRS proteins interact with other SH2-containing signaling proteins, such as the regulatory subunit of Phosphatidylinositol-3 Kinase (PI3K) p85, causing the activation of the PI3K catalytic subunit p110. PI3K then activates downstream signaling molecules like PI3K-dependent kinase (PDK) and Akt. Akt then phosphorylates BAD and protects cells by preventing caspase-mediated apoptosis. IL-9 also activates the MAPK pathway in several cell lines of lymphoid and hematopoietic origin, but the IL-9 mediated MAPK activation is weak compared to other cytokines like IL-3.

In this specification ‘γc-containing cytokine receptor-mediated signalling’ refers to signalling mediated by multimeric receptor complexes comprising γc (e.g. comprising γc and another member of the γc receptor family other than γc). ‘Signalling’ refers to signal transduction and other cellular processes governing cellular activity.

γc-containing cytokine receptor-mediated signalling is signalling mediated by a γc-containing polypeptide complex (i.e. a polypeptide complex comprising one or more γc polypeptides, and another member of the γc receptor family other than γc). Polypeptide complexes according to the present disclosure may be characterised by non-covalent, protein:protein interaction between constituent polypeptide(s)/peptide(s). In some embodiments, the association comprises electrostatic interaction (e.g. ionic bonding, hydrogen bonding) and/or Van der Waals forces.

γc-containing cytokine receptor-mediated signalling may be mediated by heteromultimeric polypeptide complexes comprising one or more γc polypeptides, and additionally comprising one or more polypeptides of the γc receptor family other than γc (e.g. IL-9Rα). In some embodiments, γc-containing cytokine receptor-mediated signalling may be mediated by a polypeptide complex forming a receptor for a γc family cytokine. For example, γc-containing cytokine receptor-mediated signalling may be mediated by a polypeptide complex forming a receptor for IL-9. In some embodiments, γc-containing cytokine receptor-mediated signalling is mediated by a polypeptide complex forming a receptor for IL-9.

In some embodiments, γc-containing cytokine receptor-mediated signalling may be mediated by a polypeptide complex comprising γc and IL-9Rα (i.e. a γc:IL-9Rα complex). As explained hereinabove, γc and IL-9Rα interact to form the IL-9 receptor. Such signalling may be referred to as γc:IL-9Rα-mediated signalling. In some embodiments, γc-containing cytokine receptor-mediated signalling may be mediated by a polypeptide complex comprising IL-9, γc and IL-9Rα (i.e. a IL-9:γc:IL-9Rα complex). Such signalling may be referred to as IL-9:γc:IL-9Rα-mediated signalling (i.e. signalling mediated by binding of IL-9 to the IL-9 receptor).

The present disclosure relates to antigen-binding molecules that selectively bind to more than one component of γc-containing cytokine receptors.

Amino acid sequences of polypeptides of a γc-containing cytokine receptor other than γc, and domains/fragments thereof are disclosed herein (e.g., SEQ ID NOs 245-255 and 258-284).

In particular, the antigen-binding molecules of the present disclosure are multispecific antigen-binding molecules comprising (i) a γc-binding moiety, and (ii) a moiety that binds to IL-9Rα). That is, the antigen binding molecule binds to (i) γc, and (ii) IL-9Rα.

Human IL-9Rα (also known as IL-9R and CD129) is the protein identified by UniProt Q01113. The canonical isoform of human IL-9Rα (Uniprot Q01113-1) has the amino acid sequence of SEQ ID NO:267.

The N-terminal 40 amino acids of SEQ ID NO:267 constitute a signal peptide (SEQ ID NO:268), and so the mature form (i.e. after processing to remove the signal peptide) of human IL-9Rα has the amino acid sequence shown in SEQ ID NO:269. Human IL-9Rα comprises an extracellular domain (SEQ ID NO:270), a transmembrane domain (SEQ ID NO:271), and a cytoplasmic domain (SEQ ID NO:272).

In this specification ‘IL-9Rα’ refers to IL-9Rα from any species, and includes isoforms, fragments, variants or homologues from any species. In some embodiments IL-9Rα is IL-9Rα from a mammal (e.g. a therian, placental, epitherian, preptotheria, archontan, primate (rhesus, cynomolgous, non-human primate or human)). In some embodiments, the IL-9Rα is human IL-9Rα.

An isoform, fragment, variant or homologue of IL-9R may display association with γc or IL-9. A fragment of IL-9Rα may have a minimum length of one of 10, 20, 30, 40, 50, 100, 150, 200, 300, 400 or 500 amino acids, and may have a maximum length of one of 20, 30, 40, 50, 100, 150, 200, 300, 400 or 500 amino acids.

In some embodiments, the IL-9Rα comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:267 or 269. In some embodiments, a fragment of IL-9Rα comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:270.

### Antigen-Binding Molecules

The present disclosure provides antigen-binding molecules capable of binding to (i.e. that bind to) γc-containing cytokine receptors, and constituent polypeptides thereof.

In aspects of the present disclosure, the antigen-binding molecules comprise a γc-binding moiety. In aspects of the present disclosure, the antigen-binding molecules comprise a moiety that binds to IL-9Rα.

In some embodiments, the antigen-binding molecule comprises an IL-9Rα-binding moiety.

In some embodiments, the antigen-binding molecule comprises a γc-binding moiety and an IL-9Rα-binding moiety.

As used herein, an ‘antigen-binding molecule’ refers to a molecule that binds to (a) given target antigen(s). The antigen-binding molecules of the present disclosure comprise one or more antigen-binding moieties, through which the antigen-binding molecule binds to its target antigen(s).

Antigen-binding moieties may comprise, or may be derived from, antibodies (i.e. immunoglobulins (Igs)) and antigen-binding fragments thereof. As used herein, ‘antibodies’ include monoclonal antibodies, polyclonal antibodies, monospecific and multispecific (e.g., bispecific, trispecific, etc.) antibodies, and antibody-derived antigen-binding molecules such as scFv, scFab, diabodies, triabodies, scFv-Fc, minibodies, single domain antibodies (e.g. VHH), etc. Antigen-binding fragments of antibodies include e.g. Fv, Fab, F(ab′)2 and F(ab′) fragments.

Single-domain antibodies (sdAbs)—also referred to variously in the art as ‘single variable domain on a heavy chain antibodies’, ‘VHHs’, ‘nanobodies’ and ‘heavy chain only antibodies (HcAbs)’—are described e.g. in Henry and MacKenzie, Front Immunol. (2018) 9:41 and Bever et al., Anal Bioanal Chem. (2016) 408(22): 5985-6002, both of which are hereby incorporated by reference in their entirety.

Single-domain antibodies are formed of a single, monomeric antibody variable domain. The first single-domain antibodies were engineered from heavy-chain antibodies found in camelids, and cartilaginous fishes also have heavy-chain antibodies.

Single-domain antibodies according to the present disclosure generally comprise three complementarity-determining regions CDRs: CDR1, CDR2 and CDR3. The three CDRs together define the paratope of the molecule, which is the part through which it binds to its target antigen.

Single-domain antibodies further comprise framework regions (FRs) either side of each CDR, which provide a scaffold for the CDRs. From N-terminus to C-terminus, single-domain antibodies comprise the following structure: N term-[FR1]-[CDR1]-[FR2]-[CDR2]-[FR3]-[CDR3]-[FR4]-C term.

Antigen-binding moieties also include target antigen-binding aptamers, e.g. nucleic acid aptamers (reviewed, for example, in Zhou and Rossi, Nat Rev Drug Discov. (2017) 16(3):181-202). In some embodiments, an antigen-binding moiety comprises or consists of an antigen-binding peptide/polypeptide, e.g. a peptide aptamer, thioredoxin, monobody, anticalin, Kunitz domain, avimer, knottin, fynomer, atrimer, DARPin, affibody, nanobody (i.e. a single-domain antibody (sdAb)), affilin, armadillo repeat protein (ArmRP), OBody or fibronectin—reviewed e.g. in Reverdatto et al., Curr Top Med Chem. (2015) 15(12):1082-1101, which is hereby incorporated by reference in its entirety (see also e.g. Boersma et al., J Biol Chem. (2011) 286:41273-85 and Emanuel et al., Mabs. (2011) 3:38-48).

In some embodiments, an antigen-binding moiety comprises, or consists of, the antigen-binding region of an antibody (e.g. an antigen-binding fragment of an antibody). Antigen-binding moieties of the antigen-binding molecules of the present disclosure may comprise the antibody heavy chain variable region (VH) and the antibody light chain variable region (VL) of an antibody that binds to the relevant target antigen of the antigen-binding moiety. The antigen-binding domain formed by a VH and a VL may also be referred to herein as an Fv region. Antigen-binding moieties of the antigen-binding molecules of the present disclosure may comprise the single variable domain (VHH) of a single-domain antibody (sdAb) that binds to the relevant target antigen of the antigen-binding moiety.

In some embodiments, an antigen-binding moiety is or comprises the Fv (e.g. provided as an scFv) of an antibody. In some embodiments, an antigen-binding moiety is or comprises the Fab region of an antibody. In some embodiments, an antigen-binding moiety is or comprises the whole antibody (i.e. comprising variable and constant regions).

An antigen-binding moiety may be, or may comprise, an antigen-binding polypeptide, or an antigen-binding polypeptide complex. An antigen-binding moiety may comprise more than one polypeptide which together form an antigen-binding moiety. The polypeptides may associate covalently or non-covalently. In some embodiments, the polypeptides form part of a larger polypeptide comprising the polypeptides (e.g. in the case of scFv comprising VH and VL, or in the case of scFab comprising VH-CH1 and VL-CL).

An antigen-binding moiety may refer to a non-covalent or covalent complex of more than one polypeptide (e.g. 2, 3, 4, 6, or 8 polypeptides), e.g. an IgG-like antigen-binding molecule comprising two heavy chain polypeptides and two light chain polypeptides.

The antigen-binding moieties of the present disclosure may be designed and prepared using the sequences of monoclonal antibodies (mAbs) capable of binding to a given target antigen (e.g. HER3). Antigen-binding regions of antibodies, such as single chain variable fragment (scFv), Fab and F(ab′)₂ fragments may also be used/provided. An ‘antigen-binding region’ is any fragment of an antibody that binds to the target for which the given antibody is specific.

Antibodies generally comprise six complementarity-determining regions (CDRs); three in the heavy chain variable (VH) region: HC-CDR1, HC-CDR2 and HC-CDR3, and three in the light chain variable (VL) region: LC-CDR1, LC-CDR2, and LC-CDR3. The six CDRs together define the paratope of the antibody, which is the part of the antibody that binds to the target antigen.

The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as those described in Kabat et al., Sequences of Proteins of Immunological Interest, 5^{th} Ed. Public Health Service, National Institutes of Health, Bethesda, MD (1991), Chothia et al., J. Mol. Biol. (1987) 196:901-917, and VBASE2, as described in Retter et al., Nucl. Acids Res. (2005) 33 (suppl 1):D671-D674. The CDRs and FRs of the VH regions and VL regions (or VHH) of the antibody clones described herein were defined according to the international IMGT (ImMunoGeneTics) information system (LeFranc et al., Nucleic Acids Res. (2015) 43 (Database issue):D413-22), which uses the IMGT V-DOMAIN numbering rules as described in Lefranc et al., Dev. Comp. Immunol. (2003) 27:55-77.

In some embodiments, an antigen-binding moiety according to the present disclosure comprises, or consists of an Fv moiety that binds to its target antigen. In some embodiments, the VH and VL regions of the Fv moiety are provided as single polypeptides joined by a linker sequence, i.e. a single chain Fv (scFv).

The VL and light chain constant (CL) region, and the VH region and heavy chain constant 1 (CH1) region of an antigen-binding region of an antibody together constitute the Fab region. In some embodiments, the antigen-binding molecule comprises a Fab moiety comprising a VH, a CH1, a VL and a CL (e.g. CK or CA). In some embodiments, the Fab moiety comprises a polypeptide comprising a VH and a CH1 (e.g. a VH-CH1 fusion polypeptide), and a polypeptide comprising a VL and a CL (e.g. a VL-CL fusion polypeptide). In some embodiments, the Fab moiety comprises a polypeptide comprising a VH and a CL (e.g. a VH-CL fusion polypeptide) and a polypeptide comprising a VL and a CH1 (e.g. a VL-CH1 fusion polypeptide); that is, in some embodiments, the Fab moiety is a CrossFab moiety. In some embodiments, the VH, CH1, VL and CL regions of the Fab moiety or CrossFab moiety are provided as single polypeptides joined by a linker sequence, i.e. as a single chain Fab (scFab) or a single chain CrossFab (scCrossFab).

In some embodiments, an antigen-binding molecule described herein comprises, or consists of, a whole antibody which binds to its target antigen. As used herein, ‘whole antibody’ refers to an antibody having a structure which is substantially similar to the structure of an immunoglobulin (Ig). Different kinds of immunoglobulins and their structures are described e.g. in Schroeder and Cavacini. J Allergy Clin Immunol. (2010) 125(202): S41-S52, which is hereby incorporated by reference in its entirety.

Immunoglobulins of type G (i.e. IgG) are −150 kDa glycoproteins comprising two heavy chains and two light chains. From N- to C-terminus, the heavy chains comprise a VH followed by a heavy chain constant region comprising three constant domains (CH1, CH2, and CH3), and similarly the light chains comprise a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

Herein, a ‘CH2 domain’ refers to an amino acid sequence corresponding to the CH2 domain of an immunoglobulin (Ig). The CH2 domain is the region of an Ig formed by positions 231 to 340 of the immunoglobulin constant domain, according to the EU numbering system (described in Edelman et al., Proc Natl Acad Sci USA. (1969) 63(1):78-85). A ‘CH3 domain’ refers to an amino acid sequence corresponding to the CH3 domain of an immunoglobulin (Ig). The CH3 domain is the region of an Ig formed by positions 341 to 447 of the immunoglobulin constant domain, according to the EU numbering system. A ‘CH2-CH3 region’ refers to an amino acid sequence corresponding to the CH2 and CH3 domains of an immunoglobulin (Ig). The CH2-CH3 region is the region of an Ig formed by positions 231 to 447 of the immunoglobulin constant domain, according to the EU numbering system.

In some embodiments described herein, one or more amino acids of an amino acid sequence referred to herein (e.g. an amino acid sequence of an antigen-binding molecule, e.g. an amino acid sequence of a CDR or VH/VL region) are substituted with another amino acid. A substitution comprises substitution of an amino acid residue with a non-identical ‘replacement’ amino acid residue. A replacement amino acid residue of a substitution according to the present disclosure may be a naturally-occurring amino acid residue (i.e. encoded by the genetic code) which is non-identical to the amino acid residue at the relevant position of the equivalent, unsubstituted amino acid sequence, selected from: alanine (Ala), arginine (Arg), asparagine (Asn), aspartic acid (Asp), cysteine (Cys), glutamine (Gln), glutamic acid (Glu), glycine (Gly), histidine (His), isoleucine (Ile): leucine (Leu), lysine (Lys), methionine (Met), phenylalanine (Phe), proline (Pro), serine (Ser), threonine (Thr), tryptophan (Trp), tyrosine (Tyr), and valine (Val). In some embodiments, a replacement amino acid may be a non-naturally occurring amino acid residue—i.e. an amino acid residue other than those recited in the preceding sentence. Examples of non-naturally occurring amino acid residues include norleucine, ornithine, norvaline, homoserine, aib, and other amino acid residue analogues such as those described in Ellman et al., Meth. Enzym. (1991) 202:301-336.

In some embodiments, a substitution may be biochemically conservative. In some embodiments, where an amino acid to be substituted is provided in one of rows 1 to 5 of the table below, the replacement amino acid of the substitution is another, non-identical amino acid provided in the same row:

| Row | Shared property | Amino acids |
|---|---|---|
| 1 | Hydrophobic | Met, Ala, Val, Leu, Ile, |
| | | Trp, Tyr, Phe, Norleucine |
| 2 | Neutral hydrophilic | Cys, Ser, Thr, Asn, Gln |
| 3 | Acidic or negatively-charged | Asp, Glu |
| 4 | Basic or positively-charged | His, Lys, Arg |
| 5 | Orientation influencing | Gly, Pro |

By way of illustration, in some embodiments wherein substitution is of a Met residue, the replacement amino acid may be selected from Ala, Val, Leu, Ile, Trp, Tyr, Phe and Norleucine.

In some embodiments, a replacement amino acid in a substitution may have the same side chain polarity as the amino acid residue it replaces. In some embodiments, a replacement amino acid in a substitution may have the same side chain charge (at pH 7.4) as the amino acid residue it replaces:

| | | Side-chain |
|---|---|---|
| | Side-chain | charge |
| Amino Acid | polarity | (pH 7.4) |
| Alanine | nonpolar | neutral |
| Arginine | basic polar | positive |
| Asparagine | polar | neutral |
| Aspartic acid | acidic polar | negative |
| Cysteine | nonpolar | neutral |
| Glutamic acid | acidic polar | negative |
| Glutamine | polar | neutral |
| Glycine | nonpolar | neutral |
| Histidine | basic polar | positive (10%) |
| | | neutral (90%) |
| Isoleucine | nonpolar | neutral |
| Leucine | nonpolar | neutral |
| Lysine | basic polar | positive |
| Methionine | nonpolar | neutral |
| Phenylalanine | nonpolar | neutral |
| Proline | nonpolar | neutral |
| Serine | polar | neutral |
| Threonine | polar | neutral |
| Tryptophan | nonpolar | neutral |
| Tyrosine | polar | neutral |
| Valine | nonpolar | neutral |

That is, in some embodiments, a nonpolar amino acid is substituted with another, non-identical nonpolar amino acid. In some embodiments, a polar amino acid is substituted with another, non-identical polar amino acid. In some embodiments, an acidic polar amino acid is substituted with another, non-identical acidic polar amino acid. In some embodiments, a basic polar amino acid is substituted with another, non-identical basic polar amino acid. In some embodiments, a neutral amino acid is substituted with another, non-identical neutral amino acid. In some embodiments, a positive amino acid is substituted with another, non-identical positive amino acid. In some embodiments, a negative amino acid is substituted with another, non-identical negative amino acid.

In some embodiments, substitution(s) may be functionally conservative. That is, in some embodiments, the substitution may not affect (or may not substantially affect) one or more functional properties (e.g. target binding) of the antigen-binding molecule comprising the substitution as compared to the equivalent unsubstituted molecule.

In some embodiments, the antigen-binding molecule comprises an antigen-binding moiety that binds to γc. In some embodiments, the antigen-binding moiety comprises the CDRs of an antibody that binds to γc. In some embodiments, the antigen-binding moiety comprises the FRs of an antibody that binds to γc. In some embodiments, the antigen-binding moiety comprises the CDRs and the FRs of an antibody that binds to γc. That is, in some embodiments the antigen-binding moiety comprises the VH region and the VL region of an antibody that binds to γc, or the VHH sequence of a single-domain antibody that binds to γc. In some embodiments, the antigen-binding moiety comprises or consists of an Fv moiety or Fab moiety comprising the VH region and the VL region of an antibody that binds to γc. Antibodies that bind to γc include e.g. REGN7257 (described in e.g. Floch et al., Hemasphere. (2022) 6 (Suppl):694-695), TUGh4 (described in e.g. Ishii et al. Int Immunol. (1994) 6(8):1273-1277) and 3E12 (described in e.g. He et al. Proc Natl Acad Sci USA. (1995) 92(12):5689-5693). The VHH antibody nb6 has also been shown to bind to γc ((Yen et al., Cell. 2022; 185(8): 1414-1430). The single-domain antibody (VHH) nb6 has also been shown to bind to γc (Yen et al., Cell. 2022; 185(8): 1414-1430). The present disclosure provides further single-domain antibodies (VHH) that bind to γc (see for example the clone listed in Table A2).

In some embodiments, the antigen-binding molecule comprises an antigen-binding moiety that binds to IL-9Rα. In some embodiments, the antigen-binding moiety comprises the CDRs of an antibody that binds to IL-9Rα. In some embodiments, the antigen-binding moiety comprises the FRs of an antibody that binds to IL-9Rα. In some embodiments, the antigen-binding moiety comprises the CDRs and the FRs of an antibody that binds to IL-9Rα. That is, in some embodiments the antigen-binding moiety comprises the VH region and the VL region of an antibody that binds to IL-9Rα. In some embodiments, the antigen-binding moiety comprises or consists of an Fv moiety or Fab moiety comprising the VH region and the VL region of an antibody that binds to IL-9Rα. Antibodies that bind to IL-9Rα include e.g. RZ-66 (described in e.g. Takatsuka et al., Nature Immunology. (2018) 19(9):1025-1034) and AH9R7 (described in e.g. Smedt et al. J Immunol. (2000) 164(4):1761-1767). The present disclosure provides further single-domain antibodies (VHH) that bind to IL-9Rα (see for example Tables A3 and B3).

The antigen-binding molecules of the present disclosure are multispecific. By ‘multispecific’ it is meant that the antigen-binding molecule binds to more than one target. In particular, the antigen-binding molecule binds to (i) γc, and (ii) IL-9Rα. It will be appreciated that the multispecific antigen-binding molecule is at least bispecific. The term ‘bispecific’ means that the antigen-binding molecule binds to at least two, distinct antigenic determinants. In some embodiments, the antigen-binding molecule is bispecific, trispecific, tetraspecific, pentaspecific, hexaspecific, heptaspecific, octaspecific, nonaspecific or decaspecific.

The multispecific antigen-binding molecules described herein display at least monovalent binding with respect to γc, and also display at least monovalent binding with respect to IL-9Rα. Binding valency refers to the number of binding sites in an antigen-binding molecule for a given antigenic determinant. For example, bispecific antigen-binding molecules in scFv-KiH-Fc, CrossMab and Duobody formats are described herein, which display monovalent binding to γc, and monovalent binding with respect to binding to IL-2Rβ. Additionally, bispecific antigen-binding molecules in tandem VHH formats are described herein, which display monovalent binding to γc, and monovalent binding with respect to binding to IL-9Rα.

Multispecific antigen-binding molecules according to the present disclosure may be provided in any suitable format, such as those formats described in Kontermann, MAbs. (2012) 4(2):182-197, which is hereby incorporated by reference in its entirety. For example, an antigen-binding molecule according to the present disclosure may be a bispecific antibody conjugate (e.g. an IgG2, F(ab′)2 or CovX-Body), a bispecific IgG or IgG-like molecule (e.g. an IgG, scFv4-Ig, IgG-scFv, scFv-IgG, DVD-Ig, IgG-sVD, sVD-IgG, 2 in 1-IgG, mAb², or Tandemab common LC), an asymmetric bispecific IgG or IgG-like molecule (e.g. a kih IgG, kih IgG common LC, CrossMab, kih IgG-scFab, mAb-Fv, charge pair or SEED-body), a small bispecific antibody molecule (e.g. a Diabody (Db), dsDb, DART, scDb, tandAbs, tandem scFv (taFv), tandem dAb/VHH, tandem VHH-VHH, tandem VHH-scFv, tandem scFV-VHH, triple body, triple head, Fab-scFv, or F(ab′)₂-scFv₂), a bispecific Fc and C_{H}3 fusion protein (e.g. a taFv-Fc, Di-diabody, scDb-CH3, scFv-Fc-scFv, HCAb-VHH, scFv-kih-Fc, or scFv-kih-CH3), or a bispecific fusion protein (e.g. a scFv2-albumin, scDb-albumin, taFv-toxin, DNL-Fab₃, DNL-Fab₄-IgG, DNL-Fab₄-IgG-cytokine₂). See in particular FIG. 2 of Kontermann, MAbs. (2012) 4(2):182-19. See also Brinkmann and Kontermann, MAbs. (2017) 9(2):182-212 (hereby incorporated by reference in its entirety), in particular FIG. 2. In some embodiments, the multispecific antigen-binding molecule is minimalistic bispecific antibody, such as a tandem scFV (i.e., scFV-scFv), a tandem VHH (i.e., VHH-VHH), or a tandem VHH-scFv antibody.

A tandem multispecific antigen-binding molecule (e.g., a tandem VHH) comprises two (or more) binding moieties (e.g., scFv and/or VHH moieties) and a linker. In some embodiments, the multispecific antigen-binding molecule is provided in a tandem format, where binding moieties are joined by a linker. Examples of tandem format antigen binding molecules include tandem scFv, tandem VHH, and tandem VHH-scFv. In some embodiments, the multispecific antigen-binding molecule is provided in a tandem scFv format, such as tandem scFv-scFv. In some embodiments, the multispecific antigen-binding molecule is provided in a tandem VHH format. In some embodiments, the multispecific antigen-binding molecule is provided in a tandem VHH-scFV format. In some embodiments, the multispecific antigen-binding molecule is provided in a tandem scFV-VHH format.

Binding moieties are joined by a linker. scFv moieties are typically in the orientation: VH-VL-linker-VH-VL or VL-VH-linker-VL-VH (from the N-terminus to the C-terminus), or VHH-Linker-VHH. Therefore, different binding moieties can be combined in multiple different ways. By way of example, two scFv molecules can be combined in at multiple different orientations. By way of example, a P2C4 scFv can be combined with a P1A3 scFv in at least the following orientations: (i) P2C4VL-P2C4VH-linker-P1A3VL-P1A3VH, (ii) P1A3VL-P1A3VH-linker-P2C4VL-P2C4VH, (iii) P2C4VH-P2C4VL-linker-P1A3VH-P1A3VL, and (iv) P1A3VH-P1A3VL-linker-P2C4VH-P2C4VL. Two VHH molecules (VHH1 and VHH2) can be combined in at least the following orientations (from the N-terminus to the C-terminus): (i) VHH1-linker-VHH2, and (ii) VHH2-linker-VHH1.

In some embodiments, the multispecific antigen-binding molecule comprises a linker between binding moieties, for example, a linker between a γc-binding moiety and a moiety that binds to a polypeptide of a γc-containing cytokine receptor other than γc (e.g., an IL-9Rα-binding moiety). Such linkers are described by Brinkmann and Kontermann (MAbs. (2017) 9(2):182-212), which is hereby incorporated by reference in its entirety. In some embodiments, the linker is a linker described in Brinkmann and Kontermann (MAbs. (2017) 9(2):182-212).

In some embodiments, the linker is an amino acid linker. In some embodiments, the linker is a flexible linker. In some embodiments, the linker is a rigid linker. In some embodiments, the linker is a short flexible linker. In some embodiments, the linker is a long rigid linker.

In some embodiments, the flexible linker is rich in small or polar amino acids such as Gly and/or Ser to provide flexibility and solubility. In some embodiments, the linker is a glycine-rich linker. In some embodiments, the linker is a serine-rich linker. In some embodiments, the linker is an amino acid linker in which at least 50% of the total amino acids are glycine amino acids, e.g. one of ≥55%, ≥60%, ≥65%, ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or 199% of the total amino acids are glycine amino acids. In some embodiments, the linker comprises or consists of a GGGGS (SEQ ID NO:240) amino acid sequence. In some embodiments, the linker comprises or consists of a GGGGSGGGS (SEQ ID NO:361) amino acid sequence. In some embodiments, the linker comprises or consists of a (G3S)4 (SEQ ID NO:362) amino acid sequence. In some embodiments, the linker comprises or consists of a GGGSG (SEQ ID NO:363) amino acid sequence.

In some embodiments, the linker sequence comprises at least one glycine residue and/or at least one serine residue. In some embodiments, the linker sequence comprises or consists of glycine and serine residues. In some embodiments, the linker sequence has the structure: (GxS)n or (GxS)nGm; wherein G=glycine, S=serine, x=3 or 4, n=2, 3, 4, 5 or 6, and m=0, 1, 2 or 3. In some embodiments, the linker sequence comprises one or more (e.g., 1, 2, 3, 4, 5 or 6) copies (e.g., in tandem) of the sequence motif G4S. In some embodiments, the linker sequence comprises or consists of (G4S)4 or (G4S)6. In some embodiments, the linker sequence has a length of 1-2, 1-3, 1-4, 1-5, 1-10, 1-15, 1-20, 1-25, or 1-30 amino acids.

In some embodiments, the linker is a rigid linker. In some embodiments, the rigid linker forms an alpha helical structure between binding moieties. Rigid linkers are discussed by Arai et al. (Protein Engineering, Design and Selection, 14(8), 2001, 529-532), which is hereby incorporated by reference in its entirety. In some embodiments, the linker is a linker described in Arai et al. (Protein Engineering, Design and Selection, 14(8), 2001, 529-532). In some embodiments, the linker comprises or consists of an A(EAAAK)₅A (SEQ ID NO:361) amino acid sequence.

In some embodiments, the linker comprises an EAAAK (SEQ ID NO:364) amino acid sequence. In some embodiments, the linker comprises or consists of an A(EAAAK)₂A (SEQ ID NO:365) amino acid sequence. In some embodiments, the linker comprises or consists of an A(EAAAK)₃A (SEQ ID NO:366) amino acid sequence. In some embodiments, the linker comprises or consists of an A(EAAAK)₄A (SEQ ID NO:367) amino acid sequence. In some embodiments, the linker comprises or consists of an A(EAAAK)₅A (SEQ ID NO:360) amino acid sequence.

In some embodiments, the linker has a length of at least 3 amino acids. In some embodiments, the linker has a maximum length of 50 amino acids. In some embodiments, the linker has a minimum length of one of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 amino acids. In some embodiments, the linker has a maximum length of one of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids.

In some embodiments, the linker has a length between 3 and 50 amino acids, 4 and 50 amino acids, 5 and 50 amino acids, 6 and 50 amino acids, 7 and 50 amino acids, 8 and 50 amino acids, 9 and 50 amino acids, 10 and 50 amino acids, 11 and 50 amino acids, 12 and 50 amino acids, 13 and 50 amino acids, 14 and 50 amino acids, 15 and 50 amino acids, 16 and 50 amino acids, 17 and 50 amino acids, 18 and 50 amino acids, 19 and 50 amino acids, or 20 and 50 amino acids.

In some embodiments, the linker has a length between 3 and 40 amino acids, 4 and 40 amino acids, 5 and 40 amino acids, 6 and 40 amino acids, 7 and 40 amino acids, 8 and 40 amino acids, 9 and 40 amino acids, 10 and 40 amino acids, 11 and 40 amino acids, 12 and 40 amino acids, 13 and 40 amino acids, 14 and 40 amino acids, 15 and 40 amino acids, 16 and 40 amino acids, 17 and 40 amino acids, 18 and 40 amino acids, 19 and 40 amino acids, or 20 and 40 amino acids.

In some embodiments, the linker has a length between 3 and 30 amino acids, 4 and 30 amino acids, 5 and 30 amino acids, 6 and 30 amino acids, 7 and 30 amino acids, 8 and 30 amino acids, 9 and 30 amino acids, 10 and 30 amino acids, 11 and 30 amino acids, 12 and 30 amino acids, 13 and 30 amino acids, 14 and 30 amino acids, 15 and 30 amino acids, 16 and 30 amino acids, 17 and 30 amino acids, 18 and 30 amino acids, 19 and 30 amino acids, or 20 and 30 amino acids.

In some embodiments, the linker has a length between 3 and 20 amino acids, 4 and 20 amino acids, 5 and 20 amino acids, 6 and 20 amino acids, 7 and 20 amino acids, 8 and 20 amino acids, 9 and 20 amino acids, 10 and 20 amino acids, 11 and 20 amino acids, 12 and 20 amino acids, 13 and 20 amino acids, 14 and 20 amino acids, 15 and 20 amino acids, 16 and 20 amino acids, 17 and 20 amino acids, 18 and 20 amino acids, or 19 and 20 amino acids.

In some embodiments, the linker has a length between 3 and 4 amino acids, 3 and 5 amino acids, 3 and 6 amino acids, 3 and 7 amino acids, 3 and 8 amino acids, 3 and 9 amino acids, 3 and 10 amino acids, 3 and 11 amino acids, 3 and 12 amino acids, 3 and 13 amino acids, 3 and 14 amino acids, 3 and 15 amino acids, 3 and 16 amino acids, 3 and 17 amino acids, 3 and 18 amino acids, 3 and 19 amino acids, or 3 and 20 amino acids.

In some embodiments, a flexible linker has a length between 3 and 12 amino acids. In some embodiments, a short flexible linker has a length between 3 and 10 amino acids. In some embodiments, a short flexible linker has a length between 3 and 8 amino acids. In some embodiments, a short flexible linker has a length between 3 and 6 amino acids.

In some embodiments, a rigid linker has a length between 10 and 44 amino acids. In some embodiments, a long rigid linker has a length between 12 and 44 amino acids. In some embodiments, a long rigid linker has a length between 17 and 44 amino acids. In some embodiments, a long rigid linker has a length between 22 and 44 amino acids. In some embodiments, a long rigid linker has a length between 27 and 44 amino acids.

The skilled person is readily able in view of their common general knowledge, and e.g. with reference to the publications referred to herein, to design and prepare multispecific (e.g. bispecific) antigen-binding molecules according to the present disclosure. Such techniques are described e.g. in Brinkmann and Kontermann, MAbs. (2017) 9(2):182-212, and Ma et al., Front Immunol. (2021) 12:626616, both of which are hereby incorporated by reference in their entirety.

Methods for producing multispecific antigen-binding molecules include chemical crosslinking of antigen-binding molecules or antibody fragments, e.g. with reducible disulphide or non-reducible thioether bonds, for example as described in Segal and Bast, (2001) Current Protocols in Immunology. Chapter 2:2.13.1-2.13.16, which is hereby incorporated by reference in its entirety. For example, N-succinimidyl-3-(-2-pyridyldithio)-propionate (SPDP) can be used to chemically crosslink e.g. Fab fragments via hinge region SH-groups, to create disulfide-linked bispecific F(ab)2 heterodimers.

Other methods for producing multispecific antigen-binding molecules include fusing antibody-producing hybridomas e.g. with polyethylene glycol, to produce a quadroma cell capable of secreting bispecific antibody, for example as described in Segal and Bast, (2001) Current Protocols in Immunology. Chapter 2:2.13.1-2.13.16.

Multispecific antigen-binding molecules according to the present disclosure can also be produced recombinantly, by expression from e.g. a nucleic acid construct encoding polypeptides for the antigen-binding molecules, for example as described in Hornig and Färber-Schwarz, Methods Mol Biol. (2012) 907:713-27, or French, Methods Mol Med. (2000) 40:333-339, the entire contents of both of which are hereby incorporated by reference.

For example, a DNA construct encoding the light and heavy chain variable domains for the two antigen-binding fragments (i.e. the light and heavy chain variable domains for the antigen-binding fragment capable of binding γc, and the light and heavy chain variable domains for the antigen-binding fragment capable of binding to another target protein), and including sequences encoding a suitable linker or dimerization domain between the antigen-binding fragments can be prepared by molecular cloning techniques. Recombinant bispecific antibody can thereafter be produced by expression (e.g. in vitro) of the construct in a suitable host cell (e.g. a mammalian host cell), and expressed recombinant bispecific antibody can then optionally be purified.

In some embodiments, the γc-binding moiety of the present disclosure comprises a polypeptide or polypeptides comprising a VH region comprising the heavy chain CDRs, and a VL region comprising the light chain CDRs, of a clone selected from: P1A3, P1A3_B3, P1A3_E8, P1A3_E9, P2B9, P1A3_B4, P1A3_FW2, P1A10, P1B6, P1C10, P1D7, P1E8, P2B2, P2B7, P2D11, P2F10, P2H4, P2D3, P1G4, P1B12, P1C7, P1A3_A, P1A3_Q, P1A3_AQ, P1A3_ANQ, P1A10_AQ, and P1A10_ANQ, as shown in Table A1 herein.

In some embodiments, the γc-binding moiety comprises a polypeptide comprising a VH region comprising HC-CDR1, HC-CDR2 and HC-CDR3 as indicated for one of binding moieties A1-1 to A1-27 in column A of Table A1, optionally wherein 1 or 2 or 3 amino acids in HC-CDR1, and/or 1 or 2 or 3 amino acids in HC-CDR2, and/or 1 or 2 or 3 amino acids in HC-CDR3 are substituted with another amino acid.

In some embodiments, the γc-binding moiety comprises a polypeptide comprising a VL region comprising LC-CDR1, LC-CDR2 and LC-CDR3 as indicated for one of binding moieties A1-1 to A1-27 in column B of Table A1, optionally wherein 1 or 2 or 3 amino acids in LC-CDR1, and/or 1 or 2 or 3 amino acids in LC-CDR2, and/or 1 or 2 or 3 amino acids in LC-CDR3 are substituted with another amino acid.

In some embodiments, the γc-binding moiety comprises a polypeptide or polypeptides comprising: (i) a VH region comprising HC-CDR1, HC-CDR2 and HC-CDR3 as indicated in column A of Table A1, and (ii) a VL region comprising LC-CDR1, LC-CDR2 and LC-CDR3 as indicated in column B of Table A1, wherein the sequences of columns A and B are selected from the same row of Table A1 (i.e., wherein the sequences of columns A and B are of the same binding moiety selected from A1-1 to A1-27).

In some embodiments, the γc-binding moiety of the present disclosure comprises a polypeptide or polypeptides comprising a VH region comprising the heavy chain FRs, and a VL region comprising the light chain FRs, of a clone selected from: P1A3, P1A3_B3, P1A3_E8, P1A3_E9, P2B9, P1A3_B4, P1A3_FW2, P1A10, P1B6, P1C10, P1D7, P1E8, P2B2, P2B7, P2D11, P2F10, P2H4, P2D3, P1G4, P1B12, P1C7, P1A3_A, P1A3_Q, P1A3_AQ, P1A3_ANQ, P1A10_AQ, and P1A10_ANQ, as shown in Table B1 herein.

In some embodiments, the γc-binding moiety comprises a polypeptide comprising a VH region comprising HC-FR1, HC-FR2, HC-FR3 and HC-FR4 as indicated for one of binding moieties B1-1 to B1-27 in column A of Table B1, optionally wherein 1 or 2 or 3 amino acids in HC-FR1, and/or 1 or 2 or 3 amino acids in HC-FR2, and/or 1 or 2 or 3 amino acids in HC-FR3, and/or 1 or 2 or 3 amino acids in HC-FR4 are substituted with another amino acid.

In some embodiments, the γc-binding moiety comprises a polypeptide comprising a VH region comprising LC-FR1, LC-FR2, LC-FR3 and LC-FR4 as indicated for one of binding moieties B1-1 to B1-27 in column B of Table B1, optionally wherein 1 or 2 or 3 amino acids in LC-FR1, and/or 1 or 2 or 3 amino acids in LC-FR2, and/or 1 or 2 or 3 amino acids in LC-FR3, and/or 1 or 2 or 3 amino acids in LC-FR4 are substituted with another amino acid.

In some embodiments, the γc-binding moiety comprises a polypeptide or polypeptides comprising: (i) a VH region comprising HC-FR1, HC-FR2, HC-FR3 and HC-FR4 as indicated in column A of Table B1, and (ii) a VL region comprising LC-FR1, LC-FR2, LC-FR3 and LC-FR4 as indicated in column B of Table B1, wherein the sequences of columns A and B are selected from the same row of Table B1 (i.e., wherein the sequences of columns A and B are of the same binding moiety selected from B1-1 to B1-27).

In some embodiments, the γc-binding moiety of the present disclosure comprises a polypeptide or polypeptides comprising a VH region, and a VL region of a clone selected from: P1A3, P1A3_B3, P1A3_E8, P1A3_E9, P2B9, P1A3_B4, P1A3_FW2, P1A10, P1B6, P1C10, P1D7, P1E8, P2B2, P2B7, P2D11, P2F10, P2H4, P2D3, P1G4, P1B12, P1C7, P1A3_A, P1A3_Q, P1A3_AQ, P1A3_ANQ, P1A10_AQ, and P1A10_ANQ, as shown in Table C1 herein.

In some embodiments, the γc-binding moiety comprises a polypeptide comprising a VH region having at least 70%, e.g. one of ≥80%, ≥85%, ≥90%, ≥91%, ≥92%, ≥93%, ≥94%, ≥95%, ≥96%, ≥97%, ≥98%, ≥99% or 100% amino acid sequence identity to an amino acid sequence indicated in column A of Table C1.

In some embodiments, the γc-binding moiety comprises a polypeptide comprising a VL region having at least 70%, e.g. one of ≥80%, ≥85%, ≥90%, ≥91%, ≥92%, ≥93%, ≥94%, ≥95%, ≥96%, ≥97%, ≥98%, ≥99% or 100% amino acid sequence identity to an amino acid sequence indicated in column B of Table C1.

In some embodiments, the γc-binding moiety of the present disclosure comprises a polypeptide or polypeptides comprising a VH region having at least 70%, e.g. one of ≥80%, ≥85%, ≥90%, ≥91%, ≥92%, 293%, ≥94%, ≥95%, ≥96%, ≥97%, ≥98%, ≥99% or 100% amino acid sequence identity to an amino acid sequence indicated in column A of Table C1, and a VL region having at least 70%, e.g. one of ≥80%, 285%, ≥90%, ≥91%, ≥92%, ≥93%, ≥94%, ≥95%, ≥96%, ≥97%, ≥98%, 199% or 100% amino acid sequence identity to an amino acid sequence indicated in column B of Table C1, wherein the sequences of columns A and B are selected from the same row of Table C1 (i.e., wherein the sequences of columns A and B are of the same binding moiety selected from C1-1 to C1-27).

In some embodiments, the antigen-binding molecule according to the present disclosure comprises: (i) a γc-binding moiety according to an embodiment described herein, and (ii) a moiety that binds to IL-9Rα.

In some embodiments, the antigen-binding molecule of the present disclosure comprises one or more regions (e.g. CH1, CH2, CH3, etc.) of an immunoglobulin heavy chain constant sequence. In some embodiments, the immunoglobulin heavy chain constant sequence is, or is derived from, the heavy chain constant sequence of an IgG (e.g. IgG1, IgG2, IgG3, IgG4), IgA (e.g. IgA1, IgA2), IgD, IgE or IgM, e.g. a human IgG (e.g. hIgG1, hIgG2, hIgG3, hIgG4), hIgA (e.g. hIgA1, hIgA2), hIgD, hIgE or hIgM. In some embodiments, the immunoglobulin heavy chain constant sequence is, or is derived from, the heavy chain constant sequence of a human IgG1 allotype (e.g. G1m1, G1m2, G1m3 or G1m17).

It will be appreciated that CH2 and/or CH3 regions may be provided with further substitutions in accordance with modification to an Fc region of the antigen-binding molecule as described herein.

In some embodiments, the antigen-binding molecule of the present disclosure comprises one or more regions of an immunoglobulin light chain constant sequence. In some embodiments, the immunoglobulin light chain constant sequence is human immunoglobulin kappa constant (IGKC; Cκ). In some embodiments, the immunoglobulin light chain constant sequence is a human immunoglobulin lambda constant (IGLC; Cλ), e.g. IGLC1, IGLC2, IGLC3, IGLC6 or IGLC7.

In one aspect, the disclosure provides an antigen-binding molecule which binds to γc and comprises a single-domain antibody sequence incorporating the CDRs of 2RGT38, as shown in Table A2 herein.

In some embodiments, the antigen-binding molecule which binds to γc comprises a single-domain antibody sequence incorporating CDR1, CDR2 and CDR3 as indicated for the binding moiety in Table A2, optionally wherein 1 or 2 or 3 amino acids in CDR1, and/or 1 or 2 or 3 amino acids in CDR2, and/or 1 or 2 or 3 amino acids in CDR3 are substituted with another amino acid.

In some embodiments, the antigen-binding molecule which binds to γc comprises a single-domain antibody sequence comprising the FRs of 2RGT38, as shown in Table B2 herein.

In some embodiments, the antigen-binding molecule comprises a single-domain antibody sequence comprising FR1, FR2, FR3 and FR4 as indicated for one of the clones in Table B2, optionally wherein 1 or 2 or 3 amino acids in FR1, and/or 1 or 2 or 3 amino acids in FR2, and/or 1 or 2 or 3 amino acids in FR3, and/or 1 or 2 or 3 amino acids in FR4 are substituted with another amino acid.

In some embodiments, the antigen-binding molecule comprises, or consists of, a single-domain antibody sequence comprising the CDRs, and the FRs of a clone 2RGT38, as shown in Tables A2 and B2.

In some embodiments, the antigen-binding molecule comprises a single-domain antibody sequence comprising the VHH sequence of 2RGT38, as shown in Table A2 herein.

In some embodiments, the antigen-binding molecule comprises a single-domain antibody sequence having at least 70%, e.g. one of ≥80%, ≥85%, ≥90%, ≥91%, ≥92%, ≥93%, ≥94%, ≥95%, ≥96%, ≥97%, 298%, ≥99% or 100% amino acid sequence identity to an amino acid sequence indicated in column A of Table A2. That is, in some embodiments, the antigen-binding molecule comprises a single-domain antibody sequence having at least 70%, e.g. one of ≥80%, ≥85%, ≥90%, ≥91%, ≥92%, ≥93%, ≥94%, 295%, ≥96%, ≥97%, ≥98%, 199% or 100% amino acid sequence identity to SEQ ID NO:377.

In another aspect, the disclosure provides an antigen-binding molecule which binds to IL-9Rα and comprises a single-domain antibody sequence incorporating the CDRs of a clone selected from: 2SAT363 and 2SAT57, as shown in Table A3 herein.

In some embodiments, the antigen-binding molecule which binds to IL-9Rα comprises a single-domain antibody sequence incorporating CDR1, CDR2 and CDR3 as indicated for one of binding moieties in Table A3, optionally wherein 1 or 2 or 3 amino acids in CDR1, and/or 1 or 2 or 3 amino acids in CDR2, and/or 1 or 2 or 3 amino acids in CDR3 are substituted with another amino acid.

In some embodiments, the antigen-binding molecule which binds to IL-9Rα of the present disclosure comprises a single-domain antibody sequence comprising the FRs of a clone selected from: 2SAT363 and 2SAT57, as shown in Table B3 herein.

In some embodiments, the antigen-binding molecule which binds to IL-9Rα comprises single-domain antibody sequence comprising FR1, FR2, FR3 and FR4 as indicated for one of binding moieties in Table B3, optionally wherein 1 or 2 or 3 amino acids in FR1, and/or 1 or 2 or 3 amino acids in FR2, and/or 1 or 2 or 3 amino acids in FR3, and/or 1 or 2 or 3 amino acids in FR4 are substituted with another amino acid.

In some embodiments, the antigen-binding molecule comprises, or consists of, a single-domain antibody sequence comprising the CDRs, and the FRs of a clone selected from: 2SAT363 and 2SAT57, as shown in Tables A3 and B3.

In some embodiments, the antigen-binding molecule which binds to IL-9Rα comprises a single-domain antibody sequence comprising the VHH sequence of a clone selected from: 2SAT363 and 2SAT57, as shown in Table A3 herein.

In some embodiments, the antigen-binding molecule which binds to IL-9Rα comprises a single-domain antibody sequence having at least 70%, e.g. one of ≥80%, ≥85%, ≥90%, ≥91%, ≥92%, ≥93%, ≥94%, 295%, ≥96%, ≥97%, ≥98%, 199% or 100% amino acid sequence identity to an amino acid sequence indicated in column A of Table A3.

It will be appreciated that an antigen-binding molecule comprising a γc-binding moiety, and an IL-9Rα-binding moiety according to present disclosure, may have a γc-binding moiety comprising a single-domain antibody sequence as defined above and/or the IL-9Rα-binding moiety comprising a single-domain antibody sequence as defined above. By way of example, in some embodiments, the γc-binding moiety comprises a single-domain antibody sequence having the CDRs of clone 2RGT38, and the IL-9Rα-binding moiety comprises a single-domain antibody sequence having the CDRs of clone 2SAT363. Exemplary combinations of γc-binding moieties and IL-9Rα-binding moieties in antigen-binding molecules according to the disclosure are shown in Table A4.

### Fc Regions

In some embodiments, the antigen-binding molecules of the present disclosure comprise an Fc region.

In some embodiments, the antigen-binding molecules of the present disclosure do not comprise an Fc region.

As used herein, an ‘Fc region’ refers to a polypeptide complex formed by interaction between two polypeptides, each polypeptide comprising the CH2-CH3 region of an immunoglobulin (Ig) heavy chain constant sequence.

Herein, a ‘CH2 region’ refers to an amino acid sequence corresponding to the CH2 region of an immunoglobulin (Ig). The CH2 region is the region of an Ig formed by positions 231 to 340 of the immunoglobulin constant region, according to the EU numbering system described in Edelman et al., Proc Natl Acad Sci USA (1969) 63(1):78-85. A ‘CH3 region’ refers to an amino acid sequence corresponding to the CH3 region of an immunoglobulin (Ig). The CH3 region is the region of an Ig formed by positions 341 to 447 of the immunoglobulin constant region, according to the EU numbering system described in Edelman et al., Proc Natl Acad Sci USA (1969) 63(1):78-85. A ‘CH2-CH3 region’ refers to an amino acid sequence corresponding to the CH2 and CH3 regions of an immunoglobulin (Ig). The CH2-CH3 region is the region of an Ig formed by positions 231 to 447 of the immunoglobulin constant region, according to the EU numbering system described in Edelman et al., Proc Natl Acad Sci USA (1969) 63(1):78-85.

In some embodiments, a CH2 region, CH3 region and/or a CH2-CH3 region according to the present disclosure corresponds to the CH2 region/CH3 region/CH2-CH3 region of an IgG (e.g. IgG1, IgG2, IgG3, IgG4), IgA (e.g. IgA1, IgA2), IgD, IgE or IgM. In some embodiments, the CH2 region, CH3 region and/or a CH2-CH3 region corresponds to the CH2 region/CH3 region/CH2-CH3 region of a human IgG (e.g. hIgG1, hIgG2, hIgG3, hIgG4), hIgA (e.g. hIgA1, hIgA2), hIgD, hIgE or hIgM. In some embodiments, the CH2 region, CH3 region and/or a CH2-CH3 region corresponds to the CH2 region/CH3 region/CH2-CH3 region of a human IgG1 allotype (e.g. G1m1, G1m2, G1m3 or G1m17).

Fc regions provide for interaction with Fc receptors and other molecules of the immune system to bring about functional effects. Fc-mediated effector functions are reviewed e.g. in Jefferis et al., Immunol Rev (1998) 163:59-76 (hereby incorporated by reference in its entirety), and are brought about through Fc-mediated recruitment and activation of immune cells (e.g. macrophages, dendritic cells, neutrophils, basophils, eosinophils, platelets, mast cells, NK cells and T cells) through interaction between the Fc region and Fc receptors expressed by the immune cells, recruitment of complement pathway components through binding of the Fc region to complement protein C1q, and consequent activation of the complement cascade. Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

Modifications to antibody Fc regions that influence Fc-mediated functions are known in the art, such as those described e.g. in Wang et al., Protein Cell (2018) 9(1):63-73, which is hereby incorporated by reference in its entirety. Exemplary Fc region modifications known to influence antibody effector function are summarised in Table 1 of Wang et al., Protein Cell (2018) 9(1):63-73. In some embodiments, the antigen-binding molecule of the present disclosure comprises an Fc region comprising modification to increase or reduce an Fc-mediated function as compared to an antigen-binding molecule comprising the corresponding unmodified Fc region.

Where an Fc region/CH2/CH3 is described as comprising modification(s) ‘corresponding to’ reference substitution(s), equivalent substitution(s) in the homologous Fc/CH2/CH3 are contemplated. By way of illustration, L234A/L235A substitutions in human IgG1 (numbered according to the EU numbering system as described in Kabat et al., Sequences of Proteins of Immunological Interest, 5th Ed. Public Health Service, National Institutes of Health, Bethesda, MD, 1991) correspond to L to A substitutions at positions 117 and 118 of the mouse Ig gamma-2A chain C region (UniProtKB: P01863-1, v1).

Where an Fc region is described as comprising a modification, the modification may be present in one or both of the polypeptide chains which together form the Fc region.

In some embodiments, the antigen-binding molecule of the present disclosure comprises an Fc region comprising modification. In some embodiments, the antigen-binding molecule of the present disclosure comprises an Fc region comprising modification in one or more of the CH2 and/or CH3 regions.

In some embodiments, the Fc region comprises modification to increase an Fc-mediated function. In some embodiments, the Fc region comprises modification to increase ADCC. In some embodiments, the Fc region comprises modification to increase ADCP. In some embodiments, the Fc region comprises modification to increase CDC. An antigen-binding molecule comprising an Fc region comprising modification to increase an Fc-mediated function (e.g. ADCC, ADCP, CDC) induces an increased level of the relevant effector function as compared to an antigen-binding molecule comprising the corresponding unmodified Fc region.

In some embodiments, the Fc region comprises modification to increase binding to an Fc receptor. In some embodiments, the Fc region comprises modification to increase binding to an Fcγ receptor. In some embodiments, the Fc region comprises modification to increase binding to one or more of FcγRI, FcγRIIa, FcγRIIb, FcγRIIc, FcγRIIIa and FcγRIIIb. In some embodiments, the Fc region comprises modification to increase binding to FcγRIIIa. In some embodiments, the Fc region comprises modification to increase binding to FcγRIIa. In some embodiments, the Fc region comprises modification to increase binding to FcγRIIb. In some embodiments, the Fc region comprises modification to increase binding to FcRn. In some embodiments, the Fc region comprises modification to increase binding to a complement protein. In some embodiments, the Fc region comprises modification to increase binding to C1q. In some embodiments, the Fc region comprises modification to promote hexamerisation of the antigen-binding molecule. In some embodiments, the Fc region comprises modification to increase antigen-binding molecule half-life. In some embodiments, the Fc region comprises modification to increase co-engagement.

In some embodiments, the Fc region comprises modification corresponding to the combination of substitutions F243L/R292P/Y300L/V3051/P396L as described in Stavenhagen et al. Cancer Res. (2007) 67:8882-8890. In some embodiments, the Fc region comprises modification corresponding to the combination of substitutions S239D/1332E or S239D/1332E/A330L as described in Lazar et al., Proc Natl Acad Sci USA. (2006) 103:4005-4010. In some embodiments, the Fc region comprises modification corresponding to the combination of substitutions S298A/E333A/K334A as described in Shields et al., J Biol Chem. (2001) 276:6591-6604. In some embodiments, the Fc region comprises modification to one of heavy chain polypeptides corresponding to the combination of substitutions L234Y/L235Q/G236W/S239M/H268D/D270E/S298A, and modification to the other heavy chain polypeptide corresponding to the combination of substitutions D270E/K326D/A330M/K334E, as described in Mimoto et al., MAbs. (2013) 5:229-236. In some embodiments, the Fc region comprises modification corresponding to the combination of substitutions G236A/S239D/1332E as described in Richards et al., Mol Cancer Ther. (2008) 7:2517-2527.

In some embodiments, the Fc region comprises modification corresponding to the combination of substitutions K326W/E333S as described in Idusogie et al. J Immunol. (2001) 166(4):2571-5. In some embodiments, the Fc region comprises modification corresponding to the combination of substitutions S267E/H268F/S324T as described in Moore et al. MAbs. (2010) 2(2):181-9. In some embodiments, the Fc region comprises modification corresponding to the combination of substitutions described in Natsume et al., Cancer Res. (2008) 68(10):3863-72. In some embodiments, the Fc region comprises modification corresponding to the combination of substitutions E345R/E430G/S440Y as described in Diebolder et al. Science (2014) 343(6176):1260-3.

In some embodiments, the Fc region comprises modification corresponding to the combination of substitutions M252Y/S254T/T256E as described in Dall'Acqua et al. J Immunol. (2002) 169:5171-5180. These so called ‘YTE’ modifications located at the CH2-CH3 interface of the Fc region have been shown to increase the binding affinity at pH 6.0 to the MHC Class I neonatal Fc receptor (FcRn), localised within the acidic endosomes of endothelial and haematopoietic cells, which increases efficient recycling of administered mAb and half-life in the plasma.

In some embodiments, the Fc region comprises modification corresponding to the combination of substitutions M428L/N434S as described in Zalevsky et al. Nat Biotechnol. (2010) 28:157-159.

In some embodiments, the Fc region comprises modification corresponding to the combination of substitutions S267E/L328F as described in Chu et al., Mol Immunol. (2008) 45:3926-3933. In some embodiments, the Fc region comprises modification corresponding to the combination of substitutions N325S/L328F as described in Shang et al. Biol Chem. (2014) 289:15309-15318.

In some embodiments, the Fc region comprises modification to reduce/prevent an Fc-mediated function. In some embodiments, the Fc region comprises modification to reduce/prevent ADCC. In some embodiments, the Fc region comprises modification to reduce/prevent ADCP. In some embodiments, the Fc region comprises modification to reduce/prevent CDC. An antigen-binding molecule comprising an Fc region comprising modification to reduce/prevent an Fc-mediated function (e.g. ADCC, ADCP, CDC) induces a reduced level of the relevant effector function as compared to an antigen-binding molecule comprising the corresponding unmodified Fc region.

In some embodiments, the Fc region comprises modification to reduce/prevent binding to an Fc receptor. In some embodiments, the Fc region comprises modification to reduce/prevent binding to an Fcγ receptor. In some embodiments, the Fc region comprises modification to reduce/prevent binding to one or more of FcγRI, FcγRIIa, FcγRIIb, FcγRIIc, FcγRIIIa and FcγRIIIb. In some embodiments, the Fc region comprises modification to reduce/prevent binding to FcγRIIIa. In some embodiments, the Fc region comprises modification to reduce/prevent binding to FcγRIIa. In some embodiments, the Fc region comprises modification to reduce/prevent binding to FcγRIIb. In some embodiments, the Fc region comprises modification to reduce/prevent binding to a complement protein. In some embodiments, the Fc region comprises modification to reduce/prevent binding to C1q. In some embodiments, the Fc region comprises modification to reduce/prevent glycosylation of the amino acid residue corresponding to N297.

In some embodiments, the Fc region is not able to induce one or more Fc-mediated functions (i.e. lacks the ability to elicit the relevant Fc-mediated function(s)). Accordingly, antigen-binding molecules comprising such Fc regions also lack the ability to induce the relevant function(s). Such antigen-binding molecules may be described as being devoid of the relevant function(s).

In some embodiments, the Fc region is not able to induce ADCC. In some embodiments, the Fc region is not able to induce ADCP. In some embodiments, the Fc region is not able to induce CDC. In some embodiments, the Fc region is not able to induce ADCC and/or is not able to induce ADCP and/or is not able to induce CDC.

In some embodiments, the Fc region is not able to bind to an Fc receptor. In some embodiments, the Fc region is not able to bind to an Fcγ receptor. In some embodiments, the Fc region is not able to bind to one or more of FcγRI, FcγRIIa, FcγRIIb, FcγRIIc, FcγRIIIa and FcγRIIIb. In some embodiments, the Fc region is not able to bind to FcγRIIIa. In some embodiments, the Fc region is not able to bind to FcγRIIa. In some embodiments, the Fc region is not able to bind to FcγRIIb. In some embodiments, the Fc region is not able to bind to FcRn. In some embodiments, the Fc region is not able to bind to a complement protein. In some embodiments, the Fc region is not able to bind to C1q. In some embodiments, the Fc region is not glycosylated at the amino acid residue corresponding to N297.

In some embodiments, the Fc region comprises modification corresponding to N297A or N297Q or N297G as described in Leabman et al., MAbs. (2013) 5:896-903. In some embodiments, the Fc region comprises modification corresponding to L235E as described in Alegre et al., J Immunol. (1992) 148:3461-3468. In some embodiments, the Fc region comprises modification corresponding to the combination of substitutions L234A/L235A or F234A/L235A as described in Xu et al., Cell Immunol. (2000) 200:16-26. In some embodiments, the Fc region comprises modification corresponding to P329A or P329G as described in Schlothauer et al., Protein Engineering, Design and Selection (2016), 29(10):457-466. In some embodiments, the Fc region comprises modification corresponding to the combination of substitutions L234A/L235A/P329G as described in Lo et al. J. Biol. Chem (2017) 292(9):3900-3908. In some embodiments, the Fc region comprises modification corresponding to the combination of substitutions described in Rother et al., Nat Biotechnol. (2007) 25:1256-1264. In some embodiments, the Fc region comprises modification corresponding to the combination of substitutions S228P/L235E as described in Newman et al., Clin. Immunol. (2001) 98:164-174. In some embodiments, the Fc region comprises modification corresponding to the combination of substitutions H268Q/V309L/A330S/P331 S as described in An et al., MAbs. (2009) 1:572-579. In some embodiments, the Fc region comprises modification corresponding to the combination of substitutions V234A/G237A/P238S/H268A/V309L/A330S/P331S as described in Vafa et al., Methods. (2014) 65:114-126. In some embodiments, the Fc region comprises modification corresponding to the combination of substitutions L234A/L235E/G237A/A330S/P331S as described in US 2015/0044231 A1.

The combination of substitutions ‘L234A/L235A’ and corresponding substitutions (such as e.g. F234A/L235A in human IgG4) are known to disrupt binding of Fc to Fcγ receptors and inhibit ADCC, ADCP, and also to reduce C1q binding and thus CDC (Schlothauer et al., Protein Engineering, Design and Selection (2016) 29(10):457-466, hereby incorporated by reference in entirety). The substitutions ‘P329G’ and ‘P329A’ reduce C1q binding (and thereby CDC). Substitution of ‘N297’ with ‘A’, ‘G’ or ‘Q’ is known to eliminate glycosylation, and thereby reduce Fc binding to C1q and Fcγ receptors, and thus CDC and ADCC. Lo et al. J. Biol. Chem (2017) 292(9):3900-3908 (hereby incorporated by reference in its entirety) reports that the combination of substitutions L234A/L235A/P329G eliminated complement binding and fixation as well as Fcγ receptor dependent, antibody-dependent, cell-mediated cytotoxicity in both murine IgG2a and human IgG1.

The combination of substitutions L234A/L235E/G237A/A330S/P331S in IgG1 Fc is disclosed in US 2015/0044231 A1 to abolish induction of phagocytosis, ADCC and CDC.

In some embodiments, the Fc region comprises modification corresponding to the substitution S228P as described in Silva et al., J Biol Chem. (2015) 290(9):5462-5469. The substitution S228P in IgG4 Fc reduces Fab-arm exchange (Fab-arm exchange can be undesirable).

In some embodiments, the Fc region comprises modification corresponding to the combination of substitutions L234A/L235A. In some embodiments, the Fc region comprises modification corresponding to the substitution P329G. In some embodiments, the Fc region comprises modification corresponding to the substitution N297Q.

In some embodiments, the Fc region comprises modification corresponding to the combination of substitutions L234A/L235A/P329G.

In some embodiments, the Fc region comprises modification corresponding to the combination of substitutions L234A/L235A/P329G/N297Q.

In some embodiments, the Fc region comprises modification corresponding to the combination of substitutions L234A/L235E/G237A/A330S/P331S.

In some embodiments, the Fc region comprises modification corresponding to the substitution S228P, e.g. in IgG4.

In some embodiments, the Fc region comprises a CH2-CH3 region comprising an amino acid difference at one or more of the following positions, relative to the amino acid sequence of a CH2-CH3 region of a reference Fc region: 234 or 235 (according to the EU numbering system). In some embodiments, the Fc region comprises a CH2-CH3 region comprising one or more of the following specified amino acid residues: A234 or A235 (according to the EU numbering system). In some embodiments, the Fc region comprises a CH2-CH3 region comprising A234 and A235. In some embodiments, the Fc region comprises a CH2-CH3 region comprising one or more of the following amino acid substitutions, relative to the amino acid sequence of a CH2-CH3 region of the reference Fc region: L234A or L235A (according to the EU numbering system). In some embodiments, the Fc region comprises a CH2-CH3 region comprising the following amino acid substitutions, relative to the amino acid sequence of a CH2-CH3 region of the reference Fc region: L234A and L235A (according to the EU numbering system).

In some embodiments—particularly embodiments in which the antigen-binding molecule is a multispecific (e.g. bispecific) antigen-binding molecule—the antigen-binding molecule comprises an Fc region comprising modification in one or more of the CH2 and CH3 regions promoting association of the Fc region. Recombinant co-expression of constituent polypeptides of an antigen-binding molecule and subsequent association leads to several possible combinations. To improve the yield of the desired combinations of polypeptides in antigen-binding molecules in recombinant production, it is advantageous to introduce in the Fc regions modification(s) promoting association of the desired combination of heavy chain polypeptides. Modifications may promote e.g. hydrophobic and/or electrostatic interaction between CH2 and/or CH3 regions of different polypeptide chains. Suitable modifications are described e.g. in Ha et al., Front Immnol. (2016) 7:394, which is hereby incorporated by reference in its entirety.

In some embodiments, the antigen-binding molecule of the present disclosure comprises an Fc region comprising paired substitutions in the CH3 regions of the Fc region according to one of the following formats, as shown in Table 1 of Ha et al., Front Immnol. (2016) 7:394: KiH, KiHs-s, HA-TF, ZW1, 7.8.60, DD-KK, EW-RVT, EW-RVTs-s, SEED or A107.

In some embodiments, the multispecific (e.g. bispecific) antigen-binding molecule of the present disclosure is provided with an Fc region comprising the ‘knob-into-hole’ or ‘KiH’ modification, e.g. as described e.g. in U.S. Pat. No. 7,695,936 and Carter, J Immunol Meth. (2001) 248:7-15. In such embodiments, one of the CH3 regions of the Fc region comprises a ‘knob’ modification, and the other CH3 region comprises a ‘hole’ modification. The ‘knob’ and ‘hole’ modifications are positioned within the respective CH3 regions so that the ‘knob’ can be positioned in the ‘hole’ in order to promote heterodimerisation (and inhibit homodimerisation) of the polypeptides and/or stabilise heterodimers. Knobs are constructed by substituting amino acids having small chains with those having larger side chains (e.g. tyrosine or tryptophan). Holes are created by substituting amino acids having large side chains with those having smaller side chains (e.g. alanine or threonine).

In some embodiments, one of the CH3 regions of the Fc region of the antigen-binding molecule of the present disclosure comprises the substitution (numbering of positions/substitutions in the Fc region herein is according to the EU numbering system as described in Kabat et al., Sequences of Proteins of Immunological Interest, 5th Ed. Public Health Service, National Institutes of Health, Bethesda, MD, 1991) T366W, and the other CH3 region of the Fc region comprises the substitution Y407V. In some embodiments, one of the CH3 regions of the Fc region of the antigen-binding molecule comprises the substitution T366W, and the other CH3 region of the Fc region comprises the substitutions T366S and L368A. In some embodiments, one of the CH3 regions of the Fc region of the antigen-binding molecule comprises the substitution T366W, and the other CH3 region of the Fc region comprises the substitutions Y407V, T366S and L368A.

In some embodiments, one of the CH3 regions comprises the substitution S354C, and the other CH3 region of the Fc region comprises the substitution Y349C. Introduction of these cysteine residues results in formation of a disulfide bridge between the two CH3 regions of the Fc region, further stabilizing the heterodimer (Carter, J Immunol Methods (2001) 248:7-15).

In some embodiments, one of the CH3 regions comprises the substitutions K392D and K409D, and the other CH3 region of the Fc region comprises the substitutions E356K and D399K. ‘DDKK’ knob-into-hole technology is described e.g. in WO 2014/131694 A1, and promotes assembly of the heavy chains providing the complementary amino acid residues.

In some embodiments, the antigen-binding molecule of the present disclosure comprises an Fc region modified as described in Labrijn et al., Proc Natl Acad Sci USA. (2013) 110(13):5145-50, referred to as ‘Duobody’ format. In some embodiments one of the CH3 regions comprises the substitution K409R, and the other CH3 region of the Fc region comprises the substitution K405L.

In some embodiments, the antigen-binding molecule of the present disclosure comprises an Fc region modified as described in Strop et al., J Mol Biol. (2012) 420(3):204-19, so-called ‘EEE-RRR’ format. In some embodiments one of the CH3 regions comprises the substitutions D221E, P228E and L368E, and the other CH3 region of the Fc region comprises the substitutions D221R, P228R and K409R.

In some embodiments, the antigen-binding molecule comprises an Fc region comprising the ‘EW-RVT’ modification described in Choi et al., Mol Cancer Ther. (2013) 12(12):2748-59. In some embodiments one of the CH3 regions comprises the substitutions K360E and K409W, and the other CH3 region of the Fc region comprises the substitutions Q347R, D399V and F405T.

In some embodiments, the antigen-binding molecule of the present disclosure comprises an Fc region comprising the ‘SEED’ modification as described in Davis et al., Protein Eng Des Sel. (2010) 23(4):195-202, in which P-strand segments of human IgG1 CH3 and IgA CH3 are exchanged.

In some embodiments, one of the CH3 regions comprises the substitutions S364H and F405A, and the other CH3 region of the Fc region comprises the substitutions Y349T and T394F (see e.g. Moore et al., MAbs (2011) 3(6):546-57).

In some embodiments, one of the CH3 regions comprises the substitutions T350V, L351Y, F405A and Y407V, and the other CH3 region of the Fc region comprises the substitutions T350V, T366L, K392L and T394W (see e.g. Von Kreudenstein et al., MAbs (2013) 5(5):646-54).

In some embodiments, one of the CH3 regions comprises the substitutions K360D, D399M and Y407A, and the other CH3 region of the Fc region comprises the substitutions E345R, Q347R, T366V and K409V (see e.g. Leaver-Fay et al., Structure (2016) 24(4):641-51).

In some embodiments, one of the CH3 regions comprises the substitutions K370E and K409W, and the other CH3 region of the Fc region comprises the substitutions E357N, D399V and F405T (see e.g. Choi et al., PLoS One (2015) 10(12):e0145349).

In some embodiments, the antigen-binding molecule of the present disclosure comprises an Fc region comprising modification to increase stability (e.g. thermostability and/or freeze-thaw stability). In some embodiments, the antigen-binding molecule comprises modification to one or more of the CH2 and CH3 regions to increase stability (e.g. thermostability and/or freeze-thaw stability).

In some embodiments, the antigen-binding molecule of the present disclosure comprises CH3 regions (e.g. within an Fc region, e.g. within CH2-CH3 regions forming an Fc region) comprising paired CH3 region ‘KiH’ or ‘KiH_{S-S}’ modifications. Such paired CH3 regions may comprise a CH3 region comprising a knob modification, and a CH3 region comprising a hole modification.

In some embodiments, a CH3 region comprising a knob modification comprises a tryptophan or tyrosine residue at position 366 (i.e. 366W or 366Y). In some embodiments, the knob modification is or comprises T366W or T366Y. In some embodiments, a CH3 region comprising a knob modification comprises 366W. In some embodiments, the knob modification is or comprises T366W.

In some embodiments, a CH3 region comprising a hole modification comprises 407V, 407A, 407S or 407T; 366S, 366V or 366A; and 368A, 368V, 368S or 368T. In some embodiments, the hole modification is or comprises Y407V, Y407A, Y407S or Y407T; T366S, T366V or T366A; and L368A, L368V, L368S or L368T. In some embodiments, a CH3 region comprising a hole modification comprises 407V, 366S and 368A. In some embodiments, the hole modification is or comprises Y407V, T366S, and L368A.

In some embodiments, the antigen-binding molecule of the present disclosure comprise CH3 region(s) (e.g. within an Fc region, e.g. within CH2-CH3 region(s) of an Fc region) comprising modification for the formation of an interchain disulfide bond (i.e. between polypeptides comprising CH2-CH3 regions forming the Fc region). Such modification may comprise the introduction of one or more cysteine residues into one or both of the CH3 regions of the constituent polypeptides of a polypeptide complex of the present disclosure. More particularly, such modification may have the result that the CH3:CH3 interface formed between the CH3 regions of polypeptides of polypeptide complexes of the present disclosure comprises a disulfide bond, formed between cysteine residues (one from each polypeptide). In some embodiments, one of the CH3 regions comprises 349C, and the other CH3 region comprises 354C. In some embodiments, one of the CH3 regions comprises Y349C, and the other CH3 region comprises S354C.

In some embodiments, a CH3 region comprising a knob modification comprises 366W and S354C. In some embodiments, a CH3 region comprising a hole modification comprises Y407V, T366S, L368A and Y349C.

### Further Antigen-Binding Moiety

In some embodiments, the antigen-binding molecule comprises a further antigen-binding moiety. In some embodiments, the further antigen-binding moiety binds to a target antigen other than a γc-containing cytokine receptor polypeptide (e.g. a target antigen which is not γc or IL-9Rα). That is, in some embodiments, the antigen-binding molecule of the present disclosure comprises (i) a γc-binding moiety, (ii) a moiety that binds to IL-9Rα, and (iii) a moiety that binds to a target antigen (e.g. an antigen that is not a γc-containing cytokine receptor polypeptide).

It will be appreciated that an effect of moiety (iii) is to localise the antigen-binding molecule to cells expressing its target. This can be useful to direct the effect of moieties (i) and (ii) of the antigen-binding molecule to cells expressing the target for moiety (iii). By way of illustration, in embodiments wherein moiety (ii) is an IL-9Rα-binding moiety and wherein moiety (iii) is a CD8-binding moiety, the effect of moiety (iii) is to target the γc:IL-9Rα receptor agonist/antagonist activity conferred by moieties (i) and (ii) to CD8+ T cells.

Moiety (iii) can also be employed to target the antigen-binding molecule to an anatomical site/tissue/organ of interest. This can be useful to direct the effect of moieties (i) and (ii) of the antigen-binding molecule to such regions. By way of illustration, in embodiments wherein moiety (ii) is an IL-9Rα-binding moiety and wherein moiety (iii) is a cancer cell antigen-binding moiety, the effect of moiety (iii) is to target the γc:IL-9Rα receptor agonist/antagonist activity conferred by moieties (i) and (ii) to γc:IL-9Rα receptor-expressing cells in the proximity of the cells expressing the cancer cell antigen.

Thus, it will be appreciated that moiety (iii) is employed to target/localise the antigen-binding molecule to, and/or increase the local concentration of the antigen-binding molecule in the proximity of, a cell comprising/expressing the target antigen for moiety (iii).

The target for moiety (iii) may be any target antigen. In some embodiments, the target antigen may be a peptide/polypeptide, glycoprotein, lipoprotein, glycan, glycolipid, lipid, or fragment thereof. The antigen is preferably expressed at the cell surface of a cell expressing the antigen.

In some embodiments, the target antigen is a disease-associated antigen or an antigen expressed by an immune cell.

A ‘disease-associated antigen’ refers to an antigen whose presence is indicative of a given disease/disease state, or an antigen for which an elevated level of the antigen is positively-correlated with a given disease/disease state. The disease-associated antigen may be an antigen whose expression is associated with the development, progression or severity of symptoms of a given disease. The disease-associated antigen may be associated with the cause or pathology of the disease, or may be expressed abnormally as a consequence of the disease. A disease-associated antigen may be an antigen of an infectious agent or pathogen, a cancer-associated antigen or an autoimmune disease-associated antigen.

In some embodiments, the disease-associated antigen is an antigen of a pathogen. The pathogen may be prokaryotic (bacteria), eukaryotic (e.g. protozoan, helminth, fungus), virus or prion. In some embodiments, the pathogen is an intracellular pathogen. In some embodiments the pathogen is a virus, e.g. a virus as described hereinabove. In some embodiments the pathogen is a bacterium.

In some embodiments, the target antigen is a cancer-associated antigen. A cancer-associated antigen is an antigen whose expression or overexpression is associated with cancer. In some embodiments, the cancer-associated antigen is a receptor molecule, e.g. a cell surface receptor. In some embodiments, the cancer-associated antigen is a cell signalling molecule, e.g. a cytokine, chemokine, interferon, interleukin or lymphokine. In some embodiments, the cancer-associated antigen is a growth factor or a hormone. In some embodiments, the cancer-associated antigen is a viral antigen. A cancer cell antigen may be abnormally expressed by a cancer cell (e.g. the cancer cell antigen may be expressed with abnormal localisation), or may be expressed with an abnormal structure by a cancer cell. A cancer cell antigen may be capable of eliciting an immune response. In some embodiments, the antigen is expressed at the cell surface of the cancer cell (i.e. the cancer cell antigen is a cancer cell surface antigen). In some embodiments, the part of the antigen which is bound by the antigen-binding molecule described herein is displayed on the external surface of the cancer cell (i.e. is extracellular). The cancer cell antigen may be a cancer-associated antigen. In some embodiments the cancer cell antigen is an antigen whose expression is associated with the development, progression or severity of symptoms of a cancer. The cancer-associated antigen may be associated with the cause or pathology of the cancer, or may be expressed abnormally as a consequence of the cancer. In some embodiments, the cancer cell antigen is an antigen whose expression is upregulated (e.g. at the RNA and/or protein level) by cells of a cancer, e.g. as compared to the level of expression by comparable non-cancerous cells (e.g. non-cancerous cells derived from the same tissue/cell type). In some embodiments, the cancer-associated antigen may be preferentially expressed by cancerous cells, and not expressed by comparable non-cancerous cells (e.g. non-cancerous cells derived from the same tissue/cell type). In some embodiments, the cancer-associated antigen may be the product of a mutated oncogene or mutated tumor suppressor gene. In some embodiments, the cancer-associated antigen may be the product of an overexpressed cellular protein, a cancer antigen produced by an oncogenic virus, an oncofetal antigen, or a cell surface glycolipid or glycoprotein.

Cancer-associated antigens are reviewed by Zarour H M, DeLeo A, Finn O J, et al. Categories of Tumor Antigens. In: Kufe D W, Pollock R E, Weichselbaum R R, et al., editors. Holland-Frei Cancer Medicine. 6th edition. Hamilton (ON): BC Decker; 2003. Cancer-associated antigens include oncofetal antigens: CEA, Immature laminin receptor, TAG-72; oncoviral antigens such as HPV E6 and E7; overexpressed proteins: BING-4, calcium-activated chloride channel 2, cyclin-B1, 9D7, Ep-CAM, EphA3, HER2/neu, telomerase, mesothelin, SAP-1, survivin; cancer-testis antigens: BAGE, CAGE, GAGE, MAGE, SAGE, XAGE, CT9, CT10, NY-ESO-1, PRAME, SSX-2; lineage restricted antigens: MART1, Gp100, tyrosinase, TRP-1/2, MC1R, prostate specific antigen; mutated antigens: P-catenin, BRCA1/2, CDK4, CML66, Fibronectin, MART-2, p53, Ras, TGF-βRII; post-translationally altered antigens: MUC1, idiotypic antigens: Ig, TCR. Other cancer cell antigens include heat-shock protein 70 (HSP70), heat-shock protein 90 (HSP90), glucose-regulated protein 78 (GRP78), vimentin, nucleolin, feto-acinar pancreatic protein (FAPP), alkaline phosphatase placental-like 2 (ALPPL-2), siglec-5, stress-induced phosphoprotein 1 (STIP1), protein tyrosine kinase 7 (PTK7), and cyclophilin B. In some embodiments the cancer cell antigen is a cancer cell antigen described in Zhao and Cao, Front Immunol. (2019) 10:2250, which is hereby incorporated by reference in its entirety.

In some embodiments, the target antigen is an immune cell surface molecule. An immune cell surface molecule is any molecule which is expressed in or at the cell membrane of an immune cell. In some embodiments, the part of the immune cell surface molecule which is bound by the antigen-binding moiety is on the external surface of the immune cell (i.e. is extracellular). The immune cell surface molecule may be expressed at the cell surface of any immune cell. In some embodiments, the immune cell may be a cell of hematopoietic origin, e.g. a neutrophil, eosinophil, basophil, dendritic cell, lymphocyte, or monocyte. The lymphocyte may be e.g. a T cell, B cell, natural killer (NK) cell, NKT cell or innate lymphoid cell (ILC), or a precursor thereof (e.g. a thymocyte or pre-B cell). The immune cell may express a CD3 polypeptide (e.g. CD3γ CD3ε CD3ζ or CD3δ), a TCR polypeptide (TCRα or TCRβ), CD27, CD28, CD4 or CD8. In some embodiments, the immune cell is a T cell, e.g. a CD3+ T cell. In some embodiments, the T cell is a CD3+, CD4+ T cell. In some embodiments, the T cell is a CD3+, CD8+ T cell. In some embodiments, the T cell is a T helper cell (TH cell). In some embodiments, the T cell is a cytotoxic T cell (e.g. a cytotoxic T lymphocyte (CTL)). In some embodiments, the immune cell is a T cell or an NK cell.

In some embodiments, the immune cell surface molecule may be a CD3-TCR complex polypeptide, e.g. TCRα, TCRβ, TCRγ, TCRδ, TRAC, TRBC1, TRBC2, TRGC1, TRGC2, TRDC, CD3ε, CD3δ, CD3γ, CD3ζ or CD3η. In some embodiments, the immune cell surface molecule is CD3, CD8, CD4 or CD28. In some embodiments, the immune cell surface molecule is a checkpoint molecule (e.g. PD-1, CTLA-4, LAG-3, TIM-3, VISTA, TIGIT or BTLA), or a ligand thereof. In some embodiments the immune cell surface molecule is a costimulatory molecule (e.g. CD28, OX40, 4-1 BB, ICOS or CD27), or a ligand thereof.

In some embodiments, the target antigen is selected from PD-1, 4-1 BB and CD8.

### Chimeric Antigen Receptors (CARs)

The present disclosure also provides Chimeric Antigen Receptors (CARs). CARs are recombinant receptors that provide both antigen-binding and T cell activating functions. CAR structure and engineering is reviewed, for example, in Dotti et al., Immunol Rev (2014) 257(1):107-126, hereby incorporated by reference in its entirety. CARs comprise an antigen-binding region linked to a cell membrane anchor region and a signalling region. An optional hinge region may provide separation between the antigen-binding region and cell membrane anchor region, and may act as a flexible linker.

The antigen-binding domain of a CAR according to the present disclosure comprises or consists of an antigen-binding molecule as described herein. Accordingly, a CAR according to the present disclosure comprises an antigen-binding molecule as described herein.

It will be appreciated that an antigen-binding molecule according to the present disclosure forms, or is comprised in, the antigen-binding domain of the CAR. Accordingly, in some embodiments, the antigen-binding molecule of the present disclosure is comprised in a CAR.

It will also be appreciated that an antigen-binding molecule according to the present disclosure may be a CAR. A CAR having an antigen-binding domain comprising or consisting of an antigen-binding molecule of the present disclosure is an antigen-binding molecule. The antigen-binding domain of the CAR of the present disclosure may be provided with any suitable format, e.g. scFv, scFab, etc.

The cell membrane anchor region is provided between the antigen-binding region and the signalling region of the CAR and provides for anchoring the CAR to the cell membrane of a cell expressing a CAR, with the antigen-binding region in the extracellular space, and signalling region inside the cell. In some embodiments, the CAR comprises a cell membrane anchor region comprising or consisting of an amino acid sequence which comprises, consists of, or is derived from, the transmembrane region amino acid sequence for one of CD3-ζ, CD4, CD8 or CD28. As used herein, a region which is ‘derived from’ a reference amino acid sequence comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the reference sequence.

The signalling region of a CAR allows for activation of the T cell. The CAR signalling regions may comprise the amino acid sequence of the intracellular domain of CD3-ζ, which provides immunoreceptor tyrosine-based activation motifs (ITAMs) for phosphorylation and activation of the CAR-expressing T cell. Signalling regions comprising sequences of other ITAM-containing proteins such as FcγRI have also been employed in CARs (Haynes et al., J Immunol. (2001) 166(1):182-187). Signalling regions of CARs may also comprise co-stimulatory sequences derived from the signalling region of co-stimulatory molecules, to facilitate activation of CAR-expressing T cells upon binding to the target protein. Suitable co-stimulatory molecules include CD28, OX40, 4-1BB, ICOS and CD27. In some cases CARs are engineered to provide for co-stimulation of different intracellular signalling pathways. For example, signalling associated with CD28 co-stimulation preferentially activates the phosphatidylinositol 3-kinase (PI3K) pathway, whereas 4-1 BB-mediated signalling is through TNF receptor associated factor (TRAF) adaptor proteins. Signalling regions of CARs therefore sometimes contain co-stimulatory sequences derived from signalling regions of more than one co-stimulatory molecule. In some embodiments, the CAR of the present disclosure comprises one or more co-stimulatory sequences comprising or consisting of an amino acid sequence which comprises, consists of, or is derived from, the amino acid sequence of the intracellular domain of one or more of CD28, OX40, 4-1 BB, ICOS and CD27.

An optional hinge region may provide separation between the antigen-binding domain and the transmembrane domain, and may act as a flexible linker. Hinge regions may be derived from IgG1. In some embodiments, the CAR of the present disclosure comprises a hinge region comprising or consisting of an amino acid sequence which comprises, consists of, or is derived from, the amino acid sequence of the hinge region of IgG1.

Also provided is a cell comprising a CAR according to the present disclosure. The CAR according to the present disclosure may be used to generate CAR-expressing immune cells, e.g. CAR-T or CAR-NK cells. Engineering of CARs into immune cells may be performed during culture, in vitro.

### Functional Properties

Antigen-binding molecules described herein may be characterised by reference to certain functional properties. In some embodiments, an antigen-binding molecule described herein may possess one or more of the following properties:
binds to γc;
binds to a polypeptide of a γc-containing cytokine receptor other than γc;
binds to IL-9Rα;
binds to γc and a polypeptide of a γc-containing cytokine receptor other than γc;
binds to γc and IL-9Rα;
binds to γc-expressing cells;
binds to cells expressing a polypeptide of a γc-containing cytokine receptor other than γc;
binds to IL-9Rα-expressing cells;
binds to cells expressing γc and a polypeptide of a γc-containing cytokine receptor other than γc;
binds to cells expressing γc and IL-9Rα;
binds to cells expressing a receptor comprising γc and a polypeptide of a γc-containing cytokine receptor other than γc;
binds to cells expressing γc:IL-9Rα receptor;
increases multimerization of γc and IL-9Rα;
decreases multimerization of γc and IL-9Rα;
increases signalling mediated by a γc-containing cytokine receptor to which the antigen-binding molecule binds (e.g. γc:IL-9Rα receptor);
increases signalling mediated by a γc:IL-9Rα receptor;
increases signalling mediated by IL-9;
decreases signalling mediated by a γc-containing cytokine receptor to which the antigen-binding molecule binds (e.g. γc:IL-9Rα receptor);
decreases signalling mediated by a γc:IL-9Rα receptor;
decreases signalling mediated by IL-9;
increases proliferation, survival and/or effector activity of cells expressing a γc-containing cytokine receptor to which the antigen-binding molecule binds (e.g. γc:IL-9Rα receptor); increases proliferation, survival and/or effector activity of cells expressing IL-9Rα; decreases proliferation, survival and/or effector activity of cells expressing a γc-containing cytokine receptor to which the antigen-binding molecule binds (e.g. γc:IL-9Rα receptor); increases proliferation, survival and/or effector activity of cells expressing IL-9Rα; reduces expression of one or more markers of immune cell exhaustion by cells expressing a γc-containing cytokine receptor to which the antigen-binding molecule binds (e.g. γc:IL-9Rα receptor);
reduces expression of one or more markers of immune cell exhaustion by cells expressing IL-9Rα;
increases expression of one or more markers of immune cell exhaustion by cells expressing a γc-containing cytokine receptor to which the antigen-binding molecule binds (e.g. γc:IL-9Rα receptor);
increases expression of one or more markers of immune cell exhaustion by cells expressing IL-9Rα;
decreases multimerization of γc and a polypeptide of a γc-containing cytokine receptor other than γc (e.g. IL-9Rα), decreases signalling mediated by a γc-containing cytokine receptor to which the antigen-binding molecule binds (e.g. γc:IL-9Rα receptor), and/or decreases proliferation, survival and/or effector activity of cells expressing a γc-containing cytokine receptor to which the antigen-binding molecule binds (e.g. γc:IL-9Rα receptor) independently of Fc-mediated function; decreases multimerization of γc and IL-9Rα, decreases signalling mediated by γc:IL-9Rα receptor, and/or decreases proliferation, survival and/or effector activity of cells expressing γc:IL-9Rα receptor, independently of Fc-mediated function;
increases cell killing/depletion of, and/or reduces the number/proportion of, cells comprising/expressing one or more of the target antigens of its constituent antigen-binding moieties (i.e. γc, and/or IL-9Rα));
increases cell killing/depletion of, and/or reduces the number/proportion of, cells comprising/expressing γc, and/or IL-9Rα;
increased stability and/or half-life compared to one or more γc family cytokines (e.g. IL-9); increased stability and/or half-life compared to IL-9;
increased upregulation of signalling mediated by a γc-containing cytokine receptor to which the antigen-binding molecule binds (e.g. γc:IL-9Rα receptor) compared to a cytokine that binds to the γc-containing cytokine receptor;
increased upregulation of signalling mediated by γc:IL-9Rα receptor compared to IL-9; decreased upregulation of signalling mediated by a γc-containing cytokine receptor to which the antigen-binding molecule binds (e.g. γc:IL-9Rα receptor) compared to a cytokine that binds to the γc-containing cytokine receptor;
decreased upregulation of signalling mediated by a γc:IL-9Rα receptor compared to IL-9; increased upregulation of proliferation, survival and/or effector activity of cells expressing a γc-containing cytokine receptor to which the antigen-binding molecule binds (e.g. γc:IL-9Rα receptor) compared to a cytokine that binds to the γc-containing cytokine receptor; increased upregulation of proliferation, survival and/or effector activity of cells expressing a γc:IL-9Rα receptor compared to IL-9;
decreased upregulation of proliferation, survival and/or effector activity of cells expressing a γc-containing cytokine receptor to which the antigen-binding molecule binds (e.g. γc:IL-9Rα receptor) compared to a cytokine that binds to the γc-containing cytokine receptor; decreased upregulation of proliferation, survival and/or effector activity of cells expressing a γc:IL-9Rα receptor compared to IL-9;
increased downregulation of expression of one or more markers of immune cell exhaustion by cells expressing a γc-containing cytokine receptor to which the antigen-binding molecule binds (e.g. γc:IL-9Rα receptor) compared to a cytokine that binds to the γc-containing cytokine receptor;
increased downregulation of expression of one or more markers of immune cell exhaustion by cells expressing a γc:IL-9Rα receptor compared to IL-9;
decreased downregulation of expression of one or more markers of immune cell exhaustion by cells expressing a γc-containing cytokine receptor to which the antigen-binding molecule binds (e.g. γc:IL-9Rα receptor) compared to a cytokine that binds to the γc-containing cytokine receptor;
decreased downregulation of expression of one or more markers of immune cell exhaustion by cells expressing a γc:IL-9Rα receptor compared to IL-9; and/or enhances anticancer activity of cancer antigen-specific immune cells, e.g. in vivo.

It will be appreciated that a given antigen-binding molecule may display more than one of the properties recited in the preceding paragraph. A given antigen-binding molecule may be evaluated for the properties recited in the preceding paragraph using suitable assays. For example, the assays may be e.g. in vitro assays, optionally cell-based assays or cell-free assays. In some embodiments, the assays may be e.g. in vivo assays, i.e. performed in non-human animals. In some embodiments, the assays may be e.g. ex vivo assays, i.e. performed using cells/tissue/an organ obtained from a subject. Such assays may be utilised to screen for antigen-binding molecules with a desired functional property.

Where assays are cell-based assays, they may comprise treating cells with a given antigen-binding molecule in order to determine whether the antigen-binding molecule displays one or more of the recited properties. Assays may employ species labelled with detectable entities in order to facilitate their detection. Assays may comprise evaluating the recited properties following treatment of cells separately with a range of quantities/concentrations of a given antigen-binding molecule (e.g. a dilution series). It will be appreciated that the cells preferably express the target antigen for the antigen-binding molecule.

Analysis of the results of such assays may comprise determining the concentration at which 50% of the maximal level of the relevant activity is attained. The concentration of a given agent at which 50% of the maximal level of the relevant activity is attained may be referred to as the ‘half-maximal effective concentration’ of the agent in relation to the relevant activity, which may also be referred to as the ‘EC₅₀’. By way of illustration, the EC₅₀ of a given antigen-binding molecule for binding to human γc may be the concentration of the antigen-binding molecule at which 50% of the maximal level of binding to human γc is achieved.

Depending on the property, the EC₅₀ may also be referred to as the ‘half-maximal inhibitory concentration’ or ‘IC₅₀’, this being the concentration of the agent at which 50% of the maximal level of inhibition of a given property is observed.

Where the functional properties of agents are compared (e.g. where the antigen binding molecule of the present disclosure are compared with other polypeptides), comparisons are performed at equivalent concentrations and/or quantity of the relevant agents.

The antigen-binding molecules and antigen-binding moieties described herein preferably display specific binding to γc and/or IL-9Rα. As used herein, ‘specific binding’ refers to binding which is selective for the antigen, and which can be discriminated from non-specific binding to non-target antigen. An antigen-binding molecule/moiety that specifically binds to a target molecule preferably binds the target with greater affinity, and/or with greater duration than it binds to other, non-target molecules.

The ability of a given polypeptide to bind specifically to a given molecule can be determined by analysis according to methods known in the art, such as by ELISA, Surface Plasmon Resonance (SPR; see e.g. Hearty et al., Methods Mol Biol. (2012) 907:411-442), Bio-Layer Interferometry (BLI; see e.g. Lad et al., J Biomol Screen (2015) 20(4):498-507), flow cytometry, or by a radiolabeled antigen-binding assay (RIA) enzyme-linked immunosorbent assay. Through such analysis binding to a given molecule can be measured and quantified. In some embodiments, the binding may be the response detected in a given assay.

In some embodiments, the extent of binding of the antigen-binding molecule/moiety to a non-target molecule is less than about 10% of the binding of the antigen-binding molecule/moiety to the target molecule as measured, e.g. by ELISA, SPR, BLI or by RIA. Alternatively, binding specificity may be reflected in terms of binding affinity where the antigen-binding molecule/moiety binds with a dissociation constant (Ko) that is at least 0.1 order of magnitude (i.e. 0.1×10ⁿ, where n is an integer representing the order of magnitude) greater than the Ko of the antigen-binding molecule towards a non-target molecule. This may optionally be one of at least 0.2, 0.3, 0.4, 0.5, 0.6, 0.7, 0.8, 0.9, 1.0, 1.5, or 2.0.

Binding affinity of an antigen-binding molecule/moiety for its target is often described in terms of its dissociation constant (K_{D}). Binding affinity can be measured by methods known in the art, such as by ELISA, Surface Plasmon Resonance (SPR; see e.g. Hearty et al., Methods Mol Biol (2012) 907:411-442; or Rich et al., Anal Biochem. (2008) 373(1):112-20), Bio-Layer Interferometry (see e.g. Lad et al., J Biomol Screen (2015) 20(4):498-507; or Concepcion et al., Comb Chem High Throughput Screen. (2009) 12(8):791-800), MicroScale Thermophoresis (MST) analysis (see e.g. Jerabek-Willemsen et al., Assay Drug Dev Technol. (2011) 9(4):342-353), or by a radiolabelled antigen-binding assay (RIA).

In some embodiments, an antigen-binding molecule/moiety described herein binds to γc and/or IL-9Rα with an affinity in the micromolar range, i.e. K_{D}=9.9×10⁻⁴ to 1×10⁻⁶ M. In some embodiments, an antigen-binding molecule/moiety described herein binds to γc and/or IL-9Rα with sub-micromolar affinity, i.e. K_{D}<1×10⁻⁶ M. In some embodiments, an antigen-binding molecule/moiety described herein binds to γc and/or IL-9Rα with an affinity in the nanomolar range, i.e. K_{D}=9.9×10⁻⁷ to 1×10⁻⁹ M. In some embodiments, an antigen-binding molecule/moiety described herein binds to γc and/or IL-9Rα with sub-nanomolar affinity, i.e. K_{D}<1×10⁻⁹ M. In some embodiments, an antigen-binding molecule/moiety described herein binds to γc and/or IL-9Rα with an affinity in the picomolar range, i.e. K_{D}=9.9×10⁻¹⁰ to 1×10⁻¹² M. In some embodiments, an antigen-binding molecule/moiety described herein binds to γc and/or IL-9Rα with sub-picomolar affinity, i.e. K_{D}<1×10⁻¹² M.

The antigen-binding molecules and antigen-binding moieties of the present disclosure may bind to a particular region of interest of their target antigen(s). For example, they may bind to a linear epitope of γc, and/or IL-9Rα, consisting of a contiguous sequence of amino acids (i.e. an amino acid primary sequence). In some embodiments, they may bind to a conformational epitope of γc and/or IL-9Rα, consisting of a discontinuous sequence of amino acids of the amino acid sequence.

The region of a given target molecule to which an antigen-binding molecule binds can be determined by the skilled person using various methods well known in the art, including X-ray co-crystallography analysis of antibody-antigen complexes, peptide scanning, mutagenesis mapping, hydrogen-deuterium exchange analysis by mass spectrometry, phage display, competition ELISA and proteolysis-based ‘protection’ methods. Such methods are described, for example, in Gershoni et al., BioDrugs (2007) 21(3):145-156, which is hereby incorporated by reference in its entirety.

The antigen-binding molecules and antigen-binding moieties preferably bind to their target antigen(s) in a region which is accessible to an antigen-binding molecule (i.e. an extracellular antigen-binding molecule) when the target antigen(s) is/are expressed at the cell surface (i.e. in or at the cell membrane). In some embodiments, the antigen-binding molecules and antigen-binding moieties are capable of binding to their target antigen(s) when they are expressed at the cell surface.

The antigen-binding molecules and antigen-binding moieties preferably bind to the extracellular domain(s) of target antigen(s). The extracellular domains of γc and/or IL-9Rα are described hereinabove.

The antigen-binding molecule may bind to γc and/or IL-9Rα-expressing cells.

Such cells include immune cells, e.g. effector immune cells. The immune cell may be a cell of hematopoietic origin, e.g. a neutrophil, eosinophil, basophil, dendritic cell, lymphocyte, or monocyte. A lymphocyte may be e.g. a T cell, B cell, NK cell, NKT cell or innate lymphoid cell (ILC), or a precursor thereof (e.g. a thymocyte or pre-B cell). The immune cell may express a CD3 polypeptide (e.g. CD3γ CD3ε CD3ζ or CD3δ), a TCR polypeptide (TCRα or TCRβ), CD27, CD28, CD4 or CD8. In some embodiments, the immune cell is a T cell, e.g. a CD3+ T cell. In some embodiments, the T cell is a CD3+, CD4+ T cell. In some embodiments, the T cell is a CD3+, CD8+ T cell. In some embodiments, the T cell is a T helper cell (TH cell). In some embodiments, the T cell is a cytotoxic T cell (e.g. a cytotoxic T lymphocyte (CTL)). In some embodiments, the immune cell is a T cell or an NK cell.

An ‘effector immune cell’ may be an immune cell displaying an effector function. An effector immune cell may be a CD8+ T cell, CD8+ cytotoxic T lymphocyte (CD8+ CTL), CD4+ T cell, CD4+ T helper cell, NK cell, IFNγ-producing cell, memory T cell, central memory T cell, antigen-experienced T cell or CD45RO+ T cell. An effector immune cell may be characterised by one or more of the following properties: granzyme B expression, IFNγ expression, CD107a expression, IL-2 expression, TNFα expression, perforin expression, granulysin expression, and/or FAS ligand (FASL) expression. In some embodiments, an effector immune cell according to the present disclosure is a granzyme B-expressing cell.

The ability of an antigen-binding molecule to bind to a given cell type (e.g. a cell expressing one or more specified molecules, e.g. selected from γc and/or IL-9Rα) can be analysed by contacting cells with the antigen-binding molecule, and detecting antigen-binding molecule bound to the cells, e.g. after a washing step to remove unbound antigen-binding molecule. The ability of an antigen-binding molecule to bind to γc and/or IL-9Rα-expressing cells can be analysed by methods such as flow cytometry and immunofluorescence microscopy.

In some embodiments, the antigen-binding molecule increases multimerization of γc and IL-9Rα. In some embodiments, the antigen-binding molecule decreases multimerization of γc and IL-9Rα.

As used herein, ‘multimerization’ refers to the formation of a multimeric polypeptide complex (i.e. formed by non-covalent, protein:protein interaction, as described hereinabove). Multimers comprise two or more polypeptides, and may e.g. be dimers, trimers, tetramers, pentamers, hexamers, heptamers, octamers, nonamers or decamers. Accordingly, multimerization may be dimerization, trimerization, tetramerization, et seq.).

It will be appreciated that the multimerization of γc and a polypeptide of a γc-containing cytokine receptor other than γc is heteromultimerization, as the constituent polypeptides of the multimer are non-identical. Thus, the multimers formed by multimerization of γc and a polypeptide of a γc-containing cytokine receptor other than γc in accordance with the present disclosure are heteromultimers, rather than homomultimers.

The antigen-binding molecules of the present disclosure may promote multimerization of γc and a polypeptide of a γc-containing cytokine receptor other than γc through binding to the respective polypeptides, through its constituent antigen-binding moieties. Binding to γc and a polypeptide of a γc-containing cytokine receptor other than γc brings the polypeptides into close physical proximity (e.g. within 50 Angstroms, e.g. within 40, 30, 25, 20, 15, 10 or 5 Angstroms), thereby facilitating their association.

Antigen-binding molecules can be analysed for their ability to increase/promote or decrease/inhibit association between two polypeptides using techniques known to the skilled person. For example, cells expressing the relevant polypeptides can be contacted in vitro with a given test antigen-binding molecule, and association of the relevant polypeptides can thereafter be analysed. Suitable techniques to be employed in the analysis include e.g. resonance energy transfer techniques such as fluorescence resonance energy transfer (FRET) and Bioluminescence Resonance Energy Transfer (BRET), using appropriate labelled interaction partners, e.g. as described in Ciruela, Curr Opin Biotechnol. (2008) 19(4):338-43. Other suitable technologies include protein-fragment complementation systems, e.g. NanoLuc and NanoBiT, which are described e.g. in Thirukkumaran et al., Front Chem. (2020) 7:938 and Dixon et al., ACS Chem Biol. (2016) 11(2):400-408.

An antigen-binding molecule according to the present disclosure may increase or decrease the level of multimerization relative to the level observed in the absence of the antigen-binding molecule, or in the presence of an appropriate control antigen-binding molecule (e.g. an antigen-binding molecule known not to influence multimerization of the relevant polypeptides). In some embodiments, an ‘increased’ level of multimerization refers to a level of multimerization which is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, 29 times or 210 times the level observed in the absence of the antigen-binding molecule, or in the presence of an appropriate control antigen-binding molecule (e.g. an antigen-binding molecule known not to influence multimerization of the relevant polypeptides). In some embodiments, a ‘decreased’ level of multimerization refers to a level of multimerization which is less than 1 times, e.g. one of e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the antigen-binding molecule, or in the presence of an appropriate control antigen-binding molecule (e.g. an antigen-binding molecule known not to influence multimerization of the relevant polypeptides).

In some embodiments, the antigen-binding molecule increases signalling mediated by a γc-containing cytokine receptor (e.g. γc:IL-9Rα receptor). It will be appreciated that the antigen-binding molecule increases signalling mediated by a γc-containing cytokine receptor to which the antigen-binding molecule binds. That is, the antigen-binding molecule increases signalling mediated by a γc-containing cytokine receptor comprising or consisting of a polypeptide complex comprising the polypeptides for which the antigen-binding molecule comprises binding moieties. By way of illustration, in embodiments wherein the antigen-binding molecule comprises (i) a γc-binding moiety and (ii) an IL-9Rα-binding moiety, the antigen-binding molecule may increase signalling through a γc-containing cytokine receptor comprising γc and IL-9Rα, e.g. the γc:IL-9Rα receptor.

Such antigen-binding molecules may variously be described as ‘upregulating’, ‘inducing’, ‘enhancing’ ‘promoting’, ‘stimulating’, ‘triggering’ or ‘potentiating’ signalling mediated by the relevant γc-containing cytokine receptor. They may also be referred to as ‘agonists’ of, or having ‘agonistic’ or ‘activating’ activity with respect to, the relevant γc-containing cytokine receptor.

In some embodiments, the antigen-binding molecule decreases signalling mediated by a γc-containing cytokine receptor (e.g. γc:IL-9Rα receptor). It will be appreciated that the antigen-binding molecule decreases signalling mediated by a γc-containing cytokine receptor to which the antigen-binding molecule binds. That is, the antigen-binding molecule decreases signalling mediated by a γc-containing cytokine receptor comprising or consisting of a polypeptide complex comprising the polypeptides for which the antigen-binding molecule comprises binding moieties. By way of illustration, in embodiments wherein the antigen-binding molecule comprises (i) a γc-binding moiety and (ii) an IL-9Rα-binding moiety, the antigen-binding molecule may decrease signalling through a γc-containing cytokine receptor comprising γc and IL-9Rα, e.g. the γc:IL-9Rα receptor. Such antigen-binding molecules may variously be described as ‘downregulating’, ‘preventing’, ‘diminishing’ ‘inhibiting’, ‘decreasing’, ‘attenuating’ ‘blocking’ or ‘reducing’ signalling mediated by the relevant γc-containing cytokine receptor. They may also be referred to as ‘antagonists’ of, or having ‘antagonistic’ or ‘inhibitory’ activity with respect to, the relevant γc-containing cytokine receptor.

Signalling mediated by a γc-containing cytokine receptor can be analysed using cells expressing the relevant receptor, e.g. using an assay for detecting and/or quantifying receptor-mediated signalling. Suitable assays include e.g. assays for detecting the phosphorylation/activity/expression of factors which are phosphorylated/activated/expressed as a consequence of signalling through the γc-containing cytokine receptor.

Such assays may comprise contacting cells expressing a given γc-containing cytokine receptor with an antigen-binding molecule according to the present disclosure. By way of illustration, an assay for investigating the ability of an antigen-binding molecule to increase γc:IL-9Rα-mediated signalling may comprise contacting cells expressing the γc:IL-9Rα receptor with an antigen-binding molecule comprising a γc-binding moiety and an IL-9Rα-binding moiety.

For example, γc-containing cytokine receptor-mediated signalling can be investigated by evaluating phosphorylation of one or more signal transduction molecules of a signal transduction pathway triggered by signalling through the relevant γc-containing cytokine receptor (e.g. the JAK/STAT, MAPK/ERK or PI3K/Akt pathways). For example, the level of γc-containing cytokine receptor-mediated signalling can be analysed by detection and/or quantification of the level of phosphorylation of STAT1, STAT3, STAT5 and/or ERK (e.g. STAT5 and/or ERK).

In some embodiments, the antigen-binding molecule increases JAK/STAT signalling mediated by a γc:IL-9Rα receptor. In some embodiments, the antigen-binding molecule increases MAPK/ERK signalling mediated by a γc:IL-9Rα receptor. In some embodiments, the antigen-binding molecule increases PI3K/Akt signalling mediated by a γc:IL-9Rα receptor.

In some embodiments, the antigen-binding molecule increases the phosphorylation of STAT1, STAT3, STAT5 and/or ERK. In some embodiments, the antigen-binding molecule increases the activation of STAT1, STAT3, STAT5 and/or ERK. In some embodiments, the antigen-binding molecule increases STAT1, STAT3, STAT5 and/or ERK activity.

In some embodiments, the antigen-binding molecule increases the phosphorylation of STAT5. In some embodiments, the antigen-binding molecule increases the activation of STAT5. In some embodiments, the antigen-binding molecule increases STAT5 activity.

In some embodiments, the antigen-binding molecule decreases JAK/STAT signalling mediated by a γc:IL-9Rα receptor. In some embodiments, the antigen-binding molecule decreases MAPK/ERK signalling mediated by a γc:IL-9Rα receptor. In some embodiments, the antigen-binding molecule decreases PI3K/Akt signalling mediated by a γc:IL-9Rα receptor.

In some embodiments, the antigen-binding molecule decreases the phosphorylation of STAT1, STAT3, STAT5 and/or ERK. In some embodiments, the antigen-binding molecule decreases the activation of STAT1, STAT3, STAT5 and/or ERK. In some embodiments, the antigen-binding molecule decreases STAT1, STAT3, STAT5 and/or ERK activity.

In some embodiments, the antigen-binding molecule decreases the phosphorylation of STAT5. In some embodiments, the antigen-binding molecule decreases the activation of STAT5. In some embodiments, the antigen-binding molecule decreases STAT5 activity.

The level of signalling mediated by a given γc-containing cytokine receptor can also be evaluated by analysing one or more correlates of signalling through the relevant receptor. For example, γc-containing cytokine receptor-mediated signalling may be investigated by detecting and/or quantifying the expression or activity of a factor whose expression/activity is upregulated or downregulated as a consequence of signalling through the relevant receptor. In some embodiments, γc-containing cytokine receptor-mediated signalling may be investigated by detecting and/or quantifying the expression of a factor whose expression is upregulated as a consequence of γc-containing cytokine receptor-mediated signalling.

The level of signalling mediated by a given γc-containing cytokine receptor can also be analysed using reporter-based methods. For example, γc-containing cytokine receptor-mediated signalling can be investigated using a reporter cell line stably expressing a luciferase reporter driven by signalling through the relevant receptor-mediated signalling. Additionally, γc-containing cytokine receptor-mediated signalling can be investigated using a reporter cell line which express a secretable reporter that can be quantitatively detected from the supernatant and can be readily measured (e.g., Cytokine Reporter Cells described and utilised in Examples 3 and 4).

In some embodiments, the antigen-binding molecule increases proliferation, survival and/or effector activity of cells expressing a γc-containing cytokine receptor to which the antigen-binding molecule binds (e.g. γc:IL-9Rα receptor). In some embodiments, the antigen-binding molecule decreases proliferation, survival and/or effector activity of cells expressing a γc-containing cytokine receptor to which the antigen-binding molecule binds (e.g. γc:IL-9Rα receptor). It will be appreciated that the increase/decrease in proliferation, survival and/or effector activity is a cellular-level functional consequence of increased/decreased signalling through the relevant γc-containing cytokine receptor.

The ability of an antigen-binding molecule to increase/decrease proliferation of cells expressing a given γc-containing cytokine receptor can be analysed by contacting cells with the antigen-binding molecule, and subsequently evaluating proliferation of the cells (i.e. after a period of time sufficient for an effect on cell proliferation/survival to be observed). Cell proliferation can be evaluated e.g. by detecting changes in number of cells over time, or by in vitro analysis of incorporation of ³H-thymidine or by CFSE dilution assay, e.g. as described in Fulcher and Wong, Immunol Cell Biol. (1999) 77(6):559-564, hereby incorporated by reference in entirety. Proliferating cells may also be identified by analysis of incorporation of 5-ethynyl-2′-deoxyuridine (EdU) by an appropriate assay, as described e.g. in Buck et al., Biotechniques. (2008) 44(7):927-9, and Sali and Mitchison, PNAS USA. (2008) 105(7):2415-2420. Survival of cells may be evaluated e.g. by labelling cells, and monitoring cell number over time.

Effector activity can be evaluated by analysing correlates of such activity. For example, the ability of an antigen-binding molecule to increase/decrease effector activity of cells expressing a given γc-containing cytokine receptor can be analysed by contacting cells with the antigen-binding molecule, and subsequently evaluating gene and/or protein expression of one or more effector molecules by the cells (i.e. after a period of time sufficient for an effect on gene and/or protein expression of such factors to be observed). Effector molecules include e.g. granzyme B, IFNγ, CD107a, IL-2, TNFα, perforin, granulysin, and FAS ligand (FASL). Gene and/or protein expression of such effector molecules may be determined by any suitable means. Gene expression can be determined e.g. by detection of mRNA encoding the relevant molecule, for example by quantitative real-time PCR (qRT-PCR). Protein expression can be determined e.g. by antibody-based methods, for example by western blot, immunohistochemistry, immunocytochemistry, flow cytometry, or ELISA.

The ability of an antigen-binding molecule to increase/decrease effector activity of cells expressing a given γc-containing cytokine receptor can also be analysed by contacting cells with the antigen-binding molecule, and subsequently evaluating the ability of the cells to kill target cells expressing an antigen for which the cells expressing the γc-containing cytokine receptor comprise a specific receptor (e.g. a TCR or CAR) (i.e. after a period of time sufficient for an effect on cell killing to be observed). Cell killing can be investigated, for example, using any of the methods reviewed in Zaritskaya et al., Expert Rev Vaccines (2011) 9(6):601-616, hereby incorporated by reference in its entirety. Examples of in vitro assays of cytotoxicity/cell killing assays include release assays such as the ⁵¹Cr release assay, the lactate dehydrogenase (LDH) release assay, the 3-(4,5-dimethylthiazol-2-yl)-2,5-diphenyl tetrazolium bromide (MTT) release assay, and the calcein-acetoxymethyl (calcein-AM) release assay. These assays measure cell killing based on the detection of factors released from lysed cells. Cell killing of a given test cell type by a given effector immune cell type can be analysed e.g. by co-culturing the test cells with the effector immune cells, and measuring the number/proportion of viable/dead (e.g. lysed) test cells after a suitable period of time. Other suitable assays include the xCELLigence real-time cytolytic in vitro potency assay described in Cerignoli et al., PLoS One. (2018) 13(3):e0193498 (hereby incorporated by reference in its entirety).

Effector activity can also be analysed in vivo, e.g. in an appropriate non-human animal model of a given disease/condition. Effector activity may be inferred by evaluating therapeutic/prophylactic effects in the relevant model, which are associated with the relevant effector activity.

Herein, an ‘increase’ or ‘decrease’ in the level of signalling/proliferation/survival/effector activity is relative to the level of relevant property displayed (i.e. by cells of the same type) in the absence of the antigen-binding molecule, or in the presence of an appropriate control antigen-binding molecule (e.g. an antigen-binding molecule known not to influence signalling mediated by the relevant γc-containing cytokine receptor/known not to influence proliferation/survival/effector activity of cells expressing the relevant γc-containing cytokine receptor). In some embodiments, an ‘increased’ level of signalling/proliferation/survival/effector activity refers to a level of signalling/proliferation/survival/effector activity which is greater than 1 times, e.g. one of ≥21.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of signalling/proliferation/survival/effector activity observed in the absence of the antigen-binding molecule, or in the presence of an appropriate control antigen-binding molecule (e.g. an antigen-binding molecule known not to influence signalling mediated by the relevant γc-containing cytokine receptor/known not to influence proliferation/survival/effector activity of cells expressing the relevant γc-containing cytokine receptor). In some embodiments, a ‘decreased’ level of signalling/proliferation/survival/effector activity refers to a level of signalling/proliferation/survival/effector activity which is less than 1 times, e.g. one of e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of signalling/proliferation/survival/effector activity observed in the absence of the antigen-binding molecule, or in the presence of an appropriate control antigen-binding molecule (e.g. an antigen-binding molecule known not to influence signalling mediated by the relevant γc-containing cytokine receptor/known not to influence proliferation/survival/effector activity of cells expressing the relevant γc-containing cytokine receptor).

In some embodiments, the antigen-binding molecule reduces the expression of one or more markers of immune cell exhaustion by cells expressing a γc-containing cytokine receptor to which the antigen-binding molecule binds (e.g. γc:IL-9Rα receptor). In some embodiments, the antigen-binding molecule increases the expression of one or more markers of immune cell exhaustion by cells expressing a γc-containing cytokine receptor to which the antigen-binding molecule binds (e.g. γc:IL-9Rα receptor). It will be appreciated that the reduction in the level of expression of the one or more markers of immune cell exhaustion is a cellular-level functional consequence of increased/reduced signalling through the relevant γc-containing cytokine receptor.

For example, the ability of an antigen-binding molecule to reduce/increase the expression of one or more markers of immune cell exhaustion by cells expressing a given γc-containing cytokine receptor can be analysed by contacting cells with the antigen-binding molecule, and subsequently evaluating gene and/or protein expression of one or more markers of immune cell exhaustion by the cells (i.e. after a period of time sufficient for an effect on gene and/or protein expression of such factors to be observed). Markers of immune cell exhaustion include e.g. immune checkpoint molecules (e.g. PD-1, CTLA-4, LAG-3, TIM-3, VISTA, TIGIT and BTLA), CD160 and CD244. In some embodiments, the cell surface expression of one or more markers of immune cell exhaustion may be evaluated, e.g. by flow cytometry.

Herein, a ‘reduction’ or ‘increase’ in the level of expression of one or more markers of immune cell exhaustion is relative to the level displayed (i.e. by cells of the same type) in the absence of the antigen-binding molecule, or in the presence of an appropriate control antigen-binding molecule (e.g. an antigen-binding molecule known not to influence expression of one or more markers of immune cell exhaustion).

In some embodiments, a ‘reduced’ level of expression of one or more markers of immune cell exhaustion refers to a level which is less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the antigen-binding molecule, or in the presence of an appropriate control antigen-binding molecule (e.g. an antigen-binding molecule known not to influence expression of one or more markers of immune cell exhaustion).

In some embodiments, an ‘increased’ level of expression of one or more markers of immune cell exhaustion refers to a level which is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, 21.7 times, ≥1.8 times, ≥1.9 times, ≥2 times, ≥3 times, ≥4 times, ≥5 times, ≥6 times, ≥7 times, ≥8 times, ≥9 times or ≥10 times the level observed in the absence of the antigen-binding molecule, or in the presence of an appropriate control antigen-binding molecule (e.g. an antigen-binding molecule known not to influence expression of one or more markers of immune cell exhaustion).

In some embodiments, the antigen-binding molecule of the present disclosure achieves its functional effects via a mechanism not involving killing/depletion of cells comprising/expressing one or more of the target antigens of its constituent antigen-binding moieties (i.e. γc, and/or IL-9Rα), e.g. Fc-mediated killing/depletion of such cells.

In some embodiments, the antigen-binding molecule of the present disclosure is able to decrease multimerization of γc and IL-9Rα, decrease signalling mediated by a γc-containing cytokine receptor to which the antigen-binding molecule binds (e.g. γc:IL-9Rα receptor), and/or decrease proliferation, survival and/or effector activity of cells expressing a γc-containing cytokine receptor to which the antigen-binding molecule binds (e.g. γc:IL-9Rα receptor) by a mechanism not requiring or involving Fc-mediated function (i.e. independently of Fc-mediated function). That is, in some embodiments, the antigen-binding molecule is able to achieve one or more of the effects recited in the preceding sentence in an Fc region-independent manner.

The ability of an antigen-binding molecule to decrease multimerization of γc and IL-9Rα, decrease signalling mediated by a γc-containing cytokine receptor to which the antigen-binding molecule binds (e.g. γc:IL-9Rα receptor), and/or decrease proliferation, survival and/or effector activity of cells expressing a γc-containing cytokine receptor to which the antigen-binding molecule binds (e.g. γc:IL-9Rα receptor) by a mechanism not requiring/involving Fc-mediated function can be evaluated e.g. by analysing the ability of the antigen-binding molecule provided in a format lacking a functional Fc region to achieve one or more of the specified effects. For example, the relevant functional property(/ies) can be investigated using an antigen-binding molecule comprising a ‘silent’ Fc region (e.g. comprising L234A, L235A and P329G substitutions), or using an antigen-binding molecule provided in a format lacking an Fc region (e.g. scFv, Fab, etc.).

In some embodiments, the antigen-binding molecule decreases multimerization of γc and IL-9Rα, decreases signalling mediated by a γc-containing cytokine receptor to which the antigen-binding molecule binds (e.g. γc:IL-9Rα receptor), and/or decreases proliferation, survival and/or effector activity of cells expressing a γc-containing cytokine receptor to which the antigen-binding molecule binds (e.g. γc:IL-9Rα receptor) by a mechanism not involving ADCC, ADCP and/or CDC.

In some embodiments, the antigen-binding molecule decreases multimerization of γc and IL-9Rα), decreases signalling mediated by a γc-containing cytokine receptor to which the antigen-binding molecule binds (e.g. γc:IL-9Rα receptor), and/or decreases proliferation, survival and/or effector activity of cells expressing a γc-containing cytokine receptor to which the antigen-binding molecule binds (e.g. γc:IL-9Rα receptor) by a mechanism not requiring binding of the antigen-binding molecule to an Fc receptor (e.g. not requiring binding of the antigen-binding molecule to an Fcγ receptor; e.g. not requiring binding of the antigen-binding molecule to one or more of FcγRI, FcγRIIa, FcγRIIb, FcγRIIc, FcγRIIIa and FcγRIIIb), not requiring binding to C1q, and/or not requiring N297 glycosylation.

In some embodiments, the antigen-binding molecule of the present disclosure does not induce ADCC, ADCP or CDC of cells comprising/expressing one or more of the target antigens of its constituent antigen-binding moieties (i.e. γc, and/or IL-9Rα)).

Antigen-binding molecules which do not induce (i.e. are not able to induce) ADCC/ADCP/CDC elicit substantially no ADCC/ADCP/CDC activity against the relevant cell type, e.g. as determined by analysis in an appropriate assay for the relevant activity. ‘Substantially no ADCC/ADCP/CDC activity’ refers to a level of ADCC/ADCP/CDC that is not significantly greater than ADCC/ADCP/CDC determined for an appropriate negative control molecule in a given assay (e.g. an antigen-binding molecule lacking an Fc region, or an antigen-binding molecule comprising a ‘silent’ Fc region (e.g. as described in Schlothauer et al., Protein Engineering, Design and Selection (2016), 29(10):457-466, which is incorporated by reference hereinabove)). ‘Substantially no activity’ may be a level of the relevant activity which is s 5 times, e.g. ≤4 times, ≤3 times, ≤2.5 times, ≤2 times or ≤1.5 times the level of activity determined for an appropriate negative control molecule in a given assay.

The ability of, and extent to which, a given antigen-binding molecule is able to induce ADCC of a given target cell type can be analysed e.g. according to the method described in Yamashita et al., Scientific Reports (2016) 6:19772 (hereby incorporated by reference in its entirety), or by ⁵¹Cr release assay as described e.g. in Jedema et al., Blood (2004) 103: 2677-82 (hereby incorporated by reference in its entirety). The ability of, and extent to which, a given antigen-binding molecule is able to induce ADCP can be analysed e.g. according to the method described in Kamen et al., J Immunol (2017) 198 (1 Supplement) 157.17 (hereby incorporated by reference in its entirety). The ability of, and extent to which, a given antigen-binding molecule is able to induce CDC can be analysed e.g. using a C1q binding assay, e.g. as described in Schlothauer et al., Protein Engineering, Design and Selection (2016), 29(10):457-466 (hereby incorporated by reference in its entirety).

In some embodiments, an antigen-binding molecule according to the present disclosure may increase (i.e. upregulate, enhance, potentiate) cell killing of cells comprising/expressing one or more of the target antigens of its constituent antigen-binding moieties (i.e. γc, and/or IL-9Rα). In some embodiments, an ‘increased’ level of cell killing refers to a level of cell killing which is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, 21.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or 210 times the level of cell killing observed in the absence of the antigen-binding molecule, or in the presence of an appropriate control antigen-binding molecule (e.g. an antigen-binding molecule known not to influence killing of such cells).

In some embodiments an antigen-binding molecule according to the present disclosure is capable of reducing the number/proportion of cells comprising/expressing one or more of the target antigens of its constituent antigen-binding moieties (i.e. γc, and/or IL-9Rα). In some embodiments, the antigen-binding molecule is capable of depleting/enhancing depletion of such cells. In some embodiments, an ‘reduced’ number/proportion of cells refers to a number/proportion of cells which is less than 1 times, e.g. one of e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the number/proportion of observed in the absence of the antigen-binding molecule, or in the presence of an appropriate control antigen-binding molecule (e.g. an antigen-binding molecule known not to influence the number/propotion of such cells).

Antigen-binding molecules according to the present disclosure may comprise one or more moieties for potentiating a reduction in the number/proportion of cells comprising/expressing one or more of the target antigens of its constituent antigen-binding moieties (i.e. γc, and/or IL-9Rα). For example, an antigen-binding molecule according to the present disclosure may e.g. comprise an Fc region and/or a drug moiety.

In some embodiments, an antigen-binding molecule according to the present disclosure comprises an Fc region capable of potentiating/directing one or more of ADCC, ADCP, CDC against, and/or potentiating formation of a MAC on or cell degranulation of, a cell comprising/expressing one or more of the target antigens of its constituent antigen-binding moieties (i.e. γc, and/or IL-9Rα).

In some embodiments, an antigen-binding molecule according to the present disclosure comprises a drug moiety. The antigen-binding molecule may be conjugated to the drug moiety. Antibody-drug conjugates are reviewed e.g. in Parslow et al., Biomedicines. 2016 September; 4(3):14 (hereby incorporated by reference in its entirety). In some embodiments, the drug moiety is or comprises a cytotoxic agent, such that the antigen-binding molecule displays cytotoxicity to a cell comprising/expressing one or more of the target antigens of its constituent antigen-binding moieties (i.e. γc, and/or IL-9Rα). In some embodiments, the drug moiety is or comprises a chemotherapeutic agent.

In some embodiments, an antigen-binding molecule according to the present disclosure comprises an immune cell-engaging moiety. In some embodiments, the antigen-binding molecule comprises a CD3 polypeptide-binding moiety (e.g. an antigen-binding domain capable of binding to a CD3 polypeptide).

In some embodiments, an antigen-binding molecule according to the present disclosure is capable of potentiating/directing T cell-mediated cytolytic activity against cell comprising/expressing one or more of the target antigens of its constituent antigen-binding moieties (i.e. γc, and/or IL-9Rα).

The antigen-binding molecules of the present disclosure possess novel and/or improved properties over γc family cytokines.

In some embodiments, antigen-binding molecules according to the present disclosure possess cytokine-like properties in terms of binding to and triggering γc-containing cytokine receptor-mediated signalling, but are moreover provided with drug- (and particularly antibody)-like biophysical and pharmacokinetic properties.

In some embodiments, the antigen-binding molecule displays increased stability and/or half-life as compared to IL-9.

As used herein, ‘stability’ may refer to resistance to degradation, aggregation and/or unfolding. A molecule that has increased stability as compared to a reference molecule may display reduced degradation/propensity to degrade, reduced aggregation/propensity to aggregate and/or reduced unfolding/propensity to unfold as compared to the reference molecule.

Degradation/aggregation may be determined by detecting and optionally quantifying degraded/aggregated/unfolded species, e.g. in a sample containing the relevant molecule. Stability may be evaluated according to methods well known in the art of molecular biology. Such methods may involve evaluating the antigen-binding molecules to determine the level of degradation (fragmentation), aggregation, unfolding and/or the proportion of degraded/aggregated/unfolded/monomer species.

Stability may be evaluated according to the methods described e.g. in Thiagarajan et al., mAbs. (2016) 8(6):1088-1097, which is hereby incorporated by reference in its entirety. Such methods include analysis by size-exclusion chromatography (SEC), to detect properly assembled molecule (referred to as the monomer), high molecular weight (HMW) species (i.e. aggregates) and/or low molecular weight (LMW) species (i.e. fragments). Other methods include analysis of onset-of-melting temperatures (Tonset), thermal unfolding temperatures (Tm), and apparent enthalpies associated with unfolding transitions, by differential scanning calorimetry (DSC); analysis of effective surface charge via zeta potential and diffusion interaction parameter (KD) analysis, and analysis of intrinsic tryptophan fluorescence by fluorescence spectroscopy.

As used herein, ‘half-life’ refers to the period of time it takes for the concentration of a given molecule to fall to half of its initial value. The half-life may be half-life in plasma (plasma half-life) or serum (serum half-life). The half-life of a given molecule may be evaluated by monitoring the level of the molecule under specified conditions, over time. Half-life can be assessed, for example, through the method of Viera and Rajewsky (Eur J Immunol. (1988) 18(2):313-6), or the method of Souders et al. (MAbs. (2015) 7(5):912-921).

In some embodiments, ‘increased’ stability/half-life relative to a given reference molecule (e.g. a given γc family cytokine) may be stability/half-life which is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, 21.6 times, ≥1.7 times, ≥1.8 times, ≥1.9 times, ≥2 times, ≥3 times, ≥4 times, ≥5 times, ≥6 times, ≥7 times, 28 times, ≥9 times or 210 times the stability/half-life of the reference molecule.

In some embodiments, the antigen-binding molecule of the present disclosure is more effective at increasing signalling mediated by a γc-containing cytokine receptor to which the antigen-binding molecule binds (e.g. γc:IL-9Rα receptor) as compared to a cytokine that binds to the γc-containing cytokine receptor. In some embodiments, the antigen-binding molecule of the present disclosure is less effective at increasing signalling mediated by a γc-containing cytokine receptor to which the antigen-binding molecule binds (e.g. γc:IL-9Rα receptor) as compared to a cytokine that binds to the γc-containing cytokine receptor.

In some embodiments the antigen-binding molecule of the present disclosure is more effective at increasing upregulation of proliferation, survival and/or effector activity of cells expressing a γc-containing cytokine receptor to which the antigen-binding molecule binds (e.g. γc:IL-9Rα receptor) compared to a cytokine that binds to the γc-containing cytokine receptor. In some embodiments the antigen-binding molecule of the present disclosure is less effective at increasing upregulation of proliferation, survival and/or effector activity of cells expressing a γc-containing cytokine receptor to which the antigen-binding molecule binds (e.g. γc:IL-9Rα receptor) compared to a cytokine that binds to the γc-containing cytokine receptor. In some embodiments the antigen-binding molecule of the present disclosure is more effective at reducing the expression of one or more markers of immune cell exhaustion by cells expressing a γc-containing cytokine receptor to which the antigen-binding molecule binds (e.g. γc:IL-9Rα receptor) compared to a cytokine that binds to the γc-containing cytokine receptor. In some embodiments the antigen-binding molecule of the present disclosure is less effective at reducing the expression of one or more markers of immune cell exhaustion by cells expressing a γc-containing cytokine receptor to which the antigen-binding molecule binds (e.g. γc:IL-9Rα receptor) compared to a cytokine that binds to the γc-containing cytokine receptor.

By way of illustration, in embodiments wherein the antigen-binding molecule comprises (i) a γc-binding moiety and (ii) an IL-9Rα-binding moiety, in some embodiments the antigen-binding molecule may increase signalling mediated by the γc:IL-9Rα receptor more effectively than IL-9, and/or may be more effective at upregulating cell proliferation, survival and/or effector activity of cells expressing the γc:IL-9Rα receptor than IL-9. Similarly, the antigen-binding molecule may be more effective at downregulating expression of one or more markers of immune cell exhaustion by cells expressing the γc:IL-9Rα receptor.

In some embodiments, the antigen-binding molecule increases signalling mediated by a γc-containing cytokine receptor (e.g. γc:IL-9Rα receptor) and/or increases cell proliferation, survival and/or effector activity of cells expressing a γc-containing cytokine receptor to which the antigen-binding molecule binds (e.g. γc:IL-9Rα receptor) with an EC₅₀ which is less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the EC₅₀ for the relevant activity displayed by a cytokine that binds to the relevant γc-containing cytokine receptor, as determined in the same assay. In some embodiments, the antigen-binding molecule increases signalling mediated by a γc-containing cytokine receptor (e.g. γc:IL-9Rα receptor) and/or increases cell proliferation, survival and/or effector activity of cells expressing a γc-containing cytokine receptor to which the antigen-binding molecule binds (e.g. γc:IL-9Rα receptor) with an EC₅₀ which is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the EC₅₀ for the relevant activity displayed by the a cytokine that binds to the relevant γc-containing cytokine receptor, as determined in the same assay.

In some embodiments, the antigen-binding molecule reduces expression of one or more markers of immune cell exhaustion by cells expressing a γc-containing cytokine receptor to which the antigen-binding molecule binds (e.g. γc:IL-9Rα receptor) with an IC₅₀ which is less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, 50.1 times, ≤0.05 times, or 50.01 times the IC₅₀ for the relevant activity displayed by a cytokine that binds to the relevant γc-containing cytokine receptor, as determined in the same assay. In some embodiments, the antigen-binding molecule reduces expression of one or more markers of immune cell exhaustion by cells expressing a γc-containing cytokine receptor to which the antigen-binding molecule binds (e.g. γc:IL-9Rα receptor) with an IC₅₀ which is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, 21.7 times, ≥1.8 times, ≥1.9 times, ≥2 times, ≥3 times, ≥4 times, ≥5 times, ≥6 times, ≥7 times, ≥8 times, ≥9 times or ≥10 times the IC₅₀ for the relevant activity displayed by the a cytokine that binds to the relevant γc-containing cytokine receptor, as determined in the same assay.

In some embodiments, an antigen-binding molecule of the present disclosure promotes anti-cancer and/or anti-infection activity in vivo, e.g. in an appropriate non-human animal model. In some embodiments, administration of the antigen-binding molecule is associated with a reduction in the number of cancer cells (e.g. a reduction in cancer burden) in vivo, e.g. as compared to an appropriate control condition. In some embodiments, administration of the antigen-binding molecule is associated with an increase in the killing of cancer cells in vivo, e.g. as compared to an appropriate control condition. In some embodiments, administration of the antigen-binding molecule is associated with a reduction in pathogen load in vivo, e.g. as compared to an appropriate control condition. In some embodiments, administration of the antigen-binding molecule is associated with a reduction in the number of cells of a pathogen and/or the number of cells infected with a pathogen in vivo, e.g. as compared to an appropriate control condition.

In some embodiments, administration of an antigen-binding molecule according to the present disclosure may be associated with one or more of the following, as compared to an appropriate control condition: inhibition of the development/progression of the cancer, a delay to/prevention of onset of the cancer, a reduction in/delay to/prevention of tumor growth, a reduction in/delay to/prevention of tissue invasion, a reduction in/delay to/prevention of metastasis, a reduction in the severity of one or more symptoms of the cancer, a reduction in the number of cancer cells, a reduction in the cancer burden, a reduction in tumour size/volume, and/or an increase in survival of subjects having the cancer (e.g. progression free survival or overall survival), e.g. as determined in an appropriate model.

It will be appreciated that the properties recited in the preceding paragraph are evaluated after a period of time sufficient for an effect associated with administration of the antigen-binding molecule to be observed.

### Linkers and Additional Sequences

The antigen-binding molecules and polypeptides of the present disclosure may additionally comprise further amino acids or sequences of amino acids.

The antigen-binding molecules and polypeptides of the present disclosure may comprise one or more linker sequences between sequences of amino acids, e.g. between sequences of amino acids forming a domain/region as described herein.

In some embodiments, a linker sequence is be provided between a VH sequence and a VL sequence, providing linkage between the VH and VL (e.g. as in an scFv molecule). In some embodiments, a linker sequence is provided between antigen-binding moieties of an antigen-binding molecule of the present disclosure, e.g. as in antigen-binding molecules comprising a polypeptide comprising tandem scFv-scFv. In some embodiments, a linker sequence is provided between an antigen-binding moiety/component thereof and a CH2CH3 region, e.g. as in antigen-binding molecules comprising a polypeptide comprising an scFv moiety linked to CH2CH3 region.

Linker sequences are known to the skilled person, and are described, for example in Chen et al., Adv Drug Deliv Rev. (2013) 65(10):1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev. (2013) 65(10):1357-1369. Flexible linker sequences often comprise high proportions of glycine and/or serine residues.

In some embodiments, the linker sequence comprises at least one glycine residue and/or at least one serine residue. In some embodiments, the linker sequence comprises or consists of glycine and serine residues. In some embodiments, the linker sequence has the structure: (GxS)n or (GxS)nGm; wherein G=glycine, S=serine, x=3 or 4, n=2, 3, 4, 5 or 6, and m=0, 1, 2 or 3. In some embodiments, the linker sequence comprises one or more (e.g. 1, 2, 3, 4, 5 or 6) copies (e.g. in tandem) of the sequence motif G4S. In some embodiments, the linker sequence comprises or consists of (G4S)4 or (G4S)6. In some embodiments, the linker sequence has a length of 1-2, 1-3, 1-4, 1-5, 1-10, 1-15, 1-20, 1-25, or 1-30 amino acids.

In some embodiments, the linker comprises or consists of a GGGGS (SEQ ID NO:240) amino acid sequence. In some embodiments, the linker comprises or consists of a GGGGSGGGGSGGGGS (SEQ ID NO:141) amino acid sequence. In some embodiments, the linker comprises a GGGGSGGGGSGGGGSGGGGS (SEQ ID NO:239) amino acid sequence. In some embodiments, the linker comprises or consists of a NSGAAA (SEQ ID NO:369) amino acid sequence.

In some embodiments, the linker between a VH sequence and a VL sequence comprises or consists of a GGGGS (SEQ ID NO:240) amino acid sequence. In some embodiments, the linker between a VH sequence and a VL sequence comprises or consists of a GGGGSGGGGSGGGGS (SEQ ID NO:141) amino acid sequence. In some embodiments, scFV comprises a GGGGSGGGGSGGGGS (SEQ ID NO:141) amino acid sequence. In some embodiments, the linker between a VH sequence and a VL sequence comprises or consists of a GGGGSGGGGSGGGGSGGGGS (SEQ ID NO:239) amino acid sequence. In some embodiments, scFV comprises a GGGGSGGGGSGGGGSGGGGS (SEQ ID NO:239) amino acid sequence.

In some embodiments, the linker sequence between antigen-binding moieties comprises or consists of a flexible linker. In some embodiments, the linker sequence between antigen-binding moieties comprises or consists of a GGGGS (SEQ ID NO:240) amino acid sequence. In some embodiments, the linker sequence between scFv molecules comprises or consists of a GGGGS (SEQ ID NO:240) amino acid sequence. In some embodiments, the linker sequence between VHH molecules comprises or consists of a GGGGS (SEQ ID NO:240) amino acid sequence. In some embodiments, the linker sequence between an scFv molecule and a VHH molecule comprises or consists of a GGGGS (SEQ ID NO:240) amino acid sequence.

In some embodiments, the linker sequence between antigen-binding moieties comprises or consists of a rigid linker. In some embodiments, the linker sequence between antigen-binding moieties comprises or consists of a EAAAK (SEQ ID NO:364) amino acid sequence. In some embodiments, the linker sequence between scFv molecules comprises or consists of a EAAAK (SEQ ID NO:364) amino acid sequence. In some embodiments, the linker sequence between VHH molecules comprises or consists of a EAAAK (SEQ ID NO:364) amino acid sequence. In some embodiments, the linker sequence between an scFv molecule and a VHH molecule comprises or consists of a EAAAK (SEQ ID NO:364) amino acid sequence.

In some embodiments, the linker sequence between antigen-binding moieties comprises or consists of a rigid linker. In some embodiments, the linker sequence between antigen-binding moieties comprises or consists of an A(EAAAK)₅A (SEQ ID NO:360) amino acid sequence amino acid sequence. In some embodiments, the linker sequence between scFv molecules comprises or consists of an A(EAAAK)₅A (SEQ ID NO:360) amino acid sequence. In some embodiments, the linker sequence between VHH molecules comprises or consists of an A(EAAAK)₅A (SEQ ID NO:360) amino acid sequence. In some embodiments, the linker sequence between an scFv molecule and a VHH molecule comprises or consists of an A(EAAAK)₅A (SEQ ID NO:360) amino acid sequence.

The antigen-binding molecules and polypeptides of the present disclosure may comprise amino acid sequence(s) to facilitate expression, folding, trafficking, processing, purification or detection of the antigen-binding molecule/polypeptide. For example, antigen-binding molecules and polypeptides of the present disclosure may additionally comprise a sequence of amino acids forming a detectable moiety, e.g. as described hereinbelow.

The antigen-binding molecules and polypeptides of the present disclosure may additionally comprise a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides. Signal peptides are known for many proteins, and are recorded in databases such as GenBank, UniProt and Ensembl, and/or can be identified/predicted e.g. using amino acid sequence analysis tools such as SignalP (Petersen et al., Nature Methods (2011) 8:785-786) or Signal-BLAST (Frank and Sippl, Bioinformatics (2008) 24:2172-2176).

The signal peptide may be present at the N-terminus of the antigen-binding molecule/polypeptide and may be present in the newly synthesised antigen-binding molecule/polypeptide. The signal peptide provides for efficient trafficking of the antigen-binding molecule/polypeptide. Signal peptides are often removed by cleavage, and thus are not comprised in the mature antigen-binding molecule/polypeptide.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., Nature Methods (2011) 8:785-786) or Signal-BLAST (Frank and Sippl, Bioinformatics (2008) 24:2172-2176).

### Labels and Conjugates

In some embodiments, the antigen-binding molecule or polypeptide of the present disclosure comprises a detectable moiety.

In some embodiments, a detectable moiety is a fluorescent label, phosphorescent label, luminescent label, immuno-detectable label (e.g. an epitope tag), radiolabel, chemical, nucleic acid or enzymatic label. The antigen-binding molecule or polypeptide may be covalently or non-covalently labelled with the detectable moiety.

Fluorescent labels include e.g. fluorescein, rhodamine, allophycocyanin, eosine and NDB, green fluorescent protein (GFP), chelates of rare earths such as europium (Eu), terbium (Tb) and samarium (Sm), tetramethyl rhodamine, Texas Red, 4-methyl umbelliferone, 7-amino-4-methyl coumarin, Cy3, and Cy5. Radiolabels include radioisotopes such as Hydrogen³, Sulfur³⁵, Carbon¹⁴, Phosphorus³², Iodine¹²³, Iodine¹²⁵, Iodine¹²⁶, Iodine¹³¹, Iodine¹³³, Bromine⁷⁷, Technetium^{99m}, Indium¹¹¹, Indium^{113m}, Gallium⁶⁷, Gallium⁶⁸, Ruthenium⁹⁵, Ruthenium⁹⁷, Ruthenium¹⁰³, Ruthenium¹⁰⁵, Mercury²⁰⁷, Mercury²⁰³, Rhenium^{99m}, Rhenium¹⁰¹, Rhenium¹⁰⁵, Scandium⁴⁷, Tellurium^{121m}, Tellurium^{122m}, Tellurium^{125m}, Thulium¹⁶⁵, Thuliuml¹⁶⁷ Thulium¹⁶⁸, Copper⁶⁷, Fluorine¹⁸, Yttrium⁹⁰, Palladium¹⁰⁰, Bismuth²¹⁷ and Antimony²¹¹. Luminescent labels include as radioluminescent, chemiluminescent (e.g. acridinium ester, luminol, isoluminol) and bioluminescent labels. Immuno-detectable labels include haptens, peptides/polypeptides, antibodies, receptors and ligands such as biotin, avidin, streptavidin or digoxigenin. Nucleic acid labels include aptamers.

In some embodiments, the antigen-binding molecule/polypeptide comprises an epitope tag, e.g. a His, (e.g. 6×His), FLAG, c-Myc, StrepTag, haemagglutinin, E, calmodulin-binding protein (CBP), glutathione-s-transferase (GST), maltose-binding protein (MBP), thioredoxin, S-peptide, T7 peptide, SH2 domain, avidin, streptavidin, and haptens (e.g. biotin, digoxigenin, dinitrophenol), optionally at the N- or C-terminus of the antigen-binding molecule/polypeptide.

In some embodiments, the antigen-binding molecule comprises a Ha tag and a His tag, optionally at the C-terminus.

In some embodiments, the antigen-binding molecule comprises a linker, a Ha tag and a His tag, optionally at the C-terminus. In some embodiments, the antigen-binding molecule comprises SEQ ID NO:368 (AAAYPYDVPDYGSHHHHHH), optionally at the C-terminus. For example, in some embodiments, the antigen-binding molecule comprises a sequence set forth in any one of SEQ ID NOs: 394-396, and further comprises SEQ ID NO:368 at the C-terminus.

In some embodiments, the antigen-binding molecule/polypeptide comprises a moiety having a detectable activity, e.g. an enzymatic moiety. Enzymatic moieties include e.g. luciferases, glucose oxidases, galactosidases (e.g. beta-galactosidase), glucorinidases, phosphatases (e.g. alkaline phosphatase), peroxidases (e.g. horseradish peroxidase) and cholinesterases.

In some embodiments, the antigen-binding molecule or polypeptide of the present disclosure comprises a chemical moiety. In some embodiments, the antigen-binding molecule/polypeptide of the present disclosure is conjugated to a chemical moiety.

The chemical moiety may be a moiety for providing a therapeutic effect, i.e. a drug moiety. A drug moiety may be a small molecule (e.g. a low molecular weight (<1000 daltons, typically between ~300-700 daltons) organic compound). Drug moieties are described e.g. in Parslow et al., Biomedicines. (2016) 4(3):14 (hereby incorporated by reference in its entirety). In some embodiments, a drug moiety may be or comprise a cytotoxic agent. In some embodiments, a drug moiety may be or comprise a chemotherapeutic agent. Drug moieties include e.g. calicheamicin, DM1, DM4, monomethylauristatin E (MMAE), monomethylauristatin F (MMAF), SN-38, doxorubicin, duocarmycin, D6.5 and PBD.

### Nucleic Acids and Vectors

The present disclosure provides a nucleic acid, or a plurality of nucleic acids, encoding an antigen-binding molecule or polypeptide according to the present disclosure. In some embodiments, the nucleic acid(s) comprise or consist of DNA and/or RNA.

An antigen-binding molecule or polypeptide according to the present disclosure may be produced within a cell by translation of RNA encoding the polypeptide(s). An antigen-binding molecule or polypeptide according to the present disclosure may be produced within a cell by transcription from nucleic acid encoding the polypeptide(s), and subsequent translation of the transcribed RNA.

In some embodiments, the nucleic acid(s) may be, or may be comprised/contained in, a vector, or a plurality of vectors. A ‘vector’ as used herein is a nucleic acid molecule used as a vehicle to transfer exogenous nucleic acid into a cell.

Accordingly, the present disclosure also provides a vector, or plurality of vectors, comprising the nucleic acid or plurality of nucleic acids according to the present disclosure. The vector may facilitate delivery of the nucleic acid(s) encoding a polypeptide according to the present disclosure to a cell. The vector may be an expression vector comprising elements required for expressing a polypeptide according to the present disclosure. The vector may comprise elements facilitating integration of the nucleic acid(s) into the genomic DNA of the cell into which the vector is introduced.

Nucleic acids and vectors according to the present disclosure may be provided in purified or isolated form, i.e. from other nucleic acid, or naturally-occurring biological material.

A vector may be a vector for expression of the nucleic acid in the cell (i.e. an expression vector). Such vectors may include a promoter sequence operably linked to a nucleotide sequence encoding an antigen-binding molecule or polypeptide according to the present disclosure. A vector may also include a termination codon (i.e. 3′ in the nucleotide sequence of the vector to the nucleotide sequence encoding the polypeptide(s)) and expression enhancers. Any suitable vectors, promoters, enhancers and termination codons known in the art may be used to express a peptide or polypeptide from a vector according to the present disclosure.

The term ‘operably linked’ may include the situation where nucleic acid encoding a polypeptide according to the present disclosure and regulatory nucleic acid sequence(s) (e.g. a promoter and/or enhancers) are covalently linked in such a way as to place the expression of the nucleic acid encoding a polypeptide under the influence or control of the regulatory nucleic acid sequence(s) (thereby forming an expression cassette). Thus, a regulatory sequence is operably linked to the selected nucleic acid sequence if the regulatory sequence is capable of effecting transcription of the selected nucleic acid sequence. The resulting transcript(s) may then be translated into the desired polypeptide(s).

Vectors contemplated in connection with the present disclosure include DNA vectors, RNA vectors, plasmids (e.g. conjugative plasmids (e.g. F plasmids), non-conjugative plasmids, R plasmids, col plasmids, episomes), viral vectors (e.g. retroviral vectors, e.g. gammaretroviral vectors (e.g. murine Leukemia virus (MLV)-derived vectors, e.g. SFG vector), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol. (2014) 32:189-225 and Morgan and Boyerinas, Biomedicines (2016) 4:9, which are both hereby incorporated by reference in their entirety. In some embodiments, a vector according to the present disclosure is a lentiviral vector.

In some embodiments, the vector may be a eukaryotic vector, i.e. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression.

Constituent polypeptides of an antigen-binding molecule according to the present disclosure may be encoded by different nucleic acids of the plurality of nucleic acids, or by different vectors of the plurality of vectors.

In some embodiments, the nucleic acid/plurality or vector/plurality comprises nucleic acid encoding an internal ribosome entry site (IRES). In some embodiments, the IRES is provided in between nucleotide sequences encoding constituent polypeptides of an antigen-binding molecule according to the present disclosure. In some embodiments, the nucleic acid/plurality or vector/plurality comprises nucleic acid permitting the two or more polypeptides to be translated separately from the same RNA transcript.

In some embodiments, constituent polypeptides of an antigen-binding molecule according to the present disclosure are encoded by nucleotide sequences provided in the same reading frame. In some embodiments, the nucleic acid/plurality or vector/plurality encodes a fusion protein of constituent polypeptides of an antigen-binding molecule. In some embodiments, the fusion protein encoded by the nucleic acid/plurality or vector/plurality comprises a cleavage site (e.g. a cleavage site as described herein) between the amino acid sequences of the constituent polypeptides of the antigen-binding molecule.

In some embodiments, transcription of nucleic acid encoding constituent polypeptides of an antigen-binding molecule is under the control of different promoters.

In some embodiments, the nucleic acid/plurality or vector/plurality is multicistronic (e.g. bicistronic, tricistronic, etc.). That is, in some embodiments the nucleic acid/plurality or vector/plurality comprises multiple polypeptide-encoding nucleotide sequences. In some embodiments, nucleic acid encoding constituent polypeptides of an antigen-binding molecule according to the present disclosure is provided in different cistrons.

### Producing the Antigen-Binding Molecules and Polypeptides

Antigen-binding molecules and polypeptides according to the present disclosure may be prepared according to methods for the production of polypeptides known to the skilled person.

Antigen-binding molecules and polypeptides may be prepared by chemical synthesis, e.g. liquid or solid phase synthesis. For example, peptides/polypeptides can be synthesised using the methods described in, for example, Chandrudu et al., Molecules (2013) 18:4373-4388, which is hereby incorporated by reference in its entirety.

Alternatively, antigen-binding molecules and polypeptides may be produced by recombinant expression. Molecular biology techniques suitable for recombinant production of polypeptides are well known in the art, such as those set out in Green and Sambrook, Molecular Cloning: A Laboratory Manual (4th Edition), Cold Spring Harbor Press, 2012, and in Nat Methods. (2008) 5(2):135-146 both of which are hereby incorporated by reference in their entirety. Methods for the recombinant production of antigen-binding molecules are also described in Frenzel et al., Front Immunol. (2013) 4:217 and Kunert and Reinhart, Appl Microbiol Biotechnol. (2016) 100:3451-3461, both of which are hereby incorporated by reference in their entirety.

In some cases, the antigen-binding molecules of the present disclosure are comprised of more than one polypeptide chain. In such cases, production of the antigen-binding molecule may comprise transcription and translation of more than one polypeptide, and subsequent association of the polypeptide chains to form the antigen-binding molecule.

For recombinant production according to the present disclosure, any cell suitable for the expression of polypeptides may be used. The cell may be a prokaryote or eukaryote. In some embodiments, the cell is a prokaryotic cell, such as a cell of archaea or bacteria. In some embodiments, the bacteria may be Gram-negative bacteria such as bacteria of the family Enterobacteriaceae, for example Escherichia coli. In some embodiments, the cell is a eukaryotic cell such as a yeast cell, a plant cell, insect cell or a mammalian cell, e.g. a cell described hereinabove.

In some cases, the cell is not a prokaryotic cell because some prokaryotic cells do not allow for the same folding or post-translational modifications as eukaryotic cells. In addition, very high expression levels are possible in eukaryotes and proteins can be easier to purify from eukaryotes using appropriate tags. Specific plasmids may also be utilised which enhance secretion of the protein into the media.

In some embodiments polypeptides may be prepared by cell-free-protein synthesis (CFPS), e.g. according to a system described in Zemella et al. Chembiochem (2015) 16(17):2420-2431, which is hereby incorporated by reference in its entirety.

Production may involve culture or fermentation of a eukaryotic cell modified to express the polypeptide(s) of interest. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide(s). Culture, fermentation and separation techniques are well known to those of skill in the art, and are described, for example, in Green and Sambrook, Molecular Cloning: A Laboratory Manual (4th Edition; incorporated by reference herein above).

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culturing the cells that express the polypeptide(s), the polypeptide(s) of interest may be isolated. Any suitable method for separating proteins from cells known in the art may be used. In order to isolate the polypeptide, it may be necessary to separate the cells from nutrient medium. If the polypeptide(s) are secreted from the cells, the cells may be separated by centrifugation from the culture media that contains the secreted polypeptide(s) of interest. If the polypeptide(s) of interest collect within the cell, protein isolation may comprise centrifugation to separate cells from cell culture medium, treatment of the cell pellet with a lysis buffer, and cell disruption e.g. by sonification, rapid freeze-thaw or osmotic lysis.

It may then be desirable to isolate the polypeptide(s) of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for distinguishing different proteins are known in the art, for example ion exchange chromatography and size chromatography. These may be used as an alternative to precipitation or may be performed subsequently to precipitation.

Once the polypeptide(s) of interest have been isolated from culture it may be desired or necessary to concentrate the polypeptide(s). A number of methods for concentrating proteins are known in the art, such as ultrafiltration or lyophilisation.

### Cells Comprising/Expressing the Antigen-Binding Molecules and Polypeptides

The present disclosure also provides a cell comprising or expressing an antigen-binding molecule or polypeptide according to the present disclosure. Also provided is a cell comprising or expressing a nucleic acid, a plurality of nucleic acids, a vector or a plurality of vectors according to the present disclosure.

It will be appreciated that where cells are referred to herein in the singular (i.e. ‘a/the cell’), pluralities/populations of such cells are also contemplated.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell is, or is derived from, a cell type commonly used for the expression of polypeptides for use in therapy in humans. Exemplary cells are described e.g. in Kunert and Reinhart, Appl Microbiol Biotechnol. (2016) 100:3451-3461 (hereby incorporated by reference in its entirety), and include e.g. CHO, HEK 293, PER.C6, NS0 and BHK cells. In preferred embodiments, the cell is, or is derived from, a CHO cell.

The present disclosure also provides a method for producing a cell comprising a nucleic acid(s) or vector(s) according to the present disclosure, comprising introducing a nucleic acid, a plurality of nucleic acids, a vector or a plurality of vectors according to the present disclosure into a cell. In some embodiments, introducing an isolated nucleic acid(s) or vector(s) according to the present disclosure into a cell comprises transformation, transfection, electroporation or transduction (e.g. retroviral transduction).

The present disclosure also provides a method for producing a cell expressing/comprising an antigen-binding molecule or polypeptide according to the present disclosure, comprising introducing a nucleic acid, a plurality of nucleic acids, a vector or a plurality of vectors according to the present disclosure in a cell. In some embodiments, the methods additionally comprise culturing the cell under conditions suitable for expression of the nucleic acid(s) or vector(s) by the cell. In some embodiments, the methods are performed in vitro.

The present disclosure also provides cells obtained or obtainable by the methods according to the present disclosure.

### Compositions

The present disclosure also provides compositions comprising the antigen-binding molecules, polypeptides, nucleic acids, expression vectors and cells described herein.

The antigen-binding molecules, polypeptides, nucleic acids, expression vectors and cells described herein may be formulated as pharmaceutical compositions or medicaments for clinical use and may comprise a pharmaceutically acceptable carrier, diluent, excipient or adjuvant. Thus, the present disclosure also provides a pharmaceutical composition/medicament comprising an antigen-binding molecule, polypeptide, nucleic acid/plurality, expression vector/plurality or cell described herein.

The compositions of the present disclosure may comprise one or more pharmaceutically-acceptable carriers (e.g. liposomes, micelles, microspheres, nanoparticles), diluents/excipients (e.g. starch, cellulose, a cellulose derivative, a polyol, dextrose, maltodextrin, magnesium stearate), adjuvants, fillers, buffers, preservatives (e.g. vitamin A, vitamin E, vitamin C, retinyl palmitate, selenium, cysteine, methionine, citric acid, sodium citrate, methyl paraben, propyl paraben), anti-oxidants (e.g. vitamin A, vitamin E, vitamin C, retinyl palmitate, selenium), lubricants (e.g. magnesium stearate, talc, silica, stearic acid, vegetable stearin), binders (e.g. sucrose, lactose, starch, cellulose, gelatin, polyethylene glycol (PEG), polyvinylpyrrolidone (PVP), xylitol, sorbitol, mannitol), stabilisers, solubilisers, surfactants (e.g., wetting agents), masking agents or colouring agents (e.g. titanium oxide).

The term ‘pharmaceutically-acceptable’ as used herein pertains to compounds, ingredients, materials, compositions, dosage forms, etc., which are, within the scope of sound medical judgment, suitable for use in contact with the tissues of the subject in question (e.g. a human subject) without excessive toxicity, irritation, allergic response, or other problem or complication, commensurate with a reasonable benefit/risk ratio. Each carrier, diluent, excipient, adjuvant, filler, buffer, preservative, anti-oxidant, lubricant, binder, stabiliser, solubiliser, surfactant, masking agent, colouring agent, flavouring agent or sweetening agent of a composition according to the present disclosure must also be ‘acceptable’ in the sense of being compatible with the other ingredients of the formulation. Suitable carriers, diluents, excipients, adjuvants, fillers, buffers, preservatives, anti-oxidants, lubricants, binders, stabilisers, solubilisers, surfactants, masking agents, colouring agents, flavouring agents or sweetening agents can be found in standard pharmaceutical texts, for example, Remington's ‘The Science and Practice of Pharmacy’ (Ed. A. Adejare), 23rd Edition (2020), Academic Press.

Compositions may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration. In some embodiments, a pharmaceutical composition/medicament may be formulated for administration by injection or infusion, or administration by ingestion.

Suitable formulations may comprise the relevant article in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments, the composition is formulated for injection or infusion, e.g. into a blood vessel, tissue/organ of interest, or a tumor.

The present disclosure also provides methods for the production of pharmaceutically-useful compositions and medicaments. Such methods may comprise one or more steps selected from: producing an antigen-binding molecule, polypeptide, nucleic acid (or plurality thereof), expression vector (or plurality thereof) or cell described herein; isolating an antigen-binding molecule, polypeptide, nucleic acid (or plurality thereof), expression vector (or plurality thereof) or cell described herein; and/or mixing an antigen-binding molecule, polypeptide, nucleic acid (or plurality thereof), expression vector (or plurality thereof) or cell described herein with a pharmaceutically-acceptable carrier, adjuvant, excipient or diluent.

For example, a further aspect of the present disclosure relates to a method of formulating or producing a medicament or pharmaceutical composition for use in the treatment of a disease/condition (e.g. a disease/condition described herein), the method comprising formulating a pharmaceutical composition or medicament by mixing an antigen-binding molecule, polypeptide, nucleic acid (or plurality thereof), expression vector (or plurality thereof) or cell described herein with a pharmaceutically acceptable carrier, adjuvant, excipient or diluent.

### Therapeutic and Prophylactic Applications

The antigen-binding molecules, CARs, nucleic acids, expression vectors, cells and compositions described herein find use in therapeutic and prophylactic methods.

The present disclosure provides an antigen-binding molecule, polypeptide, nucleic acid (or plurality thereof), expression vector (or plurality thereof), cell or composition described herein for use in a method of medical treatment or prophylaxis. Also provided is an antigen-binding molecule, polypeptide, nucleic acid (or plurality thereof), expression vector (or plurality thereof), cell or composition described herein for use in a method of treating or preventing a disease or condition described herein. Also provided is the use of an antigen-binding molecule, polypeptide, nucleic acid (or plurality thereof), expression vector (or plurality thereof), cell or composition described herein in the manufacture of a medicament for treating or preventing a disease or condition described herein. Also provided is a method of treating or preventing a disease or condition described herein, comprising administering to a subject a therapeutically or prophylactically effective amount of an antigen-binding molecule, polypeptide, nucleic acid (or plurality thereof), expression vector (or plurality thereof), cell or composition described herein.

The methods may be effective to reduce the development or progression of a disease/condition, alleviation of the symptoms of a disease/condition or reduction in the pathology of a disease/condition. The methods may be effective to prevent progression of the disease/condition, e.g. to prevent worsening of, or to slow the rate of development of, the disease/condition. In some embodiments, the methods may lead to an improvement in the disease/condition, e.g. a reduction in the symptoms of the disease/condition or reduction in some other correlate of the severity/activity of the disease/condition. In some embodiments, the methods may prevent development of the disease/condition a later stage (e.g. a chronic stage or metastasis).

‘Treatment’ may, for example, be reduction in the development or progression of a disease/condition, alleviation of the symptoms of a disease/condition or reduction in the pathology of a disease/condition. Treatment or alleviation of a disease/condition may be effective to prevent progression of the disease/condition, e.g. to prevent worsening of the condition or to slow the rate of development. In some embodiments treatment or alleviation may lead to an improvement in the disease/condition, e.g. a reduction in the symptoms of the disease/condition or reduction in some other correlate of the severity/activity of the disease/condition. Prevention of a disease/condition may refer to prevention of a worsening of the condition or prevention of the development of the disease/condition, e.g. preventing an early stage disease/condition developing to a later, chronic, stage.

It will be appreciated that the articles of the present disclosure (i.e. the antigen-binding molecules, CARs, nucleic acids, expression vectors, cells and compositions described herein) may be used for the treatment/prevention of any disease/condition that would derive therapeutic or prophylactic benefit from modulation of signalling mediated by a γc:IL-9Rα receptor, and/or from manipulation of the number/proportion of cells expressing a γc:IL-9Rα receptor.

In particular aspects and embodiments, it will be appreciated that the antigen-binding molecules of the present disclosure find use in the treatment/prevention of diseases/conditions that would derive therapeutic or prophylactic benefit from an increase in the level of signalling mediated by the γc-containing cytokine receptor to which the antigen-binding molecule binds. By way of illustration, in embodiments wherein the antigen-binding molecule comprises (i) a γc-binding moiety and (ii) an IL-9Rα-binding moiety, the antigen-binding molecule finds use in the treatment/prevention of diseases/conditions that would derive therapeutic or prophylactic benefit from an increase in signalling mediated by the γc:IL-9Rα receptor.

In particular aspects and embodiments, it will be appreciated that the antigen-binding molecules of the present disclosure find use in the treatment/prevention of diseases/conditions that would derive therapeutic or prophylactic benefit from a decrease in the level of signalling mediated by the γc-containing cytokine receptor to which the antigen-binding molecule binds. By way of illustration, in embodiments wherein the antigen-binding molecule comprises (i) a γc-binding moiety and (ii) an IL-9Rα-binding moiety, the antigen-binding molecule finds use in the treatment/prevention of diseases/conditions that would derive therapeutic or prophylactic benefit from an decrease in signalling mediated by the γc:IL-9Rα receptor, and/or diseases/conditions that would derive therapeutic or prophylactic benefit from a reduction in the number/proportion of cells comprising/expressing the γc:IL-9Rα receptor.

The articles of the present disclosure also find use in the treatment/prevention of diseases/conditions that would derive therapeutic or prophylactic benefit from the functional consequences of an increase in the level of signalling mediated by a γc:IL-9Rα receptor. For example, articles of the present disclosure find use in the treatment/prevention of diseases/conditions that would derive therapeutic or prophylactic benefit from an increase in the proliferation and/or population expansion of, and increase in the survival of and/or an increase in the number/proportion and/or activity of cells expressing a γc:IL-9Rα receptor. In particular, the articles of the present disclosure find use in the treatment/prevention of diseases/conditions that would derive therapeutic or prophylactic benefit from an increase in the number/proportion and/or activity of immune cells, e.g. effector immune cells (e.g. effector T cells and/or NK cells).

The articles of the present disclosure also find use in the treatment/prevention of diseases/conditions that would derive therapeutic or prophylactic benefit from the functional consequences of an decrease in the level of signalling mediated by a γc:IL-9Rα receptor, and/or from a the functional consequences of a reduction in the number/proportion of cells comprising/expressing a γc:IL-9Rα receptor. For example, the articles of the present disclosure find use in the treatment/prevention of diseases/conditions that would derive therapeutic or prophylactic benefit from an decrease in the proliferation and/or population expansion of, and decrease in the survival of and/or an decrease in the number/proportion and/or activity of cells expressing a γc:IL-9Rα receptor. In particular, articles of the present disclosure find use in the treatment/prevention of diseases/conditions that would derive therapeutic or prophylactic benefit from an decrease in the number/proportion and/or activity of immune cells, e.g. effector immune cells (e.g. effector T cells and/or NK cells).

The disease/condition to be treated/prevented in accordance with the present disclosure may be a disease/condition in which one or more of the following is positively-associated with the onset, development or progression of the disease/condition, and/or severity of one or more symptoms of the disease/condition, or is a risk factor for the onset, development or progression of the disease/condition: a decreased level of signalling mediated by IL-9, a decreased level of signalling mediated by γc:IL-9Rα receptor, a decreased number/proportion/level of activity of cells expressing a γc:IL-9Rα receptor, a decreased number/proportion/level of activity of lymphocytes, a decreased number/proportion/level of activity of effector immune cells, a decreased number/proportion/level of activity of T cells (e.g. effector T cells), and/or a decreased number/proportion/level of activity of NK cells.

Accordingly, in some embodiments, the disease/condition to be treated/prevented in accordance with the present disclosure is a disease/condition characterised by one or more of the following: a decreased level of signalling mediated by IL-9, a decreased level of signalling mediated by γc:IL-9Rα receptor, a decreased number/proportion/level of activity of cells expressing a γc:IL-9Rα receptor, a decreased number/proportion/level of activity of lymphocytes, a decreased number/proportion/level of activity of effector immune cells, a decreased number/proportion/level of activity of T cells (e.g. effector T cells), and/or a decreased number/proportion/level of activity of NK cells.

The disease/condition to be treated/prevented in accordance with the present disclosure may be a disease/condition in which one or more of the following is positively-associated with the onset, development or progression of the disease/condition, and/or severity of one or more symptoms of the disease/condition, or is a risk factor for the onset, development or progression of the disease/condition: an increased level of signalling mediated by IL-9, an increased level of signalling mediated by γc:IL-9Rα receptor, an increased number/proportion/level of activity of cells expressing a γc:IL-9Rα receptor, an increased number/proportion/level of activity of lymphocytes, an increased number/proportion/level of activity of effector immune cells, an increased number/proportion/level of activity of T cells (e.g. effector T cells), and/or an increased number/proportion/level of activity of NK cells. Accordingly, in some embodiments, the disease/condition to be treated/prevented in accordance with the present disclosure is a disease/condition characterised by one or more of the following: an increased level of signalling mediated by IL-9, an increased level of signalling mediated by γc:IL-9Rα receptor, an increased number/proportion/level of activity of cells expressing a γc:IL-9Rα receptor, an increased number/proportion/level of activity of lymphocytes, an increased number/proportion/level of activity of effector immune cells, an increased number/proportion/level of activity of T cells (e.g. effector T cells), and/or an increased number/proportion/level of activity of NK cells.

In the preceding four paragraphs, the ‘decrease’/‘increase’ may be relative to the level observed in the healthy, non-diseased state, e.g. as determined in a healthy control subject, and/or in equivalent non-diseased tissue.

In accordance with various aspects of the present disclosure, methods are provided which are for, or which comprise (e.g. in the context of treatment/prevention of a disease/condition described herein), one or more of the following:
increasing multimerization of γc and IL-9Rα;
decreasing multimerization of γc and IL-9Rα;
increasing signalling mediated by a γc-containing cytokine receptor to which the antigen-binding molecule binds (e.g. γc:IL-9Rα receptor);
decreasing signalling mediated by a γc-containing cytokine receptor to which the antigen-binding molecule binds (e.g. γc:IL-9Rα receptor);
increasing proliferation, survival and/or effector activity of cells expressing a γc-containing cytokine receptor to which the antigen-binding molecule binds (e.g. γc:IL-9Rα receptor);
decreasing proliferation, survival and/or effector activity of cells expressing a γc-containing cytokine receptor to which the antigen-binding molecule binds (e.g. γc:IL-9Rα receptor);
reducing expression of one or more markers of immune cell exhaustion by cells expressing a γc-containing cytokine receptor to which the antigen-binding molecule binds (e.g. γc:IL-9Rα receptor);
increasing expression of one or more markers of immune cell exhaustion by cells expressing a γc-containing cytokine receptor to which the antigen-binding molecule binds (e.g. γc:IL-9Rα receptor);
increasing cell killing/depletion of, and/or reducing the number/proportion of, cells comprising/expressing one or more of the target antigens of its constituent antigen-binding moieties (i.e. γc, and/or IL-9Rα);
and/or
enhancing anticancer activity of cancer antigen-specific immune cells.

Also provided are agents according to the present disclosure for use in such methods, and the use of agents according to the present disclosure in the manufacture of pharmaceutical compositions or medicaments for use in such methods. It will be appreciated that the methods may comprise administering an antigen-binding molecule, nucleic acid, expression vector, cell or composition described herein to a subject.

Similarly, one or more of the following may be observed in a subject following therapeutic or prophylactic intervention in accordance with the present disclosure (e.g. compared to the level prior to intervention):
an increased level of multimerization of γc and IL-9Rα;
a decreased level of multimerization of γc and IL-9Rα;
an increased level of signalling mediated by a γc-containing cytokine receptor to which the antigen-binding molecule binds (e.g. γc:IL-9Rα receptor);
a decreased level of signalling mediated by a γc-containing cytokine receptor to which the antigen-binding molecule binds (e.g. γc:IL-9Rα receptor);
an increased level of proliferation, survival and/or effector activity of cells expressing a γc-containing cytokine receptor to which the antigen-binding molecule binds (e.g. γc:IL-9Rα receptor);
a decreased level of proliferation, survival and/or effector activity of cells expressing a γc-containing cytokine receptor to which the antigen-binding molecule binds (e.g. γc:IL-9Rα receptor);
a reduced level of expression of one or more markers of immune cell exhaustion by cells expressing a γc-containing cytokine receptor to which the antigen-binding molecule binds (e.g. γc:IL-9Rα receptor);
an increased level of expression of one or more markers of immune cell exhaustion by cells expressing a γc-containing cytokine receptor to which the antigen-binding molecule binds (e.g. γc:IL-9Rα receptor);
increased cell killing/depletion of, and/or a reduced number/proportion of, cells comprising/expressing one or more of the target antigens of its constituent antigen-binding moieties (i.e. γc, and/or IL-9Rα);
and/or
enhanced anticancer activity of cancer antigen-specific immune cells.

In some embodiments, therapeutic/prophylactic intervention in accordance with the present disclosure may be described as being ‘associated with’ one or more of the effects described in the preceding paragraph. The skilled person is readily able to evaluate such properties using techniques that are routinely practiced in the art.

In some aspects and embodiments, the disease/condition to be treated/prevented in accordance with the present disclosure may be lymphocytopenia, or a disease/condition characterised by lymphocytopenia. Lymphocytopenia may be defined as a total lymphocyte count of <1000/mcL (1×10⁹/L) in adults or <3000/mcL (<3×10⁹/L) in children <2 years. Disease/conditions characterised by lymphocytopenia include e.g.: T lymphocytopenia, B lymphocytopenia, NK lymphocytopenia, idiopathic CD4+ lymphocytopenia, Human immunodeficiency virus infection and acquired immunodeficiency syndrome (HIV/AIDS), COVID-19, systemic lupus erythematosus (SLE), rheumatoid arthritis (RA), myasthenia gravis, sarcoidosis, multiple sclerosis (MS), chemotherapy-associated lymphocytopenia, severe combined immunodeficiency (SCID), Omenn syndrome, Wiskott-Aldrich syndrome and cartilage-hair hypoplasia (CHH).

In some embodiments, the disease/condition to be treated/prevented in accordance with the present disclosure is a disease/condition characterised by T cell dysfunction, a cancer, infection, or an autoimmune disease/disorder.

As used herein, ‘T cell dysfunction’ refers to a state in which normal T cell function is reduced/diminished/impaired/aberrant, with the result that a subject suffering from T cell dysfunction displays an insufficient or improper T cell-mediated immune response. T cell dysfunction is reviewed e.g. in Xia et al., Front Immunol. (2019) 10:1719 and Gao et al., Front Immunol. (2022) Sec. Autoimmune and Autoinflammatory Disorders, both of which are hereby incorporated by reference in their entirety.

In some embodiments, the T cell dysfunction may be associated with T cell anergy. T cell anergy is caused by suboptimal T cell stimulation. In some embodiments, the T cell dysfunction may be associated with T cell exhaustion. T cell exhaustion is caused by persistent T cell stimulation and/or overstimulation.

Dysfunctional T cells may be characterised by one of more of the following (i.e. as compared to normal, non-dysfunctional T cells): reduced proliferative capacity, decreased effector function, decreased expression of one or more effector molecules (e.g. selected from granzyme B, IFNγ, CD107a, IL-2, TNFα, perforin, granulysin and FASL), decreased cytotoxicity (e.g. to a cell expressing an MHC:peptide complex for which the T cell expresses a specific receptor), and/or increased expression of one or markers of T cell exhaustion (e.g. selected from PD-1, CTLA-4, LAG-3, TIM-3, VISTA, TIGIT, BTLA, CD160 and CD244).

In some embodiments, a disease/condition characterised by T cell dysfunction may be a cancer, an infectious disease (e.g. chronic infection) or an autoimmune disease.

In some embodiments, the disease/condition to be treated/prevented in accordance with the present disclosure is a cancer. As the articles of the present disclosure are useful to increase/enhance/upregulate anticancer immune responses in a subject, particularly cell-mediated anticancer immune responses, it will be appreciated that they are useful for the treatment/prevention of essentially all cancers.

A cancer in accordance with the present disclosure may be any unwanted cell proliferation (or any disease manifesting itself by unwanted cell proliferation), neoplasm or tumor. The cancer may be benign or malignant. The cancer may be primary or secondary (e.g. metastatic). A neoplasm or tumor may be any abnormal growth or proliferation of cells, and may be located in (and/or derived from cells of) any organ/tissue.

A cancer may be of cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, and/or white blood cells.

A cancer may be, or may comprise, one or more tumors. A cancer may be a glioma, medulloblastoma, meningioma, neurofibroma, ependymoma, Schwannoma, neurofibrosarcoma, astrocytoma and oligodendroglioma, melanoma, mesothelioma, myeloma, lymphoma, Non-Hodgkin's lymphoma (NHL), Hodgkin's lymphoma, cutaneous T-cell lymphoma (CTCL), leukemia, chronic myelogenous leukemia (CML), acute myeloid leukemia (AML), chronic lymphocytic leukemia (CLL), myelodysplastic syndrome (MDS), hepatoma, epidermoid carcinoma, prostate cancer, breast cancer, lung cancer, NSCLC, colon cancer, ovarian cancer, pancreatic cancer, thymic cancer, hematologic cancer or sarcoma.

In some embodiments, a cancer according to the present disclosure is selected from: a solid tumor, breast cancer, breast carcinoma, ductal carcinoma, gastric cancer, gastric carcinoma, gastric adenocarcinoma, colorectal cancer, colorectal carcinoma, colorectal adenocarcinoma, head and neck cancer, squamous cell carcinoma of the head and neck (SCCHN), lung cancer, non-small cell lung cancer, lung adenocarcinoma, squamous cell lung carcinoma, ovarian cancer, ovarian carcinoma, ovarian serous adenocarcinoma, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, esophageal cancer, esophageal adenocarcinoma, liver cancer, hepatocellular carcinoma, cholangiocarcinoma, uterine cancer, uterine corpus endometrial carcinoma, thyroid cancer, thyroid carcinoma, pheochromocytoma, paraganglioma, bladder cancer, bladder urothelial carcinoma, prostate cancer, prostate adenocarcinoma, sarcoma and thymoma.

In some embodiments, the cancer to be treated may be colon cancer, colon carcinoma, colorectal cancer, nasopharyngeal carcinoma, cervical carcinoma, oropharyngeal carcinoma, gastric carcinoma, hepatocellular carcinoma, head and neck cancer, head and neck squamous cell carcinoma (HNSCC), oral cancer, laryngeal cancer, prostate cancer, lung cancer, small cell lung cancer, non-small cell lung cancer, bladder cancer, urothelial carcinoma, melanoma, advanced melanoma, renal cell carcinoma, ovarian cancer or mesothelioma.

In some embodiments, a cancer according to the present disclosure is selected from: gastric cancer (e.g. gastric carcinoma, gastric adenocarcinoma, gastrointestinal adenocarcinoma), head and neck cancer (e.g. head and neck squamous cell carcinoma), breast cancer, ovarian cancer (e.g. ovarian carcinoma), lung cancer (e.g. NSCLC, lung adenocarcinoma, squamous lung cell carcinoma), melanoma, prostate cancer, oral cavity cancer (e.g. oropharyngeal cancer), renal cancer (e.g. renal cell carcinoma) or colorectal cancer (e.g. colorectal carcinoma), oesophageal cancer, pancreatic cancer, a solid cancer and a liquid cancer (i.e. a hematological cancer).

In some embodiments, the cancer to be treated/prevented is a primary cancer. In some embodiments, the cancer the cancer to be treated/prevented is a secondary cancer (i.e. a metastasis).

The treatment may be aimed at one or more of: delaying/preventing the onset/progression of symptoms of the cancer, reducing the severity of symptoms of the cancer, reducing the survival/growth/invasion/metastasis of cells of the cancer, reducing the number of cells of the cancer and/or increasing survival of the subject.

In some embodiments, the disease/condition to be treated/prevented in accordance with the present disclosure is an infectious disease. As the articles of the present disclosure are useful to increase/enhance/upregulate immune responses in a subject, particularly cell-mediated immune responses, it will be appreciated that they are useful for the treatment/prevention of essentially any disease caused by infection.

The infectious disease may be caused and/or characterised by e.g. bacterial, viral, fungal, or parasitic infection. In some embodiments it may be particularly desirable to treat chronic/persistent infection and/or disease caused and/or characterised by chronic/persistent infection, e.g. where such infections are associated with T cell dysfunction (e.g. T cell exhaustion). It is well established that T cell exhaustion is a state of T cell dysfunction that arises during many chronic infections (including viral, bacterial and parasitic infections), as well as in cancer (Wherry, Nature Immunology (2011) 12(6): 492-499).

Examples of bacterial infections that may be treated include infection by Bacillus spp., Bordetella pertussis, Clostridium spp., Corynebacterium spp., Vibrio chloerae, Staphylococcus spp., Streptococcus spp. Escherichia, Klebsiella, Proteus, Yersinia, Erwina, Salmonella (e.g. Salmonella typhi), Listeria sp, Helicobacter pylori, mycobacteria (e.g. Mycobacterium tuberculosis) and Pseudomonas aeruginosa. For example, the bacterial infection may be sepsis or tuberculosis.

Examples of viral infections that may be treated include infection by influenza virus, measles virus, hepatitis B virus (HBV), hepatitis C virus (HCV), human immunodeficiency virus (HIV), lymphocytic choriomeningitis virus (LCMV), Herpes simplex virus or human papilloma virus (HPV).

Examples of fungal infections that may be treated include infection by Alternaria sp, Aspergillus sp, Candida sp and Histoplasma sp. The fungal infection may be fungal sepsis or histoplasmosis.

Examples of parasitic infections that may be treated include infection by Plasmodium species (e.g. Plasmodium falciparum, Plasmodium yoeli, Plasmodium ovale, Plasmodium vivax, or Plasmodium chabaudi chabaudi). The parasitic infection may be a disease such as malaria, leishmaniasis or toxoplasmosis.

In some embodiments, the disease/condition to be treated/prevented in accordance with the present disclosure is an autoimmune disease. Lymphocytopenia and/or T cell dysfunction are characteristic features of autoimmune diseases such as systemic lupus erythematosus (SLE), rheumatoid arthritis (RA), myasthenia gravis and sarcoidosis, and would derive benefit from treatment with the articles of the present disclosure.

The articles of the present disclosure are also useful in connection with methods for the treatment/prevention of diseases/conditions comprising adoptive cell transfer (ACT), in particular ACT of immune cells (e.g. effector immune cells, e.g. T cells and/or NK cells).

Adoptive cell transfer generally refers to a process by which cells (e.g. immune cells) are obtained from a subject, typically by drawing a blood sample from which the cells are isolated. The cells are then typically modified and/or expanded, and then administered either to the same subject (in the case of adoptive transfer of autologous/autogeneic cells) or to a different subject (in the case of adoptive transfer of allogeneic cells). The treatment is typically aimed at providing a population of cells with certain desired characteristics to a subject, or increasing the frequency of such cells with such characteristics in that subject. Adoptive transfer may be performed with the aim of introducing a cell or population of cells into a subject, and/or increasing the frequency of a cell or population of cells in a subject.

Adoptive transfer of immune cells is described, for example, in Kalos and June Immunity (2013) 39(1):49-60, and Davis et al. Cancer J. (2015) 21(6):486-491, both of which are hereby incorporated by reference in their entirety. The skilled person is able to determine appropriate reagents and procedures for adoptive transfer of cells according to the present disclosure, for example by reference to Dai et al., J Nat Cancer Inst. (2016) 108(7):djv439, which is incorporated by reference in its entirety.

Adoptive cell transfer may comprise allotransplantation or autotransplantation. As used herein, ‘allotransplantation’ refers to the transplantation to a recipient subject of cells, tissues or organs which are genetically non-identical to the recipient subject. The cells, tissues or organs may be from, or may be derived from, cells, tissues or organs of a donor subject that is genetically non-identical to the recipient subject. Allotransplantation is distinct from autotransplantation, which refers to the transplantation of cells, tissues or organs which are from/derived from a donor subject genetically identical to the recipient subject (i.e. autologous material). It will be appreciated that adoptive transfer of allogeneic immune cells is a form of allotransplantation, and that adoptive transfer of autologous immune cells is a form of autotransplantation.

The articles of the present disclosure find use in the generation/expansion of populations of immune cells in vitro or ex vivo, which may then be administered to subject. In particular, the present disclosure contemplates the treatment/prevention of a disease/condition (e.g. as disease/condition as described herein) by adoptive transfer of immune cells produced (e.g. generated or expanded) in accordance with a method described herein. That is, the adoptive transferred immune cells may have been generated/expanded by culture in vitro or ex vivo in the presence of an antigen-binding molecule according to the present disclosure.

The immune cells may be immune cells as described hereinabove. It will be appreciated that the immune cells comprise a γc-containing cytokine receptor (e.g. γc:IL-9Rα receptor).

The present disclosure provides a method of treating or preventing a disease or condition in a subject, comprising:
(i) generating or expanding a population of immune cells by culture in the presence of an antigen-binding molecule according to the present disclosure, and;
(ii) administering the generated/expanded population of immune cells to a subject.

In some embodiments, adoptive transfer is of autologous cells. In some embodiments, adoptive transfer is of allogenic cells.

In some embodiments, the method may comprise one or more of the following: obtaining an immune cell-containing sample from a subject (e.g. a blood sample); isolating/purifying immune cells (e.g. PBMCs) from an immune cell-containing sample (e.g. a blood sample); generating or expanding a population of immune cells by culture (i.e. in vitro/ex vivo) in the presence of an antigen-binding molecule according to the present disclosure; collecting a population of immune cells generated or expanded by culture (i.e. in vitro/ex vivo) in the presence of an antigen-binding molecule according to the present disclosure; mixing a population of immune cells generated or expanded by culture (i.e. in vitro/ex vivo) in the presence of an antigen-binding molecule according to the present disclosure with an adjuvant, diluent, or carrier; and/or administering a population of immune cells generated or expanded by culture (i.e. in vitro/ex vivo) in the presence of an antigen-binding molecule according to the present disclosure, or a composition comprising such cells, to a subject.

In some embodiments, the method may additionally comprise administering to a subject a therapeutically or prophylactically effective amount of an antigen-binding molecule according to the present disclosure.

The skilled person is able to determine appropriate reagents and procedures for generated/expanding populations of immune cells for adoptive transfer, and for adoptive transfer of such populations for example by reference to Chia W K et al., Molecular Therapy (2014) 22(1):132-139, Kalos and June Immunity (2013) 39(1):49-60 and Cobbold et al., J Exp Med. (2005) 202:379-386.

In some aspects and embodiments, the disease/condition to be treated/prevented in accordance with the present disclosure may be a disease characterised by an increased number/proportion and/or activity of cells expressing a γc-containing cytokine receptor (e.g. γc:IL-9Rα receptor).

In some aspects and embodiments, the disease/condition to be treated/prevented in accordance with the present disclosure may be lymphocytosis, or a disease/condition characterised by lymphocytosis. Lymphocytosis may be defined as a total lymphocyte count of >3000/mcL (1×10⁹/L) in adults or >9000/mcL (<3×10⁹/L) in children <2 years.

As articles of the present disclosure are useful to decrease/reduce the number/proportion cells comprising/expressing a γc:IL-9Rα receptor in a subject, it will be appreciated that they are useful for the treatment/prevention of diseases characterised by an increase in the number of number/proportion and/or activity of cells expressing a γc:IL-9Rα receptor. As explained herein, cells comprising/expressing a γc:IL-9Rα receptor include immune cells, e.g. effector immune cells (e.g. effector T cells and/or NK cells).

Disease/conditions characterised by lymphocytosis include e.g.: lymphoproliferative diseases/conditions, cancer, leukemia (e.g. chronic lymphocytic leukemia, acute lymphocytic leukemia, acute lymphoblastic leukemia), lymphoma, infectious disease, EBV infection infectious mononucleosis, hepatitis (e.g. hepatitis A, hepatitis B, hepatitis C), CMV infection, HIV/AIDS, syphilis, pertussis, toxoplasmosis, Chagas disease, tuberculosis, brucellosis, hypothyroidism, autoimmune disease and rheumatoid arthritis.

In some embodiments, the disease/condition characterised by lymphocytosis is selected from the group consisting of: lymphoproliferative diseases/conditions, cancer, leukemia (e.g. chronic lymphocytic leukemia, acute lymphocytic leukemia, acute lymphoblastic leukemia), lymphoma, infectious disease, EBV infection infectious mononucleosis, hepatitis (e.g. hepatitis A, hepatitis B, hepatitis C), CMV infection, HIV/AIDS, syphilis, pertussis, toxoplasmosis, Chagas disease, tuberculosis, brucellosis, hypothyroidism, autoimmune disease and rheumatoid arthritis.

In some embodiments, the disease/condition characterised by lymphocytosis is selected from the group consisting of: infectious disease, EBV infection infectious mononucleosis, hepatitis (e.g. hepatitis A, hepatitis B, hepatitis C), CMV infection, HIV/AIDS, syphilis, pertussis, toxoplasmosis, Chagas disease, tuberculosis, brucellosis, hypothyroidism, autoimmune disease and rheumatoid arthritis.

In some embodiments, the disease/condition characterised by lymphocytosis is an autoimmune disease (i.e., an autoimmune disease characterised by lymphocytosis).

In some embodiments, the autoimmune disease is selected from the group consisting of: inflammatory bowel disease (IBD), Crohn's disease, Sjögren's syndrome, lupus, systemic lupus erythematosus (SLE), rheumatoid arthritis (RA), multiple sclerosis (MS), alopecia areata, psoriasis, psoriatic arthritis, myasthenia gravis, sarcoidosis, type 1 diabetes, ulcerative colitis, Addison's disease, Graves' disease, Hashimoto's thyroiditis, Autoimmune vasculitis, Pernicious anemia, Graft-versus-host disease (GVHD), vitiligo, and/or Celiac disease.

Autoimmune diseases develop when the auto-reactive B lymphocytes (autoantibodies) and T lymphocytes cause pathological and/or functional damage to the organ/tissue containing the target autoantigen(s). Signalling mediated by γc-containing cytokine receptors plays an important role in the initiation, development, maintenance and progression of autoimmune diseases. Excessive cytokine signaling can lead to inflammation, autoimmunity, and cancer. The γc family cytokines (IL-7, IL-2, IL-4, IL-9, IL-15 and/or IL-21) bind to γc-containing cytokine receptors and activate three major signalling pathways that promote cellular survival and proliferation, the PI3K-Akt pathway, the RAS-MAPK pathway, and the JAK-STAT pathway. These pathways are often upregulated in patients with autoimmune diseases, therefore there are beneficial effects associated with inhibiting/reducing γc family cytokines signalling (i.e. inhibiting/reducing signalling mediated by a γc-containing cytokine receptors). Autoimmune diseases are associated with increased cytokine signalling (e.g. signalling mediated by a γc-containing cytokine receptor). Treatment with the antigen binding molecules of the present disclosure may reduce cytokine signalling and have a positive impact on patients with autoimmune diseases. Modulating the signalling of cytokines that play critical roles in the initiation and/or effector phases of the autoimmune attack represents a strategy that has shown success. For example, several drugs block TNFα (e.g. etanercept, infliximab, adalimumab), are on the market for the treatment of autoimmune disease.

IL-9 is a Th2 cytokine that regulates multiple biological functions. Particularly, it is reported that IL-9 drives mast cell expansion during inflammation. It has recently been reported that IL-9 induces lung mast cell expansion and migration by direct effects on the proliferation of mast cell progenitors and the migration of mature mast cells (Pajulas et al., Mucosal Immunology. 2023. 16(4): 432-445).

IL-9 is known for its role in type 2 immunity, i.e., T helper type 2 (Th2) immune responses. Although type 2 immunity is beneficial in some circumstances, uncontrolled type 2 responses can lead to the development and progression of diseases and disorders such as allergies and atopic activity (Gartner et al., Pharmacology & Therapeutics. 242, 2023, 108348). T helper 2 (Th2) cells orchestrate protective type 2 immune responses, but also contribute to chronic diseases.

In some embodiments, the disease/condition to be treated in accordance with the present disclosure is a disease/condition in which a Th2 immune response is pathologically-implicated. In some embodiments, the disease/condition to be treated in accordance with the present disclosure is a disease/condition characterised by a Th2 immune response. In some embodiments, the disease/condition to be treated in accordance with the present disclosure is a disease/condition characterised by an upregulated Th2 immune response.

A disease/condition in which a Th2 immune response is pathologically-implicated is a disease/condition in which Th2 immune responses positively contribute to the pathology of a disease. In some embodiments, a Th2 immune response is positively associated with the onset, development or progression of the disease/condition, and/or severity of one or more symptoms of the disease/condition. In some embodiments, an increased level/activity of Th2 immune responses may be a risk factor for the onset, development or progression of the disease/condition.

In some embodiments, the disease/condition in which a Th2 immune response is pathologically-implicated is an autoimmune disease, an inflammatory disease, and/or an allergic disease. In some embodiments, the disease/condition in which Th2 immune responses are pathologically-implicated is an autoimmune disease. In some embodiments, the disease/condition in which Th2 immune responses are pathologically-implicated is an inflammatory disease. In some embodiments, the disease/condition in which Th2 immune responses are pathologically-implicated is an allergic disease.

In some embodiments, the disease/condition in which a Th2 immune response is pathologically-implicated is a disease/condition in which Th2 cells are pathologically-implicated. In some embodiments, the disease/condition in which a Th2 immune response is pathologically-implicated is selected from: asthma, atopic dermatitis, sinusitis, nasal polyps, allergic rhinitis, prurigo, and chronic urticaria.

A disease/condition in which a Th2 immune response is pathologically-implicated may be characterized by one or more of the following:
an increase in the number/proportion/activity of Th2 cells, e.g. as compared to the level/number/proportion/activity in the absence of the disease/condition (e.g. in a healthy subject, or in equivalent non-diseased tissue);
a decrease in the number/proportion/activity of T helper 1 (Th1) cells, e.g. as compared to the level/number/proportion/activity in the absence of the disease/condition (e.g. in a healthy subject, or in equivalent non-diseased tissue);
An increase in Th2 cell differentiation, e.g. as compared to the level/number/proportion/activity in the absence of the disease/condition (e.g. in a healthy subject, or in equivalent non-diseased tissue);
A decrease in Th1 cell differentiation, e.g. as compared to the level/number/proportion/activity in the absence of the disease/condition (e.g. in a healthy subject, or in equivalent non-diseased tissue);
An increase in inflammation, e.g. as compared to the level/number/proportion/activity in the absence of the disease/condition (e.g. in a healthy subject, or in equivalent non-diseased tissue);

In some embodiments, the disease/condition to be treated in accordance with the present disclosure is a disease/condition in which mast cells are pathologically-implicated.

Mast cells are immune cells of the myeloid lineage and are present in connective tissues throughout the body. The activation and degranulation of mast cells significantly modulates many aspects of physiological and pathological conditions in various settings. With respect to normal physiological functions, mast cells are known to regulate vasodilation, vascular homeostasis, innate and adaptive immune responses, angiogenesis, and venom detoxification. On the other hand, mast cells have also been implicated in the pathophysiology of many diseases, including allergy, asthma, anaphylaxis, gastrointestinal disorders, many types of malignancies, and cardiovascular diseases. The role of mast cells in many pathophysiological conditions is reviewed by Krystel-Whittemore et al. (Front. Immunol., 2016 Jan. 6:6:620), which is hereby incorporated by reference in its entirety..

In some embodiments, a disease/condition in which mast cells are pathologically-implicated is a disease/condition in which mast cells positively contribute to the pathology of a disease. In some embodiments, mast cells are positively or negatively associated with the onset, development or progression of the disease/condition, and/or severity of one or more symptoms of the disease/condition.

In some embodiments, an increased level/activity of mast cells may be a risk factor for the onset, development or progression of the disease/condition. In some embodiments, a decreased level/activity of mast cells may be a risk factor for the onset, development or progression of the disease/condition.

In some embodiments, the disease/condition in which mast cells are pathologically-implicated is an autoimmune disease, an inflammatory disease, and/or an allergic disease. In some embodiments, the disease/condition in which mast cells are pathologically-implicated is an autoimmune disease. In some embodiments, the disease/condition in which mast cells are pathologically-implicated is an inflammatory disease. In some embodiments, the disease/condition in which mast cells are pathologically-implicated is an allergic disease.

A disease/condition in which mast cells are pathologically-implicated may be characterized by one or more of the following:
an increase in the number/proportion/activity of mast cells, e.g. as compared to the level/number/proportion/activity in the absence of the disease/condition (e.g. in a healthy subject, or in equivalent non-diseased tissue);
a decrease in the number/proportion/activity of mast cells, e.g. as compared to the level/number/proportion/activity in the absence of the disease/condition (e.g. in a healthy subject, or in equivalent non-diseased tissue);
An increase in mast cell differentiation, e.g. as compared to the level/number/proportion/activity in the absence of the disease/condition (e.g. in a healthy subject, or in equivalent non-diseased tissue);
A decrease in mast cell differentiation, e.g. as compared to the level/number/proportion/activity in the absence of the disease/condition (e.g. in a healthy subject, or in equivalent non-diseased tissue);
An increase in mast cell activity, e.g. as compared to the level/number/proportion/activity in the absence of the disease/condition (e.g. in a healthy subject, or in equivalent non-diseased tissue);
A decrease in mast cell activity, e.g. as compared to the level/number/proportion/activity in the absence of the disease/condition (e.g. in a healthy subject, or in equivalent non-diseased tissue);
An increase in inflammation, e.g. as compared to the level/number/proportion/activity in the absence of the disease/condition (e.g. in a healthy subject, or in equivalent non-diseased tissue);

In some embodiments, the disease/condition in which mast cells are pathologically-implicated is asthma, Mast Cell Activation Syndrome (MCAS), mastocytosis, cutaneous mastocytosis, systemic mastocytosis, Mast cell leukemia (MCL), Mast cell sarcoma, and/or mastocytoma. In some embodiments, the disease/condition in which mast cells are pathologically-implicated is a disease/condition described in Leru (Cureus. 2022 February; 14(2): e22177) and/or Krystel-Whittemore et al. (Front. Immunol., 2016 Jan. 6:6:620), which are both hereby incorporated by reference in their entirety.

Immune cell responses in inflammatory diseases, or diseases characterised by inflammation, can be driven by IL-9, and signalling mediated by γc:IL-9Rα receptors.

In some embodiments, the disease/condition to be treated in accordance with the present disclosure is a disease/condition characterised by inflammation. Diseases/conditions characterised by inflammation include, but are not limited to:
Diseases/conditions affecting the respiratory system, such as sinusitis, rhinitis, pharyngitis, laryngitis, tracheitis, bronchitis, bronchiolitis, pneumonitis, pleuritis and mediastinitis;
Diseases/conditions affecting the accessory digestive organs such as hepatitis, ascending cholangitis, cholecystitis, pancreatitis and peritonitis;
Diseases/conditions affecting the cardiovascular system such as carditis, endocarditis, myocarditis, pericarditis, vasculitis, arteritis, phlebitis and capillaritis;
Diseases/conditions affecting the urinary system such as nephritis, glomerulonephritis, pyelonephritis, ureteritis, cystitis and urethritis;
Diseases/conditions affecting the nervous system such as encephalitis, myelitis, meningitis, arachnoiditis and neuritis;
Diseases/conditions affecting the musculoskeletal system such as arthritis, dermatomyositis, soft tissue, myositis, synovitis/tenosynovitis, bursitis, enthesitis, fasciitis, capsulitis, epicondylitis, tendinitis, panniculitis, osteochondritis: osteitis/osteomyelitis, spondylitis, periostitis and chondritis;
Diseases/conditions affecting the oral cavity and throat such as stomatitis, gingivitis, gingivostomatitis, periodontitis, glossitis, tonsillitis, sialadenitis, parotitis, cheilitis, pulpitis and gnathitis;
Diseases/conditions affecting the gastrointestinal system such as esophagitis, gastritis, gastroenteritis, enteritis, colitis, enterocolitis, duodenitis, ileitis, caecitis, appendicitis, proctitis and Peutz-Jeghers syndrome;
Diseases/conditions affecting the skin such as dermatitis, folliculitis, cellulitis and hidradenitis;
Diseases/conditions affecting the eye such as dacryoadenitis, scleritis, episcleritis, keratitis, retinitis, chorioretinitis, blepharitis, conjunctivitis and uveitis;
Diseases/conditions affecting the ear such as otitis externa, otitis media, labyrinthitis and mastoiditis;
Diseases/conditions of the reproductive system such as oophoritis, salpingitis, endometritis, endometriosis, parametritis, cervicitis, vaginitis, vulvitis, mastitis, orchitis, epididymitis, prostatitis, seminal vesiculitis, balanitis, posthitis, balanoposthitis, chorioamnionitis, funisitis and omphalitis;
Diseases/conditions of the endocrine system such as insulitis, hypophysitis, thyroiditis, parathyroiditis and adrenalitis;
Diseases/conditions of the lymphatic system such as lymphangitis and lymphadenitis;
Cancers, including inflammation-induced and inflammation-associated cancers, such as lung cancer (e.g. lung adenocarcinoma, lung squamous cell carcinoma), prostate cancer, hematological malignancies (e.g. multiple myeloma), pancreatic cancer, cervical cancer, stomach cancer, oesophageal cancer, head and neck cancer, colorectal cancer, colon cancer, liver cancer (e.g. hepatocellular carcinoma) and bile duct cancer.

Inflammation and its role in heath and disease is reviewed e.g. in Chen et al., Oncotarget (2018) 9(6): 7204-7218, which is hereby incorporated by reference in its entirety. Inflammation refers to the bodily response to cellular/tissue injury, and is characterised by edema, erythema (redness), heat, pain, and loss of function (stiffness and immobility) resulting from local immune, vascular and inflammatory cell responses to infection or injury. The injury may result from e.g. physical (e.g. mechanical) or chemical insult, trauma, infection, cancer or overactive/aberrant immune responses (e.g. autoimmune disease). Inflammation forms part of the innate immune response, and plays an important physiological role in wound healing and the control of infection, and contributes to the restoration of tissue homeostasis.

However, many diseases are associated with an overactive inflammatory response (i.e. excessive inflammation and/or aberrantly activated inflammation), and/or chronic (prolonged) inflammation. Herein, excessive and/or chronic inflammation may be referred to as ‘pathological inflammation’. Pathological inflammation may refer to inflammation which is implicated in (i.e. which positively contributes to) the pathology of a disease.

Inflammation to be treated/prevented in accordance with the present disclosure can be of any tissue/organ of the body. In some embodiments, the inflammation is of the lung (e.g. bronchioles, alveoli), airways (e.g. nasal cavity, oral cavity, pharynx, larynx, trachea, bronchi), heart, kidney, liver, skeletal muscle, blood vessels, eye, skin, pancreas, bowel, small intestine, large intestine, colon, joints, brain, or bone marrow. Inflammation may also occur in multiple tissues/organs at once.

In some embodiments, inflammation may be of an organ or tissue of the respiratory system, e.g. the lung (e.g. bronchioles, alveoli), or airways (e.g. nasal cavity, oral cavity, pharynx, larynx, trachea, bronchi). In some embodiments, inflammation may be of an organ or tissue of the cardiovascular system, e.g. the heart or blood vessels. In some embodiments, inflammation may be of an organ or tissue of the gastrointestinal system, e.g. of the liver, bowel, small intestine, large intestine, colon, or pancreas. In some embodiments, inflammation may be of the eye. In some embodiments, inflammation may be of the skin. In some embodiments, inflammation may be of an organ or tissue of the nervous system, e.g. the brain. In some embodiments, inflammation may be of the bone marrow. In some embodiments, inflammation may be of the joints. In some embodiments, inflammation may be of an organ or tissue of the urinary system, e.g. the kidneys. In some embodiments, inflammation may be of an organ or tissue of the musculoskeletal system, e.g. muscle tissue. In some embodiments, inflammation may be of an organ or tissue of one or more organ systems.

In some embodiments, the inflammatory disease (or disease associated with inflammation) is characterised by lymphocytosis.

In some embodiments, the disease/condition characterised by inflammation is selected from chronic inflammatory disease, arthritis, rheumatoid arthritis, juvenile arthritis, systemic juvenile idiopathic arthritis, Sjögren's syndrome, lupus, systemic lupus erythematosus, pancreatitis, thyroiditis, periodontitis, dermatitis, dermatitis, atopic dermatitis, psoriasis, Hermansky-Pudlak syndrome, Graves' disease, diabetes, type 1 diabetes, type 2 diabetes, pregnancy-associated hyperglycemia, multiple sclerosis, giant cell arteritis, Takayasu arteritis, cardiovascular disease, atherosclerosis, atrial fibrillation, ventricular fibrillation, hypertrophic cardiomyopathy, dilated cardiomyopathy, myocarditis, heart failure with preserved ejection fraction, heart failure with reduced ejection fraction, Marfan syndrome, systemic sclerosis, keloid, scleroderma, Alzheimer's disease, hippocampal atrophy, pulmonary disease, asthma, chronic obstructive pulmonary disease, pulmonary fibrosis, idiopathic pulmonary fibrosis, cystic fibrosis, hepatitis, cirrhosis, hepatotoxicity, acetaminophen-induced hepatotoxicity, alcoholic liver disease, pancreatitis, steatosis, non-alcoholic fatty liver disease, non-alcoholic steatohepatitis, cholestasis, primary biliary cholangitis, primary sclerosing cholangitis, inflammatory bowel disease, Crohn's disease, colitis, ulcerative colitis, Addison's disease, Graves' disease, Hashimoto's thyroiditis, autoimmune vasculitis, pernicious anemia, celiac disease endometriosis, stroke, nephropathy, kidney injury, acute kidney injury, nephrotoxicity, glomerulonephritis, chronic kidney disease, Alport syndrome, adult-onset Still's disease, Castleman's disease, cytokine release syndrome, retinal fibrosis, age-related macular degeneration, wet age-related macular degeneration, COVID-19, Peutz-Jeghers syndrome, a cancer, a hematologic malignancy, leukemia, plasmacytoma, Hodgkin's lymphoma, lung cancer, colorectal cancer, intestinal cancer, urinary cancer, bladder cancer, vulvar cancer, endometrial cancer, ovarian cancer, prostate cancer, pancreatic cancer, bone cancer, glioblastoma, breast cancer, stomach cancer, renal cancer, metastatic renal cell cancer, prostate cancer, skin cancer, liver cancer, hepatocellular carcinoma, frailty, age-related increase in fat mass, sarcopenia, age-related hyperlipidaemia, age-related hypertriglyceridemia, age-related hypercholesterolemia, age-related liver steatosis, age-related non-alcoholic fatty liver disease (NAFLD), age-related non-alcoholic fatty liver (NAFL), age-related non-alcoholic steatohepatitis (NASH), age-related cardiovascular disease, age-related hypertension, age-related renal disease and age-related skin disease.

In some embodiments, the disease/condition to be treated/prevented in accordance with the present disclosure is a cancer. Autoimmune diseases are often associated with cancer and malignancies, and, in contrast, some cancers are also associated with an increased risk of developing autoimmune disorders. Therefore, in some embodiments, the antigen binding molecules of the present disclosure, which inhibit signalling mediated by γc:IL-9Rα receptors and/or inhibit IL-9 signalling are useful in the treatment of cancer. In some embodiments, the cancer is associated with autoimmune disease and/or T cell dysfunction.

Administration of the articles of the present disclosure is preferably in a ‘therapeutically-effective’ or ‘prophylactically-effective’ amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's ‘The Science and Practice of Pharmacy’ (ed. A. Adejare), 23rd Edition (2020), Academic Press.

It will be appreciated that in embodiments wherein an antigen-binding molecule according to the present disclosure or a composition comprising such antigen-binding molecule is administered to a subject, it is preferably administered in an amount/quantity sufficient to cause: (i) an increase or decrease in the level of multimerization of γc and IL-9Rα, (ii) an increase or decrease in the level of signalling mediated by a γc:IL-9Rα receptor, (iii) an increase or decrease in the level of proliferation, survival and/or effector activity of cells expressing a γc:IL-9Rα receptor, (iv) an increase or decrease in the level of expression of one or more markers of immune cell exhaustion by cells expressing a γc:IL-9Rα receptor, (v) increased cell killing/depletion of, and/or a reduced number/proportion of, cells comprising/expressing a γc:IL-9Rα receptor, and/or (vi) enhanced anticancer activity of cancer antigen-specific immune cells.

Administration of the articles of the present disclosure may be topical, parenteral, systemic, intracavitary, intravenous, intra-arterial, intramuscular, intrathecal, intraocular, intravitreal, intraconjunctival, subretinal, suprachoroidal, subcutaneous, intradermal, intrathecal, oral, nasal or transdermal. Administration may be by injection or infusion. Where the articles of the present disclosure are employed for the treatment of a cancer, administration may be intratumoral.

In some aspects and embodiments in accordance with the present disclosure there may be targeted delivery of articles of the present disclosure, i.e. wherein the concentration of the relevant agent in the subject is increased in some parts of the body relative to other parts of the body. In some embodiments, the methods comprise intravenous, intra-arterial, intramuscular or subcutaneous administration and wherein the relevant article is formulated in a targeted agent delivery system. Suitable targeted delivery systems include, for example, nanoparticles, liposomes, micelles, beads, polymers, metal particles, dendrimers, antibodies, aptamers, nanotubes or micro-sized silica rods. Such systems may comprise a magnetic element to direct the agent to the desired organ or tissue. Suitable nanocarriers and delivery systems will be apparent to one skilled in the art.

In some cases, the articles of the present disclosure are formulated for targeted delivery to specific cells, a tissue, an organ and/or a tumor.

Administration of the articles of the present disclosure may be alone, or in combination with other treatments, either simultaneously or sequentially dependent upon the disease/condition to be treated. The antigen-binding molecule, cell or composition described herein and another prophylactic/therapeutic agent may be administered simultaneously or sequentially.

In some embodiments, the methods comprise additional therapeutic or prophylactic intervention. In some embodiments, the additional therapeutic or prophylactic intervention is selected from chemotherapy, immunotherapy, radiotherapy, surgery, vaccination and/or hormone therapy. In some embodiments, the additional therapeutic or prophylactic intervention comprises leukapheresis. In some embodiments, the additional therapeutic or prophylactic intervention comprises a stem cell transplant.

Simultaneous administration refers to administration of the antigen-binding molecule, polypeptide, nucleic acid (or plurality thereof), expression vector (or plurality thereof), cell or composition and therapeutic agent together, for example as a pharmaceutical composition containing both agents (combined preparation), or immediately after each other and optionally via the same route of administration, e.g. to the same artery, vein or other blood vessel. Sequential administration refers to administration of one of the antigen-binding molecule/composition or therapeutic agent followed after a given time interval by separate administration of the other agent. It is not required that the two agents are administered by the same route, although this is the case in some embodiments. The time interval may be any time interval.

In some embodiments, treatment of cancer further comprises chemotherapy and/or radiotherapy. Chemotherapy and radiotherapy respectively refer to treatment of a cancer with a drug or with ionising radiation (e.g. radiotherapy using X-rays or y-rays). The drug may be a chemical entity, e.g. small molecule pharmaceutical, antibiotic, DNA intercalator, protein inhibitor (e.g. kinase inhibitor), or a biological agent, e.g. antibody, antibody fragment, aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

Chemotherapy may involve administration of more than one drug. A drug may be administered alone or in combination with other treatments, either simultaneously or sequentially dependent upon the condition to be treated. The chemotherapy may be administered by one or more routes of administration, e.g. parenteral, intravenous injection, oral, subcutaneous, intradermal or intratumoral. The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment. The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Multiple doses of the antigen-binding molecule, polypeptide, nucleic acid (or plurality thereof), expression vector (or plurality thereof), cell or composition described herein may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

Methods of Modulating γc-Containing Cytokine Receptor-Expressing Cells

The antigen-binding molecules of the present disclosure find use in methods that involve bringing-about γc-containing cytokine receptor-mediated signalling in cells expressing γc-containing cytokine receptors, and the functional consequences thereof. Such methods include methods for, or methods comprising: generating and/or expanding populations of cells expressing a γc-containing cytokine receptor; increasing signalling mediated by a γc-containing cytokine receptor in cells expressing a γc-containing cytokine receptor; and/or increasing proliferation, survival and/or effector activity of cells expressing a γc-containing cytokine receptor.

The antigen-binding molecules of the present disclosure also find use in methods that involve inhibiting/preventing γc-containing cytokine receptor-mediated signalling in cells expressing γc-containing cytokine receptors, and/or killing/depleting such cells, and the functional consequences thereof. Such methods include, methods for, or methods comprising: reducing the number/proportion of, or depleting/killing, cells expressing a γc-containing cytokine receptor; decreasing signalling mediated by a γc-containing cytokine receptor in cells expressing a γc-containing cytokine receptor; and/or decreasing proliferation, survival and/or effector activity of cells expressing a γc-containing cytokine receptor.

Such methods generally comprise contacting a cell expressing a γc:IL-9Rα receptor with an antigen-binding molecule according to the present disclosure.

The methods may be performed in vitro or ex vivo and may comprise contacting cells expressing a γc-containing cytokine receptor in vitro or ex vivo with an antigen-binding molecule according to the present disclosure. Such methods may be particularly useful for generating/expanding populations of cells for subsequent administration to a subject, i.e. in accordance with intervention for the treatment/prevention of disease by adoptive cell transfer (ACT).

In some embodiments, methods may be performed in vivo, and may comprise administering an antigen-binding molecule according to the present disclosure to a subject. In such instances, cells expressing a γc-containing cytokine receptor may be contacted with the antigen-binding molecule in vivo, i.e. within the subject to which the antigen-binding molecule is administered.

It will be appreciated that the antigen-binding molecules of the present disclosure find use in essentially any method in which a cytokine which binds to the γc-containing cytokine receptor bound by the antigen-binding molecule binds also finds use. By way of illustration, antigen-binding molecules comprising (i) a γc-binding moiety and (ii) an IL-2Rβ-binding moiety find use in methods in which IL-2 finds use.

### Methods of Detection

The antigen-binding molecules described herein find use in methods that involve detecting γc and/or IL-9Rα. The antigen-binding molecules also find use in methods that involve detecting cells expressing γc and/or IL-9Rα. The antigen-binding molecules also find use in methods that involve detecting cells expressing a γc:IL-9Rα receptor. Such methods may be in vitro or in vivo methods. Such methods may involve detecting the bound complex of the antigen-binding molecule and γc and/or IL-9Rα; and/or cells expressing γc and/or IL-9Rα; and/or cells expressing γc:IL-9Rα receptor.

As such, a method is provided, the method comprising contacting a sample containing, or suspected to contain, γc and/or IL-9Rα; and/or cells expressing γc and/or IL-9Rα; and/or cells expressing γc:IL-9Rα receptor with an antigen-binding molecule according to the present disclosure, and detecting the formation of a complex of the antigen-binding molecule γc and/or IL-9Rα; and/or cells expressing γc and/or IL-9Rα; and/or cells expressing γc:IL-9Rα receptor.

Suitable method formats are well known in the art, including immunoassays such as sandwich assays, e.g. ELISA. The methods may involve labelling the antigen-binding molecule, or target(s), or both, with a detectable moiety, e.g. a detectable moiety as described hereinabove. In some embodiment the detectable moiety is a fluorescent label, a luminescent label, an immune-detectable label or a radio-label. In some embodiments, the detectable moiety may be selected from: a radio-nucleotide, positron-emitting radionuclide (e.g. for positron emission tomography (PET)), MRI contrast agent or fluorescent label. Analysis in vitro or in vivo may involve analysis by positron emission tomography (PET), magnetic resonance imaging (MRI), or fluorescence imaging, e.g. by detection of appropriately labelled species.

Methods of this kind may provide the basis of methods for the diagnostic and/or prognostic evaluation of a disease or condition. Such methods may be performed in vitro on a patient sample, or following processing of a patient sample. Once the sample is collected, the patient is not required to be present for the in vitro method to be performed, and therefore the method may be one which is not practised on the human or animal body.

In some embodiments the methods may involve detecting or quantifying γc and/or IL-9Rα; and/or cells expressing γc and/or IL-9Rα; and/or cells expressing γc:IL-9Rα receptor, e.g. in a patient sample. Where the method comprises quantifying the molecule/complex, the method may further comprise comparing the determined amount against a standard or reference value as part of the diagnostic or prognostic evaluation. Other diagnostic/prognostic tests may be used in conjunction with those described herein to enhance the accuracy of the diagnosis or prognosis or to confirm a result obtained by using the tests described herein.

A sample may be taken from any tissue or bodily fluid. The sample may comprise or may be derived from: a quantity of blood; a quantity of serum derived from the individual's blood which may comprise the fluid portion of the blood obtained after removal of the fibrin clot and blood cells; a tissue sample or biopsy; pleural fluid; cerebrospinal fluid (CSF); or cells isolated from said individual. In some embodiments, the sample may be obtained or derived from a tissue or tissues which are affected by the disease/condition (e.g. tissue or tissues in which symptoms of the disease manifest, or which are involved in the pathogenesis of the disease/condition).

### Subjects

A subject in accordance with the various aspects of the present disclosure may be any animal or human. Therapeutic and prophylactic applications may be in human or animals (veterinary use).

The subject to be administered with an article of the present disclosure (e.g. in accordance with therapeutic or prophylactic intervention) may be a subject in need of such intervention. The subject is preferably mammalian, more preferably human. The subject may be a non-human mammal but is more preferably human. The subject may be male or female. The subject may be a patient.

A subject may have (e.g. may have been diagnosed with) a disease or condition described herein, may be suspected of having such a disease/condition, or may be at risk of developing/contracting such a disease/condition. In embodiments according to the present disclosure, a subject may be selected for treatment according to the methods based on characterisation for one or more markers of such a disease/condition.

### Kits

The present disclosure also provides kits of parts.

In some embodiments, the kit may have at least one container having a predetermined quantity of an antigen-binding molecule, polypeptide, nucleic acid (or plurality thereof), expression vector (or plurality thereof), cell or composition described herein.

In some embodiments, the kit may comprise materials for producing an antigen-binding molecule, polypeptide, nucleic acid (or plurality thereof), expression vector (or plurality thereof), cell or composition described herein. In some embodiments, the kit of parts may comprise materials for formulating an antigen-binding molecule, polypeptide, nucleic acid (or plurality thereof), expression vector (or plurality thereof), cell or composition described herein to a pharmaceutical composition/medicament, e.g. in a composition further comprising a pharmaceutically-acceptable carrier, diluent, excipient or adjuvant.

The kit may provide the antigen-binding molecule, polypeptide, nucleic acid (or plurality thereof), expression vector (or plurality thereof), cell or composition together with instructions for administration to a patient in order to treat a specified disease/condition (e.g. a disease/condition described herein).

In some embodiments the kit may further comprise at least one container having a predetermined quantity of another therapeutic agent (e.g. as described herein). In such embodiments, the kit may also comprise a second medicament or pharmaceutical composition such that the two medicaments or pharmaceutical compositions may be administered simultaneously or separately such that they provide a combined treatment for the specific disease/condition.

The kit may further comprise reagents, buffers and/or standards required for execution of a method according to the present disclosure. Kits according to the present disclosure may include instructions for use, e.g. in the form of an instruction booklet or leaflet. The instructions may include a protocol for performing any one or more of the methods described herein.

### Sequence Identity

As used herein, ‘sequence identity’ refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Söding, J. Bioinformatics (2005) 21:951-960), T-coffee (Notredame et al. J Mol Biol. (2000) 302:205-217), Kalign (Lassmann and Sonnhammer, BMC Bioinformatics. (2005) 6(298)) and MAFFT (Katoh and Standley Molecular Biology and Evolution (2013) 30(4):772-780) software. When using such software, the default parameters, e.g. for gap penalty and extension penalty, are preferably used.

### Sequences

| SEQ | | |
|---|---|---|
| ID | | |
| NO: | DESCRIPTION | SEQUENCE |
| 1 | Anti-CD132 VH P1A3 | QVQLQQWGAGLLKPSETLSLTCAVYGGSFSGYYWSWIRQPPGKGLEWIGEINHSGSTN |
| | | YNPSLKSRATISVDTSKNQFSLKLSSVTAADTAVYYCATSPGGYSGGYFQHWGQGTLVT |
| | | VSS |
| 2 | Anti-CD132 VH P2B9 | QVQLQESGPGLVKPSETLSLTCTVSGGSISSSSYYWGWIRQPPGKGLEWIGSIYYSGST |
| | | YYNPSLKSRVTISVDTSKNQFSLKLSSVTAADTAVYYCAGDILTGYALDYWGQGTLVTVS |
| | | S |
| 3 | Anti-CD132 VHs | QVQLQQWGAGLLKPSETLSLTCAVYGGSFSGYYWSWIRQPPGKGLEWIGEINHFGSTN |
| | P1A3_B3, P1A3_B4, | YNPSLKSRATISVDTSKNQFSLKLSSVTAADTAVYYCATSPGGYSGGYFQHWGQGTLVT |
| | P1A3_E9 | VSS |
| 4 | Anti-CD132 VH P1A3_E8 | QVQLQQWGAGMLKPSETLSLTCAVYGGSFSGYYWSWIRQPPGKGLEWIGEINHFGST |
| | | NYNPSLKSRATISVDTSKNQFSLKLSSVTAADTAVYYCATSPGGYSGGYFQHWGQGTL |
| | | VTVSS |
| 5 | Anti-CD132 VH | EVQLVESGGGLVQPGGSLRLSCAASGGSFSGYYWSWVRQAPGKGLEWVSEINHSGST |
| | P1A3_FW2 | NYNPSLKSRFTISRDNSKNTLYLQMNSLRAEDTAVYYCARSPGGYSGGYFQHWGQGTL |
| | | VTVSS |
| 6 | Anti-CD132 VH P1A10 | QVQLQQSGAEVKKPGSSVKVSCKASGGTFSSYAISWVRQAPGQGLEWMGGFDPEDG |
| | | ETIYAQKFQGRVTMTEDTSTDTAYMELSSLRSEDTAVYYCATDLRIPYYYDNPWGQGTL |
| | | VTVSS |
| 7 | Anti-CD132 VH P1B6 | QVQLVQSGGGVVQPGRSLRLSCAASGFTFSSYAMHWVRQAPGKGLEWVAVISYDGSN |
| | | KYYADSVKGRFTISRDNSKNTLYLQMNSLRAEDTAVYYCARSLYYSHFDYWGQGTLVTV |
| | | SS |
| 8 | Anti-CD132 VH P1C10 | EVQLVETGPGLVKPSGTLSLTCAVSGGSISSSNWWSWVRQPPGKGLEWIGEIYHSGST |
| | | NYNPSLKSRVTISVDKSKNQFSLKLSSVTAADTAVYYCAREGPLSSSGPGAFDIWGQGT |
| | | MVTVSS |
| 9 | Anti-CD132 VH P1D7 | QVQLQESGGGVVQPGRSLRLSCAASGFTFSNYGMHWVRQAPGKGLEWVAVISYDGTN |
| | | KYYADSVKGRFTISRDNSKNTVYLQMNSLRAEDTAVYYCAKDGFDIWGQGTMVTVSS |
| 10 | Anti-CD132 VH P1E8 | EVQLVQSGGGVVQPGRSLRLSCAASGFTFSSYGMHWVRQAPGKGLEWVAVISYDGSN |
| | | KYYADSVKGRFTISRDNSKNTLYLQMNSLRAEDTAVYYCARDVYGDYGAFDYWGQGTL |
| | | VTVSS |
| 11 | Anti-CD132 VH P2B2 | QLQLQESGGGVVQPGRSLRLSCAASGFTFSSYAMHWVRQAPGKGLEWVAVISYDGGN |
| | | KYYADSVKGRFTISRDNSKNTLYLQMNSLRAEDTAVYYCAKSVAPPMDVWGKGTTVTV |
| | | SS |
| 12 | Anti-CD132 VH P2B7 | QVQLQQWGAGLLKPSETLSLTCAVYGESFSGYYWSWIRQPPGKGLEWIGEINHSGSTN |
| | | YNPSLKSRVTISVDTSKNQFSLKLSSVTAADTAVYYCARGPAGSSSSGYFDYWGQGTLV |
| | | TVSS |
| 13 | Anti-CD132 VH P2D11 | QVQLQESGPGLVKPSQTLSLTCTVSGGSISSGGYYWTWIRQHPGQGLEWIGFISWSGT |
| | | TYYNPSLKNRVTISADTSKNHFSLNLTSVTAADTAVYYCARGSGRLVWGQGTLVTVSS |
| 14 | Anti-CD132 VH P2F10 | EVQLVQSGAEVKKPGASVKVSCKASGYTFTGYYMHWVRQAPGQGLEWMGIINPSGGS |
| | | TSYAQKFQGRVTMTRDTSTSTVYMELSSLRSEDTAVYYCARADTAMGDAFDIWGQGTM |
| | | VTVSS |
| 15 | Anti-CD132 VH P2H4 | EVQLVQSGGGVVQPGRSLRLSCAASGFTFSSYAMHWVRQAPGKGLEWVAVISYDGSN |
| | | KYYADSVKGRFTISRDNSKNTLYLQMNSLRAEDTAVYYCARSIGIGAFDIWGQGTMVTV |
| | | SS |
| 16 | Anti-CD132 VH P2D3 | QVQLQQWGAGLLKPSETLSLTCTIYGGSFSGFYWSWIRQPPGKGLEWIGEINHSGSTNY |
| | | NPSLKSRVTISVDTSKNQFSLKLSSVTAADTAIYYCARGPAGSTSSGYFDHWGQGTLVT |
| | | VSS |
| 17 | Anti-CD132 VH P1G4 | QVQLQQWGAGLLKPSETLSLTCAVYGGSLSGYYWSWIRQPPGKGLEWIGEINHSGSTN |
| | | YNPSLKSRVTISVDTSKNQFSLKLSSVTAADTAVYYCARGSSSYYMDVWGKGTTVTVSS |
| 18 | Anti-CD132 VH P1B12 | QVQLQQWGAGLLKPSETLSLTCAVYGGSFSGYYWSWIRQPPGKGLEWIGEINHSGSTN |
| | | YNPSLKSRVTISVDTSKNQFSLKLSSVTAADTAVYYCARGGSAYFQHWGQGTLVTVSS |
| 19 | Anti-CD132 VH P1C7 | QVQLQQWGAGLLKPSETLSLTCAVYGGSFSGYYWSWIRQPPGKGLEWIGEINHSGSTN |
| | | YNPSLKSRVTISEDASKKQFSLTLTSVTAADTAVYYCARGPAGTGSSGYFDYWGQGTLV |
| | | TVSS |
| 20 | Anti-CD132 VL P1A3, | DVVMTQSPLSLPVTPGEPASISCRSSQSLLHSNGYNYLDWYLQKPGQSPQLLIYLGSNR |
| | P1A3_B3, P1A3_E8, | DSGVPDRFSGSGSGTDFTLKISRVEAEDVGVYYCMQGTHWPWTFGQGTKVEIK |
| | P1A3_E9, P1A3-AQ, | |
| | P1A3-ANQ | |
| 21 | Anti-CD132 VL P2B9 | SYELTQPPSMSVSPGQTARITCSGDALPKQFAFWYQQKPGQAPVLVIYKDTERPSGIPE |
| | | RFSGSSSGTTVTLTITGVQAEDEADYYCQSPDSSGTVEVFGGGTKLTVL |
| 22 | Anti-CD132 VL P1A3_B4 | DVVMTQSPLSLPVTPGESVSISCRSSQSLLHSNGYNYLDWYLQKPGQSPQLLIYLGSNR |
| | | DSGVPDRFSGSGSGTDFTLKISRVEAEDVGVYYCMQGTHWPWTFGQGTKVEIK |
| 23 | Anti-CD132 VL | DIQMTQSPSSLSASVGDRVTITCRSSQSLLHSNGYNYLDWYQQKPGKAPKLLIYLGSNR |
| | P1A3_FW2 | DSGVPSRFSGSGSGTDFTLTISSLQPEDFATYYCMQGTHWPWTFGQGTKVEIK |
| 24 | Anti-CD132 VL P1A10, | EIVLTQSPLSLPVTPGEPASISCRSSQSLLHSNGYNYLNWYLQKPGQSPQLLIYLGSDRA |
| | P1A10-AQ, P1A10-ANQ | SGVPDRFSGSGSGTDFTLKISRVEAEDVGVYYCMQALQTPTTFGGGTKVEIK |
| 25 | Anti-CD132 VL P1B6 | EIVMTQSPLSLPVTPGEPASISCRSSQSLLHSNGYNYLDWYLQKPGQSPQLLMYLVSNR |
| | | ASGVPERFSGSGSGTDFTLKISRVEAEDVGVYYCMQTLQTPLSFGQGTKLEIK |
| 26 | Anti-CD132 VL P1C10 | EIVLTQSPATLSLSPGERATLSCRASQSVSYHLAWYQQKPGQAPRLLIYDTSNRASGIPA |
| | | RFSGSGSGTDFTLTINSLEPEDFAVYYCQQRYDWPLTFGGGTKVEIK |
| 27 | Anti-CD132 VL P1D7 | DIQMTQSPSFLSASVGDRVTITCRASQSISSWLAWYQQKPGKAPKLLIYDASRLEDGVPS |
| | | RFSGTGFGTDFTFTITTLQPDDIATYYCQQYDDLPYTFGQGTTVDIK |
| 28 | Anti-CD132 VL P1E8 | DVVMTQSPVSLPVTLGQPASISCKSSQSLLYFNGNTYLSWFQQRPGQSPRRLFYQVSN |
| | | RDSGVPDRFSGSGSDTDFTLTISRVEAEDVGVYFCMQGTQWPPTFGQGTKVEIK |
| 29 | Anti-CD132 VL P2B2 | DVVMTQSPLSLPVTPGEPASISCRSSQSLLHSNGYNYLDWYLQKPGQSPHLLIYLGSNR |
| | | ASGVPDRFSGSGSGTDFTLKISRVEAEDVGVYFCMQALRTPYTFGQGTKLEIK |
| 30 | Anti-CD132 VL P2B7 | DVVMTQSPLSLPVTLGQPASISCRSSQSLVHSNGYNYLDWYLQKPGQSPQLLIYLGSNR |
| | | ASGVPDRFSGSGSGTDFTLKISRVEAEDVGVYYCLQGSHWPWTFGQGTKVEIK |
| 31 | Anti-CD132 VL P2D11 | ETTLTQSPATLSVSPGERATLSCRASQSVSSNLAWYQQKPGQAPRLLIYGASSGATGIP |
| | | DRFSGSGSGTDFTLTISRLEPEDFAVYYCQLYGSSLAFGGGTKVEIK |
| 32 | Anti-CD132 VL P2F10 | DIVMTHTPLSLPVTPGEPASISCRSSQTLFDSDDGKTYLDWYLQKPGQSPQLLMYTTSS |
| | | RASGVPDRFSGSGSGTDFTLKISRVEAEDVGVYYCMQRLQFPLTFGQGTRLEFK |
| 33 | Anti-CD132 VL P2H4 | DVVMTQSPLSLPVTPGEPASISCRATQSLLHGNGHNYLDWYLQKPGQSPQLLIYLGSNR |
| | | ASGVPDRFSGSGSGTDFTLKISRVEAEDVGVYYCMQTLETPVTFGPGTKVDIK |
| 34 | Anti-CD132 VL P2D3 | DVVMTQSPLSLPVTPGEPASISCRSSQSLLHSNGYNYLDWYLQKPGQSPQLLIYLGSNR |
| | | ASGVPDRFSGSGSGTDFTLKISRVEAEDVGVYYCMQGTHWPWTFGQGTKVEIK |
| 35 | Anti-CD132 VL P1G4 | DVVMTQSPLSLPVTPGEPASISCRSSQSLLHSNGYNYLDWYLQKPGQSPQLLIYLGSNR |
| | | ASGVPDRFSGSGSGTDFTLKISRVEAEDVGVYYCLQGTHWPWTFGQGTKVEIK |
| 36 | Anti-CD132 VL P1B12 | DVVMTQSPLSLPVTLGQPASISCRSSQSLLHSNGNNYLDWYLQKPGQSPQLLIYLGSNR |
| | | ASGVPDRFSGSGSGTDFTLKISRVEAEDVGIYYCMQGTHWPWTFGQGTKVEIE |
| 37 | Anti-CD132 VL P1C7 | EIVLTQSPLSLPVTPGEPASISCRSSQSLLHSNGYNYLDWYLQKPGQSPQLLIYLASNRA |
| | | SGVPDRFSGSGSGTDFTLKISRVEAEDVGVYYCMQGTHWPWTFGQGTKVEVK |
| 38 | HC-CDR1 Anti-CD132 | GYYWS |
| | P1A3, P1A3_B3, | |
| | P1A3_B4, P1A3_E9, | |
| | P1A3_E8, P1A3_FW2, | |
| | P2B7, P1G4, P1B12, | |
| | P1C7, P1A3-AQ, P1A3- | |
| | ANQ, P1A3-A, P1A3-Q | |
| 39 | HC-CDR1 Anti-CD132 | SYAMH |
| | P1B6, P2B2, P2H4 | |
| 40 | HC-CDR1 Anti-CD132 | SSNWWS |
| | P1C10 | |
| 41 | HC-CDR2 Anti-CD132 | EINHSGSTNYNPSLKS |
| | P1A3, P1A3_FW2, P2B7, | |
| | P2D3, P1G4, P1B12, | |
| | P1C7, P1A3-AQ, P1A3- | |
| | ANQ, P1A3-A, P1A3-Q | |
| 42 | HC-CDR2 Anti-CD132 | VISYDGSNKYYADSVKG |
| | P1B6, P1E8, P2H4 | |
| 43 | HC-CDR2 Anti-CD132 | EIYHSGSTNYNPSLKS |
| | P1C10 | |
| 44 | LC-CDR1 Anti-CD132 | RSSQSLLHSNGYNYLD |
| | P1A3, P1A3_B3, | |
| | P1A3_E8, P1A3_E9, | |
| | P1A3_B4, P1A3_FW2, | |
| | P1B6, P2B2, P2D3, P1G4, | |
| | P1C7, P1A3-A, P1A3-Q, | |
| | P1A3-AQ, P1A3-ANQ | |
| 45 | LC-CDR2 Anti-CD132 | LGSNRAS |
| | P2B2, P2B7, P2H4, P2D3, | |
| | P1G4, P1B12 | |
| 46 | LC-CDR3 Anti-CD132 | MQGTHWPWT |
| | P1A3, P1A3_B3, | |
| | P1A3_E8, P1A3_E9, | |
| | P1A3_B4, P1A3_FW2, | |
| | P2D3, P1B12, P1C7, | |
| | P1A3-AQ, P1A3-ANQ, | |
| | P1A3-A, P1A3-Q | |
| 47 | HC-CDR1 Anti-CD132 | SSSYYWG |
| | P2B9 | |
| 48 | HC-CDR1 Anti-CD132 | SYAIS |
| | P1A10, P1A10-AQ, | |
| | P1A10-ANQ | |
| 49 | HC-CDR1 Anti-CD132 | NYGMH |
| | P1D7 | |
| 50 | HC-CDR1 Anti-CD132 | SYGMH |
| | P1E8 | |
| 51 | HC-CDR1 Anti-CD132 | SGGYYWT |
| | P2D11 | |
| 52 | HC-CDR1 Anti-CD132 | GYYMH |
| | P2F10 | |
| 53 | HC-CDR1 Anti-CD132 | GFYWS |
| | P2D3 | |
| 54 | HC-CDR2 Anti-CD132 | SIYYSGSTYYNPSLK |
| | P2B9 | |
| 55 | HC-CDR2 Anti-CD132 | EINHFGSTNYNPSLKS |
| | P1A3_B3, P1A3_B4, | |
| | P1A3_E9, P1A3_E8 | |
| 56 | HC-CDR2 Anti-CD132 | GFDPEDGETIYAQKFQG |
| | P1A10, P1A10-AQ, | |
| | P1A10-ANQ | |
| 57 | P2C4_FW2/P1A3_AQ | EVQLVQSGAEVKKPGSSVKVSCKASGYTFTNYYMHWVRQAPGQGLEWMGAIMPSRG |
| | antibody (Secreted | GTSYPQKFQGRVTMTGDTSTSTVYMELSSLRSEDTAVYYCARGEYYYDSSGYYYWGQ |
| | Megakine) | GTLVTVSSGGGGSGGGGSGGGGSQSALTQPASVSGSPGQSIAISCTGTSSDIGHYDFV |
| | | SWYQQHPGTAPKLIIYDINNRPSGISNRFSGSKSDNMASLTISGLQPEDEADYYCSAYTS |
| | | SDTLVFGGGTKLTVLNSGAAAQVQLQQWGAGLLKPSETLSLTCAVYGGSFSGYYWSWI |
| | | RQPPGKGLEWIGEINHSGSTNYNPSLKSRATISVDTSKNQFSLKLSSVTAADTAVYYCAT |
| | | SPGGYSGGYFQHWGQGTLVTVSSGGGGSGGGGSGGGGSDVVMTQSPLSLPVTPGEP |
| | | ASISCRSSQSLLHSNGYNYLDWYLQKPGQSPQLLIYLGSNRDSGVPDRFSGSGSGTDFT |
| | | LKISRVEAEDVGVYYCMQGTHWPWTFGQGTKVEIK |
| 58 | HC-CDR2 Anti-CD132 | VISYDGTNKYYADSVKG |
| | P1D7 | |
| 59 | HC-CDR2 Anti-CD132 | VISYDGGNKYYADSVKG |
| | P2B2 | |
| 60 | HC-CDR2 Anti-CD132 | FISWSGTTYYNPSLKN |
| | P2D11 | |
| 61 | HC-CDR2 Anti-CD132 | IINPSGGSTSYAQKFQG |
| | P2F10 | |
| 62 | HC-CDR3 Anti-CD132 | SPGGYSGGYFQH |
| | P1A3, P1A3_B3, | |
| | P1A3_B4, P1A3_E9 | |
| | P1A3_E8, P1A3_FW2, | |
| | P1A3-AQ, P1A3-ANQ, | |
| | P1A3-A, P1A3-Q | |
| 63 | HC-CDR3 Anti-CD132 | DILTGYALDY |
| | P2B9 | |
| 64 | HC-CDR3 Anti-CD132 | DLRIPYYYDNP |
| | P1A10, P1A10-AQ, | |
| | P1A10-ANQ | |
| 65 | HC-CDR3 Anti-CD132 | SLYYSHFDY |
| | P1B6 | |
| 66 | HC-CDR3 Anti-CD132 | EGPLSSSGPGAFDI |
| | P1C10 | |
| 67 | HC-CDR3 Anti-CD132 | DGFDI |
| | P1D7 | |
| 68 | HC-CDR3 Anti-CD132 | DVYGDYGAFDY |
| | P1E8 | |
| 69 | HC-CDR3 Anti-CD132 | SVAPPMDV |
| | P2B2 | |
| 70 | HC-CDR3 Anti-CD132 | GPAGSSSSGYFDY |
| | P2B7 | |
| 71 | HC-CDR3 Anti-CD132 | GSGRLV |
| | P2D11 | |
| 72 | HC-CDR3 Anti-CD132 | ADTAMGDAFDI |
| | P2F10 | |
| 73 | HC-CDR3 Anti-CD132 | SIGIGAFDI |
| | P2H4 | |
| 74 | HC-CDR3 Anti-CD132 | GPAGSTSSGYFDH |
| | P2D3 | |
| 75 | HC-CDR3 Anti-CD132 | GSSSYYMDV |
| | P1G4 | |
| 76 | HC-CDR3 Anti-CD132 | GGSAYFQH |
| | P1B12 | |
| 77 | HC-CDR3 Anti-CD132 | GPAGTGSSGYFDY |
| | P1C7 | |
| 78 | LC-CDR1 Anti-CD132 | SGDALPKQFAF |
| | P2B9 | |
| 79 | LC-CDR1 Anti-CD132 | RSSQSLLHSNGYNYLN |
| | P1A10, P1A10-AQ, | |
| | P1A10-ANQ | |
| 80 | LC-CDR1 Anti-CD132 | RASQSVSYHLA |
| | P1C10 | |
| 81 | LC-CDR1 Anti-CD132 | RASQSISSWLA |
| | P1D7 | |
| 82 | LC-CDR1 Anti-CD132 | KSSQSLLYFNGNTYLS |
| | P1E8 | |
| 83 | LC-CDR1 Anti-CD132 | RSSQSLVHSNGYNYLD |
| | P2B7 | |
| 84 | LC-CDR1 Anti-CD132 | RASQSVSSNLA |
| | P2D11 | |
| 85 | LC-CDR1 Anti-CD132 | RSSQTLFDSDDGKTYLD |
| | P2F10 | |
| 86 | LC-CDR1 Anti-CD132 | RATQSLLHGNGHNYLD |
| | P2H4 | |
| 87 | LC-CDR1 Anti-CD132 | RSSQSLLHSNGNNYLD |
| | P1B12 | |
| 88 | LC-CDR2 Anti-CD132 | LGSNRDS |
| | P1A3, P1A3_B3, | |
| | P1A3_E8, P1A3_E9, | |
| | P1A3_B4, P1A3_FW2, | |
| | P1A3-AQ, P1A3-ANQ, | |
| | P1A3-A, P1A3-Q | |
| 89 | LC-CDR2 Anti-CD132 | KDTERPS |
| | P2B9 | |
| 90 | LC-CDR2 Anti-CD132 | LGSDRAS |
| | P1A10, P1A10-AQ, | |
| | P1A10-ANQ | |
| 91 | LC-CDR2 Anti-CD132 | LVSNRAS |
| | P1B6 | |
| 92 | LC-CDR2 Anti-CD132 | DTSNRAS |
| | P1C10 | |
| 93 | LC-CDR2 Anti-CD132 | DASRLED |
| | P1D7 | |
| 94 | LC-CDR2 Anti-CD132 | QVSNRDS |
| | P1E8 | |
| 95 | LC-CDR2 Anti-CD132 | GASSGAT |
| | P2D11 | |
| 96 | LC-CDR2 Anti-CD132 | TTSSRAS |
| | P2F10 | |
| 97 | LC-CDR2 Anti-CD132 | LASNRAS |
| | P1C7 | |
| 98 | LC-FR4 Anti-CD132 P1C7 | FGQGTKVEVK |
| 99 | LC-CDR3 Anti-CD132 | QSPDSSGTVEV |
| | P2B9 | |
| 100 | LC-CDR3 Anti-CD132 | MQALQTPTT |
| | P1A10, P1A10-AQ, | |
| | P1A10-ANQ | |
| 101 | LC-CDR3 Anti-CD132 | MQTLQTPLS |
| | P1B6 | |
| 102 | LC-CDR3 Anti-CD132 | QQRYDWPLT |
| | P1C10 | |
| 103 | LC-CDR3 Anti-CD132 | QQYDDLPYT |
| | P1D7 | |
| 104 | LC-CDR3 Anti-CD132 | MQGTQWPPT |
| | P1E8 | |
| 105 | LC-CDR3 Anti-CD132 | MQALRTPYT |
| | P2B2 | |
| 106 | LC-CDR3 Anti-CD132 | LQGSHWPWT |
| | P2B7 | |
| 107 | LC-CDR3 Anti-CD132 | QLYGSSLA |
| | P2D11 | |
| 108 | LC-CDR3 Anti-CD132 | MQRLQFPLT |
| | P2F10 | |
| 109 | LC-CDR3 Anti-CD132 | MQTLETPVT |
| | P2H4 | |
| 110 | LC-CDR3 Anti-CD132 | LQGTHWPWT |
| | P1G4 | |
| 111 | CH2 domain P2C4 | PCPAPEAAGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAK |
| 112 | CH3 domain P2C4 | GQPREPQVYTLPPCRDELTKNQVSLWCLVKGFYPSDIAVEWESNGQPENNYKTTPPVL |
| | | DSDGSFFLYSKLTVDKSRWQQGNVFSCSVMHEALHNHYTQKSLSLSPGK |
| 113 | CH2 domain P1A3 | PCPAPEAAGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAK |
| 114 | CH3 domain P1A3 | GQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSPGK |
| 115 | CH2 domain P1A10 | PCPAPEAAGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAK |
| 116 | CH3 domain P1A10 | GQPREPQVCTLPPSRDELTKNQVSLSCAVKGFYPSDIAVEWESNGQPENNYKTTPPVL |
| | | DSDGSFFLVSKLTVDKSRWQQGNVFSCSVMHEALHNHYTQKSLSLSPGK |
| 117 | Anti-CD132 clone P1A3 | QVQLQQWGAGLLKPSETLSLTCAVYGGSFSGYYWSWIRQPPGKGLEWIGEINHSGSTN |
| | | YNPSLKSRATISVDTSKNQFSLKLSSVTAADTAVYYCATSPGGYSGGYFQHWGQGTLVT |
| | | VSSGGGGSGGGGSGGGGSDVVMTQSPLSLPVTPGEPASISCRSSQSLLHSNGYNYLD |
| | | WYLQKPGQSPQLLIYLGSNRDSGVPDRFSGSGSGTDFTLKISRVEAEDVGVYYCMQGT |
| | | HWPWTFGQGTKVEIKNSGAGTAAATHTCPPCPAPEAAGGPSVFLFPPKPKDTLMISRTP |
| | | EVTCVVVDVSHEDPEVKFNWYVDGVEVHNAKTKPREEQYNSTYRVVSVLTVLHQDWLN |
| | | GKEYKCKVSNKALPAPIEKTISKAKGQPREPQVCTLPPSRDELTKNQVSLSCAVKGFYPS |
| | | DIAVEWESNGQPENNYKTTPPVLDSDGSFFLCVSKLTVDKSRWQQGNVFSCSVMHEAL |
| | | HNHYTQKSLSLSPGK |
| 118 | Anti-CD132 clone P2B9 | QVQLQESGPGLVKPSETLSLTCTVSGGSISSSSYYWGWIRQPPGKGLEWIGSIYYSGST |
| | | YYNPSLKSRVTISVDTSKNQFSLKLSSVTAADTAVYYCAGDILTGYALDYWGQGTLVTVS |
| | | SGGGGSGGGGSGGGGSSYELTQPPSMSVSPGQTARITCSGDALPKQFAFWYQQKPG |
| | | QAPVLVIYKDTERPSGIPERFSGSSSGTTVTLTITGVQAEDEADYYCQSPDSSGTVEVFG |
| | | GGTKLTVL |
| 119 | Anti-CD132 clone | QVQLQQWGAGLLKPSETLSLTCAVYGGSFSGYYWSWIRQPPGKGLEWIGEINHFGSTN |
| | P1A3_B3 | YNPSLKSRATISVDTSKNQFSLKLSSVTAADTAVYYCATSPGGYSGGYFQHWGQGTLVT |
| | | VSSGGGGSGGGGSGGGGSDVVMTQSPLSLPVTPGEPASISCRSSQSLLHSNGYNYLD |
| | | WYLQKPGQSPQLLIYLGSNRDSGVPDRFSGSGSGTDFTLKISRVEAEDVGVYYCMQGT |
| | | HWPWTFGQGTKVEIK |
| 120 | Anti-CD132 clone | QVQLQQWGAGLLKPSETLSLTCAVYGGSFSGYYWSWIRQPPGKGLEWIGEINHFGSTN |
| | P1A3_B4 | YNPSLKSRATISVDTSKNQFSLKLSSVTAADTAVYYCATSPGGYSGGYFQHWGQGTLVT |
| | | VSSGGGGSGGGGSGGGGSDVVMTQSPLSLPVTPGESVSISCRSSQSLLHSNGYNYLD |
| | | WYLQKPGQSPQLLIYLGSNRDSGVPDRFSGSGSGTDFTLKISRVEAEDVGVYYCMQGT |
| | | HWPWTFGQGTKVEIK |
| 121 | Anti-CD132 clone | QVQLQQWGAGMLKPSETLSLTCAVYGGSFSGYYWSWIRQPPGKGLEWIGEINHFGST |
| | P1A3_E8 | NYNPSLKSRATISVDTSKNQFSLKLSSVTAADTAVYYCATSPGGYSGGYFQHWGQGTL |
| | | VTVSSGGGGSGGGGSGGGGSDVVMTQSPLSLPVTPGEPASISCRSSQSLLHSNGYNY |
| | | LDWYLQKPGQSPQLLIYLGSNRDSGVPDRFSGSGSGTDFTLKISRVEAEDVGVYYCMQ |
| | | GTHWPWTFGQGTKVEIK |
| 122 | Anti-CD132 clone | QVQLQQWGAGLLKPSETLSLTCAVYGGSFSGYYWSWIRQPPGKGLEWIGEINHFGSTN |
| | P1A3_E9 | YNPSLKSRATISVDTSKNQFSLKLSSVTAADTAVYYCATSPGGYSGGYFQHWGQGTLVT |
| | | VSSGGGGSGEGGSGGGGSDVVMTQSPLSLPVTPGEPASISCRSSQSLLHSNGYNYLD |
| | | WYLQKPGQSPQLLIYLGSNRDSGVPDRFSGSGSGTDFTLKISRVEAEDVGVYYCMQGT |
| | | HWPWTFGQGTKVEIKAAAHHHHH |
| 123 | Anti-CD132 clone | EVQLVESGGGLVQPGGSLRLSCAASGGSFSGYYWSWVRQAPGKGLEWVSEINHSGST |
| | P1A3_FW2 | NYNPSLKSRFTISRDNSKNTLYLQMNSLRAEDTAVYYCARSPGGYSGGYFQHWGQGTL |
| | | VTVSSGGGGSGGGGSGGGGSDIQMTQSPSSLSASVGDRVTITCRSSQSLLHSNGYNYL |
| | | DWYQQKPGKAPKLLIYLGSNRDSGVPSRFSGSGSGTDFTLTISSLQPEDFATYYCMQGT |
| | | HWPWTFGQGTKVEIK |
| 124 | Anti-CD132 clone P1A10 | QVQLQQSGAEVKKPGSSVKVSCKASGGTFSSYAISWVRQAPGQGLEWMGGFDPEDG |
| | | ETIYAQKFQGRVTMTEDTSTDTAYMELSSLRSEDTAVYYCATDLRIPYYYDNPWGQGTL |
| | | VTVSSGGGGSGGGGSGGGGSEIVLTQSPLSLPVTPGEPASISCRSSQSLLHSNGYNYL |
| | | NWYLQKPGQSPQLLIYLGSDRASGVPDRFSGSGSGTDFTLKISRVEAEDVGVYYCMQA |
| | | LQTPTTFGGGTKVEIK |
| 125 | Anti-CD132 clone P1B6 | QVQLVQSGGGVVQPGRSLRLSCAASGFTFSSYAMHWVRQAPGKGLEWVAVISYDGSN |
| | | KYYADSVKGRFTISRDNSKNTLYLQMNSLRAEDTAVYYCARSLYYSHFDYWGQGTLVTV |
| | | SSGGGGSGGGGSGGGGSEIVMTQSPLSLPVTPGEPASISCRSSQSLLHSNGYNYLDW |
| | | YLQKPGQSPQLLMYLVSNRASGVPERFSGSGSGTDFTLKISRVEAEDVGVYYCMQTLQ |
| | | TPLSFGQGTKLEIK |
| 126 | Anti-CD132 clone P1C10 | EVQLVETGPGLVKPSGTLSLTCAVSGGSISSSNWWSWVRQPPGKGLEWIGEIYHSGST |
| | | NYNPSLKSRVTISVDKSKNQFSLKLSSVTAADTAVYYCAREGPLSSSGPGAFDIWGQGT |
| | | MVTVSSGGGGSGGGGSGGGGSEIVLTQSPATLSLSPGERATLSCRASQSVSYHLAWY |
| | | QQKPGQAPRLLIYDTSNRASGIPARFSGSGSGTDFTLTINSLEPEDFAVYYCQQRYDWP |
| | | LTFGGGTKVEIK |
| 127 | Anti-CD132 clone P1D7 | QVQLQESGGGVVQPGRSLRLSCAASGFTFSNYGMHWVRQAPGKGLEWVAVISYDGTN |
| | | KYYADSVKGRFTISRDNSKNTVYLQMNSLRAEDTAVYYCAKDGFDIWGQGTMVTVSSG |
| | | GGGSGGGGSGGGGSDIQMTQSPSFLSASVGDRVTITCRASQSISSWLAWYQQKPGKA |
| | | PKLLIYDASRLEDGVPSRFSGTGFGTDFTFTITTLQPDDIATYYCQQYDDLPYTFGQGTT |
| | | VDIK |
| 128 | Anti-CD132 clone P1E8 | EVQLVQSGGGVVQPGRSLRLSCAASGFTFSSYGMHWVRQAPGKGLEWVAVISYDGSN |
| | | KYYADSVKGRFTISRDNSKNTLYLQMNSLRAEDTAVYYCARDVYGDYGAFDYWGQGTL |
| | | VTVSSGGGGSGGGGSGGGGSDVVMTQSPVSLPVTLGQPASISCKSSQSLLYFNGNTYL |
| | | SWFQQRPGQSPRRLFYQVSNRDSGVPDRFSGSGSDTDFTLTISRVEAEDVGVYFCMQ |
| | | GTQWPPTFGQGTKVEIK |
| 129 | Anti-CD132 clone P2B2 | QLQLQESGGGVVQPGRSLRLSCAASGFTFSSYAMHWVRQAPGKGLEWVAVISYDGGN |
| | | KYYADSVKGRFTISRDNSKNTLYLQMNSLRAEDTAVYYCAKSVAPPMDVWGKGTTVTV |
| | | SSGGGGSGGGGSGGGGSDVVMTQSPLSLPVTPGEPASISCRSSQSLLHSNGYNYLDW |
| | | YLQKPGQSPHLLIYLGSNRASGVPDRFSGSGSGTDFTLKISRVEAEDVGVYFCMQALRT |
| | | PYTFGQGTKLEIK |
| 130 | Anti-CD132 clone P2B7 | QVQLQQWGAGLLKPSETLSLTCAVYGESFSGYYWSWIRQPPGKGLEWIGEINHSGSTN |
| | | YNPSLKSRVTISVDTSKNQFSLKLSSVTAADTAVYYCARGPAGSSSSGYFDYWGQGTLV |
| | | TVSSGGGGSGGGGSGGGGSDVVMTQSPLSLPVTLGQPASISCRSSQSLVHSNGYNYL |
| | | DWYLQKPGQSPQLLIYLGSNRASGVPDRFSGSGSGTDFTLKISRVEAEDVGVYYCLQG |
| | | SHWPWTFGQGTKVEIK |
| 131 | Anti-CD132 clone P2D11 | QVQLQESGPGLVKPSQTLSLTCTVSGGSISSGGYYWTWIRQHPGQGLEWIGFISWSGT |
| | | TYYNPSLKNRVTISADTSKNHFSLNLTSVTAADTAVYYCARGSGRLVWGQGTLVTVSSG |
| | | GGGSGGGGSGGGGSETTLTQSPATLSVSPGERATLSCRASQSVSSNLAWYQQKPGQ |
| | | APRLLIYGASSGATGIPDRFSGSGSGTDFTLTISRLEPEDFAVYYCQLYGSSLAFGGGTK |
| | | VEIK |
| 132 | Anti-CD132 clone P2F10 | EVQLVQSGAEVKKPGASVKVSCKASGYTFTGYYMHWVRQAPGQGLEWMGIINPSGGS |
| | | TSYAQKFQGRVTMTRDTSTSTVYMELSSLRSEDTAVYYCARADTAMGDAFDIWGQGTM |
| | | VTVSSGGGGSGGGGSGGGGSDIVMTHTPLSLPVTPGEPASISCRSSQTLFDSDDGKTY |
| | | LDWYLQKPGQSPQLLMYTTSSRASGVPDRFSGSGSGTDFTLKISRVEAEDVGVYYCMQ |
| | | RLQFPLTFGQGTRLEFK |
| 133 | Anti-CD132 clone P2H4 | EVQLVQSGGGVVQPGRSLRLSCAASGFTFSSYAMHWVRQAPGKGLEWVAVISYDGSN |
| | | KYYADSVKGRFTISRDNSKNTLYLQMNSLRAEDTAVYYCARSIGIGAFDIWGQGTMVTV |
| | | SSGGGGSGGGGSGGGGSDVVMTQSPLSLPVTPGEPASISCRATQSLLHGNGHNYLDW |
| | | YLQKPGQSPQLLIYLGSNRASGVPDRFSGSGSGTDFTLKISRVEAEDVGVYYCMQTLET |
| | | PVTFGPGTKVDIK |
| 134 | Anti-CD132 clone P2D3 | QVQLQQWGAGLLKPSETLSLTCTIYGGSFSGFYWSWIRQPPGKGLEWIGEINHSGSTNY |
| | | NPSLKSRVTISVDTSKNQFSLKLSSVTAADTAIYYCARGPAGSTSSGYFDHWGQGTLVT |
| | | VSSGGGGSGGGGSGGGGSDVVMTQSPLSLPVTPGEPASISCRSSQSLLHSNGYNYLD |
| | | WYLQKPGQSPQLLIYLGSNRASGVPDRFSGSGSGTDFTLKISRVEAEDVGVYYCMQGT |
| | | HWPWTFGQGTKVEIK |
| 135 | Anti-CD132 clone P1G4 | QVQLQQWGAGLLKPSETLSLTCAVYGGSLSGYYWSWIRQPPGKGLEWIGEINHSGSTN |
| | | YNPSLKSRVTISVDTSKNQFSLKLSSVTAADTAVYYCARGSSSYYMDVWGKGTTVTVSS |
| | | GGGGSGGGGSGGGGSDVVMTQSPLSLPVTPGEPASISCRSSQSLLHSNGYNYLDWYL |
| | | QKPGQSPQLLIYLGSNRASGVPDRFSGSGSGTDFTLKISRVEAEDVGVYYCLQGTHWP |
| | | WTFGQGTKVEIK |
| 136 | Anti-CD132 clone P1B12 | QVQLQQWGAGLLKPSETLSLTCAVYGGSFSGYYWSWIRQPPGKGLEWIGEINHSGSTN |
| | | YNPSLKSRVTISVDTSKNQFSLKLSSVTAADTAVYYCARGGSAYFQHWGQGTLVTVSSG |
| | | GGGSGGGGSGGGGSDVVMTQSPLSLPVTLGQPASISCRSSQSLLHSNGNNYLDWYLQ |
| | | KPGQSPQLLIYLGSNRASGVPDRFSGSGSGTDFTLKISRVEAEDVGIYYCMQGTHWPW |
| | | TFGQGTKVEIE |
| 137 | Anti-CD132 clone P1C7 | QVQLQQWGAGLLKPSETLSLTCAVYGGSFSGYYWSWIRQPPGKGLEWIGEINHSGSTN |
| | | YNPSLKSRVTISEDASKKQFSLTLTSVTAADTAVYYCARGPAGTGSSGYFDYWGQGTLV |
| | | TVSSGGGGSGGGGSGGGGSEIVLTQSPLSLPVTPGEPASISCRSSQSLLHSNGYNYLD |
| | | WYLQKPGQSPQLLIYLASNRASGVPDRFSGSGSGTDFTLKISRVEAEDVGVYYCMQGT |
| | | HWPWTFGQGTKVEVK |
| 138 | Linker 1 | NSGAGTAAA |
| 139 | Linker 2 | NSGAGTSGSGASGEGSGSKLAAA |
| 140 | Linker 3 | GGGGSAAA |
| 141 | Linker 4 | GGGGSGGGGSGGGGS |
| 142 | Tag | AAAHHHHHH |
| 143 | Anti-CD132 P1A3 Fab LC | GATGTTGTGATGACTCAGTCTCCACTCTCCCTGCCCGTCACCCCTGGAGAGCCGGC |
| | (VL, joint CL) | CTCCATCTCCTGCAGGTCTAGTCAGAGCCTCCTGCATAGTAATGGATACAACTATTT |
| | | GGATTGGTACCTGCAGAAGCCAGGGCAGTCTCCACAGCTCCTGATCTATTTGGGTT |
| | | CTAACCGGGACTCTGGGGTCCCAGACAGATTCAGCGGCAGTGGGTCAGGCACTGAT |
| | | TTCACACTGAAAATCAGCAGGGTGGAGGCTGAGGATGTTGGGGTTTATTACTGCATG |
| | | CAAGGTACACACTGGCCGTGGACGTTCGGCCAAGGGACCAAGGTGGAAATCAAACG |
| | | AACTGTGGCTGCACCATCTGTCTTCATCTTCCCGCCATCTGATGAGCAGTTGAAATC |
| | | TGGAACTGCCTCTGTTGTGTGCCTGCTGAATAACTTCTATCCCAGAGAGGCCAAAGT |
| | | ACAGTGGAAGGTGGATAACGCCCTCCAATCGGGTAACTCCCAGGAGAGTGTCACAG |
| | | AGCAGGACAGCAAGGACAGCACCTACAGCCTCAGCAGCACCCTGACGCTGAGCAAA |
| | | GCAGACTACGAGAAACACAAAGTCTACGCCTGCGAAGTCACCCATCAGGGCCTGAG |
| | | CTCGCCCGTCACAAAGAGCTTCAACAGGGGAGAGTGT |
| 144 | Anti-CD132 P1A3 Fab HC | CAGGTGCAGCTACAGCAGTGGGGCGCAGGACTGTTGAAGCCTTCGGAGACCCTGT |
| | (VH, joint CH1) | CCCTCACCTGCGCTGTCTATGGTGGGTCCTTCAGTGGTTACTACTGGAGCTGGATC |
| | | CGCCAGCCCCCAGGGAAGGGGCTGGAGTGGATTGGGGAAATCAATCATAGTGGAA |
| | | GCACCAACTACAACCCGTCCCTCAAGAGTCGAGCCACCATATCAGTAGACACGTCC |
| | | AAGAACCAGTTCTCCCTGAAGCTGAGCTCTGTGACCGCCGCGGACACGGCTGTGTA |
| | | TTACTGTGCGACCAGCCCGGGAGGCTATTCCGGGGGATACTTCCAGCACTGGGGC |
| | | CAGGGAACCCTGGTCACCGTCTCAAGCGCCTCCACCAAGGGCCCATCGGTCTTCCC |
| | | CCTGGCACCCTCCTCCAAGAGCACCTCTGGGGGCACAGCGGCCCTGGGCTGCCTG |
| | | GTCAAGGACTACTTCCCCGAACCGGTGACGGTGTCGTGGAACTCAGGCGCCCTGAC |
| | | CAGCGGCGTCCACACCTTCCCGGCTGTCCTACAGTCCTCAGGACTCTACTCCCTCA |
| | | GCAGCGTAGTGACCGTGCCCTCCAGCAGCTTGGGCACCCAGACCTACATCTGCAAC |
| | | GTGAATCACAAGCCCAGCAACACCAAGGTGGACAAGAAAGTTGAGCCCAAATCTTGT |
| 145 | Anti-CD132 P1A3 scFv | CAGGTCCAGCTGCAGCAGTGGGGAGCCGGCCTGCTGAAACCATCTGAAACTCTGAG |
| | and Fc with hole | CCTGACTTGCGCTGTCTACGGGGGGTCCTTCAGTGGCTACTATTGGTCATGGATCA |
| | modification | GGCAGCCCCCTGGGAAGGGACTGGAGTGGATCGGGGAAATTAACCACTCCGGATC |
| | | TACAAACTACAATCCCAGTCTGAAATCACGCGCCACCATTTCTGTGGACACCAGTAA |
| | | GAATCAGTTCAGCCTGAAGCTGAGCAGCGTGACAGCCGCTGATACCGCCGTGTACT |
| | | ATTGCGCAACCAGCCCTGGCGGATACTCCGGAGGCTATTTTCAGCATTGGGGCCAG |
| | | GGGACCCTGGTGACAGTCTCTAGTGGGGGAGGAGGGTCTGGAGGAGGAGGAAGTG |
| | | GAGGAGGAGGCTCCGACGTGGTCATGACTCAGAGCCCACTGTCCCTGCCAGTGAC |
| | | CCCCGGCGAGCCTGCTAGTATCTCATGTCGATCAAGCCAGTCACTGCTGCACAGCA |
| | | ACGGGTACAATTATCTGGATTGGTACTTGCAGAAGCCAGGCCAGTCTCCCCAGCTG |
| | | CTGATCTATCTGGGCTCCAACCGGGACTCTGGGGTGCCTGATAGATTCAGCGGCAG |
| | | CGGCTCTGGGACTGACTTTACCCTGAAAATTTCCAGAGTCGAGGCAGAAGATGTGG |
| | | GAGTCTACTATTGCATGCAGGGCACTCATTGGCCCTGGACCTTCGGACAGGGCACA |
| | | AAGGTGGAGATCAAGAACAGCGGCGCGGGCACCGCGGCCGCGACTCACACATGCC |
| | | CACCGTGCCCAGCACCTGAACTCCTGGGGGGACCGTCAGTCTTCCTCTTCCCCCCA |
| | | AAACCCAAGGACACCCTCATGATCTCCCGGACCCCTGAGGTCACATGCGTGGTGGT |
| | | GGACGTGAGCCACGAAGACCCTGAGGTCAAGTTCAACTGGTACGTGGACGGCGTG |
| | | GAGGTGCATAATGCCAAGACAAAGCCGCGGGAGGAGCAGTACAACAGCACGTACC |
| | | GTGTGGTCAGCGTCCTCACCGTCCTGCACCAGGACTGGCTGAATGGCAAGGAGTAC |
| | | AAGTGCAAGGTCTCCAACAAAGCCCTCCCAGCCCCCATCGAGAAAACCATCTCCAAA |
| | | GCCAAAGGGCAGCCCCGAGAACCACAGGTGTGCACCCTGCCCCCATCCCGGGATG |
| | | AGCTGACCAAGAACCAGGTCAGCCTGTCCTGCGCCGTCAAAGGCTTCTATCCCAGC |
| | | GACATCGCCGTGGAGTGGGAGAGCAATGGGCAGCCGGAGAACAACTACAAGACCA |
| | | CGCCTCCCGTGCTGGACTCCGACGGCTCCTTCTTCCTCGTGAGCAAGCTCACCGTG |
| | | GACAAGAGCAGGTGGCAGCAGGGGAACGTCTTCTCATGCTCCGTGATGCATGAGGC |
| | | TCTGCACAACCACTACACGCAGAAGAGCCTCTCCCTGTCTCCGGGTAAA |
| 146 | Anti-CD132 P2B9 Fab LC | TCCTATGAGCTGACTCAGCCACCCTCGATGTCAGTGTCCCCAGGACAGACGGCCAG |
| | (VL, joint CL) | GATCACCTGCTCTGGAGATGCATTGCCAAAACAATTTGCTTTTTGGTACCAGCAGAA |
| | | GCCAGGCCAGGCCCCTGTGTTGGTGATTTATAAAGACACTGAGAGGCCCTCAGGGA |
| | | TCCCTGAGCGATTCTCTGGCTCCAGCTCAGGGACAACAGTCACGTTGACCATCACT |
| | | GGAGTCCAGGCAGAAGATGAGGCTGACTATTACTGTCAATCTCCAGACAGCAGTGG |
| | | TACCGTCGAAGTGTTCGGCGGAGGGACCAAGCTGACCGTCCTAGGTCAGCCCAAG |
| | | GCTGCCCCCTCGGTCACTCTGTTCCCGCCCTCCTCTGAGGAGCTTCAAGCCAACAA |
| | | GGCCACACTGGTGTGTCTCATAAGTGACTTCTACCCGGGAGCCGTGACAGTGGCCT |
| | | GGAAGGCAGATAGCAGCCCCGTCAAGGCGGGAGTGGAGACCACCACACCCTCCAA |
| | | ACAAAGCAACAACAAGTACGCGGCCAGCAGCTACCTGAGCCTGACGCCTGAGCAGT |
| | | GGAAGTCCCACAGAAGCTACAGCTGCCAGGTCACGCATGAAGGGAGCACCGTGGA |
| | | GAAGACAGTGGCCCCTGCAGAATGT |
| 147 | Anti-CD132 P2B9 Fab HC | CAGGTGCAGCTGCAGGAGTCGGGCCCAGGACTGGTGAAGCCTTCGGAGACCCTGT |
| | (VH, joint CH1) | CCCTCACCTGCACTGTCTCTGGTGGCTCCATCAGCAGTAGTAGTTACTACTGGGGCT |
| | | GGATCCGCCAGCCCCCAGGGAAGGGGCTGGAGTGGATTGGGAGTATCTATTATAGT |
| | | GGGAGCACCTACTACAACCCGTCCCTCAAGAGTCGAGTCACCATATCCGTAGACAC |
| | | GTCCAAGAACCAGTTCTCCCTGAAGCTGAGCTCTGTGACCGCCGCAGACACGGCTG |
| | | TGTATTACTGTGCGGGCGATATTTTGACTGGTTATGCCCTTGACTACTGGGGCCAGG |
| | | GAACCCTGGTCACCGTCTCAAGCGCCTCCACCAAGGGCCCATCGGTCTTCCCCCTG |
| | | GCACCCTCCTCCAAGAGCACCTCTGGGGGCACAGCGGCCCTGGGCTGCCTGGTCA |
| | | AGGACTACTTCCCCGAACCGGTGACGGTGTCGTGGAACTCAGGCGCCCTGACCAG |
| | | CGGCGTCCACACCTTCCCGGCTGTCCTACAGTCCTCAGGACTCTACTCCCTCAGCA |
| | | GCGTAGTGACCGTGCCCTCCAGCAGCTTGGGCACCCAGACCTACATCTGCAACGTG |
| | | AATCACAAGCCCAGCAACACCAAGGTGGACAAGAAAGTTGAGCCCAAATCTTGT |
| 148 | Anti-CD132 P2B9 scFv | CAGGTGCAGCTGCAGGAAAGCGGACCCGGACTGGTGAAGCCATCTGAAACACTGA |
| | and Fc with hole | GCCTGACTTGTACCGTGAGCGGCGGAAGCATCAGCTCCTCTAGTTACTATTGGGGA |
| | modification | TGGATCAGGCAGCCCCCTGGCAAGGGGCTGGAGTGGATCGGCAGCATCTACTATA |
| | | GCGGCTCCACATACTATAACCCTAGCCTGAAATCCCGCGTGACAATCTCTGTGGACA |
| | | CTAGTAAGAATCAGTTCTCTCTGAAACTGTCAAGCGTGACCGCCGCTGATACAGCTG |
| | | TCTACTATTGCGCAGGCGACATTCTGACCGGGTACGCCCTGGATTATTGGGGACAG |
| | | GGCACTCTGGTGACCGTCTCCTCTGGAGGAGGAGGCTCAGGAGGAGGAGGGTCCG |
| | | GAGGCGGGGGAAGTTCATACGAACTGACACAGCCACCCTCTATGAGTGTGTCACCA |
| | | GGGCAGACTGCACGAATCACCTGTAGCGGAGACGCCCTGCCCAAGCAGTTCGCTTT |
| | | TTGGTATCAGCAGAAACCTGGCCAGGCTCCAGTGCTGGTCATCTATAAGGATACTGA |
| | | GCGGCCCTCTGGGATTCCTGAAAGATTCAGTGGCAGCAGCAGCGGAACCACAGTGA |
| | | CTCTGACCATTACAGGCGTGCAGGCAGAGGACGAAGCCGATTACTATTGCCAGTCC |
| | | CCCGACAGTTCAGGCACCGTGGAGGTCTTTGGCGGGGGAACAAAACTGACTGTGCT |
| | | GAACAGCGGCGCGGGCACCGCGGCCGCGACTCACACATGCCCACCGTGCCCAGCA |
| | | CCTGAACTCCTGGGGGGACCGTCAGTCTTCCTCTTCCCCCCAAAACCCAAGGACAC |
| | | CCTCATGATCTCCCGGACCCCTGAGGTCACATGCGTGGTGGTGGACGTGAGCCACG |
| | | AAGACCCTGAGGTCAAGTTCAACTGGTACGTGGACGGCGTGGAGGTGCATAATGCC |
| | | AAGACAAAGCCGCGGGAGGAGCAGTACAACAGCACGTACCGTGTGGTCAGCGTCC |
| | | TCACCGTCCTGCACCAGGACTGGCTGAATGGCAAGGAGTACAAGTGCAAGGTCTCC |
| | | AACAAAGCCCTCCCAGCCCCCATCGAGAAAACCATCTCCAAAGCCAAAGGGCAGCC |
| | | CCGAGAACCACAGGTGTGCACCCTGCCCCCATCCCGGGATGAGCTGACCAAGAAC |
| | | CAGGTCAGCCTGTCCTGCGCCGTCAAAGGCTTCTATCCCAGCGACATCGCCGTGGA |
| | | GTGGGAGAGCAATGGGCAGCCGGAGAACAACTACAAGACCACGCCTCCCGTGCTG |
| | | GACTCCGACGGCTCCTTCTTCCTCGTGAGCAAGCTCACCGTGGACAAGAGCAGGTG |
| | | GCAGCAGGGGAACGTCTTCTCATGCTCCGTGATGCATGAGGCTCTGCACAACCACT |
| | | ACACGCAGAAGAGCCTCTCCCTGTCTCCGGGTAAA |
| 149 | Anti-CD132 P1A10 Fab LC | GAAATTGTGCTGACTCAGTCTCCACTCTCCCTGCCCGTTACCCCTGGAGAGCCGGC |
| | (VL, joint CL) | CTCCATCTCCTGCAGGTCTAGTCAGAGCCTCCTGCATAGTAATGGATACAACTATTT |
| | | GAATTGGTACCTACAGAAGCCAGGGCAGTCTCCACAACTCCTGATCTATTTGGGTTC |
| | | TGATCGGGCCTCCGGGGTCCCTGACAGGTTCAGTGGCAGTGGATCAGGCACAGATT |
| | | TTACACTGAAAATCAGCAGAGTGGAGGCTGAGGATGTTGGGGTTTATTACTGCATGC |
| | | AAGCTCTACAAACCCCCACCACTTTCGGCGGAGGGACCAAGGTGGAGATCAAACGA |
| | | ACTGTGGCTGCACCATCTGTCTTCATCTTCCCGCCATCTGATGAGCAGTTGAAATCT |
| | | GGAACTGCCTCTGTTGTGTGCCTGCTGAATAACTTCTATCCCAGAGAGGCCAAAGTA |
| | | CAGTGGAAGGTGGATAACGCCCTCCAATCGGGTAACTCCCAGGAGAGTGTCACAGA |
| | | GCAGGACAGCAAGGACAGCACCTACAGCCTCAGCAGCACCCTGACGCTGAGCAAA |
| | | GCAGACTACGAGAAACACAAAGTCTACGCCTGCGAAGTCACCCATCAGGGCCTGAG |
| | | CTCGCCCGTCACAAAGAGCTTCAACAGGGGAGAGTGT |
| 150 | Anti-CD132 P1A10 Fab | CAGGTACAGCTGCAGCAGTCAGGGGCTGAGGTGAAGAAGCCTGGGTCCTCGGTGA |
| | HC (VH, joint CH1) | AGGTCTCCTGCAAGGCTTCTGGAGGCACCTTCAGCAGCTATGCTATCAGCTGGGTG |
| | | CGACAGGCCCCTGGACAAGGGCTTGAGTGGATGGGAGGTTTTGATCCTGAAGATGG |
| | | TGAAACAATCTACGCACAGAAGTTCCAGGGCAGAGTCACCATGACCGAGGACACAT |
| | | CTACAGACACAGCCTACATGGAGCTGAGCAGCCTGAGATCTGAGGACACGGCCGTG |
| | | TATTACTGTGCAACAGATCTGAGAATTCCGTATTACTATGATAACCCCTGGGGCCAG |
| | | GGCACCCTGGTCACCGTCTCAAGCGCCTCCACCAAGGGCCCATCGGTCTTCCCCCT |
| | | GGCACCCTCCTCCAAGAGCACCTCTGGGGGCACAGCGGCCCTGGGCTGCCTGGTC |
| | | AAGGACTACTTCCCCGAACCGGTGACGGTGTCGTGGAACTCAGGCGCCCTGACCAG |
| | | CGGCGTCCACACCTTCCCGGCTGTCCTACAGTCCTCAGGACTCTACTCCCTCAGCA |
| | | GCGTAGTGACCGTGCCCTCCAGCAGCTTGGGCACCCAGACCTACATCTGCAACGTG |
| | | AATCACAAGCCCAGCAACACCAAGGTGGACAAGAAAGTTGAGCCCAAATCTTGT |
| 151 | Anti-CD132 P1A10 scFv | CAGGTCCAGCTGCAGCAGAGCGGAGCCGAGGTCAAGAAGCCAGGGAGTAGCGTCA |
| | and Fc with hole | AAGTCAGTTGTAAAGCATCAGGAGGAACATTCAGCTCCTATGCAATCTCTTGGGTGC |
| | modification | GACAGGCCCCTGGACAGGGCCTGGAGTGGATGGGAGGATTCGACCCAGAGGATGG |
| | | AGAAACCATCTACGCCCAGAAGTTTCAGGGCAGAGTGACTATGACCGAAGACACAT |
| | | CTACTGATACCGCTTACATGGAGCTGTCTAGTCTGAGGAGTGAAGACACTGCCGTCT |
| | | ACTATTGCGCTACCGACCTGCGCATCCCATACTATTACGATAATCCCTGGGGGCAGG |
| | | GAACACTGGTGACTGTCTCAAGCGGAGGCGGGGGATCAGGCGGAGGAGGCAGCG |
| | | GAGGAGGAGGGTCCGAGATCGTGCTGACACAGAGTCCACTGTCACTGCCAGTCACC |
| | | CCTGGCGAACCAGCCAGTATTTCATGTCGGTCCTCTCAGAGCCTGCTGCACTCCAA |
| | | CGGGTATAATTACCTGAACTGGTACTTGCAGAAGCCTGGCCAGAGCCCTCAGCTGC |
| | | TGATCTACCTGGGCTCTGACCGAGCAAGTGGGGTGCCCGATAGATTCAGCGGCTCC |
| | | GGGTCTGGAACCGACTTTACCCTGAAGATCAGCCGGGTGGAGGCTGAAGATGTGG |
| | | GCGTCTATTACTGCATGCAGGCCCTCCAGACACCTACCACATTCGGAGGCGGGACT |
| | | AAGGTGGAGATCAAGAACAGCGGCGCGGGCACCGCGGCCGCGACTCACACATGCC |
| | | CACCGTGCCCAGCACCTGAACTCCTGGGGGGACCGTCAGTCTTCCTCTTCCCCCCA |
| | | AAACCCAAGGACACCCTCATGATCTCCCGGACCCCTGAGGTCACATGCGTGGTGGT |
| | | GGACGTGAGCCACGAAGACCCTGAGGTCAAGTTCAACTGGTACGTGGACGGCGTG |
| | | GAGGTGCATAATGCCAAGACAAAGCCGCGGGAGGAGCAGTACAACAGCACGTACC |
| | | GTGTGGTCAGCGTCCTCACCGTCCTGCACCAGGACTGGCTGAATGGCAAGGAGTAC |
| | | AAGTGCAAGGTCTCCAACAAAGCCCTCCCAGCCCCCATCGAGAAAACCATCTCCAAA |
| | | GCCAAAGGGCAGCCCCGAGAACCACAGGTGTGCACCCTGCCCCCATCCCGGGATG |
| | | AGCTGACCAAGAACCAGGTCAGCCTGTCCTGCGCCGTCAAAGGCTTCTATCCCAGC |
| | | GACATCGCCGTGGAGTGGGAGAGCAATGGGCAGCCGGAGAACAACTACAAGACCA |
| | | CGCCTCCCGTGCTGGACTCCGACGGCTCCTTCTTCCTCGTGAGCAAGCTCACCGTG |
| | | GACAAGAGCAGGTGGCAGCAGGGGAACGTCTTCTCATGCTCCGTGATGCATGAGGC |
| | | TCTGCACAACCACTACACGCAGAAGAGCCTCTCCCTGTCTCCGGGTAAA |
| 152 | Anti-CD132 P1B6 Fab LC | GAAATTGTGATGACGCAGTCTCCACTCTCCCTGCCCGTCACCCCTGGAGAGCCGGC |
| | (VL, joint CL) | CTCCATCTCCTGCAGGTCTAGTCAGAGCCTCCTGCATAGTAATGGATACAACTATTT |
| | | GGATTGGTACCTGCAGAAGCCAGGGCAGTCTCCACAGCTCCTGATGTATTTGGTTTC |
| | | TAATCGGGCCTCCGGGGTCCCTGAGAGGTTCAGTGGCAGTGGATCAGGCACAGATT |
| | | TTACACTGAAAATCAGCAGAGTGGAGGCTGAGGATGTTGGGGTTTATTACTGCATGC |
| | | AAACTCTACAAACTCCTCTCAGTTTTGGCCAGGGGACCAAGCTGGAGATCAAACGAA |
| | | CTGTGGCTGCACCATCTGTCTTCATCTTCCCGCCATCTGATGAGCAGTTGAAATCTG |
| | | GAACTGCCTCTGTTGTGTGCCTGCTGAATAACTTCTATCCCAGAGAGGCCAAAGTAC |
| | | AGTGGAAGGTGGATAACGCCCTCCAATCGGGTAACTCCCAGGAGAGTGTCACAGAG |
| | | CAGGACAGCAAGGACAGCACCTACAGCCTCAGCAGCACCCTGACGCTGAGCAAAG |
| | | CAGACTACGAGAAACACAAAGTCTACGCCTGCGAAGTCACCCATCAGGGCCTGAGC |
| | | TCGCCCGTCACAAAGAGCTTCAACAGGGGAGAGTGT |
| 153 | Anti-CD132 P1B6 Fab HC | CAGGTCCAGCTGGTACAGTCTGGGGGAGGCGTGGTCCAGCCTGGGAGGTCCCTGA |
| | (VH, joint CH1) | GACTCTCCTGTGCAGCCTCTGGATTCACCTTCAGTAGCTATGCTATGCACTGGGTCC |
| | | GCCAGGCTCCAGGCAAGGGGCTGGAGTGGGTGGCAGTTATATCATATGATGGAAGC |
| | | AATAAATACTACGCAGACTCCGTGAAGGGCCGATTCACCATCTCCAGAGACAATTCC |
| | | AAGAACACGCTGTATCTGCAAATGAACAGCCTGAGAGCCGAGGACACGGCTGTGTA |
| | | TTACTGTGCGAGAAGTCTTTACTACAGCCACTTTGACTACTGGGGCCAGGGAACCCT |
| | | GGTCACCGTCTCAAGCGCCTCCACCAAGGGCCCATCGGTCTTCCCCCTGGCACCCT |
| | | CCTCCAAGAGCACCTCTGGGGGCACAGCGGCCCTGGGCTGCCTGGTCAAGGACTA |
| | | CTTCCCCGAACCGGTGACGGTGTCGTGGAACTCAGGCGCCCTGACCAGCGGCGTC |
| | | CACACCTTCCCGGCTGTCCTACAGTCCTCAGGACTCTACTCCCTCAGCAGCGTAGT |
| | | GACCGTGCCCTCCAGCAGCTTGGGCACCCAGACCTACATCTGCAACGTGAATCACA |
| | | AGCCCAGCAACACCAAGGTGGACAAGAAAGTTGAGCCCAAATCTTGT |
| 154 | Anti-CD132 P1B6 scFv | CAGGTGCAGCTGGTCCAGAGCGGAGGAGGCGTCGTCCAGCCCGGAAGGTCACTGA |
| | and Fc with hole | GACTGTCTTGTGCCGCATCAGGATTCACTTTTAGCTCCTACGCAATGCACTGGGTGA |
| | modification | GGCAGGCCCCTGGCAAGGGGCTGGAGTGGGTGGCTGTCATCAGTTATGACGGCTC |
| | | AAACAAGTACTATGCAGATAGCGTGAAAGGGCGGTTCACCATTAGCAGAGACAACTC |
| | | CAAAAATACACTGTACCTCCAGATGAACAGCCTGCGAGCCGAAGACACAGCTGTGTA |
| | | CTATTGCGCCCGGTCTCTGTACTATAGTCACTTTGATTACTGGGGACAGGGCACCCT |
| | | GGTGACAGTCTCTAGTGGCGGGGGAGGCAGTGGAGGAGGAGGGAGCGGAGGAGG |
| | | AGGCAGCGAGATCGTGATGACTCAGTCCCCACTGTCTCTGCCAGTCACCCCTGGCG |
| | | AACCAGCATCCATTTCTTGTAGATCAAGCCAGTCACTGCTGCATAGCAACGGATACA |
| | | ATTATCTGGATTGGTACTTGCAGAAGCCTGGCCAGTCTCCTCAGCTGCTGATGTATC |
| | | TGGTGTCCAACAGGGCCTCTGGGGTCCCAGAGCGCTTCAGTGGGTCAGGAAGCGG |
| | | CACTGACTTTACCCTGAAAATCTCTCGCGTGGAGGCTGAAGATGTGGGCGTCTACTA |
| | | TTGCATGCAGACACTCCAGACTCCCCTGAGCTTCGGGCAGGGAACCAAGCTGGAGA |
| | | TCAAGAACAGCGGCGCGGGCACCGCGGCCGCGACTCACACATGCCCACCGTGCCC |
| | | AGCACCTGAACTCCTGGGGGGACCGTCAGTCTTCCTCTTCCCCCCAAAACCCAAGG |
| | | ACACCCTCATGATCTCCCGGACCCCTGAGGTCACATGCGTGGTGGTGGACGTGAGC |
| | | CACGAAGACCCTGAGGTCAAGTTCAACTGGTACGTGGACGGCGTGGAGGTGCATAA |
| | | TGCCAAGACAAAGCCGCGGGAGGAGCAGTACAACAGCACGTACCGTGTGGTCAGC |
| | | GTCCTCACCGTCCTGCACCAGGACTGGCTGAATGGCAAGGAGTACAAGTGCAAGGT |
| | | CTCCAACAAAGCCCTCCCAGCCCCCATCGAGAAAACCATCTCCAAAGCCAAAGGGC |
| | | AGCCCCGAGAACCACAGGTGTGCACCCTGCCCCCATCCCGGGATGAGCTGACCAA |
| | | GAACCAGGTCAGCCTGTCCTGCGCCGTCAAAGGCTTCTATCCCAGCGACATCGCCG |
| | | TGGAGTGGGAGAGCAATGGGCAGCCGGAGAACAACTACAAGACCACGCCTCCCGT |
| | | GCTGGACTCCGACGGCTCCTTCTTCCTCGTGAGCAAGCTCACCGTGGACAAGAGCA |
| | | GGTGGCAGCAGGGGAACGTCTTCTCATGCTCCGTGATGCATGAGGCTCTGCACAAC |
| | | CACTACACGCAGAAGAGCCTCTCCCTGTCTCCGGGTAAA |
| 155 | Anti-CD132 P1C10 Fab | GAAATTGTGCTGACTCAGTCTCCAGCCACCCTGTCTTTGTCTCCAGGGGAACGAGC |
| | LC (VL, joint CL) | CACCCTCTCCTGCAGGGCCAGTCAGAGTGTTAGTTACCACTTAGCCTGGTACCAACA |
| | | AAAACCTGGCCAGGCTCCCAGGCTCCTCATCTATGATACATCCAACAGGGCCTCTG |
| | | GCATCCCCGCCAGGTTCAGTGGCAGTGGGTCTGGGACAGACTTCACTCTCACCATC |
| | | AACAGCCTAGAGCCTGAAGATTTTGCAGTTTATTACTGTCAGCAGCGTTACGACTGG |
| | | CCTCTCACTTTCGGCGGAGGGACCAAGGTGGAGATCAAACGAACTGTGGCTGCACC |
| | | ATCTGTCTTCATCTTCCCGCCATCTGATGAGCAGTTGAAATCTGGAACTGCCTCTGTT |
| | | GTGTGCCTGCTGAATAACTTCTATCCCAGAGAGGCCAAAGTACAGTGGAAGGTGGA |
| | | TAACGCCCTCCAATCGGGTAACTCCCAGGAGAGTGTCACAGAGCAGGACAGCAAGG |
| | | ACAGCACCTACAGCCTCAGCAGCACCCTGACGCTGAGCAAAGCAGACTACGAGAAA |
| | | CACAAAGTCTACGCCTGCGAAGTCACCCATCAGGGCCTGAGCTCGCCCGTCACAAA |
| | | GAGCTTCAACAGGGGAGAGTGT |
| 156 | Anti-CD132 P1C10 Fab | GAGGTGCAGCTGGTGGAGACTGGCCCAGGACTGGTGAAGCCTTCGGGGACCCTGT |
| | HC (VH, joint CH1) | CCCTCACCTGCGCTGTCTCTGGTGGCTCCATCAGCAGTAGTAACTGGTGGAGTTGG |
| | | GTCCGCCAGCCCCCAGGGAAGGGGCTGGAGTGGATTGGGGAAATCTATCATAGTG |
| | | GGAGCACCAACTACAACCCGTCCCTCAAGAGTCGAGTCACCATATCAGTAGACAAGT |
| | | CCAAGAACCAGTTCTCCCTGAAGCTGAGCTCTGTGACCGCCGCGGACACGGCCGTG |
| | | TATTACTGTGCGAGAGAAGGGCCCCTAAGCAGCAGCGGACCGGGTGCTTTTGATAT |
| | | CTGGGGCCAAGGGACAATGGTCACCGTCTCAAGCGCCTCCACCAAGGGCCCATCG |
| | | GTCTTCCCCCTGGCACCCTCCTCCAAGAGCACCTCTGGGGGCACAGCGGCCCTGG |
| | | GCTGCCTGGTCAAGGACTACTTCCCCGAACCGGTGACGGTGTCGTGGAACTCAGGC |
| | | GCCCTGACCAGCGGCGTCCACACCTTCCCGGCTGTCCTACAGTCCTCAGGACTCTA |
| | | CTCCCTCAGCAGCGTAGTGACCGTGCCCTCCAGCAGCTTGGGCACCCAGACCTACA |
| | | TCTGCAACGTGAATCACAAGCCCAGCAACACCAAGGTGGACAAGAAAGTTGAGCCC |
| | | AAATCTTGT |
| 157 | Anti-CD132 P1C10 scFv | CAGGTCCAGCTGCAGGAATCAGGAGGGGGGGTCGTCCAGCCAGGGAGGTCACTGA |
| | and Fc with hole | GACTGTCTTGCGCCGCTTCAGGGTTCACTTTTAGCAACTACGGAATGCACTGGGTGC |
| | modification | GGCAGGCTCCCGGCAAAGGGCTGGAGTGGGTGGCAGTCATCTCTTATGACGGCAC |
| | | AAACAAGTACTATGCAGATAGTGTCAAGGGGCGGTTCACCATCAGCCGGGACAACA |
| | | GTAAAAATACAGTGTACCTCCAGATGAACAGCCTGCGGGCCGAAGATACTGCTGTCT |
| | | ACTATTGCGCCAAGGACGGGTTTGACATCTGGGGACAGGGCACTATGGTGACCGTC |
| | | AGCTCCGGCGGGGGAGGCTCAGGAGGAGGAGGGAGCGGAGGAGGAGGCAGCGA |
| | | CATTCAGATGACCCAGTCACCTAGCTTCCTGTCCGCTTCTGTGGGCGATAGGGTCAC |
| | | AATCACTTGTCGCGCCAGTCAGTCAATTTCTAGTTGGCTGGCTTGGTATCAGCAGAA |
| | | GCCCGGAAAAGCACCTAAGCTGCTGATCTATGACGCCTCCCGACTGGAGGATGGCG |
| | | TGCCAAGCAGATTCTCCGGGACAGGATTTGGCACTGACTTCACCTTTACAATCACCA |
| | | CACTCCAGCCAGACGATATTGCCACTTACTATTGCCAGCAGTACGACGATCTGCCCT |
| | | ATACCTTTGGGCAGGGAACTACCGTGGATATTAAGAACAGCGGCGCGGGCACCGCG |
| | | GCCGCGACTCACACATGCCCACCGTGCCCAGCACCTGAACTCCTGGGGGGACCGT |
| | | CAGTCTTCCTCTTCCCCCCAAAACCCAAGGACACCCTCATGATCTCCCGGACCCCTG |
| | | AGGTCACATGCGTGGTGGTGGACGTGAGCCACGAAGACCCTGAGGTCAAGTTCAAC |
| | | TGGTACGTGGACGGCGTGGAGGTGCATAATGCCAAGACAAAGCCGCGGGAGGAGC |
| | | AGTACAACAGCACGTACCGTGTGGTCAGCGTCCTCACCGTCCTGCACCAGGACTGG |
| | | CTGAATGGCAAGGAGTACAAGTGCAAGGTCTCCAACAAAGCCCTCCCAGCCCCCAT |
| | | CGAGAAAACCATCTCCAAAGCCAAAGGGCAGCCCCGAGAACCACAGGTGTGCACCC |
| | | TGCCCCCATCCCGGGATGAGCTGACCAAGAACCAGGTCAGCCTGTCCTGCGCCGTC |
| | | AAAGGCTTCTATCCCAGCGACATCGCCGTGGAGTGGGAGAGCAATGGGCAGCCGG |
| | | AGAACAACTACAAGACCACGCCTCCCGTGCTGGACTCCGACGGCTCCTTCTTCCTC |
| | | GTGAGCAAGCTCACCGTGGACAAGAGCAGGTGGCAGCAGGGGAACGTCTTCTCAT |
| | | GCTCCGTGATGCATGAGGCTCTGCACAACCACTACACGCAGAAGAGCCTCTCCCTG |
| | | TCTCCGGGTAAA |
| 158 | Anti-CD132 P1D7 Fab LC | GACATCCAGATGACCCAGTCTCCTTCCTTCCTGTCTGCATCTGTAGGAGACAGAGTC |
| | (VL, joint CL) | ACCATCACTTGCCGGGCCAGTCAGAGTATTAGTAGCTGGTTGGCCTGGTATCAGCA |
| | | GAAACCAGGGAAAGCCCCTAAACTCCTGATCTACGATGCATCCCGTTTGGAGGACG |
| | | GGGTCCCATCAAGATTCAGTGGAACTGGATTTGGGACAGATTTTACTTTCACCATTA |
| | | CCACCCTGCAGCCTGACGATATTGCGACATATTATTGTCAGCAATACGATGATCTCC |
| | | CGTACACTTTTGGCCAGGGGACCACGGTGGACATCAAACGAACTGTGGCTGCACCA |
| | | TCTGTCTTCATCTTCCCGCCATCTGATGAGCAGTTGAAATCTGGAACTGCCTCTGTT |
| | | GTGTGCCTGCTGAATAACTTCTATCCCAGAGAGGCCAAAGTACAGTGGAAGGTGGA |
| | | TAACGCCCTCCAATCGGGTAACTCCCAGGAGAGTGTCACAGAGCAGGACAGCAAGG |
| | | ACAGCACCTACAGCCTCAGCAGCACCCTGACGCTGAGCAAAGCAGACTACGAGAAA |
| | | CACAAAGTCTACGCCTGCGAAGTCACCCATCAGGGCCTGAGCTCGCCCGTCACAAA |
| | | GAGCTTCAACAGGGGAGAGTGT |
| 159 | Anti-CD132 P1D7 Fab HC | CAGGTGCAGCTGCAGGAGTCCGGGGGAGGCGTGGTCCAGCCTGGGAGGTCCCTG |
| | (VH, joint CH1) | AGACTCTCCTGTGCAGCCTCTGGATTCACCTTCAGTAACTATGGCATGCACTGGGTC |
| | | CGCCAGGCTCCAGGCAAAGGGCTGGAGTGGGTGGCAGTTATATCATATGATGGAAC |
| | | TAATAAATACTATGCAGACTCCGTGAAGGGCCGATTCACCATCTCCAGAGACAATTC |
| | | CAAGAACACGGTGTATCTGCAAATGAACAGCCTGAGAGCTGAGGACACGGCTGTGT |
| | | ATTACTGTGCGAAAGATGGTTTTGATATTTGGGGCCAAGGGACAATGGTCACCGTCT |
| | | CAAGCGCCTCCACCAAGGGCCCATCGGTCTTCCCCCTGGCACCCTCCTCCAAGAGC |
| | | ACCTCTGGGGGCACAGCGGCCCTGGGCTGCCTGGTCAAGGACTACTTCCCCGAAC |
| | | CGGTGACGGTGTCGTGGAACTCAGGCGCCCTGACCAGCGGCGTCCACACCTTCCC |
| | | GGCTGTCCTACAGTCCTCAGGACTCTACTCCCTCAGCAGCGTAGTGACCGTGCCCT |
| | | CCAGCAGCTTGGGCACCCAGACCTACATCTGCAACGTGAATCACAAGCCCAGCAAC |
| | | ACCAAGGTGGACAAGAAAGTTGAGCCCAAATCTTGT |
| 160 | Anti-CD132 P1D7 scFv | GAAGTGCAGCTGGTGGAAACTGGACCTGGACTGGTGAAGCCAAGCGGGACTCTGA |
| | and Fc with hole | GCCTGACCTGTGCCGTGAGCGGGGGAAGTATCAGCTCCTCTAACTGGTGGTCCTGG |
| | modification | GTGCGACAGCCCCCTGGCAAGGGGCTGGAGTGGATCGGCGAAATCTACCACAGCG |
| | | GGTCCACAAACTATAATCCTAGCCTGAAGAGCCGGGTGACTATCTCTGTGGACAAGA |
| | | GTAAAAATCAGTTCAGCCTGAAACTGAGTTCAGTGACAGCCGCTGATACCGCCGTGT |
| | | ACTATTGCGCCAGGGAGGGACCTCTGAGCAGCAGCGGACCAGGCGCTTTTGACATC |
| | | TGGGGGCAGGGAACTATGGTGACCGTCAGTTCAGGCGGAGGAGGCTCCGGAGGAG |
| | | GAGGGTCTGGAGGCGGGGGAAGTGAGATTGTGCTGACCCAGTCCCCCGCCACACT |
| | | GTCTCTGAGTCCTGGCGAACGGGCCACCCTGTCTTGTAGAGCTTCACAGAGCGTGT |
| | | CCTACCATCTGGCATGGTATCAGCAGAAACCAGGCCAGGCCCCCAGACTGCTGATC |
| | | TACGACACCTCAAACAGGGCTAGCGGCATTCCCGCACGCTTCTCTGGCAGTGGGTC |
| | | AGGAACAGATTTTACCCTGACAATCAATAGCCTGGAGCCAGAAGACTTCGCCGTGTA |
| | | CTATTGCCAGCAGCGCTATGATTGGCCCCTGACTTTTGGCGGGGGAACCAAGGTCG |
| | | AGATCAAGAACAGCGGCGCGGGCACCGCGGCCGCGACTCACACATGCCCACCGTG |
| | | CCCAGCACCTGAACTCCTGGGGGGACCGTCAGTCTTCCTCTTCCCCCCAAAACCCA |
| | | AGGACACCCTCATGATCTCCCGGACCCCTGAGGTCACATGCGTGGTGGTGGACGTG |
| | | AGCCACGAAGACCCTGAGGTCAAGTTCAACTGGTACGTGGACGGCGTGGAGGTGC |
| | | ATAATGCCAAGACAAAGCCGCGGGAGGAGCAGTACAACAGCACGTACCGTGTGGTC |
| | | AGCGTCCTCACCGTCCTGCACCAGGACTGGCTGAATGGCAAGGAGTACAAGTGCAA |
| | | GGTCTCCAACAAAGCCCTCCCAGCCCCCATCGAGAAAACCATCTCCAAAGCCAAAG |
| | | GGCAGCCCCGAGAACCACAGGTGTGCACCCTGCCCCCATCCCGGGATGAGCTGAC |
| | | CAAGAACCAGGTCAGCCTGTCCTGCGCCGTCAAAGGCTTCTATCCCAGCGACATCG |
| | | CCGTGGAGTGGGAGAGCAATGGGCAGCCGGAGAACAACTACAAGACCACGCCTCC |
| | | CGTGCTGGACTCCGACGGCTCCTTCTTCCTCGTGAGCAAGCTCACCGTGGACAAGA |
| | | GCAGGTGGCAGCAGGGGAACGTCTTCTCATGCTCCGTGATGCATGAGGCTCTGCAC |
| | | AACCACTACACGCAGAAGAGCCTCTCCCTGTCTCCGGGTAAA |
| 161 | Anti-CD132 P1E8 Fab LC | GATGTTGTGATGACTCAGTCTCCAGTCTCCCTGCCCGTCACCCTTGGACAGCCGGC |
| | (VL, joint CL) | CTCCATCTCCTGCAAGTCTAGTCAAAGCCTCCTTTACTTTAATGGAAACACCTACTTG |
| | | AGCTGGTTTCAGCAGAGGCCAGGCCAATCTCCACGGCGCCTATTTTATCAGGTTTCT |
| | | AACCGGGACTCTGGGGTCCCAGACAGATTCAGCGGCAGTGGGTCAGACACTGATTT |
| | | CACTCTGACCATTAGCAGGGTGGAGGCTGAAGATGTTGGAGTTTATTTCTGCATGCA |
| | | AGGAACACAGTGGCCTCCGACGTTCGGCCAAGGGACCAAGGTGGAAATCAAACGAA |
| | | CTGTGGCTGCACCATCTGTCTTCATCTTCCCGCCATCTGATGAGCAGCTGAAATCTG |
| | | GAACTGCCTCTGTTGTGTGCCTGCTGAATAACTTCTATCCCAGAGAGGCCAAAGTAC |
| | | AGTGGAAGGTGGATAACGCCCTCCAATCGGGTAACTCCCAGGAGAGTGTCACAGAG |
| | | CAGGACAGCAAGGACAGCACCTACAGCCTCAGCAGCACCCTGACGCTGAGCAAAG |
| | | CAGACTACGAGAAACACAAAGTCTACGCCTGCGAAGTCACCCATCAGGGCCTGAGC |
| | | TCGCCCGTCACAAAGAGCTTCAACAGGGGAGAGTGT |
| 162 | Anti-CD132 P1E8 Fab HC | GAGGTCCAGCTGGTGCAGTCTGGGGGAGGCGTGGTCCAGCCTGGGAGGTCCCTGA |
| | (VH, joint CH1) | GACTCTCCTGTGCAGCCTCTGGATTCACCTTCAGTAGCTATGGCATGCACTGGGTCC |
| | | GCCAGGCTCCAGGCAAGGGGCTGGAGTGGGTGGCAGTTATATCATATGATGGAAGT |
| | | AATAAATACTATGCAGACTCCGTGAAGGGCCGATTCACCATCTCCAGAGACAATTCC |
| | | AAGAACACGCTGTATCTGCAAATGAACAGCCTGAGAGCCGAGGACACGGCCGTGTA |
| | | TTACTGTGCGAGAGATGTCTACGGTGACTACGGGGCCTTTGACTACTGGGGCCAGG |
| | | GAACCCTGGTCACCGTCTCAAGCGCCTCCACCAAGGGCCCATCGGTCTTCCCCCTG |
| | | GCACCCTCCTCCAAGAGCACCTCTGGGGGCACAGCGGCCCTGGGCTGCCTGGTCA |
| | | AGGACTACTTCCCCGAACCGGTGACGGTGTCGTGGAACTCAGGCGCCCTGACCAG |
| | | CGGCGTCCACACCTTCCCGGCTGTCCTACAGTCCTCAGGACTCTACTCCCTCAGCA |
| | | GCGTAGTGACCGTGCCCTCCAGCAGCTTGGGCACCCAGACCTACATCTGCAACGTG |
| | | AATCACAAGCCCAGCAACACCAAGGTGGACAAGAAAGTTGAGCCCAAATCTTGT |
| 163 | Anti-CD132 P1E8 scFv | GAGGTCCAGCTGGTCCAGAGCGGCGGAGGGGTCGTCCAGCCCGGAAGAAGCCTGA |
| | and Fc with hole | GACTGTCCTGTGCAGCAAGTGGGTTTACATTCAGCTCCTACGGCATGCACTGGGTG |
| | modification | AGGCAGGCACCCGGCAAGGGGCTGGAGTGGGTGGCCGTCATCAGTTATGACGGCT |
| | | CAAACAAGTACTATGCCGATAGCGTGAAAGGGAGGTTCACAATTAGCCGCGACAACT |
| | | CCAAAAATACTCTGTACCTCCAGATGAACAGCCTGAGAGCCGAAGATACAGCTGTGT |
| | | ACTATTGCGCTAGGGACGTCTACGGAGATTATGGCGCATTTGACTATTGGGGACAG |
| | | GGCACTCTGGTGACCGTCTCTAGTGGAGGAGGAGGCTCAGGAGGAGGAGGGAGCG |
| | | GCGGAGGAGGCAGCGATGTGGTCATGACCCAGTCCCCAGTGTCTCTGCCAGTCACA |
| | | CTGGGACAGCCAGCATCCATCTCTTGTAAGTCAAGCCAGTCTCTGCTGTACTTCAAC |
| | | GGAAATACTTATCTGTCTTGGTTTCAGCAGCGCCCTGGCCAGAGTCCACGGAGACT |
| | | GTTCTACCAGGTGTCTAACCGAGACAGTGGCGTCCCTGATCGGTTCAGTGGGTCAG |
| | | GAAGCGACACCGATTTTACCCTGACAATCAGCCGAGTGGAGGCTGAAGACGTGGGG |
| | | GTCTATTTCTGCATGCAGGGAACACAGTGGCCCCCTACTTTTGGCCAGGGGACCAA |
| | | GGTGGAGATCAAGAACAGCGGCGCGGGCACCGCGGCCGCGACTCACACATGCCCA |
| | | CCGTGCCCAGCACCTGAACTCCTGGGGGGACCGTCAGTCTTCCTCTTCCCCCCAAA |
| | | ACCCAAGGACACCCTCATGATCTCCCGGACCCCTGAGGTCACATGCGTGGTGGTGG |
| | | ACGTGAGCCACGAAGACCCTGAGGTCAAGTTCAACTGGTACGTGGACGGCGTGGA |
| | | GGTGCATAATGCCAAGACAAAGCCGCGGGAGGAGCAGTACAACAGCACGTACCGT |
| | | GTGGTCAGCGTCCTCACCGTCCTGCACCAGGACTGGCTGAATGGCAAGGAGTACAA |
| | | GTGCAAGGTCTCCAACAAAGCCCTCCCAGCCCCCATCGAGAAAACCATCTCCAAAG |
| | | CCAAAGGGCAGCCCCGAGAACCACAGGTGTGCACCCTGCCCCCATCCCGGGATGA |
| | | GCTGACCAAGAACCAGGTCAGCCTGTCCTGCGCCGTCAAAGGCTTCTATCCCAGCG |
| | | ACATCGCCGTGGAGTGGGAGAGCAATGGGCAGCCGGAGAACAACTACAAGACCAC |
| | | GCCTCCCGTGCTGGACTCCGACGGCTCCTTCTTCCTCGTGAGCAAGCTCACCGTGG |
| | | ACAAGAGCAGGTGGCAGCAGGGGAACGTCTTCTCATGCTCCGTGATGCATGAGGCT |
| | | CTGCACAACCACTACACGCAGAAGAGCCTCTCCCTGTCTCCGGGTAAA |
| 164 | Anti-CD132 P2B2 Fab LC | GATGTTGTGATGACTCAGTCTCCACTCTCCCTGCCCGTCACCCCTGGAGAGCCGGC |
| | (VL, joint CL) | CTCCATCTCCTGCAGGTCTAGTCAGAGCCTCCTGCATAGTAATGGATACAACTATTT |
| | | GGATTGGTACCTGCAGAAGCCAGGGCAGTCTCCACACCTCCTGATCTACTTGGGTT |
| | | CTAATCGGGCCTCCGGGGTCCCTGACAGGTTCAGTGGCAGTGGATCAGGCACAGAT |
| | | TTTACACTGAAAATTAGCAGAGTGGAGGCTGAGGATGTTGGGGTTTATTTCTGCATG |
| | | CAAGCTCTACGAACTCCGTACACTTTTGGCCAGGGGACCAAGCTGGAGATCAAACG |
| | | AACTGTGGCTGCACCATCTGTCTTCATCTTCCCGCCATCTGATGAGCAGTTGAAATC |
| | | TGGAACTGCCTCTGTTGTGTGCCTGCTGAATAACTTCTATCCCAGAGAGGCCAAAGT |
| | | ACAGTGGAAGGTGGATAACGCCCTCCAATCGGGTAACTCCCAGGAGAGTGTCACAG |
| | | AGCAGGACAGCAAGGACAGCACCTACAGCCTCAGCAGCACCCTGACGCTGAGCAAA |
| | | GCAGACTACGAGAAACACAAAGTCTACGCCTGCGAAGTCACCCATCAGGGCCTGAG |
| | | CTCGCCCGTCACAAAGAGCTTCAACAGGGGAGAGTGT |
| 165 | Anti-CD132 P2B2 Fab HC | CAGCTGCAGCTGCAGGAGTCGGGGGGAGGCGTGGTCCAGCCTGGGAGGTCCCTG |
| | (VH, joint CH1) | AGACTCTCCTGTGCAGCCTCTGGATTCACCTTCAGTAGCTATGCTATGCACTGGGTC |
| | | CGCCAGGCTCCAGGCAAGGGGCTGGAGTGGGTGGCAGTTATATCATATGATGGAG |
| | | GTAATAAATACTACGCAGACTCCGTGAAGGGCCGATTCACCATCTCCAGAGACAATT |
| | | CCAAGAACACGCTGTATCTGCAAATGAACAGCCTGAGAGCTGAGGACACGGCTGTG |
| | | TATTACTGTGCGAAATCAGTGGCGCCTCCCATGGACGTCTGGGGCAAAGGGACCAC |
| | | GGTCACCGTCTCAAGCGCCTCCACCAAGGGCCCATCGGTCTTCCCCCTGGCACCCT |
| | | CCTCCAAGAGCACCTCTGGGGGCACAGCGGCCCTGGGCTGCCTGGTCAAGGACTA |
| | | CTTCCCCGAACCGGTGACGGTGTCGTGGAACTCAGGCGCCCTGACCAGCGGCGTC |
| | | CACACCTTCCCGGCTGTCCTACAGTCCTCAGGACTCTACTCCCTCAGCAGCGTAGT |
| | | GACCGTGCCCTCCAGCAGCTTGGGCACCCAGACCTACATCTGCAACGTGAATCACA |
| | | AGCCCAGCAACACCAAGGTGGACAAGAAAGTTGAGCCCAAATCTTGT |
| 166 | Anti-CD132 P2B2 scFv | CAGCTGCAGCTGCAGGAATCCGGGGGAGGCGTCGTCCAGCCAGGAAGGTCACTGA |
| | and Fc with hole | GACTGAGTTGTGCCGCAAGCGGGTTCACTTTCAGCTCCTACGCTATGCACTGGGTG |
| | modification | AGACAGGCACCCGGAAAGGGCCTGGAGTGGGTGGCAGTCATCTCTTATGACGGCG |
| | | GGAACAAGTACTATGCCGATAGTGTGAAAGGCCGGTTCACCATTAGTAGAGACAACT |
| | | CAAAAAATACACTGTACCTCCAGATGAATAGCCTGCGCGCCGAAGACACAGCTGTGT |
| | | ACTATTGCGCAAAGTCCGTGGCCCCCCCTATGGATGTCTGGGGGAAAGGAACCACA |
| | | GTGACTGTCTCTAGTGGAGGAGGAGGATCAGGCGGCGGAGGCAGCGGAGGAGGA |
| | | GGGTCCGACGTGGTCATGACTCAGTCCCCTCTGTCTCTGCCAGTGACCCCCGGCGA |
| | | GCCTGCTTCCATCTCTTGTAGGTCAAGCCAGAGCCTGCTGCACTCCAACGGGTACA |
| | | ATTATCTGGATTGGTACTTGCAGAAGCCAGGCCAGTCTCCCCATCTGCTGATCTATC |
| | | TGGGATCTAACAGGGCCAGTGGCGTGCCTGACCGCTTCAGTGGCTCAGGGAGCGG |
| | | AACTGATTTTACCCTGAAAATTAGCCGAGTCGAGGCCGAAGATGTGGGCGTCTACTT |
| | | CTGCATGCAGGCTCTGCGGACACCATATACTTTTGGCCAGGGGACCAAGCTGGAGA |
| | | TCAAGAACAGCGGCGCGGGCACCGCGGCCGCGACTCACACATGCCCACCGTGCCC |
| | | AGCACCTGAACTCCTGGGGGGACCGTCAGTCTTCCTCTTCCCCCCAAAACCCAAGG |
| | | ACACCCTCATGATCTCCCGGACCCCTGAGGTCACATGCGTGGTGGTGGACGTGAGC |
| | | CACGAAGACCCTGAGGTCAAGTTCAACTGGTACGTGGACGGCGTGGAGGTGCATAA |
| | | TGCCAAGACAAAGCCGCGGGAGGAGCAGTACAACAGCACGTACCGTGTGGTCAGC |
| | | GTCCTCACCGTCCTGCACCAGGACTGGCTGAATGGCAAGGAGTACAAGTGCAAGGT |
| | | CTCCAACAAAGCCCTCCCAGCCCCCATCGAGAAAACCATCTCCAAAGCCAAAGGGC |
| | | AGCCCCGAGAACCACAGGTGTGCACCCTGCCCCCATCCCGGGATGAGCTGACCAA |
| | | GAACCAGGTCAGCCTGTCCTGCGCCGTCAAAGGCTTCTATCCCAGCGACATCGCCG |
| | | TGGAGTGGGAGAGCAATGGGCAGCCGGAGAACAACTACAAGACCACGCCTCCCGT |
| | | GCTGGACTCCGACGGCTCCTTCTTCCTCGTGAGCAAGCTCACCGTGGACAAGAGCA |
| | | GGTGGCAGCAGGGGAACGTCTTCTCATGCTCCGTGATGCATGAGGCTCTGCACAAC |
| | | CACTACACGCAGAAGAGCCTCTCCCTGTCTCCGGGTAAA |
| 167 | Anti-CD132 P2B7 Fab LC | GATGTTGTGATGACTCAGTCTCCACTCTCCCTGCCCGTCACCCTTGGACAGCCGGC |
| | (VL, joint CL) | CTCCATCTCCTGCAGGTCTAGTCAGAGCCTCGTCCATAGTAATGGATACAACTATTT |
| | | GGACTGGTACCTGCAGAAGCCAGGGCAGTCTCCACAGCTCCTGATCTATTTGGGTT |
| | | CTAATCGGGCCTCCGGGGTCCCTGACAGGTTCAGTGGCAGTGGATCGGGCACAGA |
| | | TTTTACACTGAAAATCAGCAGAGTGGAGGCTGAGGATGTTGGGGTTTATTACTGCCT |
| | | GCAAGGTTCACACTGGCCTTGGACGTTCGGCCAAGGGACCAAGGTGGAAATCAAAC |
| | | GAACTGTGGCTGCACCATCTGTCTTCATCTTCCCGCCATCTGATGAGCAGTTGAAAT |
| | | CTGGAACTGCCTCTGTTGTGTGCCTGCTGAATAACTTCTATCCCAGAGAGGCCAAAG |
| | | TACAGTGGAAGGTGGATAACGCCCTCCAATCGGGTAACTCCCAGGAGAGTGTCACA |
| | | GAGCAGGACAGCAAGGACAGCACCTACAGCCTCAGCAGCACCCTGACGCTGAGCA |
| | | AAGCAGACTACGAGAAACACAAAGTCTACGCCTGCGAAGTCACCCATCAGGGCCTG |
| | | AGCTCGCCCGTCACAAAGAGCTTCAACAGGGGAGAGTGT |
| 168 | Anti-CD132 P2B7 Fab HC | CAGGTGCAGCTACAGCAGTGGGGCGCAGGACTGTTGAAGCCTTCGGAGACCCTGT |
| | (VH, joint CH1) | CCCTCACCTGCGCTGTCTATGGTGAGTCCTTCAGTGGTTACTACTGGAGCTGGATCC |
| | | GCCAGCCCCCAGGGAAGGGGCTGGAGTGGATTGGGGAAATCAATCATAGTGGAAG |
| | | CACCAACTACAACCCGTCCCTCAAGAGTCGAGTCACCATATCAGTAGACACGTCCAA |
| | | GAACCAGTTCTCCCTGAAGCTGAGCTCTGTGACCGCCGCGGACACGGCTGTGTATT |
| | | ACTGTGCGAGAGGCCCCGCGGGTAGCAGCTCGTCCGGCTACTTTGACTACTGGGG |
| | | CCAGGGAACCCTGGTCACCGTCTCAAGCGCCTCCACCAAGGGCCCATCGGTCTTCC |
| | | CCCTGGCACCCTCCTCCAAGAGCACCTCTGGGGGCACAGCGGCCCTGGGCTGCCT |
| | | GGTCAAGGACTACTTCCCCGAACCGGTGACGGTGTCGTGGAACTCAGGCGCCCTGA |
| | | CCAGCGGCGTCCACACCTTCCCGGCTGTCCTACAGTCCTCAGGACTCTACTCCCTC |
| | | AGCAGCGTAGTGACCGTGCCCTCCAGCAGCTTGGGCACCCAGACCTACATCTGCAA |
| | | CGTGAATCACAAGCCCAGCAACACCAAGGTGGACAAGAAAGTTGAGCCCAAATCTT |
| | | GT |
| 169 | Anti-CD132 P2B7 scFv | CAGGTCCAGCTGCAGCAGTGGGGCGCCGGACTGCTGAAACCCTCTGAAACTCTGA |
| | and Fc with hole | GCCTGACTTGTGCCGTCTATGGGGAATCCTTCTCTGGCTACTATTGGAGTTGGATCA |
| | modification | GGCAGCCCCCTGGCAAGGGGCTGGAGTGGATCGGAGAAATTAACCACAGCGGCTC |
| | | CACCAACTACAATCCATCTCTGAAAAGTCGCGTGACCATTTCCGTGGACACATCTAA |
| | | GAATCAGTTCAGCCTGAAGCTGAGCAGCGTGACAGCCGCTGATACTGCCGTCTACT |
| | | ATTGCGCACGGGGCCCCGCCGGGTCTAGTTCAAGCGGATACTTTGACTATTGGGGA |
| | | CAGGGCACCCTGGTGACAGTCTCCTCTGGCGGAGGAGGCTCCGGAGGAGGAGGGT |
| | | CTGGAGGAGGAGGAAGCGATGTGGTCATGACACAGTCACCACTGAGCCTGCCAGT |
| | | GACTCTGGGACAGCCTGCTTCTATCAGTTGTCGAAGTTCACAGAGTCTGGTCCACTC |
| | | AAACGGATACAATTATCTGGACTGGTACTTGCAGAAGCCTGGCCAGAGCCCACAGC |
| | | TGCTGATCTATCTGGGGAGCAACCGAGCTTCCGGAGTGCCCGACAGATTCTCAGGG |
| | | AGCGGCAGCGGCACTGATTTTACCCTGAAAATTAGCAGAGTGGAGGCAGAAGATGT |
| | | GGGCGTCTACTATTGCCTCCAGGGGTCCCATTGGCCTTGGACTTTCGGGCAGGGAA |
| | | CCAAGGTGGAGATCAAGAACAGCGGCGCGGGCACCGCGGCCGCGACTCACACATG |
| | | CCCACCGTGCCCAGCACCTGAACTCCTGGGGGGACCGTCAGTCTTCCTCTTCCCCC |
| | | CAAAACCCAAGGACACCCTCATGATCTCCCGGACCCCTGAGGTCACATGCGTGGTG |
| | | GTGGACGTGAGCCACGAAGACCCTGAGGTCAAGTTCAACTGGTACGTGGACGGCGT |
| | | GGAGGTGCATAATGCCAAGACAAAGCCGCGGGAGGAGCAGTACAACAGCACGTAC |
| | | CGTGTGGTCAGCGTCCTCACCGTCCTGCACCAGGACTGGCTGAATGGCAAGGAGTA |
| | | CAAGTGCAAGGTCTCCAACAAAGCCCTCCCAGCCCCCATCGAGAAAACCATCTCCA |
| | | AAGCCAAAGGGCAGCCCCGAGAACCACAGGTGTGCACCCTGCCCCCATCCCGGGA |
| | | TGAGCTGACCAAGAACCAGGTCAGCCTGTCCTGCGCCGTCAAAGGCTTCTATCCCA |
| | | GCGACATCGCCGTGGAGTGGGAGAGCAATGGGCAGCCGGAGAACAACTACAAGAC |
| | | CACGCCTCCCGTGCTGGACTCCGACGGCTCCTTCTTCCTCGTGAGCAAGCTCACCG |
| | | TGGACAAGAGCAGGTGGCAGCAGGGGAACGTCTTCTCATGCTCCGTGATGCATGAG |
| | | GCTCTGCACAACCACTACACGCAGAAGAGCCTCTCCCTGTCTCCGGGTAAA |
| 170 | Anti-CD132 P2D11 Fab | GAAACGACACTCACGCAGTCTCCAGCCACCCTGTCTGTGTCTCCAGGGGAAAGAGC |
| | LC (VL, joint CL) | CACCCTCTCCTGCAGGGCCAGTCAGAGTGTTAGCAGCAACTTAGCCTGGTACCAGC |
| | | AGAAACCTGGCCAGGCTCCCAGGCTCCTCATCTATGGTGCATCCAGCGGGGCCACT |
| | | GGCATCCCAGACAGGTTCAGTGGCAGTGGGTCTGGGACAGACTTCACTCTCACCAT |
| | | CAGCAGACTGGAGCCTGAAGATTTTGCAGTGTATTACTGTCAGCTGTATGGTAGCTC |
| | | ACTCGCTTTCGGCGGAGGGACCAAGGTGGAGATCAAACGAACTGTGGCTGCACCAT |
| | | CTGTCTTCATCTTCCCGCCATCTGATGAGCAGTTGAAATCTGGAACTGCCTCTGTTG |
| | | TGTGCCTGCTGAATAACTTCTATCCCAGAGAGGCCAAAGTACAGTGGAAGGTGGATA |
| | | ACGCCCTCCAATCGGGTAACTCCCAGGAGAGTGTCACAGAGCAGGACAGCAAGGAC |
| | | AGCACCTACAGCCTCAGCAGCACCCTGACGCTGAGCAAAGCAGACTACGAGAAACA |
| | | CAAAGTCTACGCCTGCGAAGTCACCCATCAGGGCCTGAGCTCGCCCGTCACAAAGA |
| | | GCTTCAACAGGGGAGAGTGT |
| 171 | Anti-CD132 P2D11 Fab | GAGGTCCAGCTGGTGCAGTCTGGGGCTGAGGTGAAGAAGCCTGGGTCCTCGGTGA |
| | HC (VH, joint CH1) | AGGTCTCCTGCAAGGCTTCTGGAGGCACCTTCAGCAGCTATGCTATCAGCTGGGTG |
| | | CGACAGGCCCCTGGACAAGGGCTTGAGTGGATGGGATGGATCAGCGCTTACAATG |
| | | GTGACACAAGCTACGCACAGAAGTTCCAGGGCAGAGTCACCATTACCAGGGACACA |
| | | TCCGCGAGCACAGCCTACATGGAGCTGAGCAGCCTGAGATCTGAAGACACGGCTGT |
| | | GTATTACTGTGCGAGAGATTGGGGATATTGTAGTGGTGGTAGCTGCTACCTGAACTG |
| | | GTTCGACCCCTGGGGCCAGGGAACCCTGGTCACCGTCTCAAGCGCCTCCACCAAG |
| | | GGCCCATCGGTCTTCCCCCTGGCACCCTCCTCCAAGAGCACCTCTGGGGGCACAG |
| | | CGGCCCTGGGCTGCCTGGTCAAGGACTACTTCCCCGAACCGGTGACGGTGTCGTG |
| | | GAACTCAGGCGCCCTGACCAGCGGCGTCCACACCTTCCCGGCTGTCCTACAGTCCT |
| | | CAGGACTCTACTCCCTCAGCAGCGTAGTGACCGTGCCCTCCAGCAGCTTGGGCACC |
| | | CAGACCTACATCTGCAACGTGAATCACAAGCCCAGCAACACCAAGGTGGACAAGAA |
| | | AGTTGAGCCCAAATCTTGT |
| 172 | Anti-CD132 P2D11 scFv | AGGTCCAGCTGCAGGAAAGCGGGCCAGGACTGGTCAAACCCTCACAGACACTGTCT |
| | and Fc with hole | CTGACTTGTACCGTCTCCGGGGGCTCAATCAGCTCCGGCGGGTACTATTGGACATG |
| | modification | GATCAGACAGCACCCTGGACAGGGCCTGGAGTGGATCGGGTTCATTAGCTGGTCCG |
| | | GAACCACATACTATAACCCAAGCCTGAAGAATAGGGTGACAATTTCAGCCGACACTA |
| | | GCAAAAACCATTTTTCCCTGAATCTGACCTCTGTGACAGCCGCTGATACTGCTGTCT |
| | | ACTATTGCGCACGGGGGTCCGGAAGACTGGTGTGGGGACAGGGGACTCTGGTGAC |
| | | CGTCTCTAGTGGAGGAGGAGGAAGTGGCGGAGGAGGCAGCGGAGGAGGAGGGTC |
| | | CGAGACTACCCTGACCCAGTCTCCAGCTACACTGTCTGTGAGTCCCGGCGAAAGGG |
| | | CAACCCTGAGCTGTCGCGCTTCACAGAGCGTCTCAAGCAACCTGGCATGGTATCAG |
| | | CAGAAGCCTGGCCAGGCCCCTCGACTGCTGATCTATGGGGCATCCTCTGGAGCCAC |
| | | TGGCATTCCCGACCGGTTCTCCGGATCTGGCAGTGGGACCGATTTTACACTGACCA |
| | | TCAGCCGGCTGGAGCCTGAAGACTTCGCTGTGTACTATTGCCAGCTGTACGGCAGT |
| | | TCACTGGCATTTGGAGGCGGGACAAAGGTCGAGATCAAGAACAGCGGCGCGGGCA |
| | | CCGCGGCCGCGACTCACACATGCCCACCGTGCCCAGCACCTGAACTCCTGGGGGG |
| | | ACCGTCAGTCTTCCTCTTCCCCCCAAAACCCAAGGACACCCTCATGATCTCCCGGAC |
| | | CCCTGAGGTCACATGCGTGGTGGTGGACGTGAGCCACGAAGACCCTGAGGTCAAG |
| | | TTCAACTGGTACGTGGACGGCGTGGAGGTGCATAATGCCAAGACAAAGCCGCGGGA |
| | | GGAGCAGTACAACAGCACGTACCGTGTGGTCAGCGTCCTCACCGTCCTGCACCAGG |
| | | ACTGGCTGAATGGCAAGGAGTACAAGTGCAAGGTCTCCAACAAAGCCCTCCCAGCC |
| | | CCCATCGAGAAAACCATCTCCAAAGCCAAAGGGCAGCCCCGAGAACCACAGGTGTG |
| | | CACCCTGCCCCCATCCCGGGATGAGCTGACCAAGAACCAGGTCAGCCTGTCCTGCG |
| | | CCGTCAAAGGCTTCTATCCCAGCGACATCGCCGTGGAGTGGGAGAGCAATGGGCA |
| | | GCCGGAGAACAACTACAAGACCACGCCTCCCGTGCTGGACTCCGACGGCTCCTTCT |
| | | TCCTCGTGAGCAAGCTCACCGTGGACAAGAGCAGGTGGCAGCAGGGGAACGTCTT |
| | | CTCATGCTCCGTGATGCATGAGGCTCTGCACAACCACTACACGCAGAAGAGCCTCT |
| | | CCCTGTCTCCGGGTAAA |
| 173 | Anti-CD132 P2F10 Fab LC | GATATTGTGATGACCCACACTCCACTCTCCCTGCCCGTCACCCCTGGAGAGCCGGC |
| | (VL, joint CL) | CTCCATCTCCTGCAGGTCTAGTCAGACCCTCTTCGATAGCGATGATGGAAAGACCTA |
| | | TTTGGACTGGTACCTGCAGAAGCCAGGGCAGTCTCCACAACTCCTGATGTATACCAC |
| | | TTCCTCTCGGGCCTCTGGAGTCCCAGACAGGTTCAGTGGCAGTGGGTCAGGCACTG |
| | | ATTTCACACTGAAAATCAGCAGGGTGGAGGCTGAGGATGTTGGAGTTTATTACTGCA |
| | | TGCAGCGTTTACAGTTTCCCCTCACCTTCGGCCAAGGGACACGACTGGAGTTCAAAC |
| | | GAACTGTGGCTGCACCATCTGTCTTCATCTTCCCGCCATCTGATGAGCAGTTGAAAT |
| | | CTGGAACTGCCTCTGTTGTGTGCCTGCTGAATAACTTCTATCCCAGAGAGGCCAAAG |
| | | TACAGTGGAAGGTGGATAACGCCCTCCAATCGGGTAACTCCCAGGAGAGTGTCACA |
| | | GAGCAGGACAGCAAGGACAGCACCTACAGCCTCAGCAGCACCCTGACGCTGAGCA |
| | | AAGCAGACTACGAGAAACACAAAGTCTACGCCTGCGAAGTCACCCATCAGGGCCTG |
| | | AGCTCGCCCGTCACAAAGAGCTTCAACAGGGGAGAGTGT |
| 174 | Anti-CD132 P2F10 Fab | GAGGTCCAGCTGGTGCAGTCTGGGGCTGAGGTGAAGAAGCCTGGGGCCTCAGTGA |
| | HC (VH, joint CH1) | AGGTCTCCTGCAAGGCTTCTGGATACACCTTCACCGGCTACTATATGCACTGGGTGC |
| | | GACAGGCCCCTGGACAAGGGCTTGAGTGGATGGGAATAATCAACCCTAGTGGTGGT |
| | | AGCACAAGCTACGCACAGAAGTTCCAGGGCAGAGTCACCATGACCAGGGACACGTC |
| | | CACGAGCACAGTCTACATGGAGCTGAGCAGCCTGAGATCTGAGGACACGGCCGTGT |
| | | ATTACTGTGCGAGAGCCGATACAGCTATGGGTGATGCTTTTGATATCTGGGGCCAAG |
| | | GGACAATGGTCACCGTCTCAAGCGCCTCCACCAAGGGCCCATCGGTCTTCCCCCTG |
| | | GCACCCTCCTCCAAGAGCACCTCTGGGGGCACAGCGGCCCTGGGCTGCCTGGTCA |
| | | AGGACTACTTCCCCGAACCGGTGACGGTGTCGTGGAACTCAGGCGCCCTGACCAG |
| | | CGGCGTCCACACCTTCCCGGCTGTCCTACAGTCCTCAGGACTCTACTCCCTCAGCA |
| | | GCGTAGTGACCGTGCCCTCCAGCAGCTTGGGCACCCAGACCTACATCTGCAACGTG |
| | | AATCACAAGCCCAGCAACACCAAGGTGGACAAGAAAGTTGAGCCCAAATCTTGT |
| | | GAAGTCCAGCTGGTCCAGTCAGGAGCCGAGGTCAAGAAGCCAGGGGCAAGCGTCA |
| | | AAGTCTCATGCAAAGCAAGTGGGTACACATTTACAGGCTACTATATGCACTGGGTGA |
| | | GGCAGGCTCCAGGACAGGGCCTGGAGTGGATGGGGATCATTAACCCCAGCGGCGG |
| | | GAGTACCTCATACGCACAGAAGTTCCAGGGACGGGTGACTATGACCAGAGACACAA |
| | | GCACTTCCACCGTCTATATGGAGCTGAGCAGCCTGCGATCCGAAGACACTGCCGTG |
| | | TACTATTGCGCCAGAGCCGATACCGCAATGGGCGACGCCTTTGACATCTGGGGGCA |
| | | GGGCACAATGGTGACAGTCTCTAGTGGAGGAGGAGGATCTGGAGGAGGAGGCAGT |
| 175 | Anti-CD132 P2F10 scFv | GGAGGAGGCGGGTCAGACATCGTGATGACACATACTCCACTGTCTCTGCCAGTCAC |
| | and Fc with hole | CCCTGGCGAGCCAGCCTCTATTAGTTGTCGCTCAAGCCAGACCCTGTTCGACAGTG |
| | modification | ACGATGGAAAGACATACCTGGATTGGTACTTGCAGAAACCTGGCCAGAGCCCTCAG |
| | | CTGCTGATGTACACCACATCCTCTAGGGCCTCCGGCGTGCCTGACCGCTTCTCAGG |
| | | CAGCGGGTCCGGAACTGATTTTACCCTGAAGATCAGCCGGGTGGAGGCTGAAGACG |
| | | TGGGGGTCTACTATTGCATGCAGAGACTCCAGTTCCCACTGACATTTGGCCAGGGG |
| | | ACTCGGCTGGAGTTCAAGAACAGCGGCGCGGGCACCGCGGCCGCGACTCACACAT |
| | | GCCCACCGTGCCCAGCACCTGAACTCCTGGGGGGACCGTCAGTCTTCCTCTTCCCC |
| | | CCAAAACCCAAGGACACCCTCATGATCTCCCGGACCCCTGAGGTCACATGCGTGGT |
| | | GGTGGACGTGAGCCACGAAGACCCTGAGGTCAAGTTCAACTGGTACGTGGACGGC |
| | | GTGGAGGTGCATAATGCCAAGACAAAGCCGCGGGAGGAGCAGTACAACAGCACGT |
| | | ACCGTGTGGTCAGCGTCCTCACCGTCCTGCACCAGGACTGGCTGAATGGCAAGGAG |
| | | TACAAGTGCAAGGTCTCCAACAAAGCCCTCCCAGCCCCCATCGAGAAAACCATCTCC |
| | | AAAGCCAAAGGGCAGCCCCGAGAACCACAGGTGTGCACCCTGCCCCCATCCCGGG |
| | | ATGAGCTGACCAAGAACCAGGTCAGCCTGTCCTGCGCCGTCAAAGGCTTCTATCCC |
| | | AGCGACATCGCCGTGGAGTGGGAGAGCAATGGGCAGCCGGAGAACAACTACAAGA |
| | | CCACGCCTCCCGTGCTGGACTCCGACGGCTCCTTCTTCCTCGTGAGCAAGCTCACC |
| | | GTGGACAAGAGCAGGTGGCAGCAGGGGAACGTCTTCTCATGCTCCGTGATGCATGA |
| | | GGCTCTGCACAACCACTACACGCAGAAGAGCCTCTCCCTGTCTCCGGGTAAA |
| 176 | Anti-CD132 P2H4 Fab LC | GATGTTGTGATGACTCAGTCTCCACTCTCCCTGCCCGTCACCCCTGGAGAGCCGGC |
| | (VL, joint CL) | CTCCATCTCCTGCAGGGCAACTCAGAGCCTCCTGCATGGAAATGGACACAACTATTT |
| | | GGATTGGTACCTGCAGAAGCCAGGGCAGTCTCCACAGCTCCTGATCTATTTGGGTT |
| | | CTAATCGGGCCTCCGGGGTCCCTGACAGGTTCAGTGGCAGTGGATCAGGCACAGAT |
| | | TTTACACTGAAAATCAGCAGAGTGGAGGCTGAGGATGTTGGGGTTTATTACTGCATG |
| | | CAAACTCTGGAAACTCCTGTCACTTTCGGCCCTGGGACCAAAGTGGATATCAAACGA |
| | | ACTGTGGCTGCACCATCTGTCTTCATCTTCCCGCCATCTGATGAGCAGTTGAAATCT |
| | | GGAACTGCCTCTGTTGTGTGCCTGCTGAATAACTTCTATCCCAGAGAGGCCAAAGTA |
| | | CAGTGGAAGGTGGATAACGCCCTCCAATCGGGTAACTCCCAGGAGAGTGTCACAGA |
| | | GCAGGACAGCAAGGACAGCACCTACAGCCTCAGCAGCACCCTGACGCTGAGCAAA |
| | | GCAGACTACGAGAAACACAAACTCTACGCCTGCGAAGTCACCCATCAGGGCCTGAG |
| | | CTCGCCCGTCACAAAGAGCTTCAACAGGGGAGAGTGT |
| 177 | Anti-CD132 P2H4 Fab HC | GAGGTCCAGCTGGTGCAGTCTGGGGGAGGCGTGGTCCAGCCTGGGAGGTCCCTGA |
| | (VH, joint CH1) | GACTCTCCTGTGCAGCCTCTGGATTCACCTTCAGTAGCTATGCTATGCACTGGGTCC |
| | | GCCAGGCTCCAGGCAAGGGGCTGGAGTGGGTGGCAGTTATATCATATGATGGAAGC |
| | | AATAAATACTACGCAGACTCCGTGAAGGGCCGATTCACCATCTCCAGAGACAATTCC |
| | | AAGAACACGCTGTATCTGCAAATGAACAGCCTGAGAGCTGAGGACACGGCTGTGTA |
| | | TTACTGTGCGAGGTCTATCGGTATCGGTGCTTTTGATATCTGGGGCCAAGGGACAAT |
| | | GGTCACCGTCTCAAGCGCCTCCACCAAGGGCCCATCGGTCTTCCCCCTGGCACCCT |
| | | CCTCCAAGAGCACCTCTGGGGGCACAGCGGCCCTGGGCTGCCTGGTCAAGGACTA |
| | | CTTCCCCGAACCGGTGACGGTGTCGTGGAACTCAGGCGCCCTGACCAGCGGCGTC |
| | | CACACCTTCCCGGCTGTCCTACAGTCCTCAGGACTCTACTCCCTCAGCAGCGTAGT |
| | | GACCGTGCCCTCCAGCAGCTTGGGCACCCAGACCTACATCTGCAACGTGAATCACA |
| | | AGCCCAGCAACACCAAGGTGGACAAGAAAGTTGAGCCCAAATCTTGT |
| 178 | Anti-CD132 P2H4 scFv | GAGGTCCAGCTGGTCCAGAGCGGGGGGGGGgTCGTGCAGCCTGGGAGAAGCCTGA |
| | and Fc with hole | GACTGTCCTGTGCCGCAAGCGGGTTTACTTTTAGCTCCTACGCTATGCACTGGGTGA |
| | modification | GGCAGGCACCCGGCAAGGGGCTGGAGTGGGTGGCAGTCATCTCCTATGACGGCTC |
| | | TAACAAGTACTATGCCGATAGCGTGAAAGGGCGGTTCACAATTAGTAGAGACAACTC |
| | | AAAGAACACTCTGTACCTCCAGATGAATAGCCTGCGAGCCGAAGACACTGCTGTGTA |
| | | CTATTGCGCCCGGTCCATCGGAATTGGCGCTTTTGACATCTGGGGGCAGGGCACAA |
| | | TGGTGACAGTCTCTAGTGGAGGAGGAGGCTCTGGAGGAGGAGGGAGTGGAGGAGG |
| | | AGGATCAGACGTGGTCATGACCCAGTCACCTCTGAGCCTGCCAGTGACACCTGGCG |
| | | AGCCAGCATCAATTAGCTGTAGAGCCACCCAGTCTCTGCTGCACGGCAACGGGCAT |
| | | AATTACCTGGATTGGTACTTGCAGAAGCCTGGCCAGAGTCCTCAGCTGCTGATCTAT |
| | | CTGGGGAGCAACAGGGCTTCCGGAGTGCCAGACCGCTTCTCCGGATCTGGCAGTG |
| | | GGACTGATTTTACCCTGAAAATTTCCCGCGTCGAGGCAGAAGACGTGGGAGTCTACT |
| | | ATTGCATGCAGACACTGGAAACTCCAGTGACCTTCGGACCCGGCACAAAGGTGGAC |
| | | ATCAAGAACAGCGGCGCGGGCACCGCGGCCGCGACTCACACATGCCCACCGTGCC |
| | | CAGCACCTGAACTCCTGGGGGGACCGTCAGTCTTCCTCTTCCCCCCAAAACCCAAG |
| | | GACACCCTCATGATCTCCCGGACCCCTGAGGTCACATGCGTGGTGGTGGACGTGAG |
| | | CCACGAAGACCCTGAGGTCAAGTTCAACTGGTACGTGGACGGCGTGGAGGTGCATA |
| | | ATGCCAAGACAAAGCCGCGGGAGGAGCAGTACAACAGCACGTACCGTGTGGTCAG |
| | | CGTCCTCACCGTCCTGCACCAGGACTGGCTGAATGGCAAGGAGTACAAGTGCAAGG |
| | | TCTCCAACAAAGCCCTCCCAGCCCCCATCGAGAAAACCATCTCCAAAGCCAAAGGG |
| | | CAGCCCCGAGAACCACAGGTGTGCACCCTGCCCCCATCCCGGGATGAGCTGACCA |
| | | AGAACCAGGTCAGCCTGTCCTGCGCCGTCAAAGGCTTCTATCCCAGCGACATCGCC |
| | | GTGGAGTGGGAGAGCAATGGGCAGCCGGAGAACAACTACAAGACCACGCCTCCCG |
| | | TGCTGGACTCCGACGGCTCCTTCTTCCTCGTGAGCAAGCTCACCGTGGACAAGAGC |
| | | AGGTGGCAGCAGGGGAACGTCTTCTCATGCTCCGTGATGCATGAGGCTCTGCACAA |
| | | CCACTACACGCAGAAGAGCCTCTCCCTGTCTCCGGGTAAA |
| 179 | Anti-CD132 P2D3 Fab LC | GATGTTGTGATGACTCAGTCTCCACTCTCCCTGCCCGTCACCCCTGGAGAGCCGGC |
| | (VL, joint CL) | CTCCATCTCCTGCAGGTCTAGTCAGAGCCTCCTGCATAGTAATGGATACAACTATTT |
| | | GGATTGGTACCTGCAGAAGCCAGGGCAGTCTCCACAGCTCCTGATCTATTTGGGTT |
| | | CTAATCGGGCCTCCGGGGTCCCTGACAGGTTCAGTGGCAGTGGATCAGGCACAGAT |
| | | TTTACACTGAAAATCAGCAGGGTGGAGGCTGAGGATGTTGGGGTTTATTACTGCATG |
| | | CAAGGTACACACTGGCCCTGGACGTTCGGCCAAGGGACCAAGGTGGAAATCAAACG |
| | | AACTGTGGCTGCACCATCTGTCTTCATCTTCCCGCCATCTGATGAGCAGTTGAAATC |
| | | TGGAACTGCCTCTGTTGTGTGCCTGCTGAATAATTTCTATCCCAGAGAGGCCAAAGT |
| | | ACAGTGGAAGGTGGATAACGCCCTCCAATCGGGTAACTCCCAGGAGAGTGTCACAG |
| | | AGCAGGACAGCAAGGACAGCACCTACAGCCTCAGCAGCACCCTGACGCTGAGCAAA |
| | | GCAGACTACGAGAAACACAAACTCTACGCCTGCGAAGTCACCCATCAGGGCCTGAG |
| | | CTCGCCCGTCACAAAGAGCTTCAACAGGGGAGAGTGT |
| 180 | Anti-CD132 P2D3 Fab HC | CAGGTGCAGCTACAGCAGTGGGGCGCAGGACTGTTGAAGCCTTCGGAGACCCTGT |
| | (VH, joint CH1) | CCCTCACCTGCACTATCTATGGTGGGTCCTTCAGTGGTTTCTACTGGAGCTGGATCC |
| | | GCCAGCCCCCAGGGAAGGGACTGGAGTGGATTGGGGAAATCAATCATAGTGGAAG |
| | | CACCAACTACAACCCGTCCCTCAAGAGTCGAGTCACCATATCAGTAGACACGTCCAA |
| | | GAACCAGTTCTCCCTGAAGCTGAGCTCTGTGACCGCCGCGGACACGGCTATATATT |
| | | ACTGTGCGAGAGGCCCCGCGGGATCCACCTCGTCCGGCTACTTTGACCACTGGGG |
| | | CCAGGGAACCCTGGTCACCGTCTCAAGCGCCTCCACCAAGGGCCCATCGGTCTTCC |
| | | CCCTGGCACCCTCCTCCAAGAGCACCTCTGGGGGCACAGCGGCCCTGGGCTGCCT |
| | | GGTCAAGGACTACTTCCCCGAACCGGTGACGGTGTCGTGGAACTCAGGCGCCCTGA |
| | | CCAGCGGCGTCCACACCTTCCCGGCTGTCCTACAGTCCTCAGGACTCTACTCCCTC |
| | | AGCAGCGTAGTGACCGTGCCCTCCAGCAGCTTGGGCACCCAGACCTACATCTGCAA |
| | | CGTGAATCACAAGCCCAGCAACACCAAGGTGGACAAGAAAGTTGAGCCCAAATCTT |
| | | GT |
| 181 | Anti-CD132 P2D3 scFv | CAGGTCCAGCTGCAGCAGTGGGGAGCCGGACTGCTGAAACCCTCTGAGACTCTGA |
| | and Fc with hole | GCCTGACTTGCACAATCTACGGGGGATCATTCAGCGGCTTCTACTGGTCCTGGATCA |
| | modification | GGCAGCCCCCTGGCAAGGGGCTGGAGTGGATCGGAGAAATTAACCACAGTGGCTC |
| | | AACAAACTATAATCCCAGCCTGAAATCCCGCGTGACCATCTCAGTGGACACAAGCAA |
| | | GAATCAGTTCAGCCTGAAGCTGAGCAGCGTGACAGCCGCTGATACTGCCATCTACT |
| | | ATTGCGCACGGGGCCCTGCCGGGTCCACCTCTAGTGGGTACTTTGACCATTGGGGA |
| | | CAGGGCACCCTGGTGACAGTCTCAAGCGGAGGAGGAGGCTCTGGAGGAGGAGGGA |
| | | GTGGAGGCGGGGGCAGCGATGTGGTCATGACTCAGTCTCCACTGAGTCTGCCAGT |
| | | GACCCCCGGCGAGCCTGCTAGCATCTCCTGTCGATCCTCTCAGTCCCTGCTGCACT |
| | | CTAACGGATACAATTATCTGGACTGGTACTTGCAGAAGCCAGGCCAGAGCCCCCAG |
| | | CTGCTGATCTATCTGGGGAGTAACCGGGCTTCAGGAGTGCCTGACAGATTCTCTGG |
| | | GAGTGGATCAGGCACTGATTTTACCCTGAAAATTAGCAGAGTCGAGGCAGAAGATGT |
| | | GGGCGTCTACTATTGCATGCAGGGGACTCATTGGCCCTGGACCTTTGGGCAGGGAA |
| | | CAAAGGTGGAGATCAAGAACAGCGGCGCGGGCACCGCGGCCGCGACTCACACATG |
| | | CCCACCGTGCCCAGCACCTGAACTCCTGGGGGGACCGTCAGTCTTCCTCTTCCCCC |
| | | CAAAACCCAAGGACACCCTCATGATCTCCCGGACCCCTGAGGTCACATGCGTGGTG |
| | | GTGGACGTGAGCCACGAAGACCCTGAGGTCAAGTTCAACTGGTACGTGGACGGCGT |
| | | GGAGGTGCATAATGCCAAGACAAAGCCGCGGGAGGAGCAGTACAACAGCACGTAC |
| | | CGTGTGGTCAGCGTCCTCACCGTCCTGCACCAGGACTGGCTGAATGGCAAGGAGTA |
| | | CAAGTGCAAGGTCTCCAACAAAGCCCTCCCAGCCCCCATCGAGAAAACCATCTCCA |
| | | AAGCCAAAGGGCAGCCCCGAGAACCACAGGTGTGCACCCTGCCCCCATCCCGGGA |
| | | TGAGCTGACCAAGAACCAGGTCAGCCTGTCCTGCGCCGTCAAAGGCTTCTATCCCA |
| | | GCGACATCGCCGTGGAGTGGGAGAGCAATGGGCAGCCGGAGAACAACTACAAGAC |
| | | CACGCCTCCCGTGCTGGACTCCGACGGCTCCTTCTTCCTCGTGAGCAAGCTCACCG |
| | | TGGACAAGAGCAGGTGGCAGCAGGGGAACGTCTTCTCATGCTCCGTGATGCATGAG |
| | | GCTCTGCACAACCACTACACGCAGAAGAGCCTCTCCCTGTCTCCGGGTAAA |
| 182 | Anti-CD132 P1G4 Fab LC | GATGTTGTGATGACTCAGTCTCCACTCTCCCTGCCCGTCACCCCTGGAGAGCCGGC |
| | (VL, joint CL) | CTCCATCTCCTGCAGGTCTAGTCAGAGCCTCCTGCATAGTAATGGATACAACTATTT |
| | | GGATTGGTACCTGCAGAAGCCAGGGCAGTCTCCACAGCTCCTGATCTATTTGGGTT |
| | | CTAATCGGGCCTCCGGGGTCCCTGACAGGTTCAGTGGCAGTGGATCAGGCACAGAT |
| | | TTTACACTGAAAATCAGCAGGGTGGAGGCTGAGGATGTTGGGGTTTATTACTGCCTG |
| | | CAAGGTACACATTGGCCGTGGACGTTCGGCCAGGGGACCAAGGTGGAAATCAAACG |
| | | AACTGTGGCTGCACCATCTGTCTTCATCTTCCCGCCATCTGATGAGCAGTTGAAATC |
| | | TGGAACTGCCTCTGTTGTGTGCCTGCTGAATAACTTCTATCCCAGAGAGGCCAAAGT |
| | | ACAGTGGAAGGTGGATAACGCCCTCCAATCGGGTAACTCCCAGGAGAGTGTCACAG |
| | | AGCAGGACAGCAAGGACAGCACCTACAGCCTCAGCAGCACCCTGACGCTGAGCAAA |
| | | GCAGACTACGAGAAACACAAAGTCTACGCCTGCGAAGTCACCCATCAGGGCCTGAG |
| | | CTCGCCCGTCACAAAGAGCTTCAACAGGGGAGAGTGT |
| 183 | Anti-CD132 P1G4 Fab HC | CAGGTGCAGCTACAGCAGTGGGGCGCAGGACTGTTGAAGCCTTCGGAGACCCTGT |
| | (VH, joint CH1) | CCCTCACCTGCGCTGTCTATGGTGGGTCCCTCAGTGGTTACTACTGGAGCTGGATC |
| | | CGCCAGCCCCCAGGGAAGGGGCTGGAGTGGATTGGGGAAATCAATCATAGTGGAA |
| | | GCACCAACTACAACCCATCCCTCAAGAGTCGAGTCACCATATCAGTAGACACGTCCA |
| | | AGAACCAGTTCTCCCTGAAGCTGAGCTCTGTGACCGCCGCGGACACGGCTGTGTAT |
| | | TACTGTGCGAGAGGCAGCAGCTCCTACTACATGGACGTCTGGGGCAAAGGGACCAC |
| | | GGTCACCGTCTCAAGCGCCTCCACCAAGGGCCCATCGGTCTTCCCCCTGGCACCCT |
| | | CCTCCAAGAGCACCTCTGGGGGCACAGCGGCCCTGGGCTGCCTGGTCAAGGACTA |
| | | CTTCCCCGAACCGGTGACGGTGTCGTGGAACTCAGGCGCCCTGACCAGCGGCGTC |
| | | CACACCTTCCCGGCTGTCCTACAGTCCTCAGGACTCTACTCCCTCAGCAGCGTAGT |
| | | GACCGTGCCCTCCAGCAGCTTGGGCACCCAGACCTACATCTGCAACGTGAATCACA |
| | | AGCCCAGCAACACCAAGGTGGACAAGAAAGTTGAGCCCAAATCTTGT |
| 184 | Anti-CD132 P1G4 scFv | CAGGTCCAGCTGCAGCAGTGGGGAGCCGGACTGCTGAAACCAAGCGAGACTCTGA |
| | and Fc with hole | GCCTGACTTGTGCCGTGTATGGGGGAAGCCTGTCCGGCTACTATTGGTCTTGGATC |
| | modification | AGGCAGCCCCCTGGCAAGGGGCTGGAGTGGATCGGCGAAATTAACCACTCAGGGA |
| | | GCACAAACTACAATCCCTCCCTGAAATCTCGCGTGACCATTAGCGTGGACACATCCA |
| | | AGAATCAGTTCAGCCTGAAGCTGAGCAGCGTGACAGCCGCTGACACCGCCGTGTAC |
| | | TATTGCGCCAGAGGCAGCAGCAGCTACTATATGGATGTGTGGGGAAAGGGCACCAC |
| | | AGTGACCGTCAGCTCCGGAGGAGGAGGCAGTGGAGGAGGAGGGTCCGGAGGCGG |
| | | GGGATCTGACGTGGTCATGACTCAGAGTCCTCTGTCACTGCCTGTGACCCCCGGCG |
| | | AGCCTGCATCCATCTCTTGTCGATCTAGTCAGTCTCTGCTGCACAGTAACGGCTACA |
| | | ATTATCTGGATTGGTACTTGCAGAAGCCAGGGCAGTCCCCCCAGCTGCTGATCTATC |
| | | TGGGATCAAACCGGGCTAGCGGCGTGCCTGACAGATTCAGTGGGTCAGGAAGCGG |
| | | CACTGATTTTACCCTGAAAATTAGCAGAGTCGAGGCAGAAGATGTGGGGGTCTACTA |
| | | TTGCCTCCAGGGAACTCATTGGCCCTGGACCTTTGGGCAGGGAACAAAGGTGGAGA |
| | | TCAAGAACAGCGGCGCGGGCACCGCGGCCGCGACTCACACATGCCCACCGTGCCC |
| | | AGCACCTGAACTCCTGGGGGGACCGTCAGTCTTCCTCTTCCCCCCAAAACCCAAGG |
| | | ACACCCTCATGATCTCCCGGACCCCTGAGGTCACATGCGTGGTGGTGGACGTGAGC |
| | | CACGAAGACCCTGAGGTCAAGTTCAACTGGTACGTGGACGGCGTGGAGGTGCATAA |
| | | TGCCAAGACAAAGCCGCGGGAGGAGCAGTACAACAGCACGTACCGTGTGGTCAGC |
| | | GTCCTCACCGTCCTGCACCAGGACTGGCTGAATGGCAAGGAGTACAAGTGCAAGGT |
| | | CTCCAACAAAGCCCTCCCAGCCCCCATCGAGAAAACCATCTCCAAAGCCAAAGGGC |
| | | AGCCCCGAGAACCACAGGTGTGCACCCTGCCCCCATCCCGGGATGAGCTGACCAA |
| | | GAACCAGGTCAGCCTGTCCTGCGCCGTCAAAGGCTTCTATCCCAGCGACATCGCCG |
| | | TGGAGTGGGAGAGCAATGGGCAGCCGGAGAACAACTACAAGACCACGCCTCCCGT |
| | | GCTGGACTCCGACGGCTCCTTCTTCCTCGTGAGCAAGCTCACCGTGGACAAGAGCA |
| | | GGTGGCAGCAGGGGAACGTCTTCTCATGCTCCGTGATGCATGAGGCTCTGCACAAC |
| | | CACTACACGCAGAAGAGCCTCTCCCTGTCTCCGGGTAAA |
| 185 | Anti-CD132 P1B12 Fab LC | GATGTTGTGATGACTCAGTCTCCACTCTCCCTGCCCGTCACCCTTGGTCAGCCGGC |
| | (VL, joint CL) | CTCCATCTCCTGCAGGTCTAGTCAGAGCCTCCTGCACAGTAATGGAAACAACTATTT |
| | | GGATTGGTACCTGCAGAAGCCAGGGCAGTCTCCACAGCTCCTGATCTATTTGGGTT |
| | | CTAATCGGGCCTCCGGGGTCCCTGACAGGTTCAGTGGCAGTGGATCAGGCACAGAT |
| | | TTTACACTGAAAATCAGCAGGGTGGAGGCTGAGGATGTTGGGATTTATTACTGCATG |
| | | CAAGGGACACACTGGCCTTGGACGTTCGGCCAAGGGACCAAGGTGGAAATCGAAC |
| | | GAACTGTGGCTGCACCATCTGTCTTCATCTTCCCGCCATCTGATGAGCAGTTGAAAT |
| | | CTGGAACTGCCTCTGTTGTGTGCCTGCTGAATAACTTCTATCCCAGAGAGGCCAAAG |
| | | TACAGTGGAAGGTGGATAGCGCCCTCCAATCGGGTAACTCCCAGGAGAGTGTCACA |
| | | GAGCAGGACAGCAAGGACAGCACCTACAGCCTCAGCAGCACCCTGACGCTGAGCA |
| | | AAGCAGACTACGAGAAACACAAACTCTACGCCTGCGAAGTCACCCATCAGGGCCTG |
| | | AGCTCGCCCGTCACAAAGAGCTTCAACAGGGGAGAGTGT |
| 186 | Anti-CD132 P1B12 Fab | CAGGTGCAGCTACAGCAGTGGGGCGCAGGACTGTTGAAGCCTTCGGAGACCCTGT |
| | HC (VH, joint CH1) | CCCTCACCTGCGCTGTCTATGGTGGGTCCTTCAGTGGTTACTACTGGAGCTGGATC |
| | | CGCCAGCCCCCAGGGAAGGGGCTGGAGTGGATTGGGGAAATCAATCATAGTGGAA |
| | | GCACCAACTACAACCCGTCCCTCAAGAGTCGAGTCACCATATCAGTAGACACGTCCA |
| | | AGAACCAGTTCTCCCTGAAGCTGAGCTCTGTGACCGCCGCGGACACGGCTGTGTAT |
| | | TACTGTGCGAGAGGCGGTAGCGCGTACTTCCAGCACTGGGGCCAGGGAACCCTGG |
| | | TCACCGTCTCAAGCGCCTCCACCAAGGGCCCATCGGTCTTCCCCCTGGCACCCTCC |
| | | TCCAAGAGCACCTCTGGGGGCACAGCGGCCCTGGGCTGCCTGGTCAAGGACTACT |
| | | TCCCCGAACCGGTGACGGTGTCGTGGAACTCAGGCGCCCTGACCAGCGGCGTCCA |
| | | CACCTTCCCGGCTGTCCTACAGTCCTCAGGACTCTACTCCCTCAGCAGCGTAGTGA |
| | | CCGTGCCCTCCAGCAGCTTGGGCACCCAGACCTACATCTGCAACGTGAATCACAAG |
| | | CCCAGCAACACCAAGGTGGACAAGAAAGTTGAGCCCAAATCTTGT |
| 187 | Anti-CD132 P1B12 scFv | CAGGTCCAGCTGCAGCAGTGGGGGGCCGGGCTGCTGAAACCTTCCGAAACTCTGT |
| | and Fc with hole | CTCTGACTTGTGCCGTGTATGGGGGGTCCTTTAGTGGCTACTATTGGTCATGGATCA |
| | modification | GGCAGCCCCCTGGAAAGGGCCTGGAGTGGATCGGAGAAATTAACCACTCCGGCTCT |
| | | ACAAACTACAATCCAAGTCTGAAATCACGCGTGACCATTTCTGTGGACACCAGTAAG |
| | | AATCAGTTCAGCCTGAAGCTGAGCAGCGTGACAGCCGCTGATACCGCCGTGTACTA |
| | | TTGCGCCCGAGGCGGGTCTGCTTATTTTCAGCATTGGGGGCAGGGAACCCTGGTGA |
| | | CAGTCTCTAGTGGAGGAGGAGGCAGCGGCGGAGGAGGCTCTGGAGGAGGAGGGA |
| | | GTGACGTGGTCATGACTCAGAGCCCACTGTCCCTGCCAGTGACCCTGGGACAGCCA |
| | | GCTAGTATCTCATGTAGATCAAGCCAGTCACTGCTGCACAGCAACGGCAACAATTAC |
| | | CTGGATTGGTACTTGCAGAAGCCTGGCCAGAGCCCACAGCTGCTGATCTACCTGGG |
| | | GTCCAATCGGGCATCTGGAGTGCCCGACAGATTCAGCGGCTCCGGGTCTGGAACTG |
| | | ATTTTACCCTGAAGATCAGCCGGGTGGAGGCCGAAGACGTCGGCATCTACTATTGC |
| | | ATGCAGGGGACTCATTGGCCTTGGACCTTCGGCCAGGGGACAAAAGTGGAGATCGA |
| | | AAACAGCGGCGCGGGCACCGCGGCCGCGACTCACACATGCCCACCGTGCCCAGCA |
| | | CCTGAACTCCTGGGGGGACCGTCAGTCTTCCTCTTCCCCCCAAAACCCAAGGACAC |
| | | CCTCATGATCTCCCGGACCCCTGAGGTCACATGCGTGGTGGTGGACGTGAGCCACG |
| | | AAGACCCTGAGGTCAAGTTCAACTGGTACGTGGACGGCGTGGAGGTGCATAATGCC |
| | | AAGACAAAGCCGCGGGAGGAGCAGTACAACAGCACGTACCGTGTGGTCAGCGTCC |
| | | TCACCGTCCTGCACCAGGACTGGCTGAATGGCAAGGAGTACAAGTGCAAGGTCTCC |
| | | AACAAAGCCCTCCCAGCCCCCATCGAGAAAACCATCTCCAAAGCCAAAGGGCAGCC |
| | | CCGAGAACCACAGGTGTGCACCCTGCCCCCATCCCGGGATGAGCTGACCAAGAAC |
| | | CAGGTCAGCCTGTCCTGCGCCGTCAAAGGCTTCTATCCCAGCGACATCGCCGTGGA |
| | | GTGGGAGAGCAATGGGCAGCCGGAGAACAACTACAAGACCACGCCTCCCGTGCTG |
| | | GACTCCGACGGCTCCTTCTTCCTCGTGAGCAAGCTCACCGTGGACAAGAGCAGGTG |
| | | GCAGCAGGGGAACGTCTTCTCATGCTCCGTGATGCATGAGGCTCTGCACAACCACT |
| | | ACACGCAGAAGAGCCTCTCCCTGTCTCCGGGTAAA |
| 188 | Anti-CD132 P1C7 Fab LC | GAAATTGTGCTGACTCAGTCTCCACTCTCCCTGCCCGTCACCCCTGGAGAGCCGGC |
| | (VL, joint CL) | CTCCATCTCCTGCAGGTCTAGTCAGAGCCTCCTGCATAGTAATGGATACAACTATTT |
| | | GGATTGGTACCTGCAGAAGCCAGGGCAGTCTCCACAGCTCCTGATCTATTTGGCTTC |
| | | TAATCGGGCCTCCGGGGTCCCAGACAGATTCAGCGGCAGTGGGTCAGGCACTGATT |
| | | TCACACTGAAAATCAGCAGGGTGGAGGCTGAGGATGTTGGGGTTTATTACTGCATG |
| | | CAAGGTACACACTGGCCGTGGACGTTCGGCCAAGGGACCAAGGTGGAAGTCAAAC |
| | | GAACTGTGGCTGCACCATCTGTCTTCATCTTCCCGCCATCTGATGAGCAGTTGAAAT |
| | | CTGGAACTGCCTCTGTTGTGTGCCTGCTGAATAACTTCTATCCCAGAGAGGCCAAAG |
| | | TACAGTGGAAGGTGGATAACGCCCTCCAATCGGGTAACTCCCGGGAGAGTGTCACA |
| | | GAGCAGGACAGCAAGGACAGCACCTACAGCCTCAGCAGCACCCTGACGCTGAGCA |
| | | AAGCAGACTACGAGAAACACAAAGTCTACGCCTGCGAAGTCACCCATCAGGGCCTG |
| | | AGCTCGCCCGTCACAAAGAGCTTCAACAGGGGAGAGTGT |
| 189 | Anti-CD132 P1C7 Fab HC | CAGGTGCAGCTACAGCAGTGGGGCGCAGGACTGTTGAAGCCTTCGGAGACCCTGT |
| | (VH, joint CH1) | CCCTCACCTGCGCTGTCTATGGTGGGTCCTTCAGTGGTTACTACTGGAGCTGGATC |
| | | CGCCAGCCCCCAGGGAAGGGGCTGGAGTGGATTGGGGAAATCAATCATAGTGGAA |
| | | GCACCAACTACAACCCGTCCCTCAAGAGTCGAGTCACCATATCAGAAGACGCGTCC |
| | | AAGAAGCAGTTCTCCCTGACGCTGACCTCTGTGACCGCCGCGGACACGGCTGTCTA |
| | | TTACTGTGCGAGAGGCCCCGCGGGTACCGGCTCGTCCGGCTACTTTGACTACTGGG |
| | | GCCAGGGAACCCTGGTCACCGTCTCAAGCGCCTCCACCAAGGGCCCATCGGTCTTC |
| | | CCCCTGGCACCCTCCTCCAAGAGCACCTCTGGGGGCACAGCGGCCCTGGGCTGCC |
| | | TGGTCAAGGACTACTTCCCCGAACCGGTGACGGTGTCGTGGAACTCAGGCGCCCTG |
| | | ACCAGCGGCGTCCACACCTTCCCGGCTGTCCTACAGTCCTCAGGACTCTACTCCCT |
| | | CAGCAGCGTAGTGACCGTGCCCTCCAGCAGCTTGGGCACCCAGACCTACATCTGCA |
| | | ACGTGAATCACAAGCCCAGCAACACCAAGGTGGACAAGAAAGTTGAGCCCAAATCTT |
| | | GT |
| 190 | Anti-CD132 P1C7 scFv | CAGGTCCAGCTGCAGCAGTGGGGAGCCGGACTGCTGAAGCCTAGCGAAACTCTGA |
| | and Fc with hole | GCCTGACTTGTGCTGTCTACGGAGGATCATTTAGTGGCTACTATTGGTCATGGATCA |
| | modification | GGCAGCCCCCTGGCAAGGGGCTGGAGTGGATCGGAGAAATTAACCACTCCGGCTC |
| | | TACAAACTACAATCCCAGTCTGAAATCACGCGTGACTATTTCTGAGGACGCCAGTAA |
| | | GAAACAGTTCTCCCTGACCCTGACATCTGTGACCGCCGCTGATACAGCTGTCTACTA |
| | | TTGCGCACGGGGCCCTGCCGGAACAGGCAGCTCCGGATACTTTGACTATTGGGGG |
| | | CAGGGAACTCTGGTGACCGTCTCTAGTGGCGGAGGAGGCAGTGGAGGAGGAGGGT |
| | | CCGGAGGAGGAGGATCTGAGATCGTGCTGACTCAGAGCCCACTGTCCCTGCCAGTC |
| | | ACCCCCGGCGAACCTGCCAGTATTTCATGTCGATCAAGCCAGTCACTGCTGCACAG |
| | | CAACGGATACAATTATCTGGACTGGTACTTGCAGAAGCCAGGCCAGAGCCCCCAGC |
| | | TGCTGATCTATCTGGCTTCCAATCGGGCATCTGGCGTGCCTGACAGATTCAGCGGC |
| | | TCCGGGTCTGGAACAGATTTTACTCTGAAAATTTCCAGAGTGGAGGCCGAAGATGTG |
| | | GGGGTCTACTATTGCATGCAGGGAACTCATTGGCCCTGGACCTTCGGCCAGGGGAC |
| | | AAAGGTGGAAGTCAAAAACAGCGGCGCGGGCACCGCGGCCGCGACTCACACATGC |
| | | CCACCGTGCCCAGCACCTGAACTCCTGGGGGGACCGTCAGTCTTCCTCTTCCCCCC |
| | | AAAACCCAAGGACACCCTCATGATCTCCCGGACCCCTGAGGTCACATGCGTGGTGG |
| | | TGGACGTGAGCCACGAAGACCCTGAGGTCAAGTTCAACTGGTACGTGGACGGCGTG |
| | | GAGGTGCATAATGCCAAGACAAAGCCGCGGGAGGAGCAGTACAACAGCACGTACC |
| | | GTGTGGTCAGCGTCCTCACCGTCCTGCACCAGGACTGGCTGAATGGCAAGGAGTAC |
| | | AAGTGCAAGGTCTCCAACAAAGCCCTCCCAGCCCCCATCGAGAAAACCATCTCCAAA |
| | | GCCAAAGGGCAGCCCCGAGAACCACAGGTGTGCACCCTGCCCCCATCCCGGGATG |
| | | AGCTGACCAAGAACCAGGTCAGCCTGTCCTGCGCCGTCAAAGGCTTCTATCCCAGC |
| | | GACATCGCCGTGGAGTGGGAGAGCAATGGGCAGCCGGAGAACAACTACAAGACCA |
| | | CGCCTCCCGTGCTGGACTCCGACGGCTCCTTCTTCCTCGTGAGCAAGCTCACCGTG |
| | | GACAAGAGCAGGTGGCAGCAGGGGAACGTCTTCTCATGCTCCGTGATGCATGAGGC |
| | | TCTGCACAACCACTACACGCAGAAGAGCCTCTCCCTGTCTCCGGGTAAA |
| 191 | Human CD122 (UniProt: | MAAPALSWRLPLLILLLPLATSWASAAVNGTSQFTCFYNSRANISCVWSQDGALQDTSC |
| | P14784-1, v1) | QVHAWPDRRRWNQTCELLPVSQASWACNLILGAPDSQKLTTVDIVTLRVLCREGVRWR |
| | | VMAIQDFKPFENLRLMAPISLQVVHVETHRCNISWEISQASHYFERHLEFEARTLSPGHT |
| | | WEEAPLLTLKQKQEWICLETLTPDTQYEFQVRVKPLQGEFTTWSPWSQPLAFRTKPAAL |
| | | GKDTIPWLGHLLVGLSGAFGFIILVYLLINCRNTGPWLKKVLKCNTPDPSKFFSQLSSEHG |
| | | GDVQKWLSSPFPSSSFSPGGLAPEISPLEVLERDKVTQLLLQQDKVPEPASLSSNHSLT |
| | | SCFTNQGYFFFHLPDALEIEACQVYFTYDPYSEEDPDEGVAGAPTGSSPQPLQPLSGED |
| | | DAYCTFPSRDDLLLFSPSLLGGPSPPSTAPGGSGAGEERMPPSLQERVPRDWDPQPLG |
| | | PPTPGVPDLVDFQPPPELVLREAGEEVPDAGPREGVSFPWSRPPGQGEFRALNARLPL |
| | | NTDAYLSLQELQGQDPTHLV |
| 192 | Mature form Human | AVNGTSQFTCFYNSRANISCVWSQDGALQDTSCQVHAWPDRRRWNQTCELLPVSQAS |
| | CD122 (UniProt: P14784- | WACNLILGAPDSQKLTTVDIVTLRVLCREGVRWRVMAIQDFKPFENLRLMAPISLQVVHV |
| | 1, v1 residues 27 to | ETHRCNISWEISQASHYFERHLEFEARTLSPGHTWEEAPLLTLKQKQEWICLETLTPDTQ |
| | 525) | YEFQVRVKPLQGEFTTWSPWSQPLAFRTKPAALGKDTIPWLGHLLVGLSGAFGFIILVYL |
| | | LINCRNTGPWLKKVLKCNTPDPSKFFSQLSSEHGGDVQKWLSSPFPSSSFSPGGLAPEI |
| | | SPLEVLERDKVTQLLLQQDKVPEPASLSSNHSLTSCFTNQGYFFFHLPDALEIEACQVYF |
| | | TYDPYSEEDPDEGVAGAPTGSSPQPLQPLSGEDDAYCTFPSRDDLLLFSPSLLGGPSPP |
| | | STAPGGSGAGEERMPPSLQERVPRDWDPQPLGPPTPGVPDLVDFQPPPELVLREAGE |
| | | EVPDAGPREGVSFPWSRPPGQGEFRALNARLPLNTDAYLSLQELQGQDPTHLV |
| 193 | Extracellular domain of | AVNGTSQFTCFYNSRANISCVWSQDGALQDTSCQVHAWPDRRRWNQTCELLPVSQAS |
| | Human CD122 (UniProt: | WACNLILGAPDSQKLTTVDIVTLRVLCREGVRWRVMAIQDFKPFENLRLMAPISLQVVHV |
| | P14784-1, v1 residues | ETHRCNISWEISQASHYFERHLEFEARTLSPGHTWEEAPLLTLKQKQEWICLETLTPDTQ |
| | 27 to 240) | YEFQVRVKPLQGEFTTWSPWSQPLAFRTKPAALGKDT |
| 194 | Human CD132 (UniProt: | MLKPSLPFTSLLFLQLPLLGVGLNTTILTPNGNEDTTADFFLTTMPTDSLSVSTLPLPEVQ |
| | P31785-1, v1) | CFVFNVEYMNCTWNSSSEPQPTNLTLHYWYKNSDNDKVQKCSHYLFSEEITSGCQLQK |
| | | KEIHLYQTFVVQLQDPREPRRQATQMLKLQNLVIPWAPENLTLHKLSESQLELNWNNRF |
| | | LNHCLEHLVQYRTDWDHSWTEQSVDYRHKFSLPSVDGQKRYTFRVRSRFNPLCGSAQ |
| | | HWSEWSHPIHWGSNTSKENPFLFALEAVVISVGSMGLIISLLCVYFWLERTMPRIPTLKN |
| | | LEDLVTEYHGNFSAWSGVSKGLAESLQPDYSERLCLVSEIPPKGGALGEGPGASPCNQ |
| | | HSPYWAPPCYTLKPET |
| 195 | Mature form Human | LNTTILTPNGNEDTTADFFLTTMPTDSLSVSTLPLPEVQCFVFNVEYMNCTWNSSSEPQP |
| | CD132 (UniProt: P31785- | TNLTLHYWYKNSDNDKVQKCSHYLFSEEITSGCQLQKKEIHLYQTFVVQLQDPREPRRQ |
| | 1, v1 residues 23 to | ATQMLKLQNLVIPWAPENLTLHKLSESQLELNWNNRFLNHCLEHLVQYRTDWDHSWTE |
| | 369) | QSVDYRHKFSLPSVDGQKRYTFRVRSRFNPLCGSAQHWSEWSHPIHWGSNTSKENPF |
| | | LFALEAVVISVGSMGLIISLLCVYFWLERTMPRIPTLKNLEDLVTEYHGNFSAWSGVSKGL |
| | | AESLQPDYSERLCLVSEIPPKGGALGEGPGASPCNQHSPYWAPPCYTLKPET |
| 196 | Extracellular domain of | LNTTILTPNGNEDTTADFFLTTMPTDSLSVSTLPLPEVQCFVFNVEYMNCTWNSSSEPQP |
| | Human CD132 (UniProt: | TNLTLHYWYKNSDNDKVQKCSHYLFSEEITSGCQLQKKEIHLYQTFVVQLQDPREPRRQ |
| | P31785-1, v1 residues | ATQMLKLQNLVIPWAPENLTLHKLSESQLELNWNNRFLNHCLEHLVQYRTDWDHSWTE |
| | 23 to 262) | QSVDYRHKFSLPSVDGQKRYTFRVRSRFNPLCGSAQHWSEWSHPIHWGSNTSKENPF |
| | | LFALEA |
| 197 | P1A3-AQ VH | QVQLQAWGAGLLKPSETLSLTCAVYGGSFSGYYWSWIRQPPGKGLEWIGEINHSGSTN |
| | | YNPSLKSRATISVDTSKNQFSLKLSSVTAADTAVYYCATSPGGYSGGYFQHWGQGTQV |
| | | TVSS |
| 198 | P1A3-ANQ VH | QVQLQAWGAGLLKPSETLSLTCAVYGGSFSGYYWSWIRQPPGKGLEWIGEINHSGSTN |
| | | YNPSLKSRATISVDTSKNQFSLKLSNVTAADTAVYYCATSPGGYSGGYFQHWGQGTQV |
| | | TVSS |
| 199 | P1A10-AQ VH | QVQLQASGAEVKKPGSSVKVSCKASGGTFSSYAISWVRQAPGQGLEWMGGFDPEDGE |
| | | TIYAQKFQGRVTMTEDTSTDTAYMELSSLRSEDTAVYYCATDLRIPYYYDNPWGQGTQV |
| | | TVSS |
| 200 | P1A10-ANQ VH | QVQLQASGAEVKKPGSSVKVSCKASGGTFSSYAISWVRQAPGQGLEWMGGFDPEDGE |
| | | TIYAQKFQGRVTMTEDTSTDTAYMELSNLRSEDTAVYYCATDLRIPYYYDNPWGQGTQV |
| | | TVSS |
| 201 | HC-FR1 Anti-CD132 | QVQLQAWGAGLLKPSETLSLTCAVYGGSFS |
| | P1A3-AQ, P1A3-ANQ, | |
| | P1A3-A | |
| 202 | HC-FR1 Anti-CD132 | QVQLQASGAEVKKPGSSVKVSCKASGGTFS |
| | P1A10-AQ, P1A10-ANQ | |
| 203 | HC-FR3 Anti-CD132 | RATISVDTSKNQFSLKLSNVTAADTAVYYCAT |
| | P1A3-ANQ | |
| 204 | HC-FR3 Anti-CD132 | RVTMTEDTSTDTAYMELSNLRSEDTAVYYCAT |
| | P1A10-ANQ | |
| 205 | HC-FR4 Anti-CD132 | WGQGTQVTVSS |
| | P1A3-AQ, P1A3-ANQ, | |
| | P1A3-Q, P1A10-AQ, | |
| | P1A10-ANQ | |
| 206 | P1A3-6, 108 VH | QVQLQX₁WGAGLLKPSETLSLTCAVYGGSFSGYYWSWIRQPPGKGLEWIGEINHSGST |
| | | NYNPSLKSRATISVDTSKNQFSLKLSSVTAADTAVYYCATSPGGYSGGYFQHWGQGTX₂ |
| | | VTVSS |
| | | wherein X₁ = A, V, I, L, M, F, Y or W; X₂ = Q, S, T or N. |
| 207 | P1A3-6, 82b, 108 VH | QVQLQX₃WGAGLLKPSETLSLTCAVYGGSFSGYYWSWIRQPPGKGLEWIGEINHSGST |
| | | NYNPSLKSRATISVDTSKNQFSLKLSX₄VTAADTAVYYCATSPGGYSGGYFQHWGQGT |
| | | X₅VTVSS |
| | | wherein X₃ = A, V, I, L, M, F, Y or W; X₄ = N, Q, S or T; |
| | | X₅ = Q, S, T or N. |
| 208 | P1A10-6, 108 VH | QVQLQX₆SGAEVKKPGSSVKVSCKASGGTFSSYAISWVRQAPGQGLEWMGGFDPEDG |
| | | ETIYAQKFQGRVTMTEDTSTDTAYMELSSLRSEDTAVYYCATDLRIPYYYDNPWGQGT |
| | | X₇VTVSS |
| | | wherein X₆ = A, V, I, L, M, F, Y or W; X₇ = Q, S, T or N. |
| 209 | P1A10-6, 82b, 108 VH | QVQLQX₈SGAEVKKPGSSVKVSCKASGGTFSSYAISWVRQAPGQGLEWMGGFDPEDG |
| | | ETIYAQKFQGRVTMTEDTSTDTAYMELSX₉LRSEDTAVYYCATDLRIPYYYDNPWGQGT |
| | | X₁₀VTVSS |
| | | wherein X₈ = A, V, I, L, M, F, Y or W; X₉ = N, Q, S or T; |
| | | X₁₀ = Q, S, T or N. |
| 210 | P1A3 FR1_6 | QVQLQX₂₁WGAGLLKPSETLSLTCAVYGGSFS |
| | | wherein X₂₁ = A, V, I, L, M, F, Y or W. |
| 211 | P1A10 FR1_6 | QVQLQX₂₂SGAEVKKPGSSVKVSCKASGGTFS |
| | | wherein X₂₂ = A, V, I, L, M, F, Y or W. |
| 212 | P1A3 FR3_82b | RATISVDTSKNQFSLKLSX₂₅VTAADTAVYYCAT |
| | | wherein X₂₅ = N, Q, S or T. |
| 213 | P1A10 FR3_82b | RVTMTEDTSTDTAYMELSX₂₆LRSEDTAVYYCAT |
| | | wherein X₂₆ = N, Q, S or T. |
| 214 | P1A3, P1A10, P2C4WT, | WGQGTX₂₉VTVSS |
| | P2C4FW2, P1A10 | wherein X₂₉ = Q, S, T or N. |
| | FR4_108 | |
| 215 | P1A3-A VH | QVQLQAWGAGLLKPSETLSLTCAVYGGSFSGYYWSWIRQPPGKGLEWIGEINHSGSTN |
| | | YNPSLKSRATISVDTSKNQFSLKLSSVTAADTAVYYCATSPGGYSGGYFQHWGQGTLVT |
| | | VSS |
| 216 | P1A3-Q VH | QVQLQQWGAGLLKPSETLSLTCAVYGGSFSGYYWSWIRQPPGKGLEWIGEINHSGSTN |
| | | YNPSLKSRATISVDTSKNQFSLKLSSVTAADTAVYYCATSPGGYSGGYFQHWGQGTQV |
| | | TVSS |
| 217 | P1A3-6 VH | QVQLQX₃₀WGAGLLKPSETLSLTCAVYGGSFSGYYWSWIRQPPGKGLEWIGEINHSGST |
| | | NYNPSLKSRATISVDTSKNQFSLKLSSVTAADTAVYYCATSPGGYSGGYFQHWGQGTL |
| | | VTVSS |
| | | wherein X₃₀ = A, V, I, L, M, F, Y or W. |
| 218 | P1A3-108 VH | QVQLQQWGAGLLKPSETLSLTCAVYGGSFSGYYWSWIRQPPGKGLEWIGEINHSGSTN |
| | | YNPSLKSRATISVDTSKNQFSLKLSSVTAADTAVYYCATSPGGYSGGYFQHWGQGTX₃₁ |
| | | TVSS |
| | | wherein X₃₁ = Q, S, T or N. |
| 219 | Human IgG1 constant | ASTKGPSVFPLAPSSKSTSGGTAALGCLVKDYFPEPVTVSWNSGALTSGVHTFPAVLQS |
| | region (IGHG1; | SGLYSLSSVVTVPSSSLGTQTYICNVNHKPSNTKVDKKVEPKSCDKTHTCPPCPAPELL |
| | UniProt: P01857-1, v1) | GGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPP |
| | | SRDELTKNQVSLTCLVKGFYPSDIAVEWESNGQPENNYKTTPPVLDSDGSFFLYSKLTV |
| | | DKSRWQQGNVFSCSVMHEALHNHYTQKSLSLSPGK |
| 220 | CH1 IgG1 (positions | ASTKGPSVFPLAPSSKSTSGGTAALGCLVKDYFPEPVTVSWNSGALTSGVHTFPAVLQS |
| | 1-98 of P01857-1, v1) | SGLYSLSSVVTVPSSSLGTQTYICNVNHKPSNTKVDKKV |
| 221 | Hinge IgG1 (positions | EPKSCDKTHTCP |
| | 99-110 of P01857-1, v1) | |
| 222 | CH2 IgG1 (positions | PCPAPELLGGPSVFLFPPKPKDTLMISRTPEVTCVVVDVSHEDPEVKFNWYVDGVEVHN |
| | 111-223 of P01857-1, v1) | AKTKPREEQYNSTYRVVSVLTVLHQDWLNGKEYKCKVSNKALPAPIEKTISKAK |
| 223 | CH3 IgG1 (positions | GQPREPQVYTLPPSRDELTKNQVSLTCLVKGFYPSDIAVEWESNGQPENNYKTTPPVLD |
| | 224-330 of P01857-1, v1) | SDGSFFLYSKLTVDKSRWQQGNVFSCSVMHEALHNHYTQKSLSLSPGK |
| 224 | Cκ CL (IGCK; UniProt: | RTVAAPSVFIFPPSDEQLKSGTASVVCLLNNFYPREAKVQWKVDNALQSGNSQESVTEQ |
| | P01834-1, v2) | DSKDSTYSLSSTLTLSKADYEKHKVYACEVTHQGLSSPVTKSFNRGEC |
| 225 | CH2-CH3 IgG1 (positions | PCPAPELLGGPSVFLFPPKPKDTLMISRTPEVTCVVVDVSHEDPEVKFNWYVDGVEVHN |
| | 111-330 of P01857-1, v1) | AKTKPREEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 226 | CH3 (T366W, S354C) | GQPREPQVYTLPPCRDELTKNQVSLWCLVKGFYPSDIAVEWESNGQPENNYKTTPPVL |
| | | DSDGSFFLYSKLTVDKSRWQQGNVFSCSVMHEALHNHYTQKSLSLSPGK |
| 227 | CH3 | GQPREPQVCTLPPSRDELTKNQVSLSCAVKGFYPSDIAVEWESNGQPENNYKTTPPVL |
| | (T366S, L368A, Y407V, | DSDGSFFLVSKLTVDKSRWQQGNVFSCSVMHEALHNHYTQKSLSLSPGK |
| | Y349C) | |
| 228 | CH2-CH3 (T366W, | PCPAPELLGGPSVFLFPPKPKDTLMISRTPEVTCVVVDVSHEDPEVKFNWYVDGVEVHN |
| | S354C) | AKTKPREEQYNSTYRVVSVLTVLHQDWLNGKEYKCKVSNKALPAPIEKTISKAKGQPRE |
| | | PQVYTLPPCRDELTKNQVSLWCLVKGFYPSDIAVEWESNGQPENNYKTTPPVLDSDGS |
| | | FFLYSKLTVDKSRWQQGNVFSCSVMHEALHNHYTQKSLSLSPGK |
| 229 | CH2-CH3 | PCPAPELLGGPSVFLFPPKPKDTLMISRTPEVTCVVVDVSHEDPEVKFNWYVDGVEVHN |
| | (T366S, L368A, Y407V, | AKTKPREEQYNSTYRVVSVLTVLHQDWLNGKEYKCKVSNKALPAPIEKTISKAKGQPRE |
| | Y349C) | PQVCTLPPSRDELTKNQVSLSCAVKGFYPSDIAVEWESNGQPENNYKTTPPVLDSDGSF |
| | | FLVSKLTVDKSRWQQGNVFSCSVMHEALHNHYTQKSLSLSPGK |
| 230 | CH2(LALA)-CH3 | PCPAPEAAGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAK |
| 231 | CH2(LALA)-CH3 (T366W, | PCPAPEAAGGPSVFLFPPKPKDTLMISRTPEVTCVVVDVSHEDPEVKFNWYVDGVEVH |
| | S354C) | NAKTKPREEQYNSTYRVVSVLTVLHQDWLNGKEYKCKVSNKALPAPIEKTISKAKGQPR |
| | | EPQVYTLPPCRDELTKNQVSLWCLVKGFYPSDIAVEWESNGQPENNYKTTPPVLDSDG |
| | | SFFLYSKLTVDKSRWQQGNVFSCSVMHEALHNHYTQKSLSLSPGK |
| 232 | CH2(LALA)-CH3 | PCPAPEAAGGPSVFLFPPKPKDTLMISRTPEVTCVVVDVSHEDPEVKFNWYVDGVEVH |
| | (T366S, L368A, Y407V, | NAKTKPREEQYNSTYRVVSVLTVLHQDWLNGKEYKCKVSNKALPAPIEKTISKAKGQPR |
| | Y349C) | EPQVCTLPPSRDELTKNQVSLSCAVKGFYPSDIAVEWESNGQPENNYKTTPPVLDSDG |
| | | SFFLVSKLTVDKSRWQQGNVFSCSVMHEALHNHYTQKSLSLSPGK |
| 233 | P1A3_AQ(scFv)- | QVQLQAWGAGLLKPSETLSLTCAVYGGSFSGYYWSWIRQPPGKGLEWIGEINHSGSTN |
| | CH2(LALA)- | YNPSLKSRATISVDTSKNQFSLKLSSVTAADTAVYYCATSPGGYSGGYFQHWGQGTQV |
| | CH3(T366S, L368A, | TVSSGGGGSGGGGSGGGGSDVVMTQSPLSLPVTPGEPASISCRSSQSLLHSNGYNYL |
| | Y407V, Y349C) | DWYLQKPGQSPQLLIYLGSNRDSGVPDRFSGSGSGTDFTLKISRVEAEDVGVYYCMQG |
| | | THWPWTFGQGTKVEIKNSGAGTAAATHTCPPCPAPEAAGGPSVFLFPPKPKDTLMISRT |
| | | PEVTCVVVDVSHEDPEVKFNWYVDGVEVHNAKTKPREEQYNSTYRVVSVLTVLHQDWL |
| | | NGKEYKCKVSNKALPAPIEKTISKAKGQPREPQVCTLPPSRDELTKNQVSLSCAVKGFYP |
| | | SDIAVEWESNGQPENNYKTTPPVLDSDGSFFLVSKLTVDKSRWQQGNVFSCSVMHEAL |
| | | HNHYTQKSLSLSPGK |
| 234 | P1A10(scFv)-CH2(LALA)- | QVQLQQSGAEVKKPGSSVKVSCKASGGTFSSYAISWVRQAPGQGLEWMGGFDPEDG |
| | CH3(T366S, L368A, | ETIYAQKFQGRVTMTEDTSTDTAYMELSSLRSEDTAVYYCATDLRIPYYYDNPWGQGTL |
| | Y407V, Y349C) | VTVSSASVAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGECDKTHTC |
| | | PPCPAPEAAGGPSVFLFPPKPKDTLMISRTPEVTCVVVDVSHEDPEVKFNWYVDGVEVH |
| | | NAKTKPREEQYNSTYRVVSVLTVLHQDWLNGKEYKCKVSNKALPAPIEKTISKAKGQPR |
| | | EPQVCTLPPSRDELTKNQVSLSCAVKGFYPSDIAVEWESNGQPENNYKTTPPVLDSDG |
| | | SFFLVSKLTVDKSRWQQGNVFSCSVMHEALHNHYTQKSLSLSPGK |
| 235 | P1A3(VH)-CH1- | QVQLQQWGAGLLKPSETLSLTCAVYGGSFSGYYWSWIRQPPGKGLEWIGEINHSGSTN |
| | CH2(LALA)- | YNPSLKSRATISVDTSKNQFSLKLSSVTAADTAVYYCATSPGGYSGGYFQHWGQGTLVT |
| | CH3(T366S, L368A, | VSSASTKGPSVFPLAPSSKSTSGGTAALGCLVKDYFPEPVTVSWNSGALTSGVHTFPAV |
| | Y407V, Y349C) | LQSSGLYSLSSVVTVPSSSLGTQTYICNVNHKPSNTKVDKKVEPKSCDKTHTCPPCPAP |
| | | EAAGGPSVFLFPPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAKGQPREPQVCT |
| | | LPPSRDELTKNQVSLSCAVKGFYPSDIAVEWESNGQPENNYKTTPPVLDSDGSFFLVSK |
| | | LTVDKSRWQQGNVFSCSVMHEALHNHYTQKSLSLSPGK |
| 236 | P1A3(VL)-Cκ | DVVMTQSPLSLPVTPGEPASISCRSSQSLLHSNGYNYLDWYLQKPGQSPQLLIYLGSNR |
| | | DSGVPDRFSGSGSGTDFTLKISRVEAEDVGVYYCMQGTHWPWTFGQGTKVEIKRTVAA |
| | | PSVFIFPPSDEQLKSGTASVVCLLNNFYPREAKVQWKVDNALQSGNSQESVTEQDSKD |
| | | STYSLSSTLTLSKADYEKHKVYACEVTHQGLSSPVTKSFNRGEC |
| 237 | P1A10(VH)-CH1- | QVQLQQSGAEVKKPGSSVKVSCKASGGTFSSYAISWVRQAPGQGLEWMGGFDPEDG |
| | CH2(LALA)- | ETIYAQKFQGRVTMTEDTSTDTAYMELSSLRSEDTAVYYCATDLRIPYYYDNPWGQGTL |
| | CH3(T366S, L368A, | VTVSSASTKGPSVFPLAPSSKSTSGGTAALGCLVKDYFPEPVTVSWNSGALTSGVHTFP |
| | Y407V, Y349C) | AVLQSSGLYSLSSVVTVPSSSLGTQTYICNVNHKPSNTKVDKKVEPKSCDKTHTCPPCP |
| | | APEAAGGPSVFLFPPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEKTISKAKGQPREPQV |
| | | CTLPPSRDELTKNQVSLSCAVKGFYPSDIAVEWESNGQPENNYKTTPPVLDSDGSFFLV |
| | | SKLTVDKSRWQQGNVFSCSVMHEALHNHYTQKSLSLSPGK |
| 238 | P1A10(VL)-Cκ | EIVLTQSPLSLPVTPGEPASISCRSSQSLLHSNGYNYLNWYLQKPGQSPQLLIYLGSDRA |
| | | SGVPDRFSGSGSGTDFTLKISRVEAEDVGVYYCMQALQTPTTFGGGTKVEIKRTVAAPS |
| | | VFIFPPSDEQLKSGTASVVCLLNNFYPREAKVQWKVDNALQSGNSQESVTEQDSKDST |
| | | YSLSSTLTLSKADYEKHKVYACEVTHQGLSSPVTKSFNRGEC |
| 239 | Linker 5 | GGGGSGGGGGGGGSGGGGS |
| 240 | Linker 6 (short flexible | GGGGS |
| | linker) | |
| 241 | Human CD132 (UniProt: | MLKPSLPFTSLLFLQLPLLGVG |
| | P31785) signal peptide | |
| 242 | P1A3_AQ scFv | QVQLQAWGAGLLKPSETLSLTCAVYGGSFSGYYWSWIRQPPGKGLEWIGEINHSGSTN |
| | | YNPSLKSRATISVDTSKNQFSLKLSSVTAADTAVYYCATSPGGYSGGYFQHWGQGTQV |
| | | TVSSGGGGSGGGGSGGGGSDVVMTQSPLSLPVTPGEPASISCRSSQSLLHSNGYNYL |
| | | DWYLQKPGQSPQLLIYLGSNRDSGVPDRFSGSGSGTDFTLKISRVEAEDVGVYYCMQG |
| | | THWPWTFGQGTKVEIK |
| 243 | P1A3_AQ(scFv)- | QVQLQAWGAGLLKPSETLSLTCAVYGGSFSGYYWSWIRQPPGKGLEWIGEINHSGSTN |
| | P2C4FW2(scFv)- | YNPSLKSRATISVDTSKNQFSLKLSSVTAADTAVYYCATSPGGYSGGYFQHWGQGTQV |
| | CH2(LALA)-CH3 | TVSSGGGGSGGGGSGGGGSDVVMTQSPLSLPVTPGEPASISCRSSQSLLHSNGYNYL |
| | | DWYLQKPGQSPQLLIYLGSNRDSGVPDRFSGSGSGTDFTLKISRVEAEDVGVYYCMQG |
| | | THWPWTFGQGTKVEIKNSGAGTAAAEVQLVQSGAEVKKPGASVKVSCKASGYTFTNYY |
| | | MHWVRQAPGQGLEWMGAIMPSRGGTSYPQKFQGRVTITADKSTSTAYMELSSLRSED |
| | | TAVYYCARGEYYYDSSGYYYWGQGTLVTVSSGGGGSGGGGSGGGGSQSVLTQPPSV |
| | | SGAPGQRVTISCTGTSSDIGHYDFVSWYQQLPGTAPKLLIYDINNRPSGVPDRFSGSKS |
| | | GTSASLAITGLQAEDEADYYCSAYTSSDTLVFGGGTKLTVLNSGAGTAAATHTCPPCPA |
| | | PEAAGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| 244 | P1A10(scFv)- | QVQLQQSGAEVKKPGSSVKVSCKASGGTFSSYAISWVRQAPGQGLEWMGGFDPEDG |
| | P2C4FW2(scFv)- | ETIYAQKFQGRVTMTEDTSTDTAYMELSSLRSEDTAVYYCATDLRIPYYYDNPWGQGTL |
| | CH2(LALA)-CH3 | VTVSSGGGGSGGGGSGGGGSEIVLTQSPLSLPVTPGEPASISCRSSQSLLHSNGYNYL |
| | | NWYLQKPGQSPQLLIYLGSDRASGVPDRFSGSGSGTDFTLKISRVEAEDVGVYYCMQA |
| | | LQTPTTFGGGTKVEIKNSGAGTAAAEVQLVQSGAEVKKPGASVKVSCKASGYTFTNYY |
| | | MHWVRQAPGQGLEWMGAIMPSRGGTSYPQKFQGRVTITADKSTSTAYMELSSLRSED |
| | | TAVYYCARGEYYYDSSGYYYWGQGTLVTVSSGGGGSGGGGSGGGGSQSVLTQPPSV |
| | | SGAPGQRVTISCTGTSSDIGHYDFVSWYQQLPGTAPKLLIYDINNRPSGVPDRFSGSKS |
| | | GTSASLAITGLQAEDEADYYCSAYTSSDTLVFGGGTKLTVLNSGAGTAAATHTCPPCPA |
| | | PEAAGGPSVFLFPPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTPPVLDSDGSFFLYS |
| | | KLTVDKSRWQQGNVFSCSVMHEALHNHYTQKSLSLSPGK |
| 245 | Human IL-2Rα (UniProt | MDSYLLMWGLLTFIMVPGCQAELCDDDPPEIPHATFKAMAYKEGTMLNCECKRGFRRIK |
| | P01589) | SGSLYMLCTGNSSHSSWDNQCQCTSSATRNTTKQVTPQPEEQKERKTTEMQSPMQPV |
| | | DQASLPGHCREPPPWENEATERIYHFVVGQMVYYQCVQGYRALHRGPAESVCKMTHG |
| | | KTRWTQPQLICTGEMETSQFPGEEKPQASPEGRPESETSCLVTTTDFQIQTEMAATMET |
| | | SIFTTEYQVAVAGCVFLLISVLLLSGLTWQRRQRKSRRTI |
| 246 | Human IL-2Rα | ELCDDDPPEIPHATFKAMAYKEGTMLNCECKRGFRRIKSGSLYMLCTGNSSHSSWDNQ |
| | extracellular domain | CQCTSSATRNTTKQVTPQPEEQKERKTTEMQSPMQPVDQASLPGHCREPPPWENEAT |
| | | ERIYHFVVGQMVYYQCVQGYRALHRGPAESVCKMTHGKTRWTQPQLICTGEMETSQF |
| | | PGEEKPQASPEGRPESETSCLVTTTDFQIQTEMAATMETSIFTTEYQ |
| 247 | Human IL-2Rα | VAVAGCVFLLISVLLLSGL |
| | transmembrane domain | |
| 248 | Human IL-2Rα | TWQRRQRKSRRTI |
| | cytoplasmic domain | |
| 249 | Human IL-15Rα | MAPRRARGCRTLGLPALLLLLLLRPPATRGITCPPPMSVEHADIWVKSYSLYSRERYICN |
| | canonical isoform | SGFKRKAGTSSLTECVLNKATNVAHWTTPSLKCIRDPALVHQRPAPPSTVTTAGVTPQP |
| | (Uniprot Q13261-1) | ESLSPSGKEPAASSPSSNNTAATTAAIVPGSQLMPSKSPSTGTTEISSHESSHGTPSQTT |
| | | AKNWELTASASHQPPGVYPQGHSDTTVAISTSTVLLCGLSAVSLLACYLKSRQTPPLASV |
| | | EMEAMEALPVTWGTSSRDEDLENCSHHL |
| 250 | Transmembrane | IPWLGHLLVGLSGAFGFIILVYLLI |
| | domain of Human | |
| | CD122 (UniProt: | |
| | P14784-1, v1 residues | |
| | 241 to 265) | |
| 251 | Cytoplasmic domain of | NCRNTGPWLKKVLKCNTPDPSKFFSQLSSEHGGDVQKWLSSPFPSSSFSPGGLAPEIS |
| | Human CD122 | PLEVLERDKVTQLLLQQDKVPEPASLSSNHSLTSCFTNQGYFFFHLPDALEIEACQVYFT |
| | (UniProt: P14784-1, v1 | YDPYSEEDPDEGVAGAPTGSSPQPLQPLSGEDDAYCTFPSRDDLLLFSPSLLGGPSPPS |
| | residues 266 to 551) | TAPGGSGAGEERMPPSLQERVPRDWDPQPLGPPTPGVPDLVDFQPPPELVLREAGEE |
| | | VPDAGPREGVSFPWSRPPGQGEFRALNARLPLNTDAYLSLQELQGQDPTHLV |
| 252 | Human IL-2Rα (UniProt | MDSYLLMWGLLTFIMVPGCQA |
| | P01589) signal peptide | |
| 253 | Human IL-2Rα (UniProt | ELCDDDPPEIPHATFKAMAYKEGTMLNCECKRGFRRIKSGSLYMLCTGNSSHSSWDNQ |
| | P01589) mature form | CQCTSSATRNTTKQVTPQPEEQKERKTTEMQSPMQPVDQASLPGHCREPPPWENEAT |
| | | ERIYHFVVGQMVYYQCVQGYRALHRGPAESVCKMTHGKTRWTQPQLICTGEMETSQF |
| | | PGEEKPQASPEGRPESETSCLVTTTDFQIQTEMAATMETSIFTTEYQVAVAGCVFLLISVL |
| | | LLSGLTWQRRQRKSRRTI |
| 254 | Human IL-15Rα | MAPRRARGCRTLGLPALLLLLLLRPPATRG |
| | (Uniprot Q13261) signal | |
| | peptide | |
| 255 | Human IL-15Rα | ITCPPPMSVEHADIWVKSYSLYSRERYICNSGFKRKAGTSSLTECVLNKATNVAHWTTPS |
| | (Uniprot Q13261) | LKCIRDPALVHQRPAPPSTVTTAGVTPQPESLSPSGKEPAASSPSSNNTAATTAAIVPGS |
| | mature form | QLMPSKSPSTGTTEISSHESSHGTPSQTTAKNWELTASASHQPPGVYPQGHSDTTVAIS |
| | | TSTVLLCGLSAVSLLACYLKSRQTPPLASVEMEAMEALPVTWGTSSRDEDLENCSHHL |
| 256 | Human CD132 | VVISVGSMGLIISLLCVYFWL |
| | (UniProt: P31785) | |
| | transmembrane domain | |
| 257 | Human CD132 | ERTMPRIPTLKNLEDLVTEYHGNFSAWSGVSKGLAESLQPDYSERLCLVSEIPPKGGAL |
| | (UniProt: P31785) | GEGPGASPCNQHSPYWAPPCYTLKPET |
| | cytoplasmic domain | |
| 258 | Human IL-15Rα | ITCPPPMSVEHADIWVKSYSLYSRERYICNSGFKRKAGTSSLTECVLNKATNVAHWTTPS |
| | (Uniprot Q13261) | LKCIRDPALVHQRPAPPSTVTTAGVTPQPESLSPSGKEPAASSPSSNNTAATTAAIVPGS |
| | extracellular domain | QLMPSKSPSTGTTEISSHESSHGTPSQTTAKNWELTASASHQPPGVYPQGHSDTT |
| 259 | Human IL-15Rα | VAISTSTVLLCGLSAVSLLACYL |
| | (Uniprot Q13261) | |
| | transmembrane domain | |
| 260 | Human IL-15Rα | KSRQTPPLASVEMEAMEALPVTWGTSSRDEDLENCSHHL |
| | (Uniprot Q13261) | |
| | cytoplasmic domain | |
| 261 | Human IL-4Rα | MGWLCSGLLFPVSCLVLLQVASSGNMKVLQEPTCVSDYMSISTCEWKMNGPTNCSTEL |
| | canonical isoform | RLLYQLVFLLSEAHTCIPENNGGAGCVCHLLMDDVVSADNYTLDLWAGQQLLWKGSFK |
| | (Uniprot P24394-1) | PSEHVKPRAPGNLTVHTNVSDTLLLTWSNPYPPDNYLYNHLTYAVNIWSENDPADFRIY |
| | | NVTYLEPSLRIAASTLKSGISYRARVRAWAQCYNTTWSEWSPSTKWHNSYREPFEQHLL |
| | | LGVSVSCIVILAVCLLCYVSITKIKKEWWDQIPNPARSRLVAIIIQDAQGSQWEKRSRGQE |
| | | PAKCPHWKNCLTKLLPCFLEHNMKRDEDPHKAAKEMPFQGSGKSAWCPVEISKTVLWP |
| | | ESISVVRCVELFEAPVECEEEEEVEEEKGSFCASPESSRDDFQEGREGIVARLTESLFLD |
| | | LLGEENGGFCQQDMGESCLLPPSGSTSAHMPWDEFPSAGPKEAPPWGKEQPLHLEPS |
| | | PPASPTQSPDNLTCTETPLVIAGNPAYRSFSNSLSQSPCPRELGPDPLLARHLEEVEPE |
| | | MPCVPQLSEPTTVPQPEPETWEQILRRNVLQHGAAAAPVSAPTSGYQEFVHAVEQGGT |
| | | QASAVVGLGPPGEAGYKAFSSLLASSAVSPEKCGFGASSGEEGYKPFQDLIPGCPGDP |
| | | APVPVPLFTFGLDREPPRSPQSSHLPSSSPEHLGLEPGEKVEDMPKPPLPQEQATDPLV |
| | | DSLGSGIVYSALTCHLCGHLKQCHGQEDGGQTPVMASPCCGCCCGDRSSPPTTPLRA |
| | | PDPSPGGVPLEASLCPASLAPSGISEKSKSSSSFHPAPGNAQSSSQTPKIVNFVSVGPTY |
| | | MRVS |
| 262 | Human IL-4Rα (Uniprot | MGWLCSGLLFPVSCLVLLQVASSGN |
| | P24394) signal peptide | |
| 263 | Human IL-4Rα (Uniprot | MKVLQEPTCVSDYMSISTCEWKMNGPTNCSTELRLLYQLVFLLSEAHTCIPENNGGAGC |
| | P24394) mature form | VCHLLMDDVVSADNYTLDLWAGQQLLWKGSFKPSEHVKPRAPGNLTVHTNVSDTLLLT |
| | | WSNPYPPDNYLYNHLTYAVNIWSENDPADFRIYNVTYLEPSLRIAASTLKSGISYRARVR |
| | | AWAQCYNTTWSEWSPSTKWHNSYREPFEQHLLLGVSVSCIVILAVCLLCYVSITKIKKEW |
| | | WDQIPNPARSRLVAIIIQDAQGSQWEKRSRGQEPAKCPHWKNCLTKLLPCFLEHNMKR |
| | | DEDPHKAAKEMPFQGSGKSAWCPVEISKTVLWPESISVVRCVELFEAPVECEEEEEVEE |
| | | EKGSFCASPESSRDDFQEGREGIVARLTESLFLDLLGEENGGFCQQDMGESCLLPPSG |
| | | STSAHMPWDEFPSAGPKEAPPWGKEQPLHLEPSPPASPTQSPDNLTCTETPLVIAGNP |
| | | AYRSFSNSLSQSPCPRELGPDPLLARHLEEVEPEMPCVPQLSEPTTVPQPEPETWEQIL |
| | | RRNVLQHGAAAAPVSAPTSGYQEFVHAVEQGGTQASAVVGLGPPGEAGYKAFSSLLAS |
| | | SAVSPEKCGFGASSGEEGYKPFQDLIPGCPGDPAPVPVPLFTFGLDREPPRSPQSSHLP |
| | | SSSPEHLGLEPGEKVEDMPKPPLPQEQATDPLVDSLGSGIVYSALTCHLCGHLKQCHG |
| | | QEDGGQTPVMASPCCGCCCGDRSSPPTTPLRAPDPSPGGVPLEASLCPASLAPSGISE |
| | | KSKSSSSFHPAPGNAQSSSQTPKIVNFVSVGPTYMRVS |
| 264 | Human IL-4Rα (Uniprot | MKVLQEPTCVSDYMSISTCEWKMNGPTNCSTELRLLYQLVFLLSEAHTCIPENNGGAGC |
| | P24394) extracellular | VCHLLMDDVVSADNYTLDLWAGQQLLWKGSFKPSEHVKPRAPGNLTVHTNVSDTLLLT |
| | domain | WSNPYPPDNYLYNHLTYAVNIWSENDPADFRIYNVTYLEPSLRIAASTLKSGISYRARVR |
| | | AWAQCYNTTWSEWSPSTKWHNSYREPFEQH |
| 265 | Human IL-4Rα (Uniprot | LLLGVSVSCIVILAVCLLCYVSIT |
| | P24394) | |
| | transmembrane domain | |
| 266 | Human IL-4Rα (Uniprot | KIKKEWWDQIPNPARSRLVAIIIQDAQGSQWEKRSRGQEPAKCPHWKNCLTKLLPCFLE |
| | P24394) cytoplasmic | HNMKRDEDPHKAAKEMPFQGSGKSAWCPVEISKTVLWPESISVVRCVELFEAPVECEE |
| | domain | EEEVEEEKGSFCASPESSRDDFQEGREGIVARLTESLFLDLLGEENGGFCQQDMGESC |
| | | LLPPSGSTSAHMPWDEFPSAGPKEAPPWGKEQPLHLEPSPPASPTQSPDNLTCTETPL |
| | | VIAGNPAYRSFSNSLSQSPCPRELGPDPLLARHLEEVEPEMPCVPQLSEPTTVPQPEPE |
| | | TWEQILRRNVLQHGAAAAPVSAPTSGYQEFVHAVEQGGTQASAVVGLGPPGEAGYKAF |
| | | SSLLASSAVSPEKCGFGASSGEEGYKPFQDLIPGCPGDPAPVPVPLFTFGLDREPPRSP |
| | | QSSHLPSSSPEHLGLEPGEKVEDMPKPPLPQEQATDPLVDSLGSGIVYSALTCHLCGHL |
| | | KQCHGQEDGGQTPVMASPCCGCCCGDRSSPPTTPLRAPDPSPGGVPLEASLCPASLA |
| | | PSGISEKSKSSSSFHPAPGNAQSSSQTPKIVNFVSVGPTYMRVS |
| 267 | Human IL-9Rα | MGLGRCIWEGWTLESEALRRDMGTWLLACICICTCVCLGVSVTGEGQGPRSRTFTCLT |
| | canonical isoform | NNILRIDCHWSAPELGQGSSPWLLFTSNQAPGGTHKCILRGSECTVVLPPEAVLVPSDN |
| | (Uniprot Q01113-1) | FTITFHHCMSGREQVSLVDPEYLPRRHVKLDPPSDLQSNISSGHCILTWSISPALEPMTTL |
| | | LSYELAFKKQEEAWEQAQHRDHIVGVTWLILEAFELDPGFIHEARLRVQMATLEDDVVEE |
| | | ERYTGQWSEWSQPVCFQAPQRQGPLIPPWGWPGNTLVAVSIFLLLTGPTYLLFKLSPR |
| | | VKRIFYQNVPSPAMFFQPLYSVHNGNFQTWMGAHGAGVLLSQDCAGTPQGALEPCVQ |
| | | EATALLTCGPARPWKSVALEEEQEGPGTRLPGNLSSEDVLPAGCTEWRVQTLAYLPQE |
| | | DWAPTSLTRPAPPDSEGSRSSSSSSSSNNNNYCALGCYGGWHLSALPGNTQSSGPIPA |
| | | LACGLSCDHQGLETQQGVAWVLAGHCQRPGLHEDLQGMLLPSVLSKARSWTF |
| 268 | Human IL-9Rα (Uniprot | MGLGRCIWEGWTLESEALRRDMGTWLLACICICTCVCLGV |
| | Q01113) signal peptide | |
| 269 | Human IL-9Rα (Uniprot | SVTGEGQGPRSRTFTCLTNNILRIDCHWSAPELGQGSSPWLLFTSNQAPGGTHKCILRG |
| | Q01113) mature form | SECTVVLPPEAVLVPSDNFTITFHHCMSGREQVSLVDPEYLPRRHVKLDPPSDLQSNISS |
| | | GHCILTWSISPALEPMTTLLSYELAFKKQEEAWEQAQHRDHIVGVTWLILEAFELDPGFIH |
| | | EARLRVQMATLEDDVVEEERYTGQWSEWSQPVCFQAPQRQGPLIPPWGWPGNTLVA |
| | | VSIFLLLTGPTYLLFKLSPRVKRIFYQNVPSPAMFFQPLYSVHNGNFQTWMGAHGAGVLL |
| | | SQDCAGTPQGALEPCVQEATALLTCGPARPWKSVALEEEQEGPGTRLPGNLSSEDVLP |
| | | AGCTEWRVQTLAYLPQEDWAPTSLTRPAPPDSEGSRSSSSSSSSNNNNYCALGCYGG |
| | | WHLSALPGNTQSSGPIPALACGLSCDHQGLETQQGVAWVLAGHCQRPGLHEDLQGML |
| | | LPSVLSKARSWTF |
| 270 | Human IL-9Rα (Uniprot | SVTGEGQGPRSRTFTCLTNNILRIDCHWSAPELGQGSSPWLLFTSNQAPGGTHKCILRG |
| | Q01113) extracellular | SECTVVLPPEAVLVPSDNFTITFHHCMSGREQVSLVDPEYLPRRHVKLDPPSDLQSNISS |
| | domain | GHCILTWSISPALEPMTTLLSYELAFKKQEEAWEQAQHRDHIVGVTWLILEAFELDPGFIH |
| | | EARLRVQMATLEDDVVEEERYTGQWSEWSQPVCFQAPQRQGPLIPPWGWP |
| 271 | Human IL-9Rα (Uniprot | GNTLVAVSIFLLLTGPTYLLF |
| | Q01113) | |
| | transmembrane domain | |
| 272 | Human IL-9Rα (Uniprot | KLSPRVKRIFYQNVPSPAMFFQPLYSVHNGNFQTWMGAHGAGVLLSQDCAGTPQGALE |
| | Q01113) cytoplasmic | PCVQEATALLTCGPARPWKSVALEEEQEGPGTRLPGNLSSEDVLPAGCTEWRVQTLAY |
| | domain | LPQEDWAPTSLTRPAPPDSEGSRSSSSSSSSNNNNYCALGCYGGWHLSALPGNTQSS |
| | | GPIPALACGLSCDHQGLETQQGVAWVLAGHCQRPGLHEDLQGMLLPSVLSKARSWTF |
| 273 | Human IL-21Rα | MPRGWAAPLLLLLLQGGWGCPDLVCYTDYLQTVICILEMWNLHPSTLTLTWQDQYEELK |
| | canonical isoform | DEATSCSLHRSAHNATHATYTCHMDVFHFMADDIFSVNITDQSGNYSQECGSFLLAESIK |
| | (Uniprot Q9HBE5) | PAPPFNVTVTFSGQYNISWRSDYEDPAFYMLKGKLQYELQYRNRGDPWAVSPRRKLIS |
| | | VDSRSVSLLPLEFRKDSSYELQVRAGPMPGSSYQGTWSEWSDPVIFQTQSEELKEGW |
| | | NPHLLLLLLLVIVFIPAFWSLKTHPLWRLWKKIWAVPSPERFFMPLYKGCSGDFKKWVGA |
| | | PFTGSSLELGPWSPEVPSTLEVYSCHPPRSPAKRLQLTELQEPAELVESDGVPKPSFWP |
| | | TAQNSGGSAYSEERDRPYGLVSIDTVTVLDAEGPCTWPCSCEDDGYPALDLDAGLEPS |
| | | PGLEDPLLDAGTTVLSCGCVSAGSPGLGGPLGSLLDRLKPPLADGEDWAGGLPWGGR |
| | | SPGGVSESEAGSPLAGLDMDTFDSGFVGSDCSSPVECDFTSPGDEGPPRSYLRQWVVI |
| | | PPPLSSPGPQAS |
| 274 | Human IL-21Rα | CPDLVCYTDYLQTVICILEMWNLHPSTLTLTWQDQYEELKDEATSCSLHRSAHNATHAT |
| | (Uniprot Q9HBE5) | YTCHMDVFHFMADDIFSVNITDQSGNYSQECGSFLLAESIKPAPPFNVTVTFSGQYNISW |
| | extracellular domain | RSDYEDPAFYMLKGKLQYELQYRNRGDPWAVSPRRKLISVDSRSVSLLPLEFRKDSSYE |
| | | LQVRAGPMPGSSYQGTWSEWSDPVIFQTQSEELKE |
| 275 | Human IL-21Rα | GWNPHLLLLLLLVIVFIPAFW |
| | (Uniprot Q9HBE5) | |
| | transmembrane domain | |
| 276 | Human IL-21Rα | SLKTHPLWRLWKKIWAVPSPERFFMPLYKGCSGDFKKWVGAPFTGSSLELGPWSPEVP |
| | (Uniprot Q9HBE5) | STLEVYSCHPPRSPAKRLQLTELQEPAELVESDGVPKPSFWPTAQNSGGSAYSEERDR |
| | cytoplasmic domain | PYGLVSIDTVTVLDAEGPCTWPCSCEDDGYPALDLDAGLEPSPGLEDPLLDAGTTVLSC |
| | | GCVSAGSPGLGGPLGSLLDRLKPPLADGEDWAGGLPWGGRSPGGVSESEAGSPLAGL |
| | | DMDTFDSGFVGSDCSSPVECDFTSPGDEGPPRSYLRQWVVIPPPLSSPGPQAS |
| 277 | Human IL-7Rα | MTILGTTFGMVFSLLQVVSGESGYAQNGDLEDAELDDYSFSCYSQLEVNGSQHSLTCAF |
| | canonical isoform | EDPDVNITNLEFEICGALVEVKCLNFRKLQEIYFIETKKFLLIGKSNICVKVGEKSLTCKKID |
| | (Uniprot P16871-1) | LTTIVKPEAPFDLSVVYREGANDFVVTFNTSHLQKKYVKVLMHDVAYRQEKDENKWTHV |
| | | NLSSTKLTLLQRKLQPAAMYEIKVRSIPDHYFKGFWSEWSPSYYFRTPEINNSSGEMDPI |
| | | LLTISILSFFSVALLVILACVLWKKRIKPIVWPSLPDHKKTLEHLCKKPRKNLNVSFNPESFL |
| | | DCQIHRVDDIQARDEVEGFLQDTFPQQLEESEKQRLGGDVQSPNCPSEDVVITPESFGR |
| | | DSSLTCLAGNVSACDAPILSSSRSLDCRESGKNGPHVYQDLLLSLGTTNSTLPPPFSLQS |
| | | GILTLNPVAQGQPILTSLGSNQEEAYVTMSSFYQNQ |
| 278 | Human IL-21Rα | MPRGWAAPLLLLLLQGGWG |
| | (Uniprot Q9HBE5) | |
| | signal peptide | |
| 279 | Human IL-21Rα | CPDLVCYTDYLQTVICILEMWNLHPSTLTLTWQDQYEELKDEATSCSLHRSAHNATHAT |
| | (Uniprot Q9HBE5) | YTCHMDVFHFMADDIFSVNITDQSGNYSQECGSFLLAESIKPAPPFNVTVTFSGQYNISW |
| | mature form | RSDYEDPAFYMLKGKLQYELQYRNRGDPWAVSPRRKLISVDSRSVSLLPLEFRKDSSYE |
| | | LQVRAGPMPGSSYQGTWSEWSDPVIFQTQSEELKEGWNPHLLLLLLLVIVFIPAFWSLK |
| | | THPLWRLWKKIWAVPSPERFFMPLYKGCSGDFKKWVGAPFTGSSLELGPWSPEVPSTL |
| | | EVYSCHPPRSPAKRLQLTELQEPAELVESDGVPKPSFWPTAQNSGGSAYSEERDRPYG |
| | | LVSIDTVTVLDAEGPCTWPCSCEDDGYPALDLDAGLEPSPGLEDPLLDAGTTVLSCGCV |
| | | SAGSPGLGGPLGSLLDRLKPPLADGEDWAGGLPWGGRSPGGVSESEAGSPLAGLDMD |
| | | TFDSGFVGSDCSSPVECDFTSPGDEGPPRSYLRQWVVIPPPLSSPGPQAS |
| 280 | Human IL-7Rα (Uniprot | MTILGTTFGMVFSLLQVVSG |
| | P16871) signal peptide | |
| 281 | Human IL-7Rα (Uniprot | ESGYAQNGDLEDAELDDYSFSCYSQLEVNGSQHSLTCAFEDPDVNITNLEFEICGALVE |
| | P16871) extracellular | VKCLNFRKLQEIYFIETKKFLLIGKSNICVKVGEKSLTCKKIDLTTIVKPEAPFDLSVVYREG |
| | domain | ANDFVVTFNTSHLQKKYVKVLMHDVAYRQEKDENKWTHVNLSSTKLTLLQRKLQPAAM |
| | | YEIKVRSIPDHYFKGFWSEWSPSYYFRTPEINNSSGEMD |
| 282 | Human IL-7Rα (Uniprot | PILLTISILSFFSVALLVILACVLW |
| | P16871) | |
| | transmembrane domain | |
| 283 | Human IL-7Rα (Uniprot | KKRIKPIVWPSLPDHKKTLEHLCKKPRKNLNVSFNPESFLDCQIHRVDDIQARDEVEGFL |
| | P16871) cytoplasmic | QDTFPQQLEESEKQRLGGDVQSPNCPSEDVVITPESFGRDSSLTCLAGNVSACDAPILS |
| | domain | SSRSLDCRESGKNGPHVYQDLLLSLGTTNSTLPPPFSLQSGILTLNPVAQGQPILTSLGS |
| | | NQEEAYVTMSSFYQNQ |
| 284 | Human IL-7Rα (Uniprot | ESGYAQNGDLEDAELDDYSFSCYSQLEVNGSQHSLTCAFEDPDVNITNLEFEICGALVE |
| | P16871) mature form | VKCLNFRKLQEIYFIETKKFLLIGKSNICVKVGEKSLTCKKIDLTTIVKPEAPFDLSVVYREG |
| | | ANDFVVTFNTSHLQKKYVKVLMHDVAYRQEKDENKWTHVNLSSTKLTLLQRKLQPAAM |
| | | YEIKVRSIPDHYFKGFWSEWSPSYYFRTPEINNSSGEMDPILLTISILSFFSVALLVILACVL |
| | | WKKRIKPIVWPSLPDHKKTLEHLCKKPRKNLNVSFNPESFLDCQIHRVDDIQARDEVEGF |
| | | LQDTFPQQLEESEKQRLGGDVQSPNCPSEDVVITPESFGRDSSLTCLAGNVSACDAPIL |
| | | SSSRSLDCRESGKNGPHVYQDLLLSLGTTNSTLPPPFSLQSGILTLNPVAQGQPILTSLG |
| | | SNQEEAYVTMSSFYQNQ |
| 285 | Human CD132 | APENLTLHKLSESQLELNWNNRFLNHCLEHLVQYRTDWDHSWTEQSVDYRHKFSLPSV |
| | (UniProt: P31785) | DGQKRYTFRVRSRFNPLCGSAQHWSEWSHPIHWGSNTSKE |
| | fibronectin type III | |
| | (FNIII) domain | |
| 286 | Human CD132 | WSEWS |
| | (UniProt: P31785) | |
| | WSXWS motif | |
| 287 | HC-FR1 Anti-CD132 | EVQLVQSGAEVKKPGASVKVSCKASGYTFT |
| | P2F10 | |
| 288 | HC-FR1 Anti-CD132 | EVQLVQSGGGVVQPGRSLRLSCAASGFTFS |
| | P1E8, P2H4 | |
| 289 | HC-FR1 Anti-CD132 | QVQLQQWGAGLLKPSETLSLTCAVYGGSFS |
| | P1A3, P1A3_B3, | |
| | P1A3_B4, P1A3_E9, | |
| | P1B12, P1C7, P1A3-Q | |
| 290 | HC-FR1 Anti-CD132 | QVQLQQWGAGMLKPSETLSLTCAVYGGSFS |
| | P1A3_E8 | |
| 291 | HC-FR1 Anti-CD132 | EVQLVESGGGLVQPGGSLRLSCAASGGSFS |
| | P1A3_FW2 | |
| 292 | HC-FR1 Anti-CD132 P2B9 | QVQLQESGPGLVKPSETLSLTCTVSGGSIS |
| 293 | HC-FR1 Anti-CD132 | QVQLQQSGAEVKKPGSSVKVSCKASGGTFS |
| | P1A10 | |
| 294 | HC-FR1 Anti-CD132 P1B6 | QVQLVQSGGGVVQPGRSLRLSCAASGFTFS |
| 295 | HC-FR1 Anti-CD132 | EVQLVETGPGLVKPSGTLSLTCAVSGGSIS |
| | P1C10 | |
| 296 | HC-FR1 Anti-CD132 P1D7 | QVQLQESGGGVVQPGRSLRLSCAASGFTFS |
| 297 | HC-FR1 Anti-CD132 P2B2 | QLQLQESGGGVVQPGRSLRLSCAASGFTFS |
| 298 | HC-FR1 Anti-CD132 P2B7 | QVQLQQWGAGLLKPSETLSLTCAVYGESFS |
| 299 | HC-FR1 Anti-CD132 | QVQLQESGPGLVKPSQTLSLTCTVSGGSIS |
| | P2D11 | |
| 300 | HC-FR1 Anti-CD132 P2D3 | QVQLQQWGAGLLKPSETLSLTCTIYGGSFS |
| 301 | HC-FR1 Anti-CD132 P1G4 | QVQLQQWGAGLLKPSETLSLTCAVYGGSLS |
| 302 | HC-FR2 Anti-CD132 | WVRQAPGQGLEWMG |
| | P1A10, P2F10, P1A10- | |
| | AQ, P1A10-ANQ | |
| 303 | HC-FR2 Anti-CD132 | WVRQAPGKGLEWVA |
| | P1B6, P1D7, P1E8, P2B2, | |
| | P2H4 | |
| 304 | HC-FR2 Anti-CD132 | WVRQPPGKGLEWIG |
| | P1C10 | |
| 305 | HC-FR2 Anti-CD132 | WVRQAPGKGLEWVS |
| | P1A3_FW2 | |
| 306 | HC-FR2 Anti-CD132 | WIRQPPGKGLEWIG |
| | P1A3, P2B9, P1A3_B3, | |
| | P1A3_B4, P1A3_E9, | |
| | P1A3_E8, P2B7, P2D3, | |
| | P1G4, P1B12, P1C7, | |
| | P1A3-AQ, P1A3-ANQ, | |
| | P1A3-A, P1A3-Q | |
| 307 | HC-FR2 Anti-CD132 | WIRQHPGQGLEWIG |
| | P2D11 | |
| 308 | HC-FR3 Anti-CD132 | RFTISRDNSKNTLYLQMNSLRAEDTAVYYCAR |
| | P1A3_FW2, P1B6, P1E8, | |
| | P2H4 | |
| 309 | HC-FR3 Anti-CD132 | RVTISVDTSKNQFSLKLSSVTAADTAVYYCAR |
| | P2B7, P1G4, P1B12 | |
| 310 | HC-FR3 Anti-CD132 | RATISVDTSKNQFSLKLSSVTAADTAVYYCAT |
| | P1A3, P1A3_B3, | |
| | P1A3_B4, P1A3_E9, | |
| | P1A3_E8, P1A3-AQ, | |
| | P1A3-A, P1A3-Q | |
| 311 | HC-FR3 Anti-CD132 | RVTMTEDTSTDTAYMELSSLRSEDTAVYYCAT |
| | P1A10, P1A10-AQ | |
| 312 | HC-FR3 Anti-CD132 | RVTISVDKSKNQFSLKLSSVTAADTAVYYCAR |
| | P1C10 | |
| 313 | HC-FR3 Anti-CD132 P1D7 | RFTISRDNSKNTVYLQMNSLRAEDTAVYYCAK |
| 314 | HC-FR3 Anti-CD132 P2B2 | RFTISRDNSKNTLYLQMNSLRAEDTAVYYCAK |
| 315 | HC-FR3 Anti-CD132 | RVTISADTSKNHFSLNLTSVTAADTAVYYCAR |
| | P2D11 | |
| 316 | HC-FR3 Anti-CD132 | RVTMTRDTSTSTVYMELSSLRSEDTAVYYCAR |
| | P2F10 | |
| 317 | HC-FR3 Anti-CD132 P2D3 | RVTISVDTSKNQFSLKLSSVTAADTAIYYCAR |
| 318 | HC-FR3 Anti-CD132 P1C7 | RVTISEDASKKQFSLTLTSVTAADTAVYYCAR |
| 319 | HC-FR3 Anti-CD132 P2B9 | SRVTISVDTSKNQFSLKLSSVTAADTAVYYCAG |
| 320 | HC-FR4 Anti-CD132 | WGQGTLVTVSS |
| | P1A3, P2B9, P1A3_B3, | |
| | P1A3_B4, P1A3_E9, | |
| | P1A3_E8, P1A3_FW2, | |
| | P1A10, P1B6, P1E8 | |
| | P2B7, P2D11, P2D3, | |
| | P1B12, P1C7, P1A3-A | |
| 321 | HC-FR4 Anti-CD132 | WGQGTMVTVSS |
| | P1C10, P1D7, P2F10, | |
| | P2H4 | |
| 322 | HC-FR4 Anti-CD132 | WGKGTTVTVSS |
| | P2B2, P1G4 | |
| 323 | LC-FR1 Anti-CD132 P1A3, | DVVMTQSPLSLPVTPGEPASISC |
| | P1A3_B3, P1A3_E8, | |
| | P1A3_E9, P2B2, P2H4, | |
| | P2D3, P1G4, P1A3-AQ, | |
| | P1A3-ANQ, P1A3-A, | |
| | P1A3-Q | |
| 324 | LC-FR1 Anti-CD132 | DIQMTQSPSSLSASVGDRVTITC |
| | P1A3_FW2 | |
| 325 | LC-FR1 Anti-CD132 | EIVLTQSPLSLPVTPGEPASISC |
| | P1A10, P1C7, P1A10-AQ, | |
| | P1A10-ANQ | |
| 326 | LC-FR1 Anti-CD132 | DVVMTQSPLSLPVTPGESVSISC |
| | P1A3 B4 | |
| 327 | LC-FR1 Anti-CD132 P2B9 | SYELTQPPSMSVSPGQTARITC |
| 328 | LC-FR1 Anti-CD132 | EIVLTQSPATLSLSPGERATLSC |
| | P1C10 | |
| 329 | LC-FR1 Anti-CD132 P1D7 | DIQMTQSPSFLSASVGDRVTITC |
| 330 | LC-FR1 Anti-CD132 P1E8 | DVVMTQSPVSLPVTLGQPASISC |
| 331 | LC-FR1 Anti-CD132 P2B7, | DVVMTQSPLSLPVTLGQPASISC |
| | P1B12 | |
| 332 | LC-FR1 Anti-CD132 | ETTLTQSPATLSVSPGERATLSC |
| | P2D11 | |
| 333 | LC-FR1 Anti-CD132 | DIVMTHTPLSLPVTPGEPASISC |
| | P2F10 | |
| 334 | LC-FR1 Anti-CD132 P1B6 | EIVMTQSPLSLPVTPGEPASISC |
| 335 | LC-FR2 Anti-CD132 P1A3, | WYLQKPGQSPQLLIY |
| | P1A3_B3, P1A3_E8, | |
| | P1A3_E9, P1A3_B4, | |
| | P1A10, P2B7, P2H4, | |
| | P2D3, P1G4, P1B12, | |
| | P1C7, P1A3-AQ, P1A3- | |
| | ANQ, P1A3-A, P1A3-Q, | |
| | P1A10-AQ, P1A10-ANQ | |
| 336 | LC-FR2 Anti-CD132 P2B9 | WYQQKPGQAPVLVIY |
| 337 | LC-FR2 Anti-CD132 P1B6, | WYLQKPGQSPQLLMY |
| | P2F10 | |
| 338 | LC-FR2 Anti-CD132 P1E8 | WFQQRPGQSPRRLFY |
| 339 | LC-FR2 Anti-CD132 | WYQQKPGQAPRLLIY |
| | P1C10, P2D11 | |
| 340 | LC-FR2 Anti-CD132 P2B2 | WYLQKPGQSPHLLIY |
| 341 | LC-FR3 Anti-CD132 P1A3, | GVPDRFSGSGSGTDFTLKISRVEAEDVGVYYC |
| | P1A3_B3, P1A3_E8, | |
| | P1A3_E9, P1A3_B4 | |
| | P1A10, P2B7, P2F10, | |
| | P2H4, P2D3, P1G4, P1C7, | |
| | P1A3-AQ, P1A3-ANQ, | |
| | P1A3-A, P1A3-Q, P1A10- | |
| | AQ, P1A10-ANQ | |
| 342 | LC-FR3 Anti-CD132 | GVPSRFSGSGSGTDFTLTISSLQPEDFATYYC |
| | P1A3 FW2 | |
| 343 | LC-FR3 Anti-CD132 P2B9 | GIPERFSGSSSGTTVTLTITGVQAEDEADYYC |
| 344 | LC-FR3 Anti-CD132 P1B6 | GVPERFSGSGSGTDFTLKISRVEAEDVGVYYC |
| 345 | LC-FR3 Anti-CD132 | GIPARFSGSGSGTDFTLTINSLEPEDFAVYYC |
| | P1C10 | |
| 346 | LC-FR3 Anti-CD132 P1D7 | GVPSRFSGTGFGTDFTFTITTLQPDDIATYYC |
| 347 | LC-FR3 Anti-CD132 P1E8 | GVPDRFSGSGSDTDFTLTISRVEAEDVGVYFC |
| 348 | LC-FR3 Anti-CD132 P2B2 | GVPDRFSGSGSGTDFTLKISRVEAEDVGVYFC |
| 349 | LC-FR3 Anti-CD132 | GIPDRFSGSGSGTDFTLTISRLEPEDFAVYYC |
| | P2D11 | |
| 350 | LC-FR3 Anti-CD132 | GVPDRFSGSGSGTDFTLKISRVEAEDVGIYYC |
| | P1B12 | |
| 351 | LC-FR4 Anti-CD132 | FGGGTKVEIK |
| | P1A10, P1C10, P2D11, | |
| | P1A10-AQ, P1A10-ANQ | |
| 352 | LC-FR4 Anti-CD132 P2B9 | FGGGTKLTVL |
| 353 | LC-FR4 Anti-CD132 P1B6, | FGQGTKLEIK |
| | P2B2 | |
| 354 | LC-FR4 Anti-CD132 P1A3, | FGQGTKVEIK |
| | P1A3_B3, P1A3_E8, | |
| | P1A3_E9, P1A3_B4, | |
| | P1A3_FW2, P1E8, P2B7, | |
| | P2D3, P1G4, P1A3-AQ, | |
| | P1A3-ANQ, P1A3-A, | |
| | P1A3-Q | |
| 355 | LC-FR4 Anti-CD132 P1D7 | FGQGTTVDIK |
| 356 | LC-FR4 Anti-CD132 | FGQGTRLEFK |
| | P2F10 | |
| 357 | LC-FR4 Anti-CD132 P2H4 | FGPGTKVDIK |
| 358 | LC-FR4 Anti-CD132 | FGQGTKVEIE |
| | P1B12 | |
| 359 | LC-FR2 Anti-CD132 | WYQQKPGKAPKLLIY |
| | P1A3 FW2, P1D7 | |
| 360 | Rigid linker | AEAAAKEAAAKEAAAKEAAAKEAAAKA |
| 361 | Flexible linker 2 | GGGGSGGGS |
| 362 | Flexible linker 3 | GGGGSGGGSGGGS |
| 363 | Flexible linker 4 | GGGSG |
| 364 | Rigid linker 2 | EAAAK |
| 365 | Rigid linker 3 | AEAAAKEAAAKA |
| 366 | Rigid linker 4 | AEAAAKEAAAKEAAAKA |
| 367 | Rigid linker 5 | AEAAAKEAAAKEAAAKEAAAKA |
| 368 | Linker-HA tag-His tag | AAAYPYDVPDYGSHHHHHH |
| 369 | Linker | NSGAAA |
| 370 | CDR1 anti-CD132 | GGPSGIST |
| | 2RGT38 | |
| 371 | CDR2 anti-CD132 | INWSGEST |
| | 2RGT38 | |
| 372 | CDR3 anti-CD132 | TTTRDAGTRLGSRWYGPRTSAYEN |
| | 2RGT38 | |
| 373 | FR1 anti-CD132 2RGT38 | QVQLQESGGGLVQAGGSLRLSCAAS |
| 374 | FR2 anti-CD132 2RGT38 | VAWFRQAPGKEREFVAA |
| 375 | FR3 anti-CD132 2RGT38 | NYEDSVLGRFTISRDNAKNTVFLQMNSLTPEDTAVYVC |
| 376 | FR4 anti-CD132 2RGT38 | WGQGTQVTVSS |
| 377 | VHH anti-CD132 2RGT38 | QVQLQESGGGLVQAGGSLRLSCAASGGPSGISTVAWFRQAPGKEREFVAAINWSGEST |
| | | NYEDSVLGRFTISRDNAKNTVFLQMNSLTPEDTAVYVCTTTRDAGTRLGSRWYGPRTSA |
| | | YENWGQGTQVTVSS |
| 378 | CDR1 anti-IL-9Rα | GSFFNINA |
| | 2SAT363 | |
| 379 | CDR2 anti-IL-9Rα | ITAGGST |
| | 2SAT363 | |
| 380 | CDR3 anti-IL-9Rα | AAWGTRIRDDY |
| | 2SAT363 | |
| 381 | FR1 anti-IL-9Rα 2SAT363 | QVQLQESGGGLVQAGGSLRLSCTVS |
| 382 | FR2 anti-IL-9Rα 2SAT363 | MGWYRQAPGKQRELVAT |
| 383 | FR3 anti-IL-9Rα 2SAT363 | KYEDTVKGRFTISGDSAKNTVSLQMNSLKPEDTAVYYC |
| 384 | FR4 anti-IL-9Rα 2SAT363 | WGQGTQVTVSS |
| 385 | VHH anti-IL-9Rα 2SAT363 | QVQLQESGGGLVQAGGSLRLSCTVSGSFFNINAMGWYRQAPGKQRELVATITAGGSTK |
| | | YEDTVKGRFTISGDSAKNTVSLQMNSLKPEDTAVYYCAAWGTRIRDDYWGQGTQVTVS |
| | | S |
| 386 | CDR1 anti-IL-9Rα 2SAT57 | GSIFSINA |
| 387 | CDR2 anti-IL-9Rα 2SAT57 | ISSGGST |
| 388 | CDR3 anti-IL-9Rα 2SAT57 | LVATRGGTYEY |
| 389 | FR1 anti-IL-9Rα 2SAT57 | QVQLQESGGGLVQAGGSLRLSCAAS |
| 390 | FR2 anti-IL-9Rα 2SAT57 | MGWFRQAPGKQRELVAV |
| 391 | FR3 anti-IL-9Rα 2SAT57 | NHADSVKGRFTISRDNAKNTVYLQMNSVKPEDTAVYYC |
| 392 | FR4 anti-IL-9Rα 2SAT57 | WGQGTQVTVSS |
| 393 | VHH anti-IL-9Rα 2SAT57 | QVQLQESGGGLVQAGGSLRLSCAASGSIFSINAMGWFRQAPGKQRELVAVISSGGSTN |
| | | HADSVKGRFTISRDNAKNTVYLQMNSVKPEDTAVYYCLVATRGGTYEYWGQGTQVTVS |
| | | S |
| 394 | R1ST3 | QVQLQESGGGLVQAGGSLRLSCAASGGPSGISTVAWFRQAPGKEREFVAAINWSGEST |
| | (2RGT38_2SAT57_ | NYEDSVLGRFTISRDNAKNTVFLQMNSLTPEDTAVYVCTTTRDAGTRLGSRWYGPRTSA |
| | tandem_g4s) | YENWGQGTQVTVSSggggsQVQLQESGGGLVQAGGSLRLSCAASGSIFSINAMGWFRQ |
| | | APGKQRELVAVISSGGSTNHADSVKGRFTISRDNAKNTVYLQMNSVKPEDTAVYYCLVA |
| | | TRGGTYEYWGQGTQVTVSS |
| 395 | T3SR1 | QVQLQESGGGLVQAGGSLRLSCAASGSIFSINAMGWFRQAPGKQRELVAVISSGGSTN |
| | (2SAT57_2RGT38_ | HADSVKGRFTISRDNAKNTVYLQMNSVKPEDTAVYYCLVATRGGTYEYWGQGTQVTVS |
| | tandem_g4s) | SggggsQVQLQESGGGLVQAGGSLRLSCAASGGPSGISTVAWFRQAPGKEREFVAAINW |
| | | SGESTNYEDSVLGRFTISRDNAKNTVFLQMNSLTPEDTAVYVCTTTRDAGTRLGSRWY |
| | | GPRTSAYENWGQGTQVTVSS |
| 396 | R1LT3 | QVQLQESGGGLVQAGGSLRLSCAASGGPSGISTVAWFRQAPGKEREFVAAINWSGEST |
| | (2RGT38_2SAT57_ | NYEDSVLGRFTISRDNAKNTVFLQMNSLTPEDTAVYVCTTTRDAGTRLGSRWYGPRTSA |
| | tandem_eaak5) | YENWGQGTQVTVSSaeaaakeaaakeaaakeaaakeaaakaQVQLQESGGGLVQAGGSLRLS |
| | | CAASGSIFSINAMGWFRQAPGKQRELVAVISSGGSTNHADSVKGRFTISRDNAKNTVYL |
| | | QMNSVKPEDTAVYYCLVATRGGTYEYWGQGTQVTVSS |

TABLE A1
gamma chain binding moieties

| Binding | Binding | Column A | | | Column B | | |
|---|---|---|---|---|---|---|---|
| moiety | moiety | VH | | | VL | | |
| name | ID | HC-CDR1 | HC-CDR2 | HC-CDR3 | LC-CDR1 | LC-CDR2 | LC-CDR3 |
| P1A3 | A1-1 | SEQ ID | SEQ ID | SEQ ID | SEQ ID | SEQ ID | SEQ ID |
| | | NO: 38 | NO: 41 | NO: 62 | NO: 44 | NO: 88 | NO: 46 |
| P1A3_B3 | A1-2 | SEQ ID | SEQ ID | SEQ ID | SEQ ID | SEQ ID | SEQ ID |
| | | NO: 38 | NO: 55 | NO: 62 | NO: 44 | NO: 88 | NO: 46 |
| P1A3_E8 | A1-3 | SEQ ID | SEQ ID | SEQ ID | SEQ ID | SEQ ID | SEQ ID |
| | | NO: 38 | NO: 55 | NO: 62 | NO: 44 | NO: 88 | NO: 46 |
| P1A3_E9 | A1-4 | SEQ ID | SEQ ID | SEQ ID | SEQ ID | SEQ ID | SEQ ID |
| | | NO: 38 | NO: 55 | NO: 62 | NO: 44 | NO: 88 | NO: 46 |
| P1A3_B4 | A1-5 | SEQ ID | SEQ ID | SEQ ID | SEQ ID | SEQ ID | SEQ ID |
| | | NO: 38 | NO: 55 | NO: 62 | NO: 44 | NO: 88 | NO: 46 |
| P1A3_FW2 | A1-6 | SEQ ID | SEQ ID | SEQ ID | SEQ ID | SEQ ID | SEQ ID |
| | | NO: 38 | NO: 41 | NO: 62 | NO: 44 | NO: 88 | NO: 46 |
| P2B9 | A1-7 | SEQ ID | SEQ ID | SEQ ID | SEQ ID | SEQ ID | SEQ ID |
| | | NO: 47 | NO: 54 | NO: 63 | NO: 78 | NO: 89 | NO: 99 |
| P1A10 | A1-8 | SEQ ID | SEQ ID | SEQ ID | SEQ ID | SEQ ID | SEQ ID |
| | | NO: 48 | NO: 56 | NO: 64 | NO: 79 | NO: 90 | NO: 100 |
| P1B6 | A1-9 | SEQ ID | SEQ ID | SEQ ID | SEQ ID | SEQ ID | SEQ ID |
| | | NO: 39 | NO: 42 | NO: 65 | NO: 44 | NO: 91 | NO: 101 |
| P1C10 | A1-10 | SEQ ID | SEQ ID | SEQ ID | SEQ ID | SEQ ID | SEQ ID |
| | | NO: 40 | NO: 43 | NO: 66 | NO: 80 | NO: 92 | NO: 102 |
| P1D7 | A1-11 | SEQ ID | SEQ ID | SEQ ID | SEQ ID | SEQ ID | SEQ ID |
| | | NO: 49 | NO: 58 | NO: 67 | NO: 81 | NO: 93 | NO: 103 |
| P1E8 | A1-12 | SEQ ID | SEQ ID | SEQ ID | SEQ ID | SEQ ID | SEQ ID |
| | | NO: 50 | NO: 42 | NO: 68 | NO: 82 | NO: 94 | NO: 104 |
| P2B2 | A1-13 | SEQ ID | SEQ ID | SEQ ID | SEQ ID | SEQ ID | SEQ ID |
| | | NO: 39 | NO: 59 | NO: 69 | NO: 44 | NO: 45 | NO: 105 |
| P2B7 | A1-14 | SEQ ID | SEQ ID | SEQ ID | SEQ ID | SEQ ID | SEQ ID |
| | | NO: 38 | NO: 41 | NO: 70 | NO: 83 | NO: 45 | NO: 106 |
| P2D11 | A1-15 | SEQ ID | SEQ ID | SEQ ID | SEQ ID | SEQ ID | SEQ ID |
| | | NO: 51 | NO: 60 | NO: 71 | NO: 84 | NO: 95 | NO: 107 |
| P2F10 | A1-16 | SEQ ID | SEQ ID | SEQ ID | SEQ ID | SEQ ID | SEQ ID |
| | | NO: 52 | NO: 61 | NO: 72 | NO: 85 | NO: 96 | NO: 108 |
| P2H4 | A1-17 | SEQ ID | SEQ ID | SEQ ID | SEQ ID | SEQ ID | SEQ ID |
| | | NO: 39 | NO: 42 | NO: 73 | NO: 86 | NO: 45 | NO: 109 |
| P2D3 | A1-18 | SEQ ID | SEQ ID | SEQ ID | SEQ ID | SEQ ID | SEQ ID |
| | | NO: 53 | NO: 41 | NO: 74 | NO: 44 | NO: 45 | NO: 46 |
| P1G4 | A1-19 | SEQ ID | SEQ ID | SEQ ID | SEQ ID | SEQ ID | SEQ ID |
| | | NO: 38 | NO: 41 | NO: 75 | NO: 44 | NO: 45 | NO: 110 |
| P1B12 | A1-20 | SEQ ID | SEQ ID | SEQ ID | SEQ ID | SEQ ID | SEQ ID |
| | | NO: 38 | NO: 41 | NO: 76 | NO: 87 | NO: 45 | NO: 46 |
| P1C7 | A1-21 | SEQ ID | SEQ ID | SEQ ID | SEQ ID | SEQ ID | SEQ ID |
| | | NO: 38 | NO: 41 | NO: 77 | NO: 44 | NO: 97 | NO: 46 |
| P1A3_A | A1-22 | SEQ ID | SEQ ID | SEQ ID | SEQ ID | SEQ ID | SEQ ID |
| | | NO: 38 | NO: 41 | NO: 62 | NO: 44 | NO: 88 | NO: 46 |
| P1A3_Q | A1-23 | SEQ ID | SEQ ID | SEQ ID | SEQ ID | SEQ ID | SEQ ID |
| | | NO: 38 | NO: 41 | NO: 62 | NO: 44 | NO: 88 | NO: 46 |
| P1A3_AQ | A1-24 | SEQ ID | SEQ ID | SEQ ID | SEQ ID | SEQ ID | SEQ ID |
| | | NO: 38 | NO: 41 | NO: 62 | NO: 44 | NO: 88 | NO: 46 |
| P1A3_ANQ | A1-25 | SEQ ID | SEQ ID | SEQ ID | SEQ ID | SEQ ID | SEQ ID |
| | | NO: 38 | NO: 41 | NO: 62 | NO: 44 | NO: 88 | NO: 46 |
| P1A10_AQ | A1-26 | SEQ ID | SEQ ID | SEQ ID | SEQ ID | SEQ ID | SEQ ID |
| | | NO: 48 | NO: 56 | NO: 64 | NO: 79 | NO: 90 | NO: 100 |
| P1A10_ANQ | A1-27 | SEQ ID | SEQ ID | SEQ ID | SEQ ID | SEQ ID | SEQ ID |
| | | NO: 48 | NO: 56 | NO: 64 | NO: 79 | NO: 90 | NO: 100 |

TABLE B1
gamma chain binding moieties

| Binding | Binding | Column A | | | | Column B | | | |
|---|---|---|---|---|---|---|---|---|---|
| moiety | moiety | VH | | | | VL | | | |
| name | ID | HC-FR1 | HC-FR2 | HC-FR3 | HC-FR4 | LC-FR1 | LC-FR2 | LC-FR3 | LC-FR4 |
| P1A3 | B1-1 | SEQ ID | SEQ ID | SEQ ID | SEQ ID | SEQ ID | SEQ ID | SEQ ID | SEQ ID |
| | | NO: 289 | NO: 306 | NO: 310 | NO: 320 | NO: 323 | NO: 335 | NO: 341 | NO: 354 |
| P1A3_B3 | B1-2 | SEQ ID | SEQ ID | SEQ ID | SEQ ID | SEQ ID | SEQ ID | SEQ ID | SEQ ID |
| | | NO: 289 | NO: 306 | NO: 310 | NO: 320 | NO: 323 | NO: 335 | NO: 341 | NO: 354 |
| P1A3_E8 | B1-3 | SEQ ID | SEQ ID | SEQ ID | SEQ ID | SEQ ID | SEQ ID | SEQ ID | SEQ ID |
| | | NO: 290 | NO: 306 | NO: 310 | NO: 320 | NO: 323 | NO: 335 | NO: 341 | NO: 354 |
| P1A3_E9 | B1-4 | SEQ ID | SEQ ID | SEQ ID | SEQ ID | SEQ ID | SEQ ID | SEQ ID | SEQ ID |
| | | NO: 289 | NO: 306 | NO: 310 | NO: 320 | NO: 323 | NO: 335 | NO: 341 | NO: 354 |
| P1A3_B4 | B1-5 | SEQ ID | SEQ ID | SEQ ID | SEQ ID | SEQ ID | SEQ ID | SEQ ID | SEQ ID |
| | | NO: 289 | NO: 306 | NO: 310 | NO: 320 | NO: 326 | NO: 335 | NO: 341 | NO: 354 |
| P1A3_FW2 | B1-6 | SEQ ID | SEQ ID | SEQ ID | SEQ ID | SEQ ID | SEQ ID | SEQ ID | SEQ ID |
| | | NO: 291 | NO: 305 | NO: 308 | NO: 320 | NO: 324 | NO: 359 | NO: 342 | NO: 354 |
| P2B9 | B1-7 | SEQ ID | SEQ ID | SEQ ID | SEQ ID | SEQ ID | SEQ ID | SEQ ID | SEQ ID |
| | | NO: 292 | NO: 306 | NO: 319 | NO: 320 | NO: 327 | NO: 336 | NO: 343 | NO: 352 |
| P1A10 | B1-8 | SEQ ID | SEQ ID | SEQ ID | SEQ ID | SEQ ID | SEQ ID | SEQ ID | SEQ ID |
| | | NO: 293 | NO: 302 | NO: 311 | NO: 320 | NO: 325 | NO: 335 | NO: 341 | NO: 351 |
| P1B6 | B1-9 | SEQ ID | SEQ ID | SEQ ID | SEQ ID | SEQ ID | SEQ ID | SEQ ID | SEQ ID |
| | | NO: 294 | NO: 303 | NO: 308 | NO: 320 | NO: 334 | NO: 337 | NO: 344 | NO: 353 |
| P1C10 | B1-10 | SEQ ID | SEQ ID | SEQ ID | SEQ ID | SEQ ID | SEQ ID | SEQ ID | SEQ ID |
| | | NO: 295 | NO: 304 | NO: 312 | NO: 321 | NO: 328 | NO: 339 | NO: 345 | NO: 351 |
| P1D7 | B1-11 | SEQ ID | SEQ ID | SEQ ID | SEQ ID | SEQ ID | SEQ ID | SEQ ID | SEQ ID |
| | | NO: 296 | NO: 303 | NO: 313 | NO: 321 | NO: 329 | NO: 359 | NO: 346 | NO: 355 |
| P1E8 | B1-12 | SEQ ID | SEQ ID | SEQ ID | SEQ ID | SEQ ID | SEQ ID | SEQ ID | SEQ ID |
| | | NO: 288 | NO: 303 | NO: 308 | NO: 320 | NO: 330 | NO: 338 | NO: 347 | NO: 354 |
| P2B2 | B1-13 | SEQ ID | SEQ ID | SEQ ID | SEQ ID | SEQ ID | SEQ ID | SEQ ID | SEQ ID |
| | | NO: 297 | NO: 303 | NO: 314 | NO: 322 | NO: 323 | NO: 340 | NO: 348 | NO: 353 |
| P2B7 | B1-14 | SEQ ID | SEQ ID | SEQ ID | SEQ ID | SEQ ID | SEQ ID | SEQ ID | SEQ ID |
| | | NO: 298 | NO: 306 | NO: 309 | NO: 320 | NO: 331 | NO: 335 | NO: 341 | NO: 354 |
| P2D11 | B1-15 | SEQ ID | SEQ ID | SEQ ID | SEQ ID | SEQ ID | SEQ ID | SEQ ID | SEQ ID |
| | | NO: 299 | NO: 307 | NO: 315 | NO: 320 | NO: 332 | NO: 339 | NO: 349 | NO: 351 |
| P2F10 | B1-16 | SEQ ID | SEQ ID | SEQ ID | SEQ ID | SEQ ID | SEQ ID | SEQ ID | SEQ ID |
| | | NO: 287 | NO: 302 | NO: 316 | NO: 321 | NO: 333 | NO: 337 | NO: 341 | NO: 356 |
| P2H4 | B1-17 | SEQ ID | SEQ ID | SEQ ID | SEQ ID | SEQ ID | SEQ ID | SEQ ID | SEQ ID |
| | | NO: 288 | NO: 303 | NO: 308 | NO: 321 | NO: 323 | NO: 335 | NO: 341 | NO: 357 |
| P2D3 | B1-18 | SEQ ID | SEQ ID | SEQ ID | SEQ ID | SEQ ID | SEQ ID | SEQ ID | SEQ ID |
| | | NO: 300 | NO: 306 | NO: 317 | NO: 320 | NO: 323 | NO: 335 | NO: 341 | NO: 354 |
| P1G4 | B1-19 | SEQ ID | SEQ ID | SEQ ID | SEQ ID | SEQ ID | SEQ ID | SEQ ID | SEQ ID |
| | | NO: 301 | NO: 306 | NO: 309 | NO: 322 | NO: 323 | NO: 335 | NO: 341 | NO: 354 |
| P1B12 | B1-20 | SEQ ID | SEQ ID | SEQ ID | SEQ ID | SEQ ID | SEQ ID | SEQ ID | SEQ ID |
| | | NO: 289 | NO: 306 | NO: 309 | NO: 320 | NO: 331 | NO: 335 | NO: 350 | NO: 358 |
| P1C7 | B1-21 | SEQ ID | SEQ ID | SEQ ID | SEQ ID | SEQ ID | SEQ ID | SEQ ID | SEQ ID |
| | | NO: 289 | NO: 306 | NO: 318 | NO: 320 | NO: 325 | NO: 335 | NO: 341 | NO: 98 |
| P1A3_A | B1-22 | SEQ ID | SEQ ID | SEQ ID | SEQ ID | SEQ ID | SEQ ID | SEQ ID | SEQ ID |
| | | NO: 201 | NO: 306 | NO: 310 | NO: 320 | NO: 323 | NO: 335 | NO: 341 | NO: 354 |
| P1A3_Q | B1-23 | SEQ ID | SEQ ID | SEQ ID | SEQ ID | SEQ ID | SEQ ID | SEQ ID | SEQ ID |
| | | NO: 289 | NO: 306 | NO: 310 | NO: 205 | NO: 323 | NO: 335 | NO: 341 | NO: 354 |
| P1A3_AQ | B1-24 | SEQ ID | SEQ ID | SEQ ID | SEQ ID | SEQ ID | SEQ ID | SEQ ID | SEQ ID |
| | | NO: 201 | NO: 306 | NO: 310 | NO: 205 | NO: 323 | NO: 335 | NO: 341 | NO: 354 |
| P1A3_ANQ | B1-25 | SEQ ID | SEQ ID | SEQ ID | SEQ ID | SEQ ID | SEQ ID | SEQ ID | SEQ ID |
| | | NO: 201 | NO: 306 | NO: 203 | NO: 205 | NO: 323 | NO: 335 | NO: 341 | NO: 354 |
| P1A10_AQ | B1-26 | SEQ ID | SEQ ID | SEQ ID | SEQ ID | SEQ ID | SEQ ID | SEQ ID | SEQ ID |
| | | NO: 202 | NO: 302 | NO: 311 | NO: 205 | NO: 325 | NO: 335 | NO: 341 | NO: 351 |
| P1A10_ANQ | B1-27 | SEQ ID | SEQ ID | SEQ ID | SEQ ID | SEQ ID | SEQ ID | SEQ ID | SEQ ID |
| | | NO: 202 | NO: 302 | NO: 204 | NO: 205 | NO: 325 | NO: 335 | NO: 341 | NO: 351 |

TABLE C1
gamma chain binding moieties

| Binding | Binding | Column A | Column B |
|---|---|---|---|
| moiety name | moiety ID | VH | VL |
| P1A3 | C1-1 | SEQ ID NO: 1 | SEQ ID NO: 20 |
| P1A3_B3 | C1-2 | SEQ ID NO: 3 | SEQ ID NO: 20 |
| P1A3_E8 | C1-3 | SEQ ID NO: 4 | SEQ ID NO: 20 |
| P1A3_E9 | C1-4 | SEQ ID NO: 3 | SEQ ID NO: 20 |
| P1A3_B4 | C1-5 | SEQ ID NO: 3 | SEQ ID NO: 22 |
| P1A3_FW2 | C1-6 | SEQ ID NO: 5 | SEQ ID NO: 23 |
| P2B9 | C1-7 | SEQ ID NO: 2 | SEQ ID NO: 21 |
| P1A10 | C1-8 | SEQ ID NO: 6 | SEQ ID NO: 24 |
| P1B6 | C1-9 | SEQ ID NO: 7 | SEQ ID NO: 25 |
| P1C10 | C1-10 | SEQ ID NO: 8 | SEQ ID NO: 26 |
| P1D7 | C1-11 | SEQ ID NO: 9 | SEQ ID NO: 27 |
| P1E8 | C1-12 | SEQ ID NO: 10 | SEQ ID NO: 28 |
| P2B2 | C1-13 | SEQ ID NO: 11 | SEQ ID NO: 29 |
| P2B7 | C1-14 | SEQ ID NO: 12 | SEQ ID NO: 30 |
| P2D11 | C1-15 | SEQ ID NO: 13 | SEQ ID NO: 31 |
| P2F10 | C1-16 | SEQ ID NO: 14 | SEQ ID NO: 32 |
| P2H4 | C1-17 | SEQ ID NO: 15 | SEQ ID NO: 33 |
| P2D3 | C1-18 | SEQ ID NO: 16 | SEQ ID NO: 34 |
| P1G4 | C1-19 | SEQ ID NO: 17 | SEQ ID NO: 35 |
| P1B12 | C1-20 | SEQ ID NO: 18 | SEQ ID NO: 36 |
| P1C7 | C1-21 | SEQ ID NO: 19 | SEQ ID NO: 37 |
| P1A3-A | C1-22 | SEQ ID NO: 215 | SEQ ID NO: 20 |
| P1A3-Q | C1-23 | SEQ ID NO: 216 | SEQ ID NO: 20 |
| P1A3-AQ | C1-24 | SEQ ID NO: 197 | SEQ ID NO: 20 |
| P1A3-ANQ | C1-25 | SEQ ID NO: 198 | SEQ ID NO: 20 |
| P1A10-AQ | C1-26 | SEQ ID NO: 199 | SEQ ID NO: 24 |
| P1A10-ANQ | C1-27 | SEQ ID NO: 200 | SEQ ID NO: 24 |

TABLE A2
gamma chain binding moieties

| Binding moiety | Column A | | | |
|---|---|---|---|---|
| name/clone | VHH sequence | CDR1 | CDR2 | CDR3 |
| 2RGT38 | SEQ ID NO: 377 | SEQ ID NO: 370 | SEQ ID NO: 371 | SEQ ID NO: 372 |

TABLE B2
gamma chain binding moieties

| Binding moiety | | | | |
|---|---|---|---|---|
| name/clone | FR1 | FR2 | FR3 | FR4 |
| 2RGT38 | SEQ ID NO: 373 | SEQ ID NO: 374 | SEQ ID NO: 375 | SEQ ID NO: 376 |

TABLE A3
IL-9Rα binding moieties

| Binding moiety | Column A | | | |
|---|---|---|---|---|
| name/clone | VHH sequence | CDR1 | CDR2 | CDR3 |
| 2SAT363 | SEQ ID NO: 385 | SEQ ID NO: 378 | SEQ ID NO: 379 | SEQ ID NO: 380 |
| 2SAT57 | SEQ ID NO: 393 | SEQ ID NO: 386 | SEQ ID NO: 387 | SEQ ID NO: 388 |

TABLE B3
IL-9Rα binding moieties

| Binding moiety | | | | |
|---|---|---|---|---|
| name/clone | FR1 | FR2 | FR3 | FR4 |
| 2SAT363 | SEQ ID NO: 381 | SEQ ID NO: 382 | SEQ ID NO: 383 | SEQ ID NO: 384 |
| 2SAT57 | SEQ ID NO: 389 | SEQ ID NO: 390 | SEQ ID NO: 391 | SEQ ID NO: 392 |

The present disclosure includes the combination of the aspects and preferred features described except where such a combination is clearly impermissible or expressly avoided.

The section headings used herein are for organisational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word ‘comprise,’ and variations such as ‘comprises’ and ‘comprising,’ will be understood to imply the inclusion of a stated integer or step or group of integers or steps but not the exclusion of any other integer or step or group of integers or steps.

As used herein, an amino acid sequence, or a region of a polypeptide which ‘corresponds’ to a specified reference amino acid sequence or region of a polypeptide has at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of the amino acid sequence/polypeptide/region. An amino acid sequence/region/position of a polypeptide/amino acid sequence which ‘corresponds’ to a specified reference amino acid sequence/region/position of a polypeptide/amino acid sequence can be identified by sequence alignment of the subject sequence to the reference sequence, e.g. using sequence alignment software such as ClustalOmega (Sdding, J. 2005, Bioinformatics 21, 951-960).

It must be noted that, as used in the specification and the appended claims, the singular forms ‘a,’ ‘an,’ and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about,’ it will be understood that the particular value forms another embodiment.

Where a nucleic acid sequence is disclosed herein, the reverse complement thereof is also expressly contemplated.

Methods described herein may preferably be performed in vitro. The term ‘in vitro’ is intended to encompass procedures performed with cells in culture whereas the term ‘in vivo’ is intended to encompass procedures with/on intact multi-cellular organisms.

### EXAMPLES

### Example 1: Production and Characterisation of Bispecific γc- and CD122-Binding Antibodies in WO 2017/021540 A1, WO 2019/092181 A1, WO 2020/094834 A1 and WO 2020/094836 A1

The production and characterisation of bispecific, γc- and CD122-binding antibody agonists of the intermediate-affinity γc/IL-2RP receptor is described e.g. in WO 2017/021540 A1, WO 2019/092181 A1, WO 2020/094834 A1 and WO 2020/094836 A1, which are hereby incorporated by reference in their entirety.

### 1.1 WO 2017/021540 A1

Example 1 of WO 2017/021540 A1 discloses the identification of γc-binding clones and CD122-binding clones from a human antibody phage display library via in vitro selection. Example 2 describes the production of bispecific molecules comprising a γc-binding arm and a CD122-binding arm (i.e., bispecific anti-γc, anti-CD122 antibodies). The bispecific anti-γc, anti-CD122 antibodies employed the following combinations of γc- and CD122-binding clones:

Example 3 and FIG. 21A demonstrate binding of bispecific antibodies comprising a γc-binding arm and/or a CD122-binding arm to cells expressing γc or CD122, as determined by flow cytometric analysis.

Example 4 and FIG. 20 show that bispecific molecules comprising various different combinations of γc-binding and CD122-binding arms bind to recombinant γc or CD122, and Table 1 of Example 1 summarises the affinity of binding to the respective proteins by the bispecific antibodies, as determined by Surface Plasmon Resonance analysis.

Example 5 and FIG. 21B show that a bispecific anti-γc, anti-CD122 antibody bound efficiently to CD56+NK cells, CD19+B cells and CD14+/CD16+ monocytes, but did not bind with high affinity to CD4+ or CD8+ T cells.

Example 6.1 and FIG. 22 show that treatment of NK92 cells with a bispecific anti-γc, anti-CD122 antibody in vitro induced signalling through the STAT5 and Akt pathways, which are intracellular signalling pathways known to be triggered by IL-2. The molecule did not significantly upregulate phosphorylation of ERK, suggesting that it did not activate the ERK pathway (another intracellular signalling pathway triggered by IL-2). Example 6.1 and FIG. 23 show that a bispecific anti-γc, anti-CD122 antibody triggered signalling in IL-2 receptor-expressing cells (as determined by analysis of STAT5 phosphorylation) with a different activation profile as compared to IL-2, particularly activating signalling in NK and CD8+ T cells to a greater extent as proportion of activation of signalling in Tregs.

Example 6.2 and FIG. 24 show that a bispecific anti-γc, anti-CD122 antibody promoted proliferation of NK92 cells in vitro, and that bispecific molecules comprising one of the arms and an irrelevant second arm did not stimulate such cell proliferation.

Example 6.2 and FIGS. 25B and 25C demonstrate that bispecific molecules comprising a γc-binding arm and a CD122-binding arm provided in scFv-Fc(KiH)-scFv or tandem scFv-scFv format, and provided with linkers of various different lengths (i.e. between the scFv moiety and the constant regions in the KiH format molecules, and between the two scFv moieties in the tandem scFv-scFv format molecules) induce in vitro proliferation of NK92 cells with similar efficiency.

Example 6.3 and FIG. 26 show that a bispecific anti-γc, anti-CD122 antibody stimulated IL-2 receptor-mediated signalling (as determined by analysis of STAT5 phosphorylation) in cells among cynomolgus monkey splenocytes.

Example 7.1 and FIGS. 27A to 27D demonstrate that a bispecific anti-γc, anti-CD122 antibody stimulated proliferation of non-specifically activated (i.e. anti-CD3/CD28 bead-stimulated) primary human T cells in a dose-dependent manner, yielding ratios of CD8:CD4 cells in the expanded cell populations that are comparable to those achieved following stimulation with recombinant human IL-2. Example 7.2 and FIG. 28 show that stimulation with IL-2 in this setting promoted expansion of Tregs, but that the bispecific antibody did not trigger such Treg population expansion. Example 7.3 and FIG. 29 moreover demonstrate as compared to stimulation with IL-2 in this setting, the bispecific antibody promotes expansion of effector memory CD8+ cells to a greater extent, and triggers expansion of central memory and naïve T cells to a lesser extent.

Example 8.1 and FIGS. 30A to 30D demonstrate that a bispecific anti-γc, anti-CD122 antibody stimulated proliferation of primary human T cells from within a population of LCL-stimulated PBMCs obtained from an EBV-seropositive donor. The molecule elicited an increase in the number of CD8+ T cells to a greater extent than recombinant IL-2, and consequently yielded a greater CD8:CD4 T cell ratio. Example 8.2 and FIGS. 31A to 31C show that in this setting, the bispecific antibody favoured the expansion of CD8+ T cells over CD8+ memory cells as compared to IL-2, and also resulting in increased expansion of the CD8+PD-1+ subset and less expansion of Tregs, as compared to stimulation with IL-2. Example 8.3 and FIG. 32 demonstrate that T cells expanded by stimulation with the bispecific antibody elicit cytotoxicity against LCLs.

Example 9 and FIGS. 33A, 33B, 34A and 34B disclose the generation of framework region variants of a γc-binding clone and a CD122-binding clone that have higher thermostability relative to their respective parental clones. FIGS. 35A and 35B show that the preferred thermostable variant clones retained dose-dependent binding to their respective target antigens.

Example 10 and FIGS. 36A, 36B, 37A and 37B show that scFv-Fc(KiH)-scFv format molecules comprising different short linkers between the scFv moieties and hinge regions bound to recombinant γc or CD122 in ELISAs with similar affinity, irrespective of the sequence of the short linker, and with similar or improved affinity than the parental molecule.

Example 11 and FIGS. 38A and 38B show that antigen-specific CD8+ T cells are expanded to a greater extent from PBMCs of EBV-positive seropositive donors stimulated in vitro with LCLs in the presence of a bispecific molecule comprising a γc-binding arm and a CD122-binding arm, than in stimulations instead performed in the presence of recombinant human IL-2. Stimulations employing the bispecific antibody also achieved expanded populations having a higher CD8:CD4 T cell ratio than those using IL-2. FIGS. 39A and 39B show that in both antigen-specific and non-specific stimulation settings, the use of a bispecific anti-γc, anti-CD122 antibody was associated with significantly less expansion of Tregs than when IL-2 was used.

Example 12 discloses that intravenous administration of a bispecific anti-γc, anti-CD122 antibody to cynomolgous macaques was associated with marked proliferation of CD4+ and CD8+ T cell, and NK cell populations (FIGS. 41A to 41C, FIGS. 42A and 42B), and was not associated with a significant increase in the levels of inflammatory cytokines (FIGS. 40A to 40E). Expansion of the CD4+ and CD8+ T cells and NK cells was observed after a single dose of the bispecific antibody, where continuous infusion/repeat doses of recombinant IL-2 were required to achieve such expansion, suggesting that the bispecific antibody had a longer half-life than IL-2.

### 1.2 WO 2019/092181 A1

Example 1 of WO 2019/092181 A1 discloses the production of bispecific anti-γc, anti-CD122 antibodies, in various different formats (particularly scFv-FcKiH-scFv format, CrossMab format and Duobody format).

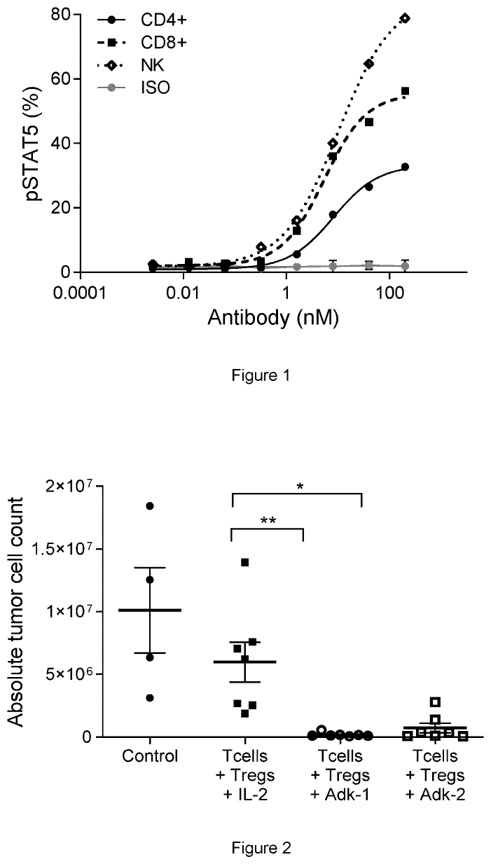

Example 2.1 and FIGS. 1A and 1B report the ability of bispecific anti-γc, anti-CD122 antibodies comprising different γc-binding and a CD122-binding clones and provided in different antibody formats to bind to recombinant γc or CD122, as determined by ELISA. Example 2.2 discloses binding kinetics for two different bispecific anti-γc, anti-CD122 antibodies for binding to recombinant γc or CD122, as determined by Biolayer Interferometry.

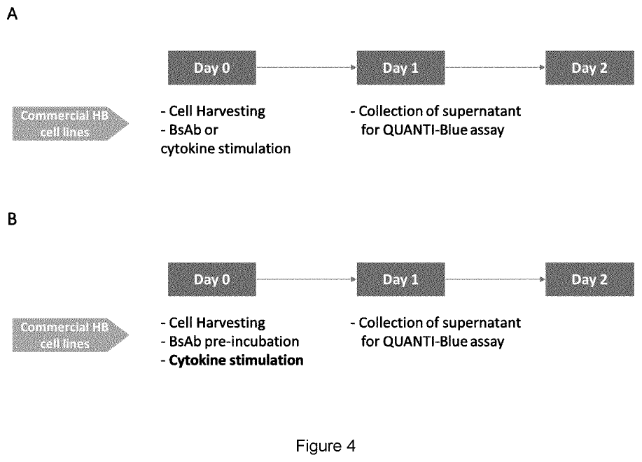

Example 2.3 and FIGS. 2A to 2C show that the bispecific antibodies display binding to cells transfected with constructs for expressing human γc and human CD122, but not to cells transfected with a construct for expressing human IL-2Rα (CD25), as determined by flow cytometric analysis. Example 2.5 and FIGS. 4A and 4B show that the bispecific antibodies also display binding to cells transfected with constructs for expressing rhesus γc and rhesus CD122.

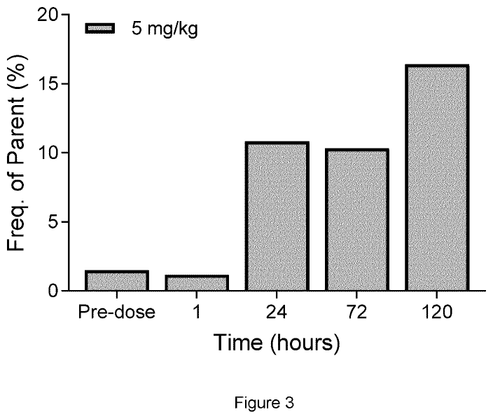

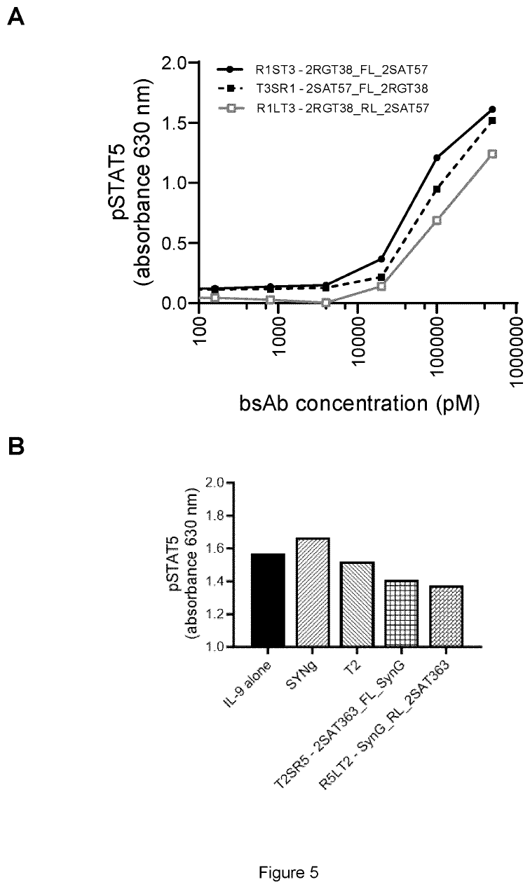

Example 2.4 and FIGS. 3A and 3B show that the bispecific antibodies displayed binding to primary human T cell subsets, while Example 2.6 and FIG. 5 show that the bispecific antibodies displayed binding to cynomolgous macaque T cell subsets.

Example 3.1 and FIG. 6A show that bispecific anti-γc, anti-CD122 antibodies comprising different γc- and CD122-binding clones promoted proliferation of NK92 cells in vitro, and FIG. 6B shows that bispecific anti-γc, anti-CD122 antibodies comprising different γc-binding clones and provided in different formats promoted proliferation of NK92 cells in vitro.

Example 3.2 and FIGS. 7A to 7L demonstrate that bispecific anti-γc, anti-CD122 antibodies stimulated proliferation of non-specifically activated (i.e. anti-CD3/CD28-stimulated) primary human CD8+ T cells, while causing only minimal expansion of Tregs. The absolute number of Tregs were 10-fold lower following treatment with the bispecific anti-γc, anti-CD122 antibodies as compared to treatment with IL-2.

CD8+ T effector memory cells responded the most to simulation with the bispecific anti-γc, anti-CD122 antibodies, and proliferation of CD4+ T effector memory cells was also observed. FIGS. 8A to 8G show that the bispecific anti-γc, anti-CD122 antibodies dose-dependent proliferation of non-specifically activated T cells, with more pronounced expansion of CD8+ T cells than CD4+ T cells. The bispecific anti-γc, anti-CD122 antibodies did not induce significant proliferation of Tregs, and they yielded expanded populations with higher ratios of CD8+ T cells to Tregs as compared to those expanded with IL-2 or IL-15. FIG. 8H shows that pre-expanded human Tregs were not expanded by treatment with the bispecific anti-γc, anti-CD122 antibodies, whereas they expanded in a dose-dependent fashion in response to treatment with IL-2 or IL-15. Example 3.3 and FIGS. 9A to 91 similarly disclose preferential expansion of CD8+ T cells over Tregs, and CD4+ T cell proliferation following treatment of non-specifically activated primary human PBMCs treated with the bispecific anti-γc, anti-CD122 antibodies.

Example 3.4 and FIGS. 10A to 10G demonstrate that bispecific anti-γc, anti-CD122 antibodies stimulated in vitro proliferation of antigen-specific (particularly EBV-specific) CD4+ and CD8+ T cells in a dose-dependent manner, and to also induce proliferation of NK cells within the virus-specific T cell population.

Example 3.5 and FIGS. 11A to 11K show that a bispecific anti-γc, anti-CD122 antibody stimulated dose-dependent proliferation of non-specifically activated (i.e. anti-CD3/CD28-stimulated) cynomolgous CD4+ effector memory T cells, CD8+Naïve T cells, CD8+ effector memory T cells and NK cells. The bispecific anti-γc, anti-CD122 antibodies did not induce proliferation of cynomolgous Tregs, whereas IL-2 did.

Example 3.6 and FIGS. 12A to 12N similarly show that bispecific anti-γc, anti-CD122 antibodies induce in vitro proliferation of all cynomolgous CD4+ and CD8+ T cell subsets in a non-specific activation setting, with preferential expansion of CD8+ T cells over CD4+ T cells.

Example 3.7 and FIGS. 27A to 27D demonstrate that bispecific anti-γc, anti-CD122 antibodies also induce proliferation of pre-activated NK cells, in a dose-dependent manner.

Example 3.8 and FIGS. 28A to 28D further illustrate that bispecific anti-γc, anti-CD122 antibodies induce proliferation of CAR-expressing T cells, with CD8+ CAR-T cells appearing to be more responsive to such stimulation than CD4+ CAR-T cells.

Example 4.1 and FIG. 13 show that bispecific anti-γc, anti-CD122 antibodies induced STAT5 phosphorylation in NK92 cells, while Example 4.2 and FIGS. 14A to 14H similarly show that a bispecific anti-γc, anti-CD122 antibody induced STAT5 phosphorylation in primary human T cells and NK cells, in a dose-dependent manner. Example 4.3 and FIGS. 15A to 15C show that bispecific anti-γc, anti-CD122 antibodies induced STAT5 phosphorylation in non-specifically activated (i.e. anti-CD3/CD28-stimulated) primary human CD4+, CD8+ T cell subsets, and also in NK cells, in a dose-dependent manner. Example 4.6 and FIGS. 18A to 18C show induction of STAT5 phosphorylation by the bispecific molecules in EBV-specific T cells. Examples 4.4 and 4.5 and FIG. 16 and show the time-dependency of induction of STAT5 phosphorylation by the bispecific anti-γc, anti-CD122 antibodies in NK92 cells and primary human T cells.

Example 4.7 and FIG. 19 show that bispecific anti-γc, anti-CD122 antibodies did not influence signalling through the IL-4 receptor.

Examples 5.1 and 5.2 and FIGS. 20A to 20K and 21A to 21C show that unlike IL-2, the bispecific anti-γc, anti-CD122 antibodies did not induce significant proliferation of non-activated PBMCs or non-activated T cells, and so treatment with such antibodies may be associated with reduced toxicity relative to treatment with IL-2.

Example 6 and FIG. 22 show that a bispecific anti-γc, anti-CD122 antibody has much longer serum half-life than IL-2. Blood levels peaked at 1 hr post injection of cynomolgous macaques, and the bispecific antibody remained detectable until 120 h post-injection.

Example 7 and FIGS. 23A, 23B, 24A and 24B show that expression of γc and CD122 is upregulated on non-specifically activated T cells and EBV-specific T cells.

Example 8 describes the production of a bispecific anti-γc, anti-CD122 antibody in Duobody format.

Example 9 and FIGS. 25 and 26A to 261 show that administration of bispecific anti-γc, anti-CD122 antibodies to mice having an EBV-positive human B cell cancer and treated with EBV-specific human T cells was associated with an increased number of human CD3+, CD4+ and CD8+ T cells, compared to administration of IL-2 or isotype control antibody, and the CD3+ cells also displayed lower expression of PD-1. The mice administered bispecific anti-γc, anti-CD122 antibodies moreover had a lower organ tumor burden compared to mice administered IL-2 or isotype control antibody.

Example 10 and FIGS. 29 and 30A to 30J show that administration of bispecific anti-γc, anti-CD122 antibodies to mice having an EBV-positive human B cell cancer and treated with EBV-specific human T cells was associated with an increased number of circulating virus-specific T cells at various different time points, compared to administration of IL-2 or isotype control antibody. The bispecific antibody-treated mice also had elevated numbers of human CD3+, CD4+ and CD8+ T cells in various tissues, and treatment was also associated with higher CD8:CD4 T cell ratios, relative to those observed in mice instead treated with IL-2 or isotype control antibody. The mice administered bispecific anti-γc, anti-CD122 antibodies again had a lower organ tumor burden compared to mice administered IL-2 or isotype control antibody, and higher numbers of CD8+ T cells of mice administered bispecific anti-γc, anti-CD122 antibodies were shown to be expressing effector molecules (IFNγ, CD107a, perforin) relative to CD8+ T cells from mice administered IL-2 or isotype control antibody.

### 1.3 WO 2020/094834 A1

Example 10 and FIGS. 28A to 28D of WO 2020/094834 A1 describe the production bispecific anti-γc, anti-CD122 antibodies comprising different γc- and CD122-binding moieties and provided in different formats, and the results of analysis of their stability.

Examples 11.1 and 13.2 and FIGS. 29A, 29B and 40 show that bispecific anti-γc, anti-CD122 antibodies comprising different γc- and CD122-binding moieties and provided in different formats induce proliferation of NK92 cells in vitro.

Example 11.2 and FIGS. 30A to 30D and FIGS. 31A to 31D show that bispecific anti-γc, anti-CD122 antibodies comprising different γc- and CD122-binding moieties and provided in different formats stimulate proliferation of non-specifically activated (i.e. anti-CD3/CD28-stimulated) primary CD4+ and CD8+ human T cells in a dose-dependent fashion. Tregs proliferated much less, such that treatment with the bispecific anti-γc, anti-CD122 antibodies yielded expanded populations with a much greater CD8+ T cell:Treg ration than populations expanded by treatment with IL-2.

Examples 12 and 13.3, and FIGS. 32 to 35 and 41 to 44 demonstrate that bispecific anti-γc, anti-CD122 antibodies comprising different γc- and CD122-binding moieties and provided in different formats are thermostable and retain their biological activity after being subjected to incubation for up to 28 days at temperatures up to 37° C. FIGS. 37A and 37B show that bispecific anti-γc, anti-CD122 antibodies subjected to freeze-thaw treatment similarly retain their biological activity.

### 1.4 WO 2020/094836 A1

Examples 10.1, 10.2 and 11.1, and FIGS. 27A to 27J and FIGS. 38A and 38B of WO 2020/094834 A1 describe the production of bispecific anti-γc, anti-CD122 antibodies comprising different γc- and CD122-binding moieties and provided in different formats, and the results of analysis of their stability.

Examples 10.3 and 11.2 and FIGS. 28A, 28B and 39 show that bispecific anti-γc, anti-CD122 antibodies comprising different γc- and CD122-binding moieties and provided in different formats induce proliferation of NK92 cells in vitro. Example 10.3 and FIGS. 29A and 29B show that the bispecific antibodies stimulated proliferation of non-specifically activated (i.e. anti-CD3/CD28-stimulated) primary human T cells.

Examples 10.4 and 11.3 and FIGS. 30 to 34, and FIGS. 40 to 43 demonstrate that bispecific anti-γc, anti-CD122 antibodies comprising different γc- and CD122-binding moieties and provided in different formats are thermostable and retain their biological activity after being subjected to incubation for up to 28 days at temperatures up to 37° C.

Example 10.5 and FIGS. 35 to 37 show that bispecific anti-γc, anti-CD122 antibodies subjected to freeze-thaw treatment are similarly resistant to degradation/aggregation, and retain their biological activity.

### Example 2: Further Characterisation of Bispecific γc- and CD122-Binding Antibodies

### 2.1 Dose-Dependent STAT5 Phosphorylation

Human CD8, NK, and CD4 cells were contacted with agonist bispecific γc- and CD122-binding antibodies at different concentrations, and STAT5 signalling was analysed.

Human PBMCs were pre-activated with anti-CD3/CD28 and were then cultured with agonist bispecific γc- and IL-2Rβ-binding antibodies (Adk-1) for 30 min, and the levels of pSTAT5 were measure using flow cytometry. Adk-1 induced STAT5 phosphorylation within CD4+ T, CD8+ T and NK cell populations in a dose-dependent manner.

Results are shown in FIG. 1. It can be seen that the percentage of phosphorylated STAT5 (pSTAT5) increases with the increasing concentration of bispecific γc- and IL-2Rβ-binding antibodies, demonstrating stimulation of human CD8, NK and CD4 cells, with minimal effect on CD4+ Treg (DNS). This demonstrates that contact the bispecific γc- and CD122-binding antibodies led to an increase in STAT5 signalling.

### 2.2 Tumor Killing by EBV Specific T Cells

EBV-BLCLs were generated by infecting PBMCs from EBV-seropositive donors with EBV supernatant. They were injected subcutaneously (SC) into flanks of NSG mice to establish a highly aggressive and metastatic disease that closely mimics EBV-driven lymphoid malignancies in humans, with dissemination to the spleen, liver and tertiary lymph nodes (typically absent in NSG mice). Mice were started on the specified treatment regimen once the growth at the injection side became palpable (typically 6-8 mm). T cells were produced by in vitro stimulation of PBMCs from the same EBV-seropositive donor as above, with irradiated EBV-BLCLs. Tregs were expanded according to the protocol described in Section 3.4 to ensure sufficient numbers for injection. Peripheral blood was collected from the same EBV-seropositive donor and CD4+CD25+ natural Tregs were purified using commercial magnetic beads. Purified Tregs were cultured with K562 feeder cells engineered to express 4-1BBL (CD137L), CD86 and the high affinity Fc Receptor CD64. K562 cells were irradiated at 100 Gray and pre-loaded with anti-CD3 mAb before purified Tregs were added at a Tregs: K562 ratio of 2:1 to 1:1. Tregs were re-stimulated once with irradiated K562 feeder cells in the same ratio after 7 days. Recombinant IL-2 (300 IU/ml) and Rapamycin (100 nM) was added and maintained for the culture duration. For the in vivo model, EBV-BCL tumor-engrafted mice were treated with EBV specific T cells and Treg cells in the presence of IL-2 or agonist bispecific γc- and IL-2Rβ-binding antibodies (Adk-1 or Adk-2). IL-2 preferentially stimulates Tregs and hence prevents elimination of tumor. On the other hand, agonist bispecific γc- and CD122-binding antibodies do not stimulate Tregs and yield clearance of tumors in vivo.

### 2.3 NHP T-Cell Proliferation In Vivo

Cynomolgus macaques were injected by i.v. with a single dose of agonist bispecific γc- and IL-2Rβ-binding antibodies at 5 mg/kg through the femoral artery. Blood was collected pre-dose, and at 1 h, 24 h, 72 h and 120 h post-injection. Expression of the proliferation marker Ki67 was used as a pharmacodynamic marker of immune stimulation in T cells. CD8 T cell proliferation occurred as early as 24 hours, persisting up to 120 hours post-dose.

### Example 3: Generation of γc-Specific VHH Antibodies

### 3.1 Immunization & Library Construction

Two llamas were subcutaneously immunized bi-weekly for 6 times, each time with about 100 μg of recombinant human interleukin 2 receptor gamma IL2Rγ (hIL2Rγ) fused to a His-tag. Gerbu P was used as adjuvant for all immunizations. Four and eight days after the 4th immunization, about 100 ml anticoagulated blood was collected from the llamas for lymphocyte preparation followed by library generation. Similarly, 4 and 8 days following the last immunization (6th immunization) about 100 ml anticoagulated blood was collected from the llamas for lymphocyte preparation and library generation. The general health status (eating habit, body temperature, etc.) of the animals was monitored regularly, by a certified vet, throughout the immunization period. The animals remained healthy during the whole procedure and showed no sign of discomfort.

For each animal's collected blood samples an independent VHH (Nanobody) library was constructed from four sets of llama lymphocytes to screen for the presence of antigen-specific Nanobodies. To this end, for each blood sampling timepoint (after 4 and after 6 immunizations), total RNA from peripheral blood lymphocytes retrieved at 4 d.p.i. was mixed 1/1 (weight ratio) with total RNA from peripheral blood lymphocytes retrieved at 8 d.p.i. This RNA mixture was used as template for first strand cDNA synthesis with an oligo(dT) primer. A mix of the cDNA prepared after 4 immunizations (4+8 d.p.i.) and the cDNA prepared after 6 immunizations (4+8 d.p.i.) was used to amplify the VHH encoding sequences by PCR. These sequences were digested with SapI and cloned into the SapI site of the phagemid vector pMECS-GG. In pMECS-GG vector, the Nanobody sequence is followed by a linker, HA tag and His6 tag (AAAYPYDVPDYGSHHHHHH, SEQ ID NO:368). Electro-competent E. coli TG1 cells were transformed with the recombinant pMECS-GG vector resulting in 2 independent VHH (Nanobody) libraries. The VHH library obtained from the first animal was called Core 216. The Core 216 library consists of about 9.7×10⁸ independent transformants, with about 92% of transformants harboring the vector with the right insert size. The same approach used for the second animal resulted in a library designated as Core 217. The Core 217 library consists of about 1.3×10⁸ independent transformants, with about 87% of transformants harboring the vector with the right insert size.

### 3.2 Isolation of Human IL2Rγ-Specific Nanobodies

The Core 216 and Core 217 libraries were panned on solid-phase coated human IL2Rγ-His (100 μg/ml in 100 mM NaHCO3 pH 8.2) using combinatorial panning in which each library is panned separately for the first round of panning. The phage outputs from these initial 1st rounds were mixed and used for a 2nd and a 3rd round of panning. The enrichment for antigen-specific phages was assessed after each round of panning by comparing the number of phagemid particles eluted from antigen-coated wells with the number of phagemid particles eluted from negative control (uncoated blocked) wells. These experiments suggested that the phage population was enriched for antigen-specific phages about 3-fold, 50-fold and 400-fold after the combined 1st rounds, the 2nd and the 3rd round, respectively.

In total, 380 colonies (285 from round 2 and 95 from round 3) were randomly selected and analyzed by ELISA for the presence of antigen-specific Nanobodies in their periplasmic extracts (ELISA using crude periplasmic extracts including soluble Nanobodies). The antigen used for panning and ELISA screening was the same as the one used for immunization (Sino Biological, Cat. No. 10555-H08H), using uncoated blocked wells as negative control (blank). Out of these 380 colonies tested by ELISA, 285 colonies scored positive for human IL2Rγ-His. Based on sequence data of the positive colonies, 54 different Nanobodies were distinguished, belonging to 11 different CDR3 groups (B-cell lineages).

One human γc-specific VHH antibody (2RGT38) was selected for further characterisation.

### Example 4: Generation of IL-9Rα-Specific VHH Antibodies

### 4.1 Immunization & Library Construction

A llama was subcutaneously immunized bi-weekly for 6 times, each time with about 100 μg of recombinant human interleukin 9 receptor alpha (hIL9R) fused, at the c-terminal, to a human IgG1 Fc (hIL9R-Fc).

The process described in Example 3.1 was followed.

Electro-competent E. coli TG1 cells were transformed with the recombinant pMECS-GG vector resulting in the VHH (Nanobody) library called Core 216. The Core 216 library consists of about 9.7×108 independent transformants, with about 92% of transformants harboring the vector with the right insert size.

### 4.2 Isolation of Human IL-9R-Specific Nanobodies

The Core 216 library was panned on solid-phase coated hIL9R-Fc (100 pg/ml in 100 mM NaHCO₃ pH 8.2). The enrichment for antigen-specific phages was assessed after each round of panning by comparing the number of phagemid particles eluted from antigen-coated wells with the number of phagemid particles eluted from negative control (uncoated blocked) wells. These experiments suggested that the phage population was enriched for antigen-specific phages about 20-fold and 70-fold after the 2^{nd} and 3^{rd} round, respectively.

In total, 570 colonies (285 from round 2 and 285 from round 3) were randomly selected and analyzed by ELISA for the presence of antigen-specific Nanobodies in their periplasmic extracts (ELISA using crude periplasmic extracts including soluble Nanobodies). The antigen used for panning and ELISA screening was the same as the one used for immunization (Acro Biosystems, Cat. No. ILR-H5251), using uncoated blocked wells as negative control (blank) and hIgG1-Fc (Sino Biological, Cat. No. 10702-HNAH) coated wells as reference for Fc specific clones. Out of these 570 colonies tested by ELISA, 90 colonies scored positive for hIL9R-Fc only. Based on sequence data of the positive colonies, 29 different Nanobodies were distinguished, belonging to 14 different CDR3 groups (B-cell lineages)

Two hIL-9Rα-specific VHH antibodies (2SAT363 and 2SAT57) were selected for further characterisation.

### Example 5: Bispecific γc- and IL-9Rα-Binding Antibodies

Single domain antibodies (VHH) are the smallest antigen binding domains derived from a camelid heavy-chain only antibodies. VHH offers a number of advantages over conventional antibodies that make them a desirable choice for therapeutics, as well as a versatile format for engineering bi- and trispecific molecules (Muyldermans S. Annu Rev Biochem 2013:82:775-97). Lacking a light chain, single domain antibodies have a substantially smaller molecular weight, which contributes to improved pharmacokinetics, greater stability, and easier humanization. Crucially, single domain antibodies retain the diverse binding specificity associated with antibodies of all formats. While the combinatorial nature of conventional antibodies can make sampling this diversity difficult, often necessitating the use of single cell or hybridoma techniques, the diversity of a single domain antibody response can be efficiently captured in phage display libraries (Maass D. et al. J Immunol Methods. 2007 Jul. 31; 324 (1-2): 13-25). The libraries can be interrogated for function-modifying antibodies in vitro, which permits significantly greater screening throughput, as well as diversity in the pool of antibodies that can be screened.

The function of bi- and trispecific antibodies is much greater than a simple sum of their parts. Their development presents a number of challenges not typically encountered in traditional therapeutic antibody development. The increasing molecular weight that comes from the conjugation of antibody molecules is, however, much more elegantly mitigated by employing single domain antibodies (Steeland S. Drug Discovery Today, Volume 21, Issue 7, July 2016, Pages 1076-1113). Issues of stability are less likely to be encountered, and therefore production and formulation are likely to be significantly easier. Furthermore, the risk of adverse effects due to aggregation is less likely when employing more stable formats such as single domain antibodies. Successful deployment of bi- and trispecific antibodies demands multiple high affinity selective antibodies be obtained for multiple targets, and subsequently engineered into a single biologically effective molecule. The biophysical superiority of the single domain antibody, coupled with its relative ease of screening and engineering make it an ideal format for this role.

### 5.1 Generation of Bispecific γc- and IL-9Rα-Binding Antibodies

Novel bispecific antibodies comprising different combinations of a γc-binding arm (2RGT38 or VHH clone nb6), and an IL-9Rα-binding arm (2SAT363 or 2SAT57) were generated, using different linkers.

Examples 3 and 4 herein describe the generation of γc-specific VHH (2RGT38) and IL-9Rα-specific VHH (2SAT363 and 2SAT57), respectively.

SynG comprises the antigen binding region of nb6 (Yen et al., Cell. 2022; 185(8): 1414-1430), the sequence of which is available from the Protein Data Bank (nanobody gamma-nb6; PDB ID: 7S2R).

The following bispecific γc- and IL-9Rα-binding antibodies were generated. Their identifying names are underlined, and indicate their constituent components. The identifying names of tandem VHH antibodies also indicate their orientation, i.e., VHH-linker-VHH (from the N-terminus to the C-terminus).

Two linkers were explored. The first linker is a short flexible linker (G4S, i.e. of amino acid sequence: GGGGS, SEQ ID NO:240, abbreviated herein as “FL” for flexible linker or “S” for short), and the second linker is a long rigid linker (“RL” for rigid linker or “L” for long) of amino acid sequence

(SEQ ID NO: 360)
AEAAAKEAAAKEAAAKEAAAKEAAAKA.

TABLE A4
bispecific antibodies

| Bispecific | N-terminal | | C-terminal |
|---|---|---|---|
| antibody Name | binding moiety | Linker | binding moiety |
| R1ST3 | 2RGT38 (R1) | FL | 2SAT57 (T3) |
| T3SR1 | 2SAT57 (T3) | FL | 2RGT38 (R1) |
| R1LT3 | 2RGT38 (R1) | RL | 2SAT57 (T3) |
| T2SR5 | 2SAT363 (T2) | FL | SynG (R5) |
| R5LT2 | SynG (R5) | RL | 2SAT363 (T2) |

### 5.2 Bispecific VHH Generation—Experimental Details

A general schematic overview of the cell signalling (STAT5 phosphorylation) assays performed is provided in FIG. 4. Further details are provided within the below text.

Bispecific VHH antibodies targeting IL-9Rα/γc were generated through overlapping PCR, incorporating either a short flexible linker (GGGGS, SEQ ID NO:240) or a long rigid linker (AEAAAKEAAAKEAAAKEAAAKEAAAKA, SEQ ID NO:360) sequence between individual VHH antibodies. The resulting full-length PCR fragments were subsequently cloned into linearized pMECS-GG vector using either T4 DNA ligase or ligation-free cloning kit, followed by electroporation into WK6 competent cells. Clones were screened by colony PCR and sequences were confirmed by Sanger DNA sequencing.

For bispecific VHH antibody expression in bacteria, overnight WK6 bacteria cultures were diluted 50-fold into fresh SB medium supplemented with 0.1% glucose and ampicillin, followed by incubation at 37° C. with shaking until OD600 reaches 0.6. Expression of target bispecific VHHs was then induced by addition of 1 mM IPTG, followed by overnight incubation at 30° C. with shaking.

Periplasmic extraction (PPE) of VHH antibodies were accomplished using the TES method. Briefly, the induced bacteria cultures were first pelleted at 2500 g for 10 min at 4C. For each 25 ml original culture, the cell pellet was resuspended with 0.6 ml of ice-cold TES buffer (200 mM Tris-HCl, pH 8.0; 0.5 mM EDTA; 0.5 M sucrose) and incubated on ice for 1 hour with shaking, after which 0.9 ml of TES/4 (TES buffer diluted 4× with water) was added and the mixture was incubated on ice for another 1 hour with shaking. After incubation, the suspensions were centrifuged at maximal speed for 5 min at 4C. The supernatant with a total volume of 1.5 ml was then carefully collected and sterile filtered for subsequent purification with magnetic nickel beads.

During purification, 200 μl magnetic nickel beads were added into 1.5 ml PPE extraction and incubated at room temperature for 2 hours with shaking. The protein-bound beads were then washed three times with phosphate buffer containing 20 mM imidazole to remove non-specifically bound molecules. Finally, the target bispecific VHHs were eluted with 100 μl of 500 mM imidazole and buffered-exchanged into sterile PBS with spin columns.

### 5.3 Dose-Dependent STAT5 Phosphorylation

### Agonist Assay

HEK-Blue IL-9 Cells were cultured, harvested, and then stimulated with a range of concentrations of bispecific γc- and IL-9Rα-binding antibodies (bsAb) or control antibodies for 24 hours, before supernatant was collected and analysed through the QUANTI-Blue assay to quantify the level of STAT5 phosphorylation after stimulation.

Results are shown in are shown in FIG. 5A. Bispecific antibodies R1ST3, T3SR1 and R1LT3 demonstrated agonist activity, with treatment resulting in an upregulation of STAT5 phosphorylation (FIG. 5A). It can be seen that the percentage of phosphorylated STAT5 (pSTAT5) increases with the increasing concentration of bispecific γc- and IL-9Rα-binding antibodies, demonstrating that contact of bispecific γc- and IL-9Rα-binding antibodies led to an increase in STAT5 signalling.

These data demonstrate that agonistic bispecific γc- and IL-9Rα-binding antibodies are capable of upregulating signalling mediated by a γc:IL-9Rα receptor.

### Antagonist Assay

HEK-Blue IL-9 Cells were cultured, harvested, and then stimulated with bispecific γc- and IL-9Rα-binding antibodies (bsAb) for 15 min, before being stimulated with IL-9 (5 pM). Supernatant was collected and analysed through the QUANTI-Blue assay to quantify the level of STAT5 phosphorylation after stimulation. The control antibodies include: monospecific IL-9Rα-binding VHH antibody 2SAT363 and monospecific γc-binding VHH antibody SynG.

Results are shown in FIG. 5B. Bispecific γc- and IL-9Rα-binding antibodies demonstrated antagonist activity, with treatment (at 500 nM) resulting in downregulation of STAT5 phosphorylation (FIG. 5B).

These data demonstrate that antagonistic bispecific γc- and IL-9Rα-binding antibodies are capable of inhibiting signalling mediated by a γc:IL-9Rα receptor.

1. An antigen-binding molecule, optionally isolated, comprising:
(i) a γc-binding moiety, and
(ii) an IL-9Rα-binding moiety.

2. The antigen-binding molecule according to claim 1, wherein the antigen-binding molecule is an agonist of a γc-containing cytokine receptor, or an antagonist of an γc-containing cytokine receptor.

3. The antigen-binding molecule according to claim 1 or claim 2, wherein the antigen-binding molecule increases signalling mediated by a γc-containing cytokine receptor to which the antigen-binding molecule binds, or decreases signalling mediated by a γc-containing cytokine receptor to which the antigen-binding molecule binds.

4. The antigen-binding molecule according to any one of claims 1 to 3, wherein the antigen-binding molecule increases signalling mediated by a γc:IL-9Rα receptor, or decreases signalling mediated by a γc:IL-9Rα receptor.

5. The antigen-binding molecule according to any one of claims 1 to 4, wherein the IL-9Rα-binding moiety comprises a single-domain antibody sequence incorporating the following CDRs:
(i) CDR1 having the amino acid sequence of SEQ ID NO:378
CDR2 having the amino acid sequence of SEQ ID NO:379
CDR3 having the amino acid sequence of SEQ ID NO:380; or
(ii) CDR1 having the amino acid sequence of SEQ ID NO:386
CDR2 having the amino acid sequence of SEQ ID NO:387
CDR3 having the amino acid sequence of SEQ ID NO:388.

6. The antigen-binding molecule according to claim 5, wherein the IL-9Rα-binding moiety comprises, or consists of, an amino acid sequence having at least 70% sequence identity to the amino acid sequence of SEQ ID NO:385 or 393.

7. The antigen-binding molecule according to claim 5 or claim 6, wherein the IL-9Rα-binding moiety comprises a single-domain antibody sequence incorporating the following FRs:
(i) FR1 having the amino acid sequence of SEQ ID NO:381
FR2 having the amino acid sequence of SEQ ID NO:382
FR3 having the amino acid sequence of SEQ ID NO:383
FR4 having the amino acid sequence of SEQ ID NO:384; or
(ii) FR1 having the amino acid sequence of SEQ ID NO:389
FR2 having the amino acid sequence of SEQ ID NO:390
FR3 having the amino acid sequence of SEQ ID NO:391
FR4 having the amino acid sequence of SEQ ID NO:392.

8. The antigen-binding molecule according to any one of claims 1-7, wherein the γc-binding moiety comprises a single-domain antibody sequence incorporating the following CDRs:
CDR1 having the amino acid sequence of SEQ ID NO:370
CDR2 having the amino acid sequence of SEQ ID NO:371
CDR3 having the amino acid sequence of SEQ ID NO:372.

9. The antigen-binding molecule according to claim 8, wherein the γc-binding moiety comprises, or consists of, an amino acid sequence having at least 70% sequence identity to the amino acid sequence of SEQ ID NO:377.

10. The antigen-binding molecule according to claim 8 or claim 9, wherein the γc-binding moiety comprises a single-domain antibody sequence incorporating the following FRs:
FR1 having the amino acid sequence of SEQ ID NO:373
FR2 having the amino acid sequence of SEQ ID NO:374
FR3 having the amino acid sequence of SEQ ID NO:375
FR4 having the amino acid sequence of SEQ ID NO:376.

11. The antigen-binding molecule according to any one of claims 1 to 10, wherein the antigen-binding molecule further comprises:
(iii) an antigen-binding moiety that binds to a target antigen other than a γc-containing cytokine receptor polypeptide.

12. The antigen-binding molecule according to claim 11, wherein the target antigen other than a γc-containing cytokine receptor polypeptide is a disease-associated antigen or an antigen expressed by an immune cell.

13. A chimeric antigen receptor (CAR), comprising an antigen-binding molecule according to any one of claims 1 to 12.

14. A nucleic acid, or a plurality of nucleic acids, optionally isolated, encoding an antigen-binding molecule according to any one of claims 1 to 12, or a CAR according to claim 13.

15. An expression vector, or a plurality of expression vectors, comprising a nucleic acid or a plurality of nucleic acids according to claim 14.

16. A cell comprising an antigen-binding molecule according to any one of claims 1 to 12, a CAR according to claim 13, a nucleic acid or a plurality of nucleic acids according to claim 14, or an expression vector or a plurality of expression vectors according to claim 15.

17. A method comprising culturing a cell according to claim 15 under conditions suitable for expression of an antigen-binding molecule or CAR by the cell.

18. A composition comprising an antigen-binding molecule according to any one of claims 1 to 12, a CAR according to claim 13, a nucleic acid or a plurality of nucleic acids according to claim 14, an expression vector or a plurality of expression vectors according to claim 15, or a cell according to claim 16, and a pharmaceutically acceptable carrier, diluent, excipient or adjuvant.

19. An antigen-binding molecule according to any one of claims 1 to 12, a CAR according to claim 13, a nucleic acid or a plurality of nucleic acids according to claim 14, an expression vector or a plurality of expression vectors according to claim 15, a cell according to claim 16, or a composition according to claim 18, for use in a method of treatment or prophylaxis.

20. Use of an antigen-binding molecule according to any one of claims 1 to 12, a CAR according to claim 13, a nucleic acid or a plurality of nucleic acids according to claim 14, an expression vector or a plurality of expression vectors according to claim 15, a cell according to claim 16, or a composition according to claim 18, in the manufacture of a medicament for use in a method of treatment or prophylaxis.

21. A method of treatment or prophylaxis, comprising administering to a subject in need thereof a therapeutically or prophylactically effective amount of an antigen-binding molecule according to any one of claims 1 to 12, a CAR according to claim 13, a nucleic acid or a plurality of nucleic acids according to claim 14, an expression vector or a plurality of expression vectors according to claim 15, a cell according to claim 16, or a composition according to claim 18.

22. The antigen-binding molecule, nucleic acid or plurality thereof, expression vector or plurality thereof, cell, or composition for use according to claim 19, the use according to claim 20 or the method according to claim 21, wherein the method of treatment or prophylaxis is a method of treating or preventing a disease/condition characterised by T cell dysfunction, a cancer, an infectious disease, or an autoimmune disease.

23. The antigen-binding molecule, nucleic acid or plurality thereof, expression vector or plurality thereof, cell, or composition for use, the use or the method according to claim 22, wherein the cancer is selected from the group consisting of: colon cancer, colon carcinoma, colorectal cancer, nasopharyngeal carcinoma, cervical carcinoma, oropharyngeal carcinoma, gastric carcinoma, hepatocellular carcinoma, head and neck cancer, head and neck squamous cell carcinoma (HNSCC), oral cancer, laryngeal cancer, prostate cancer, lung cancer, small cell lung cancer, non-small cell lung cancer, bladder cancer, urothelial carcinoma, melanoma, advanced melanoma, renal cell carcinoma, ovarian cancer or mesothelioma.

24. The antigen-binding molecule, nucleic acid or plurality thereof, expression vector or plurality thereof, cell, or composition for use according to claim 19, the use according to claim 20 or the method according to claim 21, wherein the method of treatment or prophylaxis is a method of treating or preventing a disease/condition in which a Th2 immune response is pathologically-implicated.

25. An in vitro complex, optionally isolated, comprising an antigen-binding molecule according to any one of claims 1 to 12, a CAR according to claim 13, bound to γc and a polypeptide of a γc-containing cytokine receptor other than γc.

26. A method for generating or expanding a population of cells expressing a γc-containing cytokine receptor, comprising contacting a cell expressing a γc-containing cytokine receptor in vitro, in vivo or ex vivo with an antigen-binding molecule according to any one of claims 1 to 12.

27. A method for increasing the proliferation, survival and/or effector activity of a cell expressing a γc-containing cytokine receptor, comprising contacting a cell expressing a γc-containing cytokine receptor in vitro, in vivo or ex vivo with an antigen-binding molecule according to any one of claims 1 to 12.

28. A method for reducing the number/proportion of cells expressing a γc-containing cytokine receptor, comprising contacting a cell expressing a γc-containing cytokine receptor in vitro, in vivo or ex vivo with an antigen-binding molecule according to any one of claims 1 to 12, or a cell comprising a CAR according to claim 13.

29. A method for decreasing the proliferation, survival and/or effector activity of a cell expressing a γc-containing cytokine receptor, comprising contacting a cell expressing a γc-containing cytokine receptor in vitro, in vivo or ex vivo with an antigen-binding molecule according to any one of claims 1 to 12.

30. The method according to any one of claims 26 to 29, wherein the cell is an effector immune cell.

31. The method according to any one of claims 26 to 30, wherein the cell is a T cell or a NK cell.

32. The method according to any one of claims 26 to 31, wherein the γc-containing cytokine receptor is a γc:IL-9Rα receptor.

33. A method of promoting heteromultimerization of γc and IL-9Rα, comprising contacting γc and IL-9Rα in vitro, in vivo or ex vivo with an antigen-binding molecule according to any one of claims 1 to 12, or a CAR according to claim 13.

34. A method of inhibiting heteromultimerization of γc and IL-9Rα, comprising contacting γc and IL-9Rα in vitro, in vivo or ex vivo with an antigen-binding molecule according to any one of claims 1 to 12, or a CAR according to claim 13.


SEQUENCE LISTING (561 sequences)

SEQ ID NO: 1 - protein, 120 aa, synthetic construct
QVQLQQWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTLVTVSS

SEQ ID NO: 2 - protein, 120 aa, synthetic construct
QVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAGDILTGYALDYWGQGTLVTVSS

SEQ ID NO: 3 - protein, 120 aa, synthetic construct
QVQLQQWGAGLLKPSETLSLTCAVYGGSFSGYYWSWIRQPPGKGLEWIGEINHFGSTNYNPSLKSRATISVDTSKNQFSLKLSSVTAADTAVYYCATSPGGYSGGYFQHWGQGTLVTVSS

SEQ ID NO: 4 - protein, 120 aa, synthetic construct
QVQLQQWGAGMLKPSETLSLTCAVYGGSFSGYYWSWIRQPPGKGLEWIGEINHFGSTNYNPSLKSRATISVDTSKNQFSLKLSSVTAADTAVYYCATSPGGYSGGYFQHWGQGTLVTVSS

SEQ ID NO: 5 - protein, 120 aa, synthetic construct
EVQLVESGGGLVQPGGSLRLSCAASGGSFSGYYWSWVRQAPGKGLEWVSEINHSGSTNYNPSLKSRFTISRDNSKNTLYLQMNSLRAEDTAVYYCARSPGGYSGGYFQHWGQGTLVTVSS

SEQ ID NO: 6 - protein, 120 aa, synthetic construct
QVQLQQSGAEVKKPGSSVKVSCKASGGTFSSYAISWVRQAPGQGLEWMGGFDPEDGETIYAQKFQGRVTMTEDTSTDTAYMELSSLRSEDTAVYYCATDLRIPYYYDNPWGQGTLVTVSS

SEQ ID NO: 7 - protein, 118 aa, synthetic construct
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARSLYYSHFDYWGQGTLVTVSS

SEQ ID NO: 8 - protein, 123 aa, synthetic construct
EVQLVETGPGLVKPSGTLSLTCAVSGGSISSSNWWSWVRQPPGKGLEWIGEIYHSGSTNYNPSLKSRVTISVDKSKNQFSLKLSSVTAADTAVYYCAREGPLSSSGPGAFDIWGQGTMVTVSS

SEQ ID NO: 9 - protein, 114 aa, synthetic construct
QVQLQESGGGVVQPGRSLRLSCAASGFTFSNYGMHWVRQAPGKGLEWVAVISYDGTNKYYADSVKGRFTISRDNSKNTVYLQMNSLRAEDTAVYYCAKDGFDIWGQGTMVTVSS

SEQ ID NO: 10 - protein, 120 aa, synthetic construct
EVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCARDVYGDYGAFDYWGQGTLVTVSS

SEQ ID NO: 11 - protein, 117 aa, synthetic construct
QLQLQESGGGVVQPGRSLRLSCAASGFTFSSYAMHWVRQAPGKGLEWVAVISYDGGNKYYADSVKGRFTISRDNSKNTLYLQMNSLRAEDTAVYYCAKSVAPPMDVWGKGTTVTVSS

SEQ ID NO: 12 - protein, 121 aa, synthetic construct
QVQLQQWGAGLLKPSETLSLTCAVYGESFSGYYWSWIRQPPGKGLEWIGEINHSGSTNYNPSLKSRVTISVDTSKNQFSLKLSSVTAADTAVYYCARGPAGSSSSGYFDYWGQGTLVTVSS

SEQ ID NO: 13 - protein, 116 aa, synthetic construct
QVQLQESGPGLVKPSQTLSLTCTVSGGSISSGGYYWTWIRQHPGQGLEWIGFISWSGTTYYNPSLKNRVTISADTSKNHFSLNLTSVTAADTAVYYCARGSGRLVWGQGTLVTVSS

SEQ ID NO: 14 - protein, 120 aa, synthetic construct
EVQLVQSGAEVKKPGASVKVSCKASGYTFTGYYMHWVRQAPGQGLEWMGIINPSGGSTSYAQKFQGRVTMTRDTSTSTVYMELSSLRSEDTAVYYCARADTAMGDAFDIWGQGTMVTVSS

SEQ ID NO: 15 - protein, 118 aa, synthetic construct
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARSIGIGAFDIWGQGTMVTVSS

SEQ ID NO: 16 - protein, 121 aa, synthetic construct
QVQLQQWGAGLLKPSETLSLTCTIYGGSFSGFYWSWIRQPPGKGLEWIGEINHSGSTNYNPSLKSRVTISVDTSKNQFSLKLSSVTAADTAIYYCARGPAGSTSSGYFDHWGQGTLVTVSS

SEQ ID NO: 17 - protein, 117 aa, synthetic construct
QVQLQQWGAGLLKPSETLSLTCAVYGGSLSGYYWSWIRQPPGKGLEWIGEINHSGSTNYNPSLKSRVTISVDTSKNQFSLKLSSVTAADTAVYYCARGSSSYYMDVWGKGTTVTVSS

SEQ ID NO: 18 - protein, 116 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGGSAYFQHWGQGTLVTVSS

SEQ ID NO: 19 - protein, 121 aa, synthetic construct
QVQLQQWGAGLLKPSETLSLTCAVYGGSFSGYYWSWIRQPPGKGLEWIGEINHSGSTNYNPSLKSRVTISEDASKKQFSLTLTSVTAADTAVYYCARGPAGTGSSGYFDYWGQGTLVTVSS

SEQ ID NO: 20 - protein, 112 aa, synthetic construct
DVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 21 - protein, 108 aa, synthetic construct
SYELTQPPSMSVSPGQTARITCSGDALPKQFAFWYQQKPGQAPVLVIYKDTERPSGIPERFSGSSSGTTVTLTITGVQAEDEADYYCQSPDSSGTVEVFGGGTKLTVL

SEQ ID NO: 22 - protein, 112 aa, synthetic construct
DVVMTQSPLSLPVTPGESVSISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 23 - protein, 112 aa, synthetic construct
DIQMTQSPSSLSASVGDRVTITCRSSQSLLHSNGYNYLDWYQQKPGKAPKLLIYLGSNRDSGVPSRFSGSGSGTDFTLTISSLQPEDFATYYCMQGTHWPWTFGQGTKVEIK

SEQ ID NO: 24 - protein, 112 aa, synthetic construct
EIVLTQSPLSLPVTPGEPASISCRSSQSLLHSNGYNYLNWYLQKPGQSPQLLIYLGSDRASGVPDRFSGSGSGTDFTLKISRVEAEDVGVYYCMQALQTPTTFGGGTKVEIK

SEQ ID NO: 25 - protein, 112 aa, synthetic construct
EIVMTQSPLSLPVTPGEPASISCRSSQSLLHSNGYNYLDWYLQKPGQSPQLLMYLVSNRASGVPERFSGSGSGTDFTLKISRVEAEDVGVYYCMQTLQTPLSFGQGTKLEIK

SEQ ID NO: 26 - protein, 107 aa, synthetic construct
EIVLTQSPATLSLSPGERATLSCRASQSVSYHLAWYQQKPGQAPRLLIYDTSNRASGIPARFSGSGSGTDFTLTINSLEPEDFAVYYCQQRYDWPLTFGGGTKVEIK

SEQ ID NO: 27 - protein, 107 aa, synthetic construct
DIQMTQSPSFLSASVGDRVTITCRASQSISSWLAWYQQKPGKAPKLLIYDASRLEDGVPSRFSGTGFGTDFTFTITTLQPDDIATYYCQQYDDLPYTFGQGTTVDIK

SEQ ID NO: 28 - protein, 112 aa, synthetic construct
DVVMTQSPVSLPVTLGQPASISCKSSQSLLYFNGNTYLSWFQQRPGQSPRRLFYQVSNRDSGVPDRFSGSGSDTDFTLTISRVEAEDVGVYFCMQGTQWPPTFGQGTKVEIK

SEQ ID NO: 29 - protein, 112 aa, synthetic construct
DVVMTQSPLSLPVTPGEPASISCRSSQSLLHSNGYNYLDWYLQKPGQSPHLLIYLGSNRASGVPDRFSGSGSGTDFTLKISRVEAEDVGVYFCMQALRTPYTFGQGTKLEIK

SEQ ID NO: 30 - protein, 112 aa, synthetic construct
DVVMTQSPLSLPVTLGQPASISCRSSQSLVHSNGYNYLDWYLQKPGQSPQLLIYLGSNRASGVPDRFSGSGSGTDFTLKISRVEAEDVGVYYCLQGSHWPWTFGQGTKVEIK

SEQ ID NO: 31 - protein, 106 aa, synthetic construct
ETTLTQSPATLSVSPGERATLSCRASQSVSSNLAWYQQKPGQAPRLLIYGASSGATGIPDRFSGSGSGTDFTLTISRLEPEDFAVYYCQLYGSSLAFGGGTKVEIK

SEQ ID NO: 32 - protein, 113 aa, synthetic construct
DIVMTHTPLSLPVTPGEPASISCRSSQTLFDSDDGKTYLDWYLQKPGQSPQLLMYTTSSRASGVPDRFSGSGSGTDFTLKISRVEAEDVGVYYCMQRLQFPLTFGQGTRLEFK

SEQ ID NO: 33 - protein, 112 aa, synthetic construct
DVVMTQSPLSLPVTPGEPASISCRATQSLLHGNGHNYLDWYLQKPGQSPQLLIYLGSNRASGVPDRFSGSGSGTDFTLKISRVEAEDVGVYYCMQTLETPVTFGPGTKVDIK

SEQ ID NO: 34 - protein, 112 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPWTFGQGTKVEIK

SEQ ID NO: 35 - protein, 112 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCLQGTHWPWTFGQGTKVEIK

SEQ ID NO: 36 - protein, 112 aa, synthetic construct
DVVMTQSPLSLPVTLGQPASISCRSSQSLLHSNGNNYLDWYLQKPGQSPQLLIYLGSNRASGVPDRFSGSGSGTDFTLKISRVEAEDVGIYYCMQGTHWPWTFGQGTKVEIE

SEQ ID NO: 37 - protein, 112 aa, synthetic construct
EIVLTQSPLSLPVTPGEPASISCRSSQSLLHSNGYNYLDWYLQKPGQSPQLLIYLASNRASGVPDRFSGSGSGTDFTLKISRVEAEDVGVYYCMQGTHWPWTFGQGTKVEVK

SEQ ID NO: 38 - protein, 5 aa, synthetic construct
GYYWS

SEQ ID NO: 39 - protein, 5 aa, synthetic construct
SYAMH

SEQ ID NO: 40 - protein, 6 aa, synthetic construct
SSNWWS

SEQ ID NO: 41 - protein, 16 aa, synthetic construct
EINHSGSTNYNPSLKS

SEQ ID NO: 42 - protein, 17 aa, synthetic construct
VISYDGSNKYYADSVKG

SEQ ID NO: 43 - protein, 16 aa, synthetic construct
EIYHSGSTNYNPSLKS

SEQ ID NO: 44 - protein, 16 aa, synthetic construct
RSSQSLLHSNGYNYLD

SEQ ID NO: 45 - protein, 7 aa, synthetic construct
LGSNRAS

SEQ ID NO: 46 - protein, 9 aa, synthetic construct
MQGTHWPWT

SEQ ID NO: 47 - protein, 7 aa, synthetic construct
SSSYYWG

SEQ ID NO: 48 - protein, 5 aa, synthetic construct
SYAIS

SEQ ID NO: 49 - protein, 5 aa, synthetic construct
NYGMH

SEQ ID NO: 50 - protein, 5 aa, synthetic construct
SYGMH

SEQ ID NO: 51 - protein, 7 aa, synthetic construct
SGGYYWT

SEQ ID NO: 52 - protein, 5 aa, synthetic construct
GYYMH

SEQ ID NO: 53 - protein, 5 aa, synthetic construct
GFYWS

SEQ ID NO: 54 - protein, 15 aa, synthetic construct
SIYYSGSTYYNPSLK

SEQ ID NO: 55 - protein, 16 aa, synthetic construct
EINHFGSTNYNPSLKS

SEQ ID NO: 56 - protein, 17 aa, synthetic construct
GFDPEDGETIYAQKFQG

SEQ ID NO: 57 - protein, 499 aa, synthetic construct
EVQLVQSGAEVKKPGSSVKVSCKASGYTFTNYYMHWVRQAPGQGLEWMGAIMPSRGGTSYPQKFQGRVTMTGDTSTSTVYMELSSLRSEDTAVYYCARGEYYYDSSGYYYWGQGTLVTVSSGGGGSGGGGSGGGGSQSALTQPASVSGSPGQSIAISCTGTSSDIGHYDFVSWYQQHPGTAPKLIIYDINNRPSGISNRFSGSKSDNMASLTISGLQPEDEADYYCSAYTSSDTLVFGGGTKLTVLNSGAAAQVQLQQWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTLVTVSSGGGGSGGGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 58 - protein, 17 aa, synthetic construct
VISYDGTNKYYADSVKG

SEQ ID NO: 59 - protein, 17 aa, synthetic construct
VISYDGGNKYYADSVKG

SEQ ID NO: 60 - protein, 16 aa, synthetic construct
FISWSGTTYYNPSLKN

SEQ ID NO: 61 - protein, 17 aa, synthetic construct
IINPSGGSTSYAQKFQG

SEQ ID NO: 62 - protein, 12 aa, synthetic construct
SPGGYSGGYFQH

SEQ ID NO: 63 - protein, 10 aa, synthetic construct
DILTGYALDY

SEQ ID NO: 64 - protein, 11 aa, synthetic construct
DLRIPYYYDNP

SEQ ID NO: 65 - protein, 9 aa, synthetic construct
SLYYSHFDY

SEQ ID NO: 66 - protein, 14 aa, synthetic construct
EGPLSSSGPGAFDI

SEQ ID NO: 67 - protein, 5 aa, synthetic construct
DGFDI

SEQ ID NO: 68 - protein, 11 aa, synthetic construct
DVYGDYGAFDY

SEQ ID NO: 69 - protein, 8 aa, synthetic construct
SVAPPMDV

SEQ ID NO: 70 - protein, 13 aa, synthetic construct
GPAGSSSSGYFDY

SEQ ID NO: 71 - protein, 6 aa, synthetic construct
GSGRLV

SEQ ID NO: 72 - protein, 11 aa, synthetic construct
ADTAMGDAFDI

SEQ ID NO: 73 - protein, 9 aa, synthetic construct
SIGIGAFDI

SEQ ID NO: 74 - protein, 13 aa, synthetic construct
GPAGSTSSGYFDH

SEQ ID NO: 75 - protein, 9 aa, synthetic construct
GSSSYYMDV

SEQ ID NO: 76 - protein, 8 aa, synthetic construct
GGSAYFQH

SEQ ID NO: 77 - protein, 13 aa, synthetic construct
GPAGTGSSGYFDY

SEQ ID NO: 78 - protein, 11 aa, synthetic construct
SGDALPKQFAF

SEQ ID NO: 79 - protein, 16 aa, synthetic construct
RSSQSLLHSNGYNYLN

SEQ ID NO: 80 - protein, 11 aa, synthetic construct
RASQSVSYHLA

SEQ ID NO: 81 - protein, 11 aa, synthetic construct
RASQSISSWLA

SEQ ID NO: 82 - protein, 16 aa, synthetic construct
KSSQSLLYFNGNTYLS

SEQ ID NO: 83 - protein, 16 aa, synthetic construct
RSSQSLVHSNGYNYLD

SEQ ID NO: 84 - protein, 11 aa, synthetic construct
RASQSVSSNLA

SEQ ID NO: 85 - protein, 17 aa, synthetic construct
RSSQTLFDSDDGKTYLD

SEQ ID NO: 86 - protein, 16 aa, synthetic construct
RATQSLLHGNGHNYLD

SEQ ID NO: 87 - protein, 16 aa, synthetic construct
RSSQSLLHSNGNNYLD

SEQ ID NO: 88 - protein, 7 aa, synthetic construct
LGSNRDS

SEQ ID NO: 89 - protein, 7 aa, synthetic construct
KDTERPS

SEQ ID NO: 90 - protein, 7 aa, synthetic construct
LGSDRAS

SEQ ID NO: 91 - protein, 7 aa, synthetic construct
LVSNRAS

SEQ ID NO: 92 - protein, 7 aa, synthetic construct
DTSNRAS

SEQ ID NO: 93 - protein, 7 aa, synthetic construct
DASRLED

SEQ ID NO: 94 - protein, 7 aa, synthetic construct
QVSNRDS

SEQ ID NO: 95 - protein, 7 aa, synthetic construct
GASSGAT

SEQ ID NO: 96 - protein, 7 aa, synthetic construct
TTSSRAS

SEQ ID NO: 97 - protein, 7 aa, synthetic construct
LASNRAS

SEQ ID NO: 98 - protein, 10 aa, synthetic construct
FGQGTKVEVK

SEQ ID NO: 99 - protein, 11 aa, synthetic construct
QSPDSSGTVEV

SEQ ID NO: 100 - protein, 9 aa, synthetic construct
MQALQTPTT

SEQ ID NO: 101 - protein, 9 aa, synthetic construct
MQTLQTPLS

SEQ ID NO: 102 - protein, 9 aa, synthetic construct
QQRYDWPLT

SEQ ID NO: 103 - protein, 9 aa, synthetic construct
QQYDDLPYT

SEQ ID NO: 104 - protein, 9 aa, synthetic construct
MQGTQWPPT

SEQ ID NO: 105 - protein, 9 aa, synthetic construct
MQALRTPYT

SEQ ID NO: 106 - protein, 9 aa, synthetic construct
LQGSHWPWT

SEQ ID NO: 107 - protein, 8 aa, synthetic construct
QLYGSSLA

SEQ ID NO: 108 - protein, 9 aa, synthetic construct
MQRLQFPLT

SEQ ID NO: 109 - protein, 9 aa, synthetic construct
MQTLETPVT

SEQ ID NO: 110 - protein, 9 aa, synthetic construct
LQGTHWPWT

SEQ ID NO: 111 - protein, 113 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 112 - protein, 107 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 113 aa, synthetic construct; identical to SEQ ID NO: 111

SEQ ID NO: 114 - protein, 107 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 115 - protein, 113 aa, synthetic construct; identical to SEQ ID NO: 111

SEQ ID NO: 116 - protein, 107 aa, synthetic construct; identical to SEQ ID NO: 114

SEQ ID NO: 117 - protein, 482 aa, synthetic construct
QVQLQQWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTLVTVSSGGGGSGGGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIKNSGAGTAAATHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLCVSKLTVDKSRWQQGNVFSCSVMHEALHNHYTQKSLSLSPGK

SEQ ID NO: 118 - protein, 243 aa, synthetic construct
QVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAGDILTGYALDYWGQGTLVTVSSGGGGSGGGGSGGGGSSYELTQPPSMSVSPGQTARITCSGDALPKQFAFWYQQKPGQAPVLVIYKDTERPSGIPERFSGSSSGTTVTLTITGVQAEDEADYYCQSPDSSGTVEVFGGGTKLTVL

SEQ ID NO: 119 - protein, 247 aa, synthetic construct
QVQLQQWGAGLLKPSETLSLTCAVYGGSFSGYYWSWIRQPPGKGLEWIGEINHFGSTNYNPSLKSRATISVDTSKNQFSLKLSSVTAADTAVYYCATSPGGYSGGYFQHWGQGTLVTVSSGGGGSGGGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 120 - protein, 247 aa, synthetic construct
QVQLQQWGAGLLKPSETLSLTCAVYGGSFSGYYWSWIRQPPGKGLEWIGEINHFGSTNYNPSLKSRATISVDTSKNQFSLKLSSVTAADTAVYYCATSPGGYSGGYFQHWGQGTLVTVSSGGGGSGGGGSGGGGSDVVMTQSPLSLPVTPGESVSISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 121 - protein, 247 aa, synthetic construct
QVQLQQWGAGMLKPSETLSLTCAVYGGSFSGYYWSWIRQPPGKGLEWIGEINHFGSTNYNPSLKSRATISVDTSKNQFSLKLSSVTAADTAVYYCATSPGGYSGGYFQHWGQGTLVTVSSGGGGSGGGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 122 - protein, 255 aa, synthetic construct
QVQLQQWGAGLLKPSETLSLTCAVYGGSFSGYYWSWIRQPPGKGLEWIGEINHFGSTNYNPSLKSRATISVDTSKNQFSLKLSSVTAADTAVYYCATSPGGYSGGYFQHWGQGTLVTVSSGGGGSGEGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIKAAAHHHHH

SEQ ID NO: 123 - protein, 247 aa, synthetic construct
EVQLVESGGGLVQPGGSLRLSCAASGGSFSGYYWSWVRQAPGKGLEWVSEINHSGSTNYNPSLKSRFTISRDNSKNTLYLQMNSLRAEDTAVYYCARSPGGYSGGYFQHWGQGTLVTVSSGGGGSGGGGSGGGGSDIQMTQSPSSLSASVGDRVTITCRSSQSLLHSNGYNYLDWYQQKPGKAPKLLIYLGSNRDSGVPSRFSGSGSGTDFTLTISSLQPEDFATYYCMQGTHWPWTFGQGTKVEIK

SEQ ID NO: 124 - protein, 247 aa, synthetic construct
QVQLQQSGAEVKKPGSSVKVSCKASGGTFSSYAISWVRQAPGQGLEWMGGFDPEDGETIYAQKFQGRVTMTEDTSTDTAYMELSSLRSEDTAVYYCATDLRIPYYYDNPWGQGTLVTVSSGGGGSGGGGSGGGGSEIVLTQSPLSLPVTPGEPASISCRSSQSLLHSNGYNYLNWYLQKPGQSPQLLIYLGSDRASGVPDRFSGSGSGTDFTLKISRVEAEDVGVYYCMQALQTPTTFGGGTKVEIK

SEQ ID NO: 125 - protein, 245 aa, synthetic construct
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARSLYYSHFDYWGQGTLVTVSSGGGGSGGGGSGGGGSEIVMTQSPLSLPVTPGEPASISCRSSQSLLHSNGYNYLDWYLQKPGQSPQLLMYLVSNRASGVPERFSGSGSGTDFTLKISRVEAEDVGVYYCMQTLQTPLSFGQGTKLEIK

SEQ ID NO: 126 - protein, 245 aa, synthetic construct
EVQLVETGPGLVKPSGTLSLTCAVSGGSISSSNWWSWVRQPPGKGLEWIGEIYHSGSTNYNPSLKSRVTISVDKSKNQFSLKLSSVTAADTAVYYCAREGPLSSSGPGAFDIWGQGTMVTVSSGGGGSGGGGSGGGGSEIVLTQSPATLSLSPGERATLSCRASQSVSYHLAWYQQKPGQAPRLLIYDTSNRASGIPARFSGSGSGTDFTLTINSLEPEDFAVYYCQQRYDWPLTFGGGTKVEIK

SEQ ID NO: 127 - protein, 236 aa, synthetic construct
QVQLQESGGGVVQPGRSLRLSCAASGFTFSNYGMHWVRQAPGKGLEWVAVISYDGTNKYYADSVKGRFTISRDNSKNTVYLQMNSLRAEDTAVYYCAKDGFDIWGQGTMVTVSSGGGGSGGGGSGGGGSDIQMTQSPSFLSASVGDRVTITCRASQSISSWLAWYQQKPGKAPKLLIYDASRLEDGVPSRFSGTGFGTDFTFTITTLQPDDIATYYCQQYDDLPYTFGQGTTVDIK

SEQ ID NO: 128 - protein, 247 aa, synthetic construct
EVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCARDVYGDYGAFDYWGQGTLVTVSSGGGGSGGGGSGGGGSDVVMTQSPVSLPVTLGQPASISCKSSQSLLYFNGNTYLSWFQQRPGQSPRRLFYQVSNRDSGVPDRFSGSGSDTDFTLTISRVEAEDVGVYFCMQGTQWPPTFGQGTKVEIK

SEQ ID NO: 129 - protein, 244 aa, synthetic construct
QLQLQESGGGVVQPGRSLRLSCAASGFTFSSYAMHWVRQAPGKGLEWVAVISYDGGNKYYADSVKGRFTISRDNSKNTLYLQMNSLRAEDTAVYYCAKSVAPPMDVWGKGTTVTVSSGGGGSGGGGSGGGGSDVVMTQSPLSLPVTPGEPASISCRSSQSLLHSNGYNYLDWYLQKPGQSPHLLIYLGSNRASGVPDRFSGSGSGTDFTLKISRVEAEDVGVYFCMQALRTPYTFGQGTKLEIK

SEQ ID NO: 130 - protein, 248 aa, synthetic construct
QVQLQQWGAGLLKPSETLSLTCAVYGESFSGYYWSWIRQPPGKGLEWIGEINHSGSTNYNPSLKSRVTISVDTSKNQFSLKLSSVTAADTAVYYCARGPAGSSSSGYFDYWGQGTLVTVSSGGGGSGGGGSGGGGSDVVMTQSPLSLPVTLGQPASISCRSSQSLVHSNGYNYLDWYLQKPGQSPQLLIYLGSNRASGVPDRFSGSGSGTDFTLKISRVEAEDVGVYYCLQGSHWPWTFGQGTKVEIK

SEQ ID NO: 131 - protein, 237 aa, synthetic construct
QVQLQESGPGLVKPSQTLSLTCTVSGGSISSGGYYWTWIRQHPGQGLEWIGFISWSGTTYYNPSLKNRVTISADTSKNHFSLNLTSVTAADTAVYYCARGSGRLVWGQGTLVTVSSGGGGSGGGGSGGGGSETTLTQSPATLSVSPGERATLSCRASQSVSSNLAWYQQKPGQAPRLLIYGASSGATGIPDRFSGSGSGTDFTLTISRLEPEDFAVYYCQLYGSSLAFGGGTKVEIK

SEQ ID NO: 132 - protein, 248 aa, synthetic construct
EVQLVQSGAEVKKPGASVKVSCKASGYTFTGYYMHWVRQAPGQGLEWMGIINPSGGSTSYAQKFQGRVTMTRDTSTSTVYMELSSLRSEDTAVYYCARADTAMGDAFDIWGQGTMVTVSSGGGGSGGGGSGGGGSDIVMTHTPLSLPVTPGEPASISCRSSQTLFDSDDGKTYLDWYLQKPGQSPQLLMYTTSSRASGVPDRFSGSGSGTDFTLKISRVEAEDVGVYYCMQRLQFPLTFGQGTRLEFK

SEQ ID NO: 133 - protein, 245 aa, synthetic construct
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARSIGIGAFDIWGQGTMVTVSSGGGGSGGGGSGGGGSDVVMTQSPLSLPVTPGEPASISCRATQSLLHGNGHNYLDWYLQKPGQSPQLLIYLGSNRASGVPDRFSGSGSGTDFTLKISRVEAEDVGVYYCMQTLETPVTFGPGTKVDIK

SEQ ID NO: 134 - protein, 248 aa, synthetic construct
QVQLQQWGAGLLKPSETLSLTCTIYGGSFSGFYWSWIRQPPGKGLEWIGEINHSGSTNYNPSLKSRVTISVDTSKNQFSLKLSSVTAADTAIYYCARGPAGSTSSGYFDHWGQGTLVTVSSGGGGSGGGGSGGGGSDVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPWTFGQGTKVEIK

SEQ ID NO: 135 - protein, 244 aa, synthetic construct
QVQLQQWGAGLLKPSETLSLTCAVYGGSLSGYYWSWIRQPPGKGLEWIGEINHSGSTNYNPSLKSRVTISVDTSKNQFSLKLSSVTAADTAVYYCARGSSSYYMDVWGKGTTVTVSSGGGGSGGGGSGGGGSDVVMTQSPLSLPVTPGEPASISCRSSQSLLHSNGYNYLDWYLQKPGQSPQLLIYLGSNRASGVPDRFSGSGSGTDFTLKISRVEAEDVGVYYCLQGTHWPWTFGQGTKVEIK

SEQ ID NO: 136 - protein, 243 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGGSAYFQHWGQGTLVTVSSGGGGSGGGGSGGGGSDVVMTQSPLSLPVTLGQPASISCRSSQSLLHSNGNNYLDWYLQKPGQSPQLLIYLGSNRASGVPDRFSGSGSGTDFTLKISRVEAEDVGIYYCMQGTHWPWTFGQGTKVEIE

SEQ ID NO: 137 - protein, 248 aa, synthetic construct
QVQLQQWGAGLLKPSETLSLTCAVYGGSFSGYYWSWIRQPPGKGLEWIGEINHSGSTNYNPSLKSRVTISEDASKKQFSLTLTSVTAADTAVYYCARGPAGTGSSGYFDYWGQGTLVTVSSGGGGSGGGGSGGGGSEIVLTQSPLSLPVTPGEPASISCRSSQSLLHSNGYNYLDWYLQKPGQSPQLLIYLASNRASGVPDRFSGSGSGTDFTLKISRVEAEDVGVYYCMQGTHWPWTFGQGTKVEVK

SEQ ID NO: 138 - protein, 9 aa, synthetic construct
NSGAGTAAA

SEQ ID NO: 139 - protein, 23 aa, synthetic construct
NSGAGTSGSGASGEGSGSKLAAA

SEQ ID NO: 140 - protein, 8 aa, synthetic construct
GGGGSAAA

SEQ ID NO: 141 - protein, 15 aa, synthetic construct
GGGGSGGGGSGGGGS

SEQ ID NO: 142 - protein, 9 aa, synthetic construct
AAAHHHHHH

SEQ ID NO: 143 - DNA, 657 bp, synthetic construct
  source 1..657: mol_type = other DNA
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaaccgggactctggggtcccagacagattcagcggcagtgggtcaggcactgatttcacactgaaaatcagcagggtggaggctgaggatgttggggtttattactgcatgcaaggtacacactggccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 144 - DNA, 669 bp, synthetic construct
  source 1..669: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagccaccatatcagtagacacgtccaagaaccagttctccctgaagctgagctctgtgaccgccgcggacacggctgtgtattactgtgcgaccagcccgggaggctattccgggggatacttccagcactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 145 - DNA, 1443 bp, synthetic construct
  source 1..1443: mol_type = other DNA
caggtccagctgcagcagtggggagccggcctgctgaaaccatctgaaactctgagcctgacttgcgctgtctacggggggtccttcagtggctactattggtcatggatcaggcagccccctgggaagggactggagtggatcggggaaattaaccactccggatctacaaactacaatcccagtctgaaatcacgcgccaccatttctgtggacaccagtaagaatcagttcagcctgaagctgagcagcgtgacagccgctgataccgccgtgtactattgcgcaaccagccctggcggatactccggaggctattttcagcattggggccaggggaccctggtgacagtctctagtgggggaggagggtctggaggaggaggaagtggaggaggaggctccgacgtggtcatgactcagagcccactgtccctgccagtgacccccggcgagcctgctagtatctcatgtcgatcaagccagtcactgctgcacagcaacgggtacaattatctggattggtacttgcagaagccaggccagtctccccagctgctgatctatctgggctccaaccgggactctggggtgcctgatagattcagcggcagcggctctgggactgactttaccctgaaaatttccagagtcgaggcagaagatgtgggagtctactattgcatgcagggcactcattggccctggaccttcggacagggcacaaaggtggagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 146 - DNA, 639 bp, synthetic construct
  source 1..639: mol_type = other DNA
tcctatgagctgactcagccaccctcgatgtcagtgtccccaggacagacggccaggatcacctgctctggagatgcattgccaaaacaatttgctttttggtaccagcagaagccaggccaggcccctgtgttggtgatttataaagacactgagaggccctcagggatccctgagcgattctctggctccagctcagggacaacagtcacgttgaccatcactggagtccaggcagaagatgaggctgactattactgtcaatctccagacagcagtggtaccgtcgaagtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctgcagaatgt

SEQ ID NO: 147 - DNA, 669 bp, synthetic construct
  source 1..669: mol_type = other DNA
caggtgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgggcgatattttgactggttatgcccttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 148 - DNA, 1431 bp, synthetic construct
  source 1..1431: mol_type = other DNA
caggtgcagctgcaggaaagcggacccggactggtgaagccatctgaaacactgagcctgacttgtaccgtgagcggcggaagcatcagctcctctagttactattggggatggatcaggcagccccctggcaaggggctggagtggatcggcagcatctactatagcggctccacatactataaccctagcctgaaatcccgcgtgacaatctctgtggacactagtaagaatcagttctctctgaaactgtcaagcgtgaccgccgctgatacagctgtctactattgcgcaggcgacattctgaccgggtacgccctggattattggggacagggcactctggtgaccgtctcctctggaggaggaggctcaggaggaggagggtccggaggcgggggaagttcatacgaactgacacagccaccctctatgagtgtgtcaccagggcagactgcacgaatcacctgtagcggagacgccctgcccaagcagttcgctttttggtatcagcagaaacctggccaggctccagtgctggtcatctataaggatactgagcggccctctgggattcctgaaagattcagtggcagcagcagcggaaccacagtgactctgaccattacaggcgtgcaggcagaggacgaagccgattactattgccagtcccccgacagttcaggcaccgtggaggtctttggcgggggaacaaaactgactgtgctgaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 149 - DNA, 657 bp, synthetic construct
  source 1..657: mol_type = other DNA
gaaattgtgctgactcagtctccactctccctgcccgttacccctggagagccggcctccatctcctgcaggtctagtcagagcctcctgcatagtaatggatacaactatttgaattggtacctacagaagccagggcagtctccacaactcctgatctatttgggttctgatcgggcctccggggtccctgacaggttcagtggcagtggatcaggcacagattttacactgaaaatcagcagagtggaggctgaggatgttggggtttattactgcatgcaagctctacaaacccccaccactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 150 - DNA, 669 bp, synthetic construct
  source 1..669: mol_type = other DNA
caggtacagctgcagcagtcaggggctgaggtgaagaagcctgggtcctcggtgaaggtctcctgcaaggcttctggaggcaccttcagcagctatgctatcagctgggtgcgacaggcccctggacaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcaacagatctgagaattccgtattactatgataacccctggggccagggcaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 151 - DNA, 1443 bp, synthetic construct
  source 1..1443: mol_type = other DNA
caggtccagctgcagcagagcggagccgaggtcaagaagccagggagtagcgtcaaagtcagttgtaaagcatcaggaggaacattcagctcctatgcaatctcttgggtgcgacaggcccctggacagggcctggagtggatgggaggattcgacccagaggatggagaaaccatctacgcccagaagtttcagggcagagtgactatgaccgaagacacatctactgataccgcttacatggagctgtctagtctgaggagtgaagacactgccgtctactattgcgctaccgacctgcgcatcccatactattacgataatccctgggggcagggaacactggtgactgtctcaagcggaggcgggggatcaggcggaggaggcagcggaggaggagggtccgagatcgtgctgacacagagtccactgtcactgccagtcacccctggcgaaccagccagtatttcatgtcggtcctctcagagcctgctgcactccaacgggtataattacctgaactggtacttgcagaagcctggccagagccctcagctgctgatctacctgggctctgaccgagcaagtggggtgcccgatagattcagcggctccgggtctggaaccgactttaccctgaagatcagccgggtggaggctgaagatgtgggcgtctattactgcatgcaggccctccagacacctaccacattcggaggcgggactaaggtggagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 152 - DNA, 657 bp, synthetic construct
  source 1..657: mol_type = other DNA
gaaattgtgatgacgcagtctccactctccctgcccgtcacccctggagagccggcctccatctcctgcaggtctagtcagagcctcctgcatagtaatggatacaactatttggattggtacctgcagaagccagggcagtctccacagctcctgatgtatttggtttctaatcgggcctccggggtccctgagaggttcagtggcagtggatcaggcacagattttacactgaaaatcagcagagtggaggctgaggatgttggggtttattactgcatgcaaactctacaaactcctctcagttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 153 - DNA, 663 bp, synthetic construct
  source 1..663: mol_type = other DNA
caggtccagctggtacagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagccgaggacacggctgtgtattactgtgcgagaagtctttactacagccactttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 154 - DNA, 1437 bp, synthetic construct
  source 1..1437: mol_type = other DNA
caggtgcagctggtccagagcggaggaggcgtcgtccagcccggaaggtcactgagactgtcttgtgccgcatcaggattcacttttagctcctacgcaatgcactgggtgaggcaggcccctggcaaggggctggagtgggtggctgtcatcagttatgacggctcaaacaagtactatgcagatagcgtgaaagggcggttcaccattagcagagacaactccaaaaatacactgtacctccagatgaacagcctgcgagccgaagacacagctgtgtactattgcgcccggtctctgtactatagtcactttgattactggggacagggcaccctggtgacagtctctagtggcgggggaggcagtggaggaggagggagcggaggaggaggcagcgagatcgtgatgactcagtccccactgtctctgccagtcacccctggcgaaccagcatccatttcttgtagatcaagccagtcactgctgcatagcaacggatacaattatctggattggtacttgcagaagcctggccagtctcctcagctgctgatgtatctggtgtccaacagggcctctggggtcccagagcgcttcagtgggtcaggaagcggcactgactttaccctgaaaatctctcgcgtggaggctgaagatgtgggcgtctactattgcatgcagacactccagactcccctgagcttcgggcagggaaccaagctggagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 155 - DNA, 642 bp, synthetic construct
  source 1..642: mol_type = other DNA
gaaattgtgctgactcagtctccagccaccctgtctttgtctccaggggaacgagccaccctctcctgcagggccagtcagagtgttagttaccacttagcctggtaccaacaaaaacctggccaggctcccaggctcctcatctatgatacatccaacagggcctctggcatccccgccaggttcagtggcagtgggtctgggacagacttcactctcaccatcaacagcctagagcctgaagattttgcagtttattactgtcagcagcgttacgactggcctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 156 - DNA, 678 bp, synthetic construct
  source 1..678: mol_type = other DNA
gaggtgcagctggtggagactggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagagaagggcccctaagcagcagcggaccgggtgcttttgatatctggggccaagggacaatggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 157 - DNA, 1410 bp, synthetic construct
  source 1..1410: mol_type = other DNA
caggtccagctgcaggaatcaggagggggggtcgtccagccagggaggtcactgagactgtcttgcgccgcttcagggttcacttttagcaactacggaatgcactgggtgcggcaggctcccggcaaagggctggagtgggtggcagtcatctcttatgacggcacaaacaagtactatgcagatagtgtcaaggggcggttcaccatcagccgggacaacagtaaaaatacagtgtacctccagatgaacagcctgcgggccgaagatactgctgtctactattgcgccaaggacgggtttgacatctggggacagggcactatggtgaccgtcagctccggcgggggaggctcaggaggaggagggagcggaggaggaggcagcgacattcagatgacccagtcacctagcttcctgtccgcttctgtgggcgatagggtcacaatcacttgtcgcgccagtcagtcaatttctagttggctggcttggtatcagcagaagcccggaaaagcacctaagctgctgatctatgacgcctcccgactggaggatggcgtgccaagcagattctccgggacaggatttggcactgacttcacctttacaatcaccacactccagccagacgatattgccacttactattgccagcagtacgacgatctgccctatacctttgggcagggaactaccgtggatattaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 158 - DNA, 642 bp, synthetic construct
  source 1..642: mol_type = other DNA
gacatccagatgacccagtctccttccttcctgtctgcatctgtaggagacagagtcaccatcacttgccgggccagtcagagtattagtagctggttggcctggtatcagcagaaaccagggaaagcccctaaactcctgatctacgatgcatcccgtttggaggacggggtcccatcaagattcagtggaactggatttgggacagattttactttcaccattaccaccctgcagcctgacgatattgcgacatattattgtcagcaatacgatgatctcccgtacacttttggccaggggaccacggtggacatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 159 - DNA, 651 bp, synthetic construct
  source 1..651: mol_type = other DNA
caggtgcagctgcaggagtccgggggaggcgtggtccagcctgggaggtccctgagactctcctgtgcagcctctggattcaccttcagtaactatggcatgcactgggtccgccaggctccaggcaaagggctggagtgggtggcagttatatcatatgatggaactaataaatactatgcagactccgtgaagggccgattcaccatctccagagacaattccaagaacacggtgtatctgcaaatgaacagcctgagagctgaggacacggctgtgtattactgtgcgaaagatggttttgatatttggggccaagggacaatggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 160 - DNA, 1437 bp, synthetic construct
  source 1..1437: mol_type = other DNA
gaagtgcagctggtggaaactggacctggactggtgaagccaagcgggactctgagcctgacctgtgccgtgagcgggggaagtatcagctcctctaactggtggtcctgggtgcgacagccccctggcaaggggctggagtggatcggcgaaatctaccacagcgggtccacaaactataatcctagcctgaagagccgggtgactatctctgtggacaagagtaaaaatcagttcagcctgaaactgagttcagtgacagccgctgataccgccgtgtactattgcgccagggagggacctctgagcagcagcggaccaggcgcttttgacatctgggggcagggaactatggtgaccgtcagttcaggcggaggaggctccggaggaggagggtctggaggcgggggaagtgagattgtgctgacccagtcccccgccacactgtctctgagtcctggcgaacgggccaccctgtcttgtagagcttcacagagcgtgtcctaccatctggcatggtatcagcagaaaccaggccaggcccccagactgctgatctacgacacctcaaacagggctagcggcattcccgcacgcttctctggcagtgggtcaggaacagattttaccctgacaatcaatagcctggagccagaagacttcgccgtgtactattgccagcagcgctatgattggcccctgacttttggcgggggaaccaaggtcgagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 161 - DNA, 657 bp, synthetic construct
  source 1..657: mol_type = other DNA
gatgttgtgatgactcagtctccagtctccctgcccgtcacccttggacagccggcctccatctcctgcaagtctagtcaaagcctcctttactttaatggaaacacctacttgagctggtttcagcagaggccaggccaatctccacggcgcctattttatcaggtttctaaccgggactctggggtcccagacagattcagcggcagtgggtcagacactgatttcactctgaccattagcagggtggaggctgaagatgttggagtttatttctgcatgcaaggaacacagtggcctccgacgttcggccaagggaccaaggtggaaatcaaacgaactgtggctgcaccatctgtcttcatcttcccgccatctgatgagcagctgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 162 - DNA, 669 bp, synthetic construct
  source 1..669: mol_type = other DNA
gaggtccagctggtgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagccgaggacacggccgtgtattactgtgcgagagatgtctacggtgactacggggcctttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 163 - DNA, 1443 bp, synthetic construct
  source 1..1443: mol_type = other DNA
gaggtccagctggtccagagcggcggaggggtcgtccagcccggaagaagcctgagactgtcctgtgcagcaagtgggtttacattcagctcctacggcatgcactgggtgaggcaggcacccggcaaggggctggagtgggtggccgtcatcagttatgacggctcaaacaagtactatgccgatagcgtgaaagggaggttcacaattagccgcgacaactccaaaaatactctgtacctccagatgaacagcctgagagccgaagatacagctgtgtactattgcgctagggacgtctacggagattatggcgcatttgactattggggacagggcactctggtgaccgtctctagtggaggaggaggctcaggaggaggagggagcggcggaggaggcagcgatgtggtcatgacccagtccccagtgtctctgccagtcacactgggacagccagcatccatctcttgtaagtcaagccagtctctgctgtacttcaacggaaatacttatctgtcttggtttcagcagcgccctggccagagtccacggagactgttctaccaggtgtctaaccgagacagtggcgtccctgatcggttcagtgggtcaggaagcgacaccgattttaccctgacaatcagccgagtggaggctgaagacgtgggggtctatttctgcatgcagggaacacagtggccccctacttttggccaggggaccaaggtggagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 164 - DNA, 657 bp, synthetic construct
  source 1..657: mol_type = other DNA
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacacctcctgatctacttgggttctaatcgggcctccggggtccctgacaggttcagtggcagtggatcaggcacagattttacactgaaaattagcagagtggaggctgaggatgttggggtttatttctgcatgcaagctctacgaactccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 165 - DNA, 660 bp, synthetic construct
  source 1..660: mol_type = other DNA
cagctgcagctgcaggagtcggggggaggcgtggtccagcctgggaggtccctgagactctcctgtgcagcctctggattcaccttcagtagctatgctatgcactgggtccgccaggctccaggcaaggggctggagtgggtggcagttatatcatatgatggaggtaataaatactacgcagactccgtgaagggccgattcaccatctccagagacaattccaagaacacgctgtatctgcaaatgaacagcctgagagctgaggacacggctgtgtattactgtgcgaaatcagtggcgcctcccatggacgtctggggcaaagggaccacggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 166 - DNA, 1434 bp, synthetic construct
  source 1..1434: mol_type = other DNA
cagctgcagctgcaggaatccgggggaggcgtcgtccagccaggaaggtcactgagactgagttgtgccgcaagcgggttcactttcagctcctacgctatgcactgggtgagacaggcacccggaaagggcctggagtgggtggcagtcatctcttatgacggcgggaacaagtactatgccgatagtgtgaaaggccggttcaccattagtagagacaactcaaaaaatacactgtacctccagatgaatagcctgcgcgccgaagacacagctgtgtactattgcgcaaagtccgtggccccccctatggatgtctgggggaaaggaaccacagtgactgtctctagtggaggaggaggatcaggcggcggaggcagcggaggaggagggtccgacgtggtcatgactcagtcccctctgtctctgccagtgacccccggcgagcctgcttccatctcttgtaggtcaagccagagcctgctgcactccaacgggtacaattatctggattggtacttgcagaagccaggccagtctccccatctgctgatctatctgggatctaacagggccagtggcgtgcctgaccgcttcagtggctcagggagcggaactgattttaccctgaaaattagccgagtcgaggccgaagatgtgggcgtctacttctgcatgcaggctctgcggacaccatatacttttggccaggggaccaagctggagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 167 - DNA, 657 bp, synthetic construct
  source 1..657: mol_type = other DNA
gatgttgtgatgactcagtctccactctccctgcccgtcacccttggacagccggcctccatctcctgcaggtctagtcagagcctcgtccatagtaatggatacaactatttggactggtacctgcagaagccagggcagtctccacagctcctgatctatttgggttctaatcgggcctccggggtccctgacaggttcagtggcagtggatcgggcacagattttacactgaaaatcagcagagtggaggctgaggatgttggggtttattactgcctgcaaggttcacactggccttggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 168 - DNA, 672 bp, synthetic construct
  source 1..672: mol_type = other DNA
caggtgcagctacagcagtggggcgcaggactgttgaagccttcggagaccctgtccctcacctgcgctgtctatggtgagtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagaggccccgcgggtagcagctcgtccggctactttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 169 - DNA, 1446 bp, synthetic construct
  source 1..1446: mol_type = other DNA
caggtccagctgcagcagtggggcgccggactgctgaaaccctctgaaactctgagcctgacttgtgccgtctatggggaatccttctctggctactattggagttggatcaggcagccccctggcaaggggctggagtggatcggagaaattaaccacagcggctccaccaactacaatccatctctgaaaagtcgcgtgaccatttccgtggacacatctaagaatcagttcagcctgaagctgagcagcgtgacagccgctgatactgccgtctactattgcgcacggggccccgccgggtctagttcaagcggatactttgactattggggacagggcaccctggtgacagtctcctctggcggaggaggctccggaggaggagggtctggaggaggaggaagcgatgtggtcatgacacagtcaccactgagcctgccagtgactctgggacagcctgcttctatcagttgtcgaagttcacagagtctggtccactcaaacggatacaattatctggactggtacttgcagaagcctggccagagcccacagctgctgatctatctggggagcaaccgagcttccggagtgcccgacagattctcagggagcggcagcggcactgattttaccctgaaaattagcagagtggaggcagaagatgtgggcgtctactattgcctccaggggtcccattggccttggactttcgggcagggaaccaaggtggagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 170 - DNA, 639 bp, synthetic construct
  source 1..639: mol_type = other DNA
gaaacgacactcacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccagcggggccactggcatcccagacaggttcagtggcagtgggtctgggacagacttcactctcaccatcagcagactggagcctgaagattttgcagtgtattactgtcagctgtatggtagctcactcgctttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 171 - DNA, 687 bp, synthetic construct
  source 1..687: mol_type = other DNA
gaggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggatggatcagcgcttacaatggtgacacaagctacgcacagaagttccagggcagagtcaccattaccagggacacatccgcgagcacagcctacatggagctgagcagcctgagatctgaagacacggctgtgtattactgtgcgagagattggggatattgtagtggtggtagctgctacctgaactggttcgacccctggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 172 - DNA, 1412 bp, synthetic construct
  source 1..1412: mol_type = other DNA
aggtccagctgcaggaaagcgggccaggactggtcaaaccctcacagacactgtctctgacttgtaccgtctccgggggctcaatcagctccggcgggtactattggacatggatcagacagcaccctggacagggcctggagtggatcgggttcattagctggtccggaaccacatactataacccaagcctgaagaatagggtgacaatttcagccgacactagcaaaaaccatttttccctgaatctgacctctgtgacagccgctgatactgctgtctactattgcgcacgggggtccggaagactggtgtggggacaggggactctggtgaccgtctctagtggaggaggaggaagtggcggaggaggcagcggaggaggagggtccgagactaccctgacccagtctccagctacactgtctgtgagtcccggcgaaagggcaaccctgagctgtcgcgcttcacagagcgtctcaagcaacctggcatggtatcagcagaagcctggccaggcccctcgactgctgatctatggggcatcctctggagccactggcattcccgaccggttctccggatctggcagtgggaccgattttacactgaccatcagccggctggagcctgaagacttcgctgtgtactattgccagctgtacggcagttcactggcatttggaggcgggacaaaggtcgagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 173 - DNA, 660 bp, synthetic construct
  source 1..660: mol_type = other DNA
gatattgtgatgacccacactccactctccctgcccgtcacccctggagagccggcctccatctcctgcaggtctagtcagaccctcttcgatagcgatgatggaaagacctatttggactggtacctgcagaagccagggcagtctccacaactcctgatgtataccacttcctctcgggcctctggagtcccagacaggttcagtggcagtgggtcaggcactgatttcacactgaaaatcagcagggtggaggctgaggatgttggagtttattactgcatgcagcgtttacagtttcccctcaccttcggccaagggacacgactggagttcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 174 - DNA, 669 bp, synthetic construct
  source 1..669: mol_type = other DNA
gaggtccagctggtgcagtctggggctgaggtgaagaagcctggggcctcagtgaaggtctcctgcaaggcttctggatacaccttcaccggctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagccgatacagctatgggtgatgcttttgatatctggggccaagggacaatggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 175 - DNA, 1446 bp, synthetic construct
  source 1..1446: mol_type = other DNA
gaagtccagctggtccagtcaggagccgaggtcaagaagccaggggcaagcgtcaaagtctcatgcaaagcaagtgggtacacatttacaggctactatatgcactgggtgaggcaggctccaggacagggcctggagtggatggggatcattaaccccagcggcgggagtacctcatacgcacagaagttccagggacgggtgactatgaccagagacacaagcacttccaccgtctatatggagctgagcagcctgcgatccgaagacactgccgtgtactattgcgccagagccgataccgcaatgggcgacgcctttgacatctgggggcagggcacaatggtgacagtctctagtggaggaggaggatctggaggaggaggcagtggaggaggcgggtcagacatcgtgatgacacatactccactgtctctgccagtcacccctggcgagccagcctctattagttgtcgctcaagccagaccctgttcgacagtgacgatggaaagacatacctggattggtacttgcagaaacctggccagagccctcagctgctgatgtacaccacatcctctagggcctccggcgtgcctgaccgcttctcaggcagcgggtccggaactgattttaccctgaagatcagccgggtggaggctgaagacgtgggggtctactattgcatgcagagactccagttcccactgacatttggccaggggactcggctggagttcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 176 - DNA, 657 bp, synthetic construct
  source 1..657: mol_type = other DNA
gatgttgtgatgactcagtctccactctccctgcccgtcacccctggagagccggcctccatctcctgcagggcaactcagagcctcctgcatggaaatggacacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaaactctggaaactcctgtcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 177 - DNA, 663 bp, synthetic construct
  source 1..663: mol_type = other DNA
gaggtccagctggtgcagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgaggtctatcggtatcggtgcttttgatatctggggccaagggacaatggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 178 - DNA, 1437 bp, synthetic construct
  source 1..1437: mol_type = other DNA
gaggtccagctggtccagagcggggggggggtcgtgcagcctgggagaagcctgagactgtcctgtgccgcaagcgggtttacttttagctcctacgctatgcactgggtgaggcaggcacccggcaaggggctggagtgggtggcagtcatctcctatgacggctctaacaagtactatgccgatagcgtgaaagggcggttcacaattagtagagacaactcaaagaacactctgtacctccagatgaatagcctgcgagccgaagacactgctgtgtactattgcgcccggtccatcggaattggcgcttttgacatctgggggcagggcacaatggtgacagtctctagtggaggaggaggctctggaggaggagggagtggaggaggaggatcagacgtggtcatgacccagtcacctctgagcctgccagtgacacctggcgagccagcatcaattagctgtagagccacccagtctctgctgcacggcaacgggcataattacctggattggtacttgcagaagcctggccagagtcctcagctgctgatctatctggggagcaacagggcttccggagtgccagaccgcttctccggatctggcagtgggactgattttaccctgaaaatttcccgcgtcgaggcagaagacgtgggagtctactattgcatgcagacactggaaactccagtgaccttcggacccggcacaaaggtggacatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 179 - DNA, 657 bp, synthetic construct
  source 1..657: mol_type = other DNA
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagggtggaggctgaggatgttggggtttattactgcatgcaaggtacacactggccctggacgttcggccaagggaccaaggtggaaatcaaacgaactgtggctgcaccatctgtcttcatcttcccgccatctgatgagcagttgaaatctggaactgcctctgttgtgtgcctgctgaataatttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 180 - DNA, 672 bp, synthetic construct
  source 1..672: mol_type = other DNA
caggtgcagctacagcagtggggcgcaggactgttgaagccttcggagaccctgtccctcacctgcactatctatggtgggtccttcagtggtttctactggagctggatccgccagcccccagggaagggactggagtggattggggaaatcaatcatagtggaagcaccaactacaacccgtccctcaagagtcgagtcaccatatcagtagacacgtccaagaaccagttctccctgaagctgagctctgtgaccgccgcggacacggctatatattactgtgcgagaggccccgcgggatccacctcgtccggctactttgaccactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 181 - DNA, 1446 bp, synthetic construct
  source 1..1446: mol_type = other DNA
caggtccagctgcagcagtggggagccggactgctgaaaccctctgagactctgagcctgacttgcacaatctacgggggatcattcagcggcttctactggtcctggatcaggcagccccctggcaaggggctggagtggatcggagaaattaaccacagtggctcaacaaactataatcccagcctgaaatcccgcgtgaccatctcagtggacacaagcaagaatcagttcagcctgaagctgagcagcgtgacagccgctgatactgccatctactattgcgcacggggccctgccgggtccacctctagtgggtactttgaccattggggacagggcaccctggtgacagtctcaagcggaggaggaggctctggaggaggagggagtggaggcgggggcagcgatgtggtcatgactcagtctccactgagtctgccagtgacccccggcgagcctgctagcatctcctgtcgatcctctcagtccctgctgcactctaacggatacaattatctggactggtacttgcagaagccaggccagagcccccagctgctgatctatctggggagtaaccgggcttcaggagtgcctgacagattctctgggagtggatcaggcactgattttaccctgaaaattagcagagtcgaggcagaagatgtgggcgtctactattgcatgcaggggactcattggccctggacctttgggcagggaacaaaggtggagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 182 - DNA, 657 bp, synthetic construct
  source 1..657: mol_type = other DNA
gatgttgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagggtggaggctgaggatgttggggtttattactgcctgcaaggtacacattggccgtggacgttcggccaggggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 183 - DNA, 660 bp, synthetic construct
  source 1..660: mol_type = other DNA
caggtgcagctacagcagtggggcgcaggactgttgaagccttcggagaccctgtccctcacctgcgctgtctatggtgggtccctcagtggttactactggagctggatccgccagcccccagggaaggggctggagtggattggggaaatcaatcatagtggaagcaccaactacaacccatccctcaagagtcgagtcaccatatcagtagacacgtccaagaaccagttctccctgaagctgagctctgtgaccgccgcggacacggctgtgtattactgtgcgagaggcagcagctcctactacatggacgtctggggcaaagggaccacggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 184 - DNA, 1434 bp, synthetic construct
  source 1..1434: mol_type = other DNA
caggtccagctgcagcagtggggagccggactgctgaaaccaagcgagactctgagcctgacttgtgccgtgtatgggggaagcctgtccggctactattggtcttggatcaggcagccccctggcaaggggctggagtggatcggcgaaattaaccactcagggagcacaaactacaatccctccctgaaatctcgcgtgaccattagcgtggacacatccaagaatcagttcagcctgaagctgagcagcgtgacagccgctgacaccgccgtgtactattgcgccagaggcagcagcagctactatatggatgtgtggggaaagggcaccacagtgaccgtcagctccggaggaggaggcagtggaggaggagggtccggaggcgggggatctgacgtggtcatgactcagagtcctctgtcactgcctgtgacccccggcgagcctgcatccatctcttgtcgatctagtcagtctctgctgcacagtaacggctacaattatctggattggtacttgcagaagccagggcagtccccccagctgctgatctatctgggatcaaaccgggctagcggcgtgcctgacagattcagtgggtcaggaagcggcactgattttaccctgaaaattagcagagtcgaggcagaagatgtgggggtctactattgcctccagggaactcattggccctggacctttgggcagggaacaaaggtggagatcaagaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 185 - DNA, 657 bp, synthetic construct
  source 1..657: mol_type = other DNA
gatgttgtgatgactcagtctccactctccctgcccgtcacccttggtcagccggcctccatctcctgcaggtctagtcagagcctcctgcacagtaatggaaacaactatttggattggtacctgcagaagccagggcagtctccacagctcctgatctatttgggttctaatcgggcctccggggtccctgacaggttcagtggcagtggatcaggcacagattttacactgaaaatcagcagggtggaggctgaggatgttgggatttattactgcatgcaagggacacactggccttggacgttcggccaagggaccaaggtggaaatcgaacgaactgtggctgcaccatctgtcttcatcttcccgccatctgatgagcagttgaaatctggaactgcctctgttgtgtgcctgctgaataacttctatcccagagaggccaaagtacagtggaaggtggatagcgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 186 - DNA, 657 bp, synthetic construct
  source 1..657: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagaggcggtagcgcgtacttccagcactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 187 - DNA, 1431 bp, synthetic construct
  source 1..1431: mol_type = other DNA
caggtccagctgcagcagtggggggccgggctgctgaaaccttccgaaactctgtctctgacttgtgccgtgtatggggggtcctttagtggctactattggtcatggatcaggcagccccctggaaagggcctggagtggatcggagaaattaaccactccggctctacaaactacaatccaagtctgaaatcacgcgtgaccatttctgtggacaccagtaagaatcagttcagcctgaagctgagcagcgtgacagccgctgataccgccgtgtactattgcgcccgaggcgggtctgcttattttcagcattgggggcagggaaccctggtgacagtctctagtggaggaggaggcagcggcggaggaggctctggaggaggagggagtgacgtggtcatgactcagagcccactgtccctgccagtgaccctgggacagccagctagtatctcatgtagatcaagccagtcactgctgcacagcaacggcaacaattacctggattggtacttgcagaagcctggccagagcccacagctgctgatctacctggggtccaatcgggcatctggagtgcccgacagattcagcggctccgggtctggaactgattttaccctgaagatcagccgggtggaggccgaagacgtcggcatctactattgcatgcaggggactcattggccttggaccttcggccaggggacaaaagtggagatcgaaaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 188 - DNA, 657 bp, synthetic construct
  source 1..657: mol_type = other DNA
gaaattgtgctgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttggcttctaatcgggcctccggggtcccagacagattcagcggcagtgggtcaggcactgatttcacactgaaaatcagcagggtggaggctgaggatgttggggtttattactgcatgcaaggtacacactggccgtggacgttcggccaagggaccaaggtggaagtcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccgggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 189 - DNA, 672 bp, synthetic construct
  source 1..672: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagaagacgcgtccaagaagcagttctccctgacgctgacctctgtgaccgccgcggacacggctgtctattactgtgcgagaggccccgcgggtaccggctcgtccggctactttgactactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 190 - DNA, 1446 bp, synthetic construct
  source 1..1446: mol_type = other DNA
caggtccagctgcagcagtggggagccggactgctgaagcctagcgaaactctgagcctgacttgtgctgtctacggaggatcatttagtggctactattggtcatggatcaggcagccccctggcaaggggctggagtggatcggagaaattaaccactccggctctacaaactacaatcccagtctgaaatcacgcgtgactatttctgaggacgccagtaagaaacagttctccctgaccctgacatctgtgaccgccgctgatacagctgtctactattgcgcacggggccctgccggaacaggcagctccggatactttgactattgggggcagggaactctggtgaccgtctctagtggcggaggaggcagtggaggaggagggtccggaggaggaggatctgagatcgtgctgactcagagcccactgtccctgccagtcacccccggcgaacctgccagtatttcatgtcgatcaagccagtcactgctgcacagcaacggatacaattatctggactggtacttgcagaagccaggccagagcccccagctgctgatctatctggcttccaatcgggcatctggcgtgcctgacagattcagcggctccgggtctggaacagattttactctgaaaatttccagagtggaggccgaagatgtgggggtctactattgcatgcagggaactcattggccctggaccttcggccaggggacaaaggtggaagtcaaaaacagcggcgcgggcaccgcggccgcgactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 191 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 192 - protein, 525 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 193 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 194 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 195 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 196 - protein, 240 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 197 - protein, 120 aa, synthetic construct
QVQLQAWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTQVTVSS

SEQ ID NO: 198 - protein, 120 aa, synthetic construct
QVQLQAWGAGLLKPSETLSLTCAVYGGSFSGYYWSWIRQPPGKGLEWIGEINHSGSTNYNPSLKSRATISVDTSKNQFSLKLSNVTAADTAVYYCATSPGGYSGGYFQHWGQGTQVTVSS

SEQ ID NO: 199 - protein, 120 aa, synthetic construct
QVQLQASGAEVKKPGSSVKVSCKASGGTFSSYAISWVRQAPGQGLEWMGGFDPEDGETIYAQKFQGRVTMTEDTSTDTAYMELSSLRSEDTAVYYCATDLRIPYYYDNPWGQGTQVTVSS

SEQ ID NO: 200 - protein, 120 aa, synthetic construct
QVQLQASGAEVKKPGSSVKVSCKASGGTFSSYAISWVRQAPGQGLEWMGGFDPEDGETIYAQKFQGRVTMTEDTSTDTAYMELSNLRSEDTAVYYCATDLRIPYYYDNPWGQGTQVTVSS

SEQ ID NO: 201 - protein, 30 aa, synthetic construct
QVQLQAWGAGLLKPSETLSLTCAVYGGSFS

SEQ ID NO: 202 - protein, 30 aa, synthetic construct
QVQLQASGAEVKKPGSSVKVSCKASGGTFS

SEQ ID NO: 203 - protein, 32 aa, synthetic construct
RATISVDTSKNQFSLKLSNVTAADTAVYYCAT

SEQ ID NO: 204 - protein, 32 aa, synthetic construct
RVTMTEDTSTDTAYMELSNLRSEDTAVYYCAT

SEQ ID NO: 205 - protein, 11 aa, synthetic construct
WGQGTQVTVSS

SEQ ID NO: 206 - protein, 120 aa, synthetic construct
  VARIANT 6: note = X can be A, V, I, L, M, F, Y or W
  VARIANT 115: note = X can be Q, S, T or N
QVQLQXWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTXVTVSS

SEQ ID NO: 207 - protein, 120 aa, synthetic construct
  VARIANT 6: note = X can be A, V, I, L, M, F, Y or W
  VARIANT 115: note = X can be Q, S, T or N
  VARIANT 84: note = X can be N, Q, S or T
QVQLQXWGAGLLKPSETLSLTCAVYGGSFSGYYWSWIRQPPGKGLEWIGEINHSGSTNYNPSLKSRATISVDTSKNQFSLKLSXVTAADTAVYYCATSPGGYSGGYFQHWGQGTXVTVSS

SEQ ID NO: 208 - protein, 120 aa, synthetic construct
  VARIANT 6: note = X can be A, V, I, L, M, F, Y or W
  VARIANT 115: note = X can be Q, S, T or N
QVQLQXSGAEVKKPGSSVKVSCKASGGTFSSYAISWVRQAPGQGLEWMGGFDPEDGETIYAQKFQGRVTMTEDTSTDTAYMELSSLRSEDTAVYYCATDLRIPYYYDNPWGQGTXVTVSS

SEQ ID NO: 209 - protein, 120 aa, synthetic construct
  VARIANT 6: note = X can be A, V, I, L, M, F, Y or W
  VARIANT 115: note = X can be Q, S, T or N
  VARIANT 85: note = X can be N, Q, S or T
QVQLQXSGAEVKKPGSSVKVSCKASGGTFSSYAISWVRQAPGQGLEWMGGFDPEDGETIYAQKFQGRVTMTEDTSTDTAYMELSXLRSEDTAVYYCATDLRIPYYYDNPWGQGTXVTVSS

SEQ ID NO: 210 - protein, 30 aa, synthetic construct
  VARIANT 6: note = X can be A, V, I, L, M, F, Y or W
QVQLQXWGAGLLKPSETLSLTCAVYGGSFS

SEQ ID NO: 211 - protein, 30 aa, synthetic construct
  VARIANT 6: note = X can be A, V, I, L, M, F, Y or W
QVQLQXSGAEVKKPGSSVKVSCKASGGTFS

SEQ ID NO: 212 - protein, 32 aa, synthetic construct
  VARIANT 19: note = X can be N, Q, S or T
RATISVDTSKNQFSLKLSXVTAADTAVYYCAT

SEQ ID NO: 213 - protein, 32 aa, synthetic construct
  VARIANT 19: note = X can be N, Q, S or T
RVTMTEDTSTDTAYMELSXLRSEDTAVYYCAT

SEQ ID NO: 214 - protein, 11 aa, synthetic construct
  VARIANT 6: note = X can be Q, S, T or N
WGQGTXVTVSS

SEQ ID NO: 215 - protein, 120 aa, synthetic construct
QVQLQAWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTLVTVSS

SEQ ID NO: 216 - protein, 120 aa, synthetic construct
QVQLQQWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTQVTVSS

SEQ ID NO: 217 - protein, 120 aa, synthetic construct
  VARIANT 6: note = X can be A, V, I, L, M, F, Y or W
QVQLQXWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTLVTVSS

SEQ ID NO: 218 - protein, 119 aa, synthetic construct
  VARIANT 115: note = X can be Q, S, T or N
QVQLQQWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTXTVSS

SEQ ID NO: 219 - protein, 330 aa, Homo sapiens [sequence omitted]

SEQ ID NO: 220 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 221 - protein, 12 aa, Homo sapiens
EPKSCDKTHTCP

SEQ ID NO: 222 - protein, 113 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 223 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 107 aa, Homo sapiens [sequence omitted]

SEQ ID NO: 225 - protein, 220 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 107 aa, synthetic construct; identical to SEQ ID NO: 112

SEQ ID NO: 227 - protein, 107 aa, synthetic construct; identical to SEQ ID NO: 114

SEQ ID NO: 228 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 230 - protein, 113 aa, synthetic construct; identical to SEQ ID NO: 111

SEQ ID NO: 231 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 232 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 233 - protein, 481 aa, synthetic construct
QVQLQAWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTQVTVSSGGGGSGGGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIKNSGAGTAAATHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 234 - protein, 454 aa, synthetic construct
QVQLQQSGAEVKKPGSSVKVSCKASGGTFSSYAISWVRQAPGQGLEWMGGFDPEDGETIYAQKFQGRVTMTEDTSTDTAYMELSSLRSEDTAVYYCATDLRIPYYYDNPWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 235 - protein, 450 aa, synthetic construct
QVQLQQWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 236 - protein, 219 aa, synthetic construct
DVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 450 aa, synthetic construct
QVQLQQSGAEVKKPGSSVKVSCKASGGTFSSYAISWVRQAPGQGLEWMGGFDPEDGETIYAQKFQGRVTMTEDTSTDTAYMELSSLRSEDTAVYYCATDLRIPYYYDNPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 238 - protein, 219 aa, synthetic construct
EIVLTQSPLSLPVTPGEPASISCRSSQSLLHSNGYNYLNWYLQKPGQSPQLLIYLGSDRASGVPDRFSGSGSGTDFTLKISRVEAEDVGVYYCMQALQTPTTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 239 - protein, 20 aa, synthetic construct
GGGGSGGGGSGGGGSGGGGS

SEQ ID NO: 240 - protein, 5 aa, synthetic construct
GGGGS

SEQ ID NO: 241 - protein, 22 aa, Homo sapiens
MLKPSLPFTSLLFLQLPLLGVG

SEQ ID NO: 242 - protein, 247 aa, synthetic construct
QVQLQAWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTQVTVSSGGGGSGGGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIK

SEQ ID NO: 243 - protein, 736 aa, synthetic construct
QVQLQAWGAGLLKPSETLSLTCAVYGGSFSGYYWSWIRQPPGKGLEWIGEINHSGSTNYNPSLKSRATISVDTSKNQFSLKLSSVTAADTAVYYCATSPGGYSGGYFQHWGQGTQVTVSSGGGGSGGGGSGGGGSDVVMTQSPLSLPVTPGEPASISCRSSQSLLHSNGYNYLDWYLQKPGQSPQLLIYLGSNRDSGVPDRFSGSGSGTDFTLKISRVEAEDVGVYYCMQGTHWPWTFGQGTKVEIKNSGAGTAAAEVQLVQSGAEVKKPGASVKVSCKASGYTFTNYYMHWVRQAPGQGLEWMGAIMPSRGGTSYPQKFQGRVTITADKSTSTAYMELSSLRSEDTAVYYCARGEYYYDSSGYYYWGQGTLVTVSSGGGGSGGGGSGGGGSQSVLTQPPSVSGAPGQRVTISCTGTSSDIGHYDFVSWYQQLPGTAPKLLIYDINNRPSGVPDRFSGSKSGTSASLAITGLQAEDEADYYCSAYTSSDTLVFGGGTKLTVLNSGAGTAAATHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 736 aa, synthetic construct
QVQLQQSGAEVKKPGSSVKVSCKASGGTFSSYAISWVRQAPGQGLEWMGGFDPEDGETIYAQKFQGRVTMTEDTSTDTAYMELSSLRSEDTAVYYCATDLRIPYYYDNPWGQGTLVTVSSGGGGSGGGGSGGGGSEIVLTQSPLSLPVTPGEPASISCRSSQSLLHSNGYNYLNWYLQKPGQSPQLLIYLGSDRASGVPDRFSGSGSGTDFTLKISRVEAEDVGVYYCMQALQTPTTFGGGTKVEIKNSGAGTAAAEVQLVQSGAEVKKPGASVKVSCKASGYTFTNYYMHWVRQAPGQGLEWMGAIMPSRGGTSYPQKFQGRVTITADKSTSTAYMELSSLRSEDTAVYYCARGEYYYDSSGYYYWGQGTLVTVSSGGGGSGGGGSGGGGSQSVLTQPPSVSGAPGQRVTISCTGTSSDIGHYDFVSWYQQLPGTAPKLLIYDINNRPSGVPDRFSGSKSGTSASLAITGLQAEDEADYYCSAYTSSDTLVFGGGTKLTVLNSGAGTAAATHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 245 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 246 - protein, 219 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 247 - protein, 19 aa, Homo sapiens
VAVAGCVFLLISVLLLSGL

SEQ ID NO: 248 - protein, 13 aa, Homo sapiens
TWQRRQRKSRRTI

SEQ ID NO: 249 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 250 - protein, 25 aa, Homo sapiens
IPWLGHLLVGLSGAFGFIILVYLLI

SEQ ID NO: 251 - protein, 286 aa, Homo sapiens
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 252 - protein, 21 aa, Homo sapiens
MDSYLLMWGLLTFIMVPGCQA

SEQ ID NO: 253 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 254 - protein, 30 aa, Homo sapiens
MAPRRARGCRTLGLPALLLLLLLRPPATRG

SEQ ID NO: 255 - protein, 237 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 256 - protein, 21 aa, Homo sapiens
VVISVGSMGLIISLLCVYFWL

SEQ ID NO: 257 - protein, 86 aa, Homo sapiens
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 258 - protein, 175 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 259 - protein, 23 aa, Homo sapiens
VAISTSTVLLCGLSAVSLLACYL

SEQ ID NO: 260 - protein, 39 aa, Homo sapiens
KSRQTPPLASVEMEAMEALPVTWGTSSRDEDLENCSHHL

SEQ ID NO: 261 - protein, 825 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 262 - protein, 25 aa, Homo sapiens
MGWLCSGLLFPVSCLVLLQVASSGN

SEQ ID NO: 263 - protein, 800 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 264 - protein, 207 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 265 - protein, 24 aa, Homo sapiens
LLLGVSVSCIVILAVCLLCYVSIT

SEQ ID NO: 266 - protein, 569 aa, Homo sapiens
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 267 - protein, 521 aa, Homo sapiens
MGLGRCIWEGWTLESEALRRDMGTWLLACICICTCVCLGVSVTGEGQGPRSRTFTCLTNNILRIDCHWSAPELGQGSSPWLLFTSNQAPGGTHKCILRGSECTVVLPPEAVLVPSDNFTITFHHCMSGREQVSLVDPEYLPRRHVKLDPPSDLQSNISSGHCILTWSISPALEPMTTLLSYELAFKKQEEAWEQAQHRDHIVGVTWLILEAFELDPGFIHEARLRVQMATLEDDVVEEERYTGQWSEWSQPVCFQAPQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 268 - protein, 40 aa, Homo sapiens
MGLGRCIWEGWTLESEALRRDMGTWLLACICICTCVCLGV

SEQ ID NO: 269 - protein, 481 aa, Homo sapiens
SVTGEGQGPRSRTFTCLTNNILRIDCHWSAPELGQGSSPWLLFTSNQAPGGTHKCILRGSECTVVLPPEAVLVPSDNFTITFHHCMSGREQVSLVDPEYLPRRHVKLDPPSDLQSNISSGHCILTWSISPALEPMTTLLSYELAFKKQEEAWEQAQHRDHIVGVTWLILEAFELDPGFIHEARLRVQMATLEDDVVEEERYTGQWSEWSQPVCFQAPQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 270 - protein, 230 aa, Homo sapiens
SVTGEGQGPRSRTFTCLTNNILRIDCHWSAPELGQGSSPWLLFTSNQAPGGTHKCILRGSECTVVLPPEAVLVPSDNFTITFHHCMSGREQVSLVDPEYLPRRHVKLDPPSDLQSNISSGHCILTWSISPALEPMTTLLSYELAFKKQEEAWEQAQHRDHIVGVTWLILEAFELDPGFIHEARLRVQMATLEDDVVEEERYTGQWSEWSQPVCFQAPQRQGPLIPPWGWP

SEQ ID NO: 271 - protein, 21 aa, Homo sapiens
GNTLVAVSIFLLLTGPTYLLF

SEQ ID NO: 272 - protein, 230 aa, Homo sapiens
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 273 - protein, 538 aa, Homo sapiens
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 274 - protein, 213 aa, Homo sapiens
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 275 - protein, 21 aa, Homo sapiens
GWNPHLLLLLLLVIVFIPAFW

SEQ ID NO: 276 - protein, 285 aa, Homo sapiens
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 277 - protein, 459 aa, Homo sapiens
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 278 - protein, 19 aa, Homo sapiens
MPRGWAAPLLLLLLQGGWG

SEQ ID NO: 279 - protein, 519 aa, Homo sapiens
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 280 - protein, 20 aa, Homo sapiens
MTILGTTFGMVFSLLQVVSG

SEQ ID NO: 281 - protein, 219 aa, Homo sapiens
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMD

SEQ ID NO: 282 - protein, 25 aa, Homo sapiens
PILLTISILSFFSVALLVILACVLW

SEQ ID NO: 283 - protein, 195 aa, Homo sapiens
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 284 - protein, 439 aa, Homo sapiens
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 285 - protein, 98 aa, Homo sapiens
APENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKE

SEQ ID NO: 286 - protein, 5 aa, Homo sapiens
WSEWS

SEQ ID NO: 287 - protein, 30 aa, synthetic construct
EVQLVQSGAEVKKPGASVKVSCKASGYTFT

SEQ ID NO: 288 - protein, 30 aa, synthetic construct
EVQLVQSGGGVVQPGRSLRLSCAASGFTFS

SEQ ID NO: 289 - protein, 30 aa, synthetic construct
QVQLQQWGAGLLKPSETLSLTCAVYGGSFS

SEQ ID NO: 290 - protein, 30 aa, synthetic construct
QVQLQQWGAGMLKPSETLSLTCAVYGGSFS

SEQ ID NO: 291 - protein, 30 aa, synthetic construct
EVQLVESGGGLVQPGGSLRLSCAASGGSFS

SEQ ID NO: 292 - protein, 30 aa, synthetic construct
QVQLQESGPGLVKPSETLSLTCTVSGGSIS

SEQ ID NO: 293 - protein, 30 aa, synthetic construct
QVQLQQSGAEVKKPGSSVKVSCKASGGTFS

SEQ ID NO: 294 - protein, 30 aa, synthetic construct
QVQLVQSGGGVVQPGRSLRLSCAASGFTFS

SEQ ID NO: 295 - protein, 30 aa, synthetic construct
EVQLVETGPGLVKPSGTLSLTCAVSGGSIS

SEQ ID NO: 296 - protein, 30 aa, synthetic construct
QVQLQESGGGVVQPGRSLRLSCAASGFTFS

SEQ ID NO: 297 - protein, 30 aa, synthetic construct
QLQLQESGGGVVQPGRSLRLSCAASGFTFS

SEQ ID NO: 298 - protein, 30 aa, synthetic construct
QVQLQQWGAGLLKPSETLSLTCAVYGESFS

SEQ ID NO: 299 - protein, 30 aa, synthetic construct
QVQLQESGPGLVKPSQTLSLTCTVSGGSIS

SEQ ID NO: 300 - protein, 30 aa, synthetic construct
QVQLQQWGAGLLKPSETLSLTCTIYGGSFS

SEQ ID NO: 301 - protein, 30 aa, synthetic construct
QVQLQQWGAGLLKPSETLSLTCAVYGGSLS

SEQ ID NO: 302 - protein, 14 aa, synthetic construct
WVRQAPGQGLEWMG

SEQ ID NO: 303 - protein, 14 aa, synthetic construct
WVRQAPGKGLEWVA

SEQ ID NO: 304 - protein, 14 aa, synthetic construct
WVRQPPGKGLEWIG

SEQ ID NO: 305 - protein, 14 aa, synthetic construct
WVRQAPGKGLEWVS

SEQ ID NO: 306 - protein, 14 aa, synthetic construct
WIRQPPGKGLEWIG

SEQ ID NO: 307 - protein, 14 aa, synthetic construct
WIRQHPGQGLEWIG

SEQ ID NO: 308 - protein, 32 aa, synthetic construct
RFTISRDNSKNTLYLQMNSLRAEDTAVYYCAR

SEQ ID NO: 309 - protein, 32 aa, synthetic construct
RVTISVDTSKNQFSLKLSSVTAADTAVYYCAR

SEQ ID NO: 310 - protein, 32 aa, synthetic construct
RATISVDTSKNQFSLKLSSVTAADTAVYYCAT

SEQ ID NO: 311 - protein, 32 aa, synthetic construct
RVTMTEDTSTDTAYMELSSLRSEDTAVYYCAT

SEQ ID NO: 312 - protein, 32 aa, synthetic construct
RVTISVDKSKNQFSLKLSSVTAADTAVYYCAR

SEQ ID NO: 313 - protein, 32 aa, synthetic construct
RFTISRDNSKNTVYLQMNSLRAEDTAVYYCAK

SEQ ID NO: 314 - protein, 32 aa, synthetic construct
RFTISRDNSKNTLYLQMNSLRAEDTAVYYCAK

SEQ ID NO: 315 - protein, 32 aa, synthetic construct
RVTISADTSKNHFSLNLTSVTAADTAVYYCAR

SEQ ID NO: 316 - protein, 32 aa, synthetic construct
RVTMTRDTSTSTVYMELSSLRSEDTAVYYCAR

SEQ ID NO: 317 - protein, 32 aa, synthetic construct
RVTISVDTSKNQFSLKLSSVTAADTAIYYCAR

SEQ ID NO: 318 - protein, 32 aa, synthetic construct
RVTISEDASKKQFSLTLTSVTAADTAVYYCAR

SEQ ID NO: 319 - protein, 33 aa, synthetic construct
SRVTISVDTSKNQFSLKLSSVTAADTAVYYCAG

SEQ ID NO: 320 - protein, 11 aa, synthetic construct
WGQGTLVTVSS

SEQ ID NO: 321 - protein, 11 aa, synthetic construct
WGQGTMVTVSS

SEQ ID NO: 322 - protein, 11 aa, synthetic construct
WGKGTTVTVSS

SEQ ID NO: 323 - protein, 23 aa, synthetic construct
DVVMTQSPLSLPVTPGEPASISC

SEQ ID NO: 324 - protein, 23 aa, synthetic construct
DIQMTQSPSSLSASVGDRVTITC

SEQ ID NO: 325 - protein, 23 aa, synthetic construct
EIVLTQSPLSLPVTPGEPASISC

SEQ ID NO: 326 - protein, 23 aa, synthetic construct
DVVMTQSPLSLPVTPGESVSISC

SEQ ID NO: 327 - protein, 22 aa, synthetic construct
SYELTQPPSMSVSPGQTARITC

SEQ ID NO: 328 - protein, 23 aa, synthetic construct
EIVLTQSPATLSLSPGERATLSC

SEQ ID NO: 329 - protein, 23 aa, synthetic construct
DIQMTQSPSFLSASVGDRVTITC

SEQ ID NO: 330 - protein, 23 aa, synthetic construct
DVVMTQSPVSLPVTLGQPASISC

SEQ ID NO: 331 - protein, 23 aa, synthetic construct
DVVMTQSPLSLPVTLGQPASISC

SEQ ID NO: 332 - protein, 23 aa, synthetic construct
ETTLTQSPATLSVSPGERATLSC

SEQ ID NO: 333 - protein, 23 aa, synthetic construct
DIVMTHTPLSLPVTPGEPASISC

SEQ ID NO: 334 - protein, 23 aa, synthetic construct
EIVMTQSPLSLPVTPGEPASISC

SEQ ID NO: 335 - protein, 15 aa, synthetic construct
WYLQKPGQSPQLLIY

SEQ ID NO: 336 - protein, 15 aa, synthetic construct
WYQQKPGQAPVLVIY

SEQ ID NO: 337 - protein, 15 aa, synthetic construct
WYLQKPGQSPQLLMY

SEQ ID NO: 338 - protein, 15 aa, synthetic construct
WFQQRPGQSPRRLFY

SEQ ID NO: 339 - protein, 15 aa, synthetic construct
WYQQKPGQAPRLLIY

SEQ ID NO: 340 - protein, 15 aa, synthetic construct
WYLQKPGQSPHLLIY

SEQ ID NO: 341 - protein, 32 aa, synthetic construct
GVPDRFSGSGSGTDFTLKISRVEAEDVGVYYC

SEQ ID NO: 342 - protein, 32 aa, synthetic construct
GVPSRFSGSGSGTDFTLTISSLQPEDFATYYC

SEQ ID NO: 343 - protein, 32 aa, synthetic construct
GIPERFSGSSSGTTVTLTITGVQAEDEADYYC

SEQ ID NO: 344 - protein, 32 aa, synthetic construct
GVPERFSGSGSGTDFTLKISRVEAEDVGVYYC

SEQ ID NO: 345 - protein, 32 aa, synthetic construct
GIPARFSGSGSGTDFTLTINSLEPEDFAVYYC

SEQ ID NO: 346 - protein, 32 aa, synthetic construct
GVPSRFSGTGFGTDFTFTITTLQPDDIATYYC

SEQ ID NO: 347 - protein, 32 aa, synthetic construct
GVPDRFSGSGSDTDFTLTISRVEAEDVGVYFC

SEQ ID NO: 348 - protein, 32 aa, synthetic construct
GVPDRFSGSGSGTDFTLKISRVEAEDVGVYFC

SEQ ID NO: 349 - protein, 32 aa, synthetic construct
GIPDRFSGSGSGTDFTLTISRLEPEDFAVYYC

SEQ ID NO: 350 - protein, 32 aa, synthetic construct
GVPDRFSGSGSGTDFTLKISRVEAEDVGIYYC

SEQ ID NO: 351 - protein, 10 aa, synthetic construct
FGGGTKVEIK

SEQ ID NO: 352 - protein, 10 aa, synthetic construct
FGGGTKLTVL

SEQ ID NO: 353 - protein, 10 aa, synthetic construct
FGQGTKLEIK

SEQ ID NO: 354 - protein, 10 aa, synthetic construct
FGQGTKVEIK

SEQ ID NO: 355 - protein, 10 aa, synthetic construct
FGQGTTVDIK

SEQ ID NO: 356 - protein, 10 aa, synthetic construct
FGQGTRLEFK

SEQ ID NO: 357 - protein, 10 aa, synthetic construct
FGPGTKVDIK

SEQ ID NO: 358 - protein, 10 aa, synthetic construct
FGQGTKVEIE

SEQ ID NO: 359 - protein, 15 aa, synthetic construct
WYQQKPGKAPKLLIY

SEQ ID NO: 360 - protein, 27 aa, synthetic construct
AEAAAKEAAAKEAAAKEAAAKEAAAKA

SEQ ID NO: 361 - protein, 9 aa, synthetic construct
GGGGSGGGS

SEQ ID NO: 362 - protein, 13 aa, synthetic construct
GGGGSGGGSGGGS

SEQ ID NO: 363 - protein, 5 aa, synthetic construct
GGGSG

SEQ ID NO: 364 - protein, 5 aa, synthetic construct
EAAAK

SEQ ID NO: 365 - protein, 12 aa, synthetic construct
AEAAAKEAAAKA

SEQ ID NO: 366 - protein, 17 aa, synthetic construct
AEAAAKEAAAKEAAAKA

SEQ ID NO: 367 - protein, 22 aa, synthetic construct
AEAAAKEAAAKEAAAKEAAAKA

SEQ ID NO: 368 - protein, 19 aa, synthetic construct
AAAYPYDVPDYGSHHHHHH

SEQ ID NO: 369 - protein, 6 aa, synthetic construct
NSGAAA

SEQ ID NO: 370 - protein, 8 aa, synthetic construct
GGPSGIST

SEQ ID NO: 371 - protein, 8 aa, synthetic construct
INWSGEST

SEQ ID NO: 372 - protein, 24 aa, synthetic construct
TTTRDAGTRLGSRWYGPRTSAYEN

SEQ ID NO: 373 - protein, 25 aa, synthetic construct
QVQLQESGGGLVQAGGSLRLSCAAS

SEQ ID NO: 374 - protein, 17 aa, synthetic construct
VAWFRQAPGKEREFVAA

SEQ ID NO: 375 - protein, 38 aa, synthetic construct
NYEDSVLGRFTISRDNAKNTVFLQMNSLTPEDTAVYVC

SEQ ID NO: 376 - protein, 11 aa, synthetic construct
WGQGTQVTVSS

SEQ ID NO: 377 - protein, 131 aa, synthetic construct
QVQLQESGGGLVQAGGSLRLSCAASGGPSGISTVAWFRQAPGKEREFVAAINWSGESTNYEDSVLGRFTISRDNAKNTVFLQMNSLTPEDTAVYVCTTTRDAGTRLGSRWYGPRTSAYENWGQGTQVTVSS

SEQ ID NO: 378 - protein, 8 aa, synthetic construct
GSFFNINA

SEQ ID NO: 379 - protein, 7 aa, synthetic construct
ITAGGST

SEQ ID NO: 380 - protein, 11 aa, synthetic construct
AAWGTRIRDDY

SEQ ID NO: 381 - protein, 25 aa, synthetic construct
QVQLQESGGGLVQAGGSLRLSCTVS

SEQ ID NO: 382 - protein, 17 aa, synthetic construct
MGWYRQAPGKQRELVAT

SEQ ID NO: 383 - protein, 38 aa, synthetic construct
KYEDTVKGRFTISGDSAKNTVSLQMNSLKPEDTAVYYC

SEQ ID NO: 384 - protein, 11 aa, synthetic construct
WGQGTQVTVSS

SEQ ID NO: 385 - protein, 117 aa, synthetic construct
QVQLQESGGGLVQAGGSLRLSCTVSGSFFNINAMGWYRQAPGKQRELVATITAGGSTKYEDTVKGRFTISGDSAKNTVSLQMNSLKPEDTAVYYCAAWGTRIRDDYWGQGTQVTVSS

SEQ ID NO: 386 - protein, 8 aa, synthetic construct
GSIFSINA

SEQ ID NO: 387 - protein, 7 aa, synthetic construct
ISSGGST

SEQ ID NO: 388 - protein, 11 aa, synthetic construct
LVATRGGTYEY

SEQ ID NO: 389 - protein, 25 aa, synthetic construct
QVQLQESGGGLVQAGGSLRLSCAAS

SEQ ID NO: 390 - protein, 17 aa, synthetic construct
MGWFRQAPGKQRELVAV

SEQ ID NO: 391 - protein, 38 aa, synthetic construct
NHADSVKGRFTISRDNAKNTVYLQMNSVKPEDTAVYYC

SEQ ID NO: 392 - protein, 11 aa, synthetic construct
WGQGTQVTVSS

SEQ ID NO: 393 - protein, 117 aa, synthetic construct
QVQLQESGGGLVQAGGSLRLSCAASGSIFSINAMGWFRQAPGKQRELVAVISSGGSTNHADSVKGRFTISRDNAKNTVYLQMNSVKPEDTAVYYCLVATRGGTYEYWGQGTQVTVSS

SEQ ID NO: 394 - protein, 253 aa, synthetic construct
QVQLQESGGGLVQAGGSLRLSCAASGGPSGISTVAWFRQAPGKEREFVAAINWSGESTNYEDSVLGRFTISRDNAKNTVFLQMNSLTPEDTAVYVCTTTRDAGTRLGSRWYGPRTSAYENWGQGTQVTVSSGGGGSQVQLQESGGGLVQAGGSLRLSCAASGSIFSINAMGWFRQAPGKQRELVAVISSGGSTNHADSVKGRFTISRDNAKNTVYLQMNSVKPEDTAVYYCLVATRGGTYEYWGQGTQVTVSS

SEQ ID NO: 395 - protein, 253 aa, synthetic construct
QVQLQESGGGLVQAGGSLRLSCAASGSIFSINAMGWFRQAPGKQRELVAVISSGGSTNHADSVKGRFTISRDNAKNTVYLQMNSVKPEDTAVYYCLVATRGGTYEYWGQGTQVTVSSGGGGSQVQLQESGGGLVQAGGSLRLSCAASGGPSGISTVAWFRQAPGKEREFVAAINWSGESTNYEDSVLGRFTISRDNAKNTVFLQMNSLTPEDTAVYVCTTTRDAGTRLGSRWYGPRTSAYENWGQGTQVTVSS

SEQ ID NO: 396 - protein, 275 aa, synthetic construct
QVQLQESGGGLVQAGGSLRLSCAASGGPSGISTVAWFRQAPGKEREFVAAINWSGESTNYEDSVLGRFTISRDNAKNTVFLQMNSLTPEDTAVYVCTTTRDAGTRLGSRWYGPRTSAYENWGQGTQVTVSSAEAAAKEAAAKEAAAKEAAAKEAAAKAQVQLQESGGGLVQAGGSLRLSCAASGSIFSINAMGWFRQAPGKQRELVAVISSGGSTNHADSVKGRFTISRDNAKNTVYLQMNSVKPEDTAVYYCLVATRGGTYEYWGQGTQVTVSS

SEQ ID NO: 397-436 - skipped (40 entries)

SEQ ID NO: 437 - protein, 369 aa, Homo sapiens; identical to SEQ ID NO: 194

SEQ ID NO: 438 - protein, 347 aa, Homo sapiens; identical to SEQ ID NO: 195

SEQ ID NO: 439 - protein, 240 aa, Homo sapiens; identical to SEQ ID NO: 196

SEQ ID NO: 440-502 - skipped (63 entries)

SEQ ID NO: 503 - protein, 22 aa, Homo sapiens
MLKPSLPFTSLLFLQLPLLGVG

SEQ ID NO: 504-517 - skipped (14 entries)

SEQ ID NO: 518 - protein, 21 aa, Homo sapiens
VVISVGSMGLIISLLCVYFWL

SEQ ID NO: 519 - protein, 86 aa, Homo sapiens; identical to SEQ ID NO: 257

SEQ ID NO: 520-546 - skipped (27 entries)

SEQ ID NO: 547 - protein, 98 aa, Homo sapiens; identical to SEQ ID NO: 285

SEQ ID NO: 548 - protein, 5 aa, Homo sapiens
WSEWS

SEQ ID NO: 549 - protein, 5 aa, synthetic construct
  VARIANT 3: note = X can be any amino acid
WSXWS

SEQ ID NO: 550 - protein, 16 aa, synthetic construct
GGGSGGGSGGGSGGGS

SEQ ID NO: 551 - protein, 4 aa, synthetic construct
  VARIANT >4: note = The entire sequence of amino acids 1-4 can be repeated two to six times
GGGS

SEQ ID NO: 552 - protein, 5 aa, synthetic construct
  VARIANT >5: note = The entire sequence of amino acids 1-5 can be repeated two to six times
GGGGS

SEQ ID NO: 553 - protein, 5 aa, synthetic construct
  VARIANT 1..4: note = The residues 1-4 can be repeated two to six times
GGGSG

SEQ ID NO: 554 - protein, 6 aa, synthetic construct
  VARIANT 1..4: note = The residues 1-4 can be repeated two to six times
GGGSGG

SEQ ID NO: 555 - protein, 7 aa, synthetic construct
  VARIANT 1..4: note = The residues 1-4 can be repeated two to six times
GGGSGGG

SEQ ID NO: 556 - protein, 6 aa, synthetic construct
  VARIANT 1..5: note = The residues 1-5 can be repeated two to six times
GGGGSG

SEQ ID NO: 557 - protein, 7 aa, synthetic construct
  VARIANT 1..5: note = The residues 1-5 can be repeated two to six times
GGGGSGG

SEQ ID NO: 558 - protein, 8 aa, synthetic construct
  VARIANT 1..5: note = The residues 1-5 can be repeated two to six times
GGGGSGGG

SEQ ID NO: 559 - protein, 5 aa, synthetic construct
  VARIANT >5: note = The entire sequence of amino acids 1-5 can be repeated one or more times
GGGGS

SEQ ID NO: 560 - protein, 30 aa, synthetic construct
GGGGSGGGGSGGGGSGGGGSGGGGSGGGGS

SEQ ID NO: 561 - protein, 6 aa, synthetic construct
HHHHHH